(19) United States
(12) Patent Application Publication
(10) Pub. No.: US 2007/0192883 A1
(43) Pub. Date: Aug. 16, 2007

(54) TAT-028 and methods of assessing and treating cancer

(76) Inventors: Daniel Chelsky, Westmount (CA); Paul E. Kearney, Montreal (CA); Eustache Paramithiotis, Boucherville (CA); Lyes Hamaidi, Ville Saint-Laurent (CA); Leslie H. Kondejewski, Saint Lazare (CA); Joel Lanoix, Montreal (CA); Patrice Hugo, Montreal (CA)

Correspondence Address: CLARK & ELBING LLP, 101 FEDERAL STREET, BOSTON, MA, 02110, US

(21) Appl. No.: 11/655,858

(22) Filed: Jan. 19, 2007

Related U.S. Application Data
(60) Provisional application No. 60/761,002, filed on Jan. 19, 2006.

(51) Int. Cl. A01K 67/027; C12Q 1/68; G01N 33/574; A61K 39/395; A61K 48/00; A61K 39/00; C07K 14/82; C07K 16/30

(52) U.S. Cl. 800/14; 435/69.1; 435/320.1; 435/325; 424/144.1; 530/350; 530/388.1; 514/44; 435/6; 435/7.23

(57) ABSTRACT

Surprisingly, the present inventors have discovered that expression of TAT-028 protein in human patients is associated with cancer, and that the overexpressed protein is present in plasma membrane fractions. Thus, the present inventors have discovered that TAT-028 is associated with abnormal development and growth, and may be useful as a target for the identification of anti-cancer compounds, including antibodies for use in immunotherapy. Accordingly, the present invention provides methods for the identification of compounds that inhibit TAT-028 expression or activity, comprising: contacting a candidate compound with a TAT-028 and detecting the presence or absence of binding between said compound and said TAT-028, or detecting a change in TAT-028 expression or activity. Methods are also included for the identification of compounds that modulate TAT-028 expression or activity, comprising: administering a compound to a cell or cell population, and detecting a change in TAT-028 expression or activity. The methods of the invention are useful for the identification of anti-cancer compounds.

### CROSS-REFERENCE TO RELATED APPLICATIONS

This application claims benefit of U.S. Provisional Application No. 60/761,002, filed Jan. 19, 2006, which is hereby incorporated by reference.

### FIELD OF THE INVENTION

The present inventors have discovered that increased expression of TAT-028 protein in human patients is associated with lung tumors as compared to adjacent normal tissue. Thus, the present inventors have discovered that TAT-028 is associated with abnormal development and growth, and can be used as a target for the identification of potential anti-cancer compounds, including antibodies for use in immunotherapy.

### BACKGROUND

In 2000, worldwide, there were more than 10 million cases of cancer identified, and over 6 million cancer-related deaths. 23% of all deaths in the United States in 2000 were cancer-related. Lung cancer makes up a significant proportion of that statistic, as lung cancer is the most common cancer, with 900,000 new cases each year in men and 330,000 in women, and is the most commonly fatal cancer in the United States, accounting for 13% of cancer diagnoses and 29% of all cancer deaths. In fact, lung cancer deaths in the US are greater than the combined deaths attributed to lung, breast and prostate cancers, despite being only the third most common cancer behind breast and prostate. Currently, about 13.5% of Americans will have lung cancer at some point in their life (1 in 13 men, 1 in 17 women). Hospital time is still significant for non-fatal cases.

Treatment for lung cancer remains unsatisfactory in terms of mortality, recurrence after treatment, and invasiveness. Surgery is the most common treatment for some forms of lung cancer. 50% of those having a Stage I non-small cell carcinoma removed without resorting to a lobectomy have been shown to develop a recurrence. 50% of all lung cancers are not resectable at time of diagnosis. An additional 25% are not completely resectable intraoperatively. The five-year survival rate for lung cancer is only 15.2% and the overall mortality rate for those diagnosed is 86%. Patients and their physicians choosing non-surgical treatments as follow-up, in place of, or in conjunction with, surgery must also weigh the benefits of therapy versus the side effects of the treatment: even successful current treatments, although benefiting the patient overall, can have a profound negative impact on a survivor's health and quality of life.

Some tumors also become refractory to treatments leading to recurrent or metastatic disease, which is often incurable. Indeed, cancers can have diverse etiologies with resultant differing patterns of protein expression, which can dictate response to treatment. The identification of common suitable targets or antigens for therapy of lung cancer has become increasingly important—both as initial therapies and as therapies for cancers that have become refractory to other treatments.

The diagnosis of lung cancer itself remains problematic. When diagnosed early at a localized stage, 5 year survivability is 49.4%, yet only 15% of lung cancers are diagnosed while still localized. New predictive non-invasive markers are needed. Current blood-based biomarkers that can be used in the diagnosis and monitoring of disease, such as the carcinoembryonic antigen (CEA), are not fully reliable. The identification of new proteins overexpressed in lung cancer might provide further opportunities for such diagnostics, as well as screening methods to determine the most appropriate treatment.

Thus, both the diagnosis and treatment of lung cancer remains problematic, and there is a need in the art for improved methods of detecting and treating lung cancers. Immunotherapy and the use of tumor-related antigens for diagnostics and treatment have previously provided new approaches, but there remains a scarcity of credible antigen targets suitable for treating lung cancer.

To date there do not appear to be any published demonstrations of overexpression of the TAT-028 protein on the plasma membrane of lung cancer tumor tissue. The prior art does not show a cancer-associated alteration of TAT-028 protein expression at the plasma membrane, nor does it show the potential usefulness of TAT-028 in an immunotherapeutic approach to cancer.

### BRIEF SUMMARY OF THE INVENTION

The inventors have identified TAT-028 protein from a peptide unique to its sequence (peptide #1) using highly accurate mass spectrometric and bioinformatic methods on highly enriched and pure plasma membrane samples derived from viable epithelial cells of fresh human lung cancer tumor tissue and matched adjacent normal tissue. The inventors have discovered that Tumor Antigen Target-028 (TAT-028) is frequently overexpressed at the cell surface in lung cancers as compared to adjacent normal tissue. These results robustly indicate the viability of TAT-028 protein as a potential target for immunotherapy based on its localization to the plasma membrane and its reproducibly elevated expression level in lung cancer tissue relative to normal tissue in a percentage of patients exceeding that of other current cancer immunotherapies. The present invention relates to compositions of and methods of use for the TAT-028 protein and its encoding nucleic acids. The invention also features methods for identifying TAT-028 interactors and modulators for use as diagnostic tools or therapeutic tools for identifying and targeting of cancer cells, and for regulating TAT-028 function, such as in the treatment of disease. The invention further relates to methods and compositions useful in the prophylaxis, diagnosis, treatment and management of various cancers that express TAT-028, in particular lung cancer. Such methods include the production, compositions, and uses of antibodies, vaccines, antigen presenting cells that express TAT-028, T cells specific for cells expressing TAT-028, and immunotherapy.

Accordingly, the present invention provides a substantially pure TAT-028 polypeptide or a fragment thereof and nucleic acid sequences useful in carrying out the methods of the invention. Substantially pure or isolated polypeptides of the invention (TAT-028 polypeptides): a) comprise or consist of the amino acid sequence of SEQ ID NO: 1; b) comprise or consist of the amino acid sequence of SEQ ID NO: 3; c) comprise or consist of the amino acid sequence of SEQ ID NO: 5; d) comprise or consist of the amino acid sequence of SEQ ID NO: 7; e) comprise or consist of the amino acid sequence of SEQ ID NO: 9; f) comprise or consist of the amino acid sequence of SEQ ID NO: 11; g) comprise or consist of the amino acid sequence of SEQ ID NO: 13; h) comprise or consist of the amino acid sequence of SEQ ID NO: 15; i) comprise or consist of the amino acid sequence of SEQ ID NO: 17; j) comprise or consist of the amino acid sequence of SEQ ID NO: 19; k) are derivatives having one or more amino acid substitutions, modifications, deletions or insertions relative to the amino acid sequence of SEQ ID NO: 19 and have at least 60% identity, preferably 65%, 70%, 75%, 80%, 85%, 90%, 95% or more (e.g., are substantially identical), over the length of the sequence; 1) are fragments of a polypeptide having the amino acid sequence of SEQ ID NO: 19, which are at least four amino acids long and have at least 60% identity over the length of the fragment; m) comprise additional amino acid sequence for coupling to a coupling agent; n) comprise a terminal cysteine as an additional amino acid sequence for coupling to a coupling agent; or o) comprise additional amino acid sequences facilitating purification, wherein such additional sequences comprises a myc, FLAG, HIS, HA, GST, affinity or epitope tag. In desirable embodiments, the TAT-028 polypeptide is from a mammal, preferably a human.

The TAT-028 polypeptide of the invention may be in a composition suitable for inducing an immune response in a subject, which includes a substantially pure TAT-028 polypeptide or fragment thereof in a pharmaceutically acceptable carrier.

The present invention also provides substantially pure or isolated nucleic acid molecules (TAT-028 nucleic acids, such as mammalian (e.g., human) nucleic acids) that: a) comprise or consist of the DNA sequence of SEQ ID NO:20 or its RNA equivalent; b) comprise or consist of the DNA sequence of SEQ ID NO: 2 or its RNA equivalent; c) comprise or consist of the DNA sequence of SEQ ID NO: 4 or its RNA equivalent; d) comprise or consist of the DNA sequence of SEQ ID NO: 6 or its RNA equivalent; e) comprise or consist of the DNA sequence of SEQ ID NO: 8 or its RNA equivalent; f) comprise or consist of the DNA sequence of SEQ ID NO: 10 or its RNA equivalent; g) comprise or consist of the DNA sequence of SEQ ID NO: 12 or its RNA equivalent; h) comprise or consist of the DNA sequence of SEQ ID NO: 14 or its RNA equivalent; i) comprise or consist of the DNA sequence of SEQ ID NO: 16, any of SEQ ID NOS:50-58, or its RNA equivalent; j) comprise or consist of the DNA sequence of SEQ ID NO: 18 or its RNA equivalent; l) have a sequence which is complementary to the sequences of (a), (b),(c), (d), (e), (f), (g), (h), (i), (j), (k) and/or (l); m) have a sequence which codes for a polypeptide as defined in (a) to (o) of the previous paragraph; n) comprise or consist of a gDNA sequence per (m); o) comprise or consist of a promoter associated with (n); p) have a sequence which consists essentially of any of (a), (b), (c), (d), (e), (f), (g), (h), (i), (j), (k), (l), (m), (n) and (o); q) have a sequence which is substantially identical to (e.g., at least 60%, 65%, 70%, 75%, 80%, 85%, 90%, 95%, 99%, or 100% identity) to any of (a), (b), (c), (d), (e), (f), (g), (h), (i), (j), (k), (l), (m), (n), (o) and (p); r) are fragments of (a), (b), (c), (d), (e), (f), (g), (h), (i), (j), (k), (l), (m), (n), (o), (p) or (q), which are at least six (e.g., ten) nucleotides in length; s) are sequences per (a), (b), (c), (d), (e), (f), (g), (h), (i), (j), (k), (l), (m), (n), (o), (p), (q) and/or (r) which also comprise transcriptional and/or translational regulatory elements; or t) are sequences per (a), (b), (c), (d), (e), (f), (g), (h), (i), (j), (k),(l), (m), (n), (o), (p), (q), (r) and/or (s) which are part of a vector, plasmid, virus-based vector, or artificial chromosome. In some embodiments, the nucleic acid molecules hybridize under high stringency conditions to at least a portion of a TAT-028 nucleic acid. In some embodiments, the nucleic acid (e.g., an RNAi molecule) is complementary (e.g., 95% sequence identifity) to at least a portion of the TAT-028 nucleic acid (e.g., the TAT-028 coding region) and is capable of reducing the levels of a TAT-028 nucleic acid or protein molecules in a cell expressing the TAT-028 nucleic acid. The invention also provides for vectors, host cells, and non-human transgenic animals (e.g., a mouse) that contain one or more of the nucleic acids, and methods for expressing and purifying the polypeptides of the invention therefrom. The non human human transgenic animal may have a mutation in an allele encoding a TAT-028 polypeptide. The invention also features a cell from the non-transgenic human animal.

Nucleic acids of the invention also include probes having at least 60% (e.g., 70%, 80%, 90%, 95%, or 100%) nucleic acid sequence identity to a sequence encoding a TAT-028 polypeptide or a fragment thereof, where the fragment encodes at least six contiguous amino acids and the probe hybridizes under high stringency conditions to at least a portion of a TAT-028 nucleic acid molecule. The invention also features kits including such primers.

Nucleic acids of the invention may also be in a composition (e.g., suitable for inducing an immune response in a subject), which includes a nucleic acid molecule of the invention and a pharmaceutically acceptable carrier. The composition may be administered to a subject to prevent or treat a cellular proliferative disease (e.g., a cancer such as lung cancer).

The invention also features a pharmaceutical composition including a ribozyme that cleaves a TAT-028 nucleic acid molecule and a pharmaceutically acceptable carrier. The composition may be administered to a subject to prevent or treat a cellular proliferative disease (e.g., a cancer such as lung cancer).

The invention further provides pharmaceutical compositions (e.g., for inducing an immune response), which include a TAT-028 polypeptide (e.g., substantially pure or isolated) as described above and a pharmaceutically acceptable carrier. The composition may be administered to a subject to prevent or treat a cellular proliferative disease (e.g., a cancer such as lung cancer). Additional compositions for inducing an immune response are also provided, which include an isolated polypeptide of TAT-028 as described above and a non-specific immune response enhancer, e.g., an adjuvant. Further, compositions for inducing an immune response, including a nucleic acid encoding the isolated polypeptide, as described above, and a pharmaceutically acceptable carrier are provided. Compositions including a compound that binds a TAT-028 polypeptide (e.g., an antibody or TAT-028 binding fragment thereof) in a pharmaceutically acceptable carrier are also provided. The composition may be administered to a subject to prevent or treat a cellular proliferative disease (e.g., a cancer such as lung cancer).

The invention also features a method of inducing an immune response to a TAT-028 polypeptide. The method includes providing a TAT-028 polypeptide (e.g., those described above) and contacting the polypeptide with an immune system cell (e.g., at least one T cell antigen, at least one B cell antigen, or at least one antigen presenting cell antigen). The polypeptide may be accompanied by an adjuvant. The invention also features a method inducing an immune response in a subject by administering a composition including a TAT-028 polypeptide or nucleic acid to the subject.

The invention also provides for antibodies, functionally-active fragments, derivatives or analogues thereof (herein, TAT-028 antibodies), which specifically bind a TAT-028 polypeptide (e.g., polypeptides including the amino acid sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 38, SEQ ID NO: 39, SEQ ID NO: 40, SEQ ID NO: 41, or SEQ ID NO: 42), where the antibodies may be monoclonal, polyclonal, single-chain, chimeric, humanized, fully-humanized, human, bispecific, or any combination thereof. Preferred antibody fragments include a Fab fragment, a F(ab)′2 fragment, or an Fv fragment. The antibodies can also be conjugated to a therapeutic moiety, detectable label, second antibody or a fragment thereof, a cytotoxic agent, or cytokine. The invention also provides isolated cells, hybridomas, non-human transgenic animals, or plants that produce the antibodies or fragments thereof.

The invention also provides for TAT-028 antibody-related proteins and nucleic acids. These include proteins comprising or consisting of the antigen-binding region of an antibody or fragment thereof, wherein the protein may be conjugated to a therapeutic moiety, detectable label, second antibody or a fragment thereof, a cytotoxic agent or cytokine. The antibody-related proteins also include TAT-028-binding proteins that are derivatives having one or more amino acid substitutions, modifications, deletions or insertions relative to a TAT-028 antibody or fragment thereof and which retain at least 10%, preferably 20%, 30%, 40%, 50%, 60%, 70%, 80%, 90% or more, of the binding activity of the antibody, wherein TAT-028-binding protein may be conjugated to a therapeutic moiety, detectable label, second antibody or a fragment thereof, a cytotoxic agent or cytokine. The invention also features isolated nucleic acid molecules which: a) have a sequence which codes for a TAT-028 antibody or fragment thereof, a TAT-028-binding protein, or a protein comprising or consisting of the antigen-binding region of an antibody or fragment thereof; b) comprise or consist of a gDNA sequence per (a); c) have a sequence which consists essentially of any of those of (a) or (b); d) have a sequence which shows substantial identity with any of those of (a), (b), and (c); e) are a fragment of (a), (b), (c), or (d), which is at least ten nucleotides in length; f) are a sequence per (a), (b), (c), (d), and/or (e) which also comprises transcriptional and/or translational regulatory elements; or g) are a sequence per (a), (b), (c), (d), (e), and/or (f) which is part of a vector, plasmid, virus-based vector, or artificial chromosome. The invention also provides for host cells that contain one or more of the nucleic acids, and methods for expressing and purifying the polypeptides of the invention therefrom.

The invention also features a method for detecting the presence of a mutant TAT-028 polypeptide in a sample. The method includes contacting the sample with an antibody that specifically binds to a mutant TAT-028 polypeptide and assaying for binding of the antibody to the mutant polypeptide.

The invention also features a method of detecting the presence of a TAT-028 nucleic acid in a sample including contacting the sample with a probe of the invention.

Methods for selecting a TAT-028 binding molecule, such as an antibody, antibody-related protein, small molecule, or TAT-028 polypeptide are also provided. In one embodiment, the invention features a method (e.g., for selecting an antibody that binds with high binding affinity to a mammalian TAT-028) that includes the steps of: (a) providing a TAT-028 peptide or a peptide comprising a TAT-028 polypeptide, optionally coupled to an immunogenic carrier; and (b) contacting the TAT-028 polypeptide with a candidate compound (e.g., a TAT-028 binding molecule such as an antibody), under conditions that allow for complex formation between the TAT-028 polypeptide and the TAT-028 binding molecule, thereby selecting a TAT-028 binding molecule that binds (e.g., with high binding affinity) to a mammalian TAT-028.

The invention also provides for assays for detecting the presence of TAT-028 polypeptide or a TAT-028 nucleic acid in a sample (e.g., a biological sample) comprising steps of: contacting the sample with a TAT-028 binding molecule (e.g., specifically binds to a TAT-028 polypeptide or TAT-028 nucleic acid such as an antibody); and, detecting the binding of TAT-028 polypeptide or TAT-028 nucleic acid in the sample thereto. The invention additionally provides for a diagnostic kit comprising a capture reagent specific for a TAT-028 polypeptide, reagents, and instructions for use. Such methods and kits can also be used to detect a mutant TAT-028 polypeptide or nucleic acid in a sample.

The invention also features a method for diagnosing an increased likelihood of developing a TAT-028-related disease or condition (e.g., a cellular proliferative disease, for example, cancer such as lung cancer) in a test subject. The method includes analyzing nucleic acid molecules (e.g., by restriction fragment length polymorphism (RFLP) analysis) of the test subject (e.g., a mammal such as a human) to determine whether the test subject contains a mutation in a TAT-028 gene, where the presence of the mutation is an indication that the test subject has an increased likelihood of developing a TAT-028-related disease. The method may further include a step of using nucleic acid molecule primers specific for the TAT-028 gene for nucleic acid molecule amplification by the polymerase chain reaction (e.g., further including a step of sequencing TAT-028 nucleic acid molecules from the test subject).

The invention also provides for diagnostic methods including a method of screening for and/or diagnosis of a cellular proliferative disease in a subject, and/or monitoring the effectiveness of therapy, which includes the step of detecting and/or quantifying in a biological sample obtained from the subject: (i) a TAT-028 polypeptide or (ii) a TAT-028 nucleic acid molecule. The polypeptide or nucleic acid may be compared to a reference range or a control sample, preferably one that was previously determined. The step of detecting may include: a) contacting the sample with a capture reagent that is specific for a TAT-028 polypeptide and b) detecting whether binding has occurred between the capture reagent and the polypeptide in the sample. Step (b) may further comprise detecting the captured polypeptide using a directly or indirectly labeled detection reagent. The capture reagent in these methods of screening and/or diagnosis may be immobilized on a solid phase and/or the TAT-028 polypeptide may be detected and/or quantified using an antibody that recognizes a TAT-028 polypeptide. The diagnostic methods can also be used to detect a mutant TAT-028 polypeptide or nucleic acid that is associated with a cellular proliferative disease. For nucleic acids, the methods can include analyzing the sequence or the restriction fragment length (e.g., by restriction fragment length polymorphism analysis) of the nucleic acids of the test subject and comparing it to the sequence or the restriction fragment length of a TAT-028 nucleic acid molecule. Detection of a mutation can indicate that the test subject has an increased likelihood of developing a cellular proliferative disease (e.g., cancer).

The invention further provides a method of identifying a compound that binds to a TAT-028 polypeptide (e.g., useful for screening for anti-cellular proliferative disease agents that interact with a TAT-028 polypeptide). The method includes: contacting the polypeptide with a candidate agent and determining whether or not the candidate agent interacts with the polypeptide. Also provided are comparative methods for identifying a candidate compound for the treatment of cellular proliferative diseases that includes: measuring the binding of a TAT-028 binding molecule to a TAT-028 polypeptide in the presence of a test compound and measuring the binding of the TAT-028 binding molecule to a TAT-028 polypeptide in the absence of the test compound; where the level of binding of the TAT-028 binding molecule to a TAT-028 polypeptide in the presence of the test compound that is altered (e.g., increased or decreased) from the level of binding of the TAT-028 binding molecule to a TAT-028 polypeptide in the absence of the test compound is an indication that the test compound is a potential therapeutic compound for the treatment of a cellular proliferative disease.

The invention further provides a method for identifying a compound for diagnosing a cellular proliferative disease. The method includes: measuring the binding of a TAT-028 binding molecule to a TAT-028 polypeptide in the presence of a test compound and measuring the binding of the TAT-028 binding molecule to a TAT-028 polypeptide in the absence of the test compound; wherein a level of binding of the TAT-028 binding molecule to a TAT-028 polypeptide in the presence of the test compound that is altered (e.g., increased or decreased) from the level of binding of the TAT-028 binding molecule to a TAT-028 polypeptide in the absence of the test compound is an indication that the test compound is a potential compound for diagnosing a cellular proliferative disease. The determination of interaction between the candidate agent and TAT-028 polypeptide can include quantitatively or qualitatively detecting binding of the candidate agent and the polypeptide.

Additionally, the invention provides a method identifying a compound that modulate the expression or activity of a TAT-028 polypeptide and/or the expression of a TAT-028 nucleic acid molecule which may be useful for screening for anti-cellular proliferative disease agents. The method includes contacting said TAT-028 nucleic acid molecule or polypeptide with the compound, and determining the effect of said compound on said TAT-028 expression or activity. The method may also involve comparing the expression or activity of the TAT-028 polypeptide and/or the expression of the TAT-028 nucleic acid molecule, in the presence of a candidate agent with the respective expression or activity in the absence of the candidate agent or in the presence of a control agent; and determining whether the candidate agent causes a change (e.g., increase or decrease) in the expression or activity of the TAT-028 polypeptide and/or the expression of the TAT-028 nucleic acid molecule. The expression or activity level of the TAT-028 polypeptide and/or the expression level of the nucleic acid molecule may be compared with a reference range, preferably a predetermined reference range, or a control sample. This method may additionally include selecting an agent that modulates the expression or activity of the TAT-028 polypeptide and/or the expression of the TAT-028 nucleic acid molecule for further testing, or for therapeutic or prophylactic use as an anti-cellular proliferative disease agent. The invention also provides agents, identified by these methods, which modulate the expression or activity of the TAT-028 polypeptide or TAT-028 nucleic acid molecule.

The invention also features a method for identifying a compound that can be used to treat or to prevent a cellular proliferative disease (e.g., cancer such as lung cancer). The method includes contacting an organism having an increased level of expression of a TAT-028 polypeptide and having a phenotype characteristic of a cellular proliferative disease with the compound, and determining the effect of the compound on the phenotype, where detection of an improvement in the phenotype indicates the identification of a compound that can be used to treat or to prevent a cellular proliferative disease.

The invention also features a method for treating or preventing a cellular proliferative disease (e.g., cancer such as lung cancer) in a subject including administering to the subject a compound identified using any method described herein.

The invention also provides for the use or manufacture of medicaments for the treatment of a cellular proliferative disease, including the use of a TAT-028 polypeptide a TAT-028 nucleic acid molecule, a TAT-028 antibody, or any compound identified using any method described herein in the manufacture of a medicament for the treatment of a cellular proliferative disease, such as lung cancer. The use of vaccines in the manufacture of a medicament for the treatment of a cellular proliferative disease, and the use of an agent which interacts with, or modulates the expression or activity of a TAT-028 polypeptide or the expression of a TAT-028 nucleic acid in the manufacture of a medicament for the treatment of a cellular proliferative disease are also provided.

The invention also provides a kit for the analysis of a TAT-028 nucleic acid molecule that includes a TAT-028 nucleic acid molecule probe for analyzing the nucleic acid molecule of a test subject. The invention also provides a kit for the analysis of a TAT-028 polypeptide that includes an antibody or a TAT-028 binding protein for analyzing the TAT-028 polypeptide of a test subject.

Pharmaceutical compositions provided by the invention include substances that modulate the status of cells that expresses TAT-028. Such pharmaceutical compositions may include a TAT-028 polypeptide and a physiologically acceptable carrier. They may also comprise a TAT-028 antibody or fragment thereof, a TAT-028-binding protein, or a protein comprising or consisting of the antigen-binding region of a TAT-028 antibody or fragment thereof that specifically binds to a TAT-028 polypeptide, and a physiologically acceptable carrier. Pharmaceutical compositions of the invention provided also include pharmaceutical compositions comprising any one or more of the following: a TAT-028 polynucleotide and a physiologically acceptable carrier; a ribozyme capable of cleaving a TAT-028 polynucleotide and a physiologically acceptable carrier; and a polynucleotide that encodes a TAT-028 antibody or fragment thereof, a TAT-028-binding protein, or a protein comprising or consisting of the antigen-binding region of a TAT-028 antibody or fragment thereof that specifically binds to a TAT-028 polypeptide and a physiologically acceptable carrier.

The invention provides treatments for a cellular proliferative disease that include a therapeutically effective amount of at least one of the pharmaceutical compositions or medicaments of the invention. The invention also provides a method of delivering a cytotoxic agent to a cell that expresses TAT-028. The method includes conjugating the cytotoxic agent to TAT-028 antibody or fragment thereof that specifically binds to a TAT-028 epitope and exposing the cell to the antibody-agent conjugate.

In preferred embodiments of any of the above methods, the cellular proliferative disease is cancer. The preferred cancer is lung cancer.

The invention also provides methods for preventing or ameliorating the effect of a TAT-028 deficiency that includes administering to a subject having a TAT-028 deficiency, a therapeutically effective amount of a compound (e.g., a functional TAT-028 polypeptide) to prevent or ameliorate the TAT-028 deficiency. The invention further provides methods for preventing or ameliorating the effect of a TAT-028 excess that includes administering to a subject having a TAT-028 excess, a therapeutically effective amount of a compound (e.g., a TAT-028 antibody or TAT-028 binding fragment thereof) to prevent or ameliorate the TAT-028 excess.

The compositions and methods of the invention are useful for the identification, manufacture, and modification of anti-cellular proliferative disease compounds and anti-cancer compounds, cellular proliferative disease diagnostics, cancer diagnostics, cellular proliferative disease treatments and cancer treatments, as well as other utilities. The compositions and methods of the invention provide the following advantages in addition to others not enumerated here: TAT-028 is a novel target for diagnostic, prognostic, theranostic, and preventative methods for cellular proliferative diseases, such as cancer, in particular lung cancer. Furthermore, TAT-028 antibodies, TAT-028 antibody-related proteins, TAT-028 interacting proteins, and anti-cancer compounds described herein provide tools for identifying additional potential diagnostics, therapies, and compounds for treatment of cellular proliferative diseases.

### BRIEF DESCRIPTION OF THE DRAWINGS

FIG. 1. Reproducibility of peptide matching across samples. This figure shows an experiment that was conducted using a complex human tissue sample. The sample was solubilized and fractionated by 1D SDS polyacrylamide gel electrophoresis (PAGE). The gels were cut into 24 equal bands and each band digested with trypsin to obtain peptides for analysis by nano-liquid chromatography-mass spectrometry (LC-MS). Each peptide fraction was injected 15 times onto a reverse phase capillary nano-liquid chromatography C₁₈ column, coupled by electrospray to a QTOF (quadrapole time of flight) mass spectrometer. Peptide maps were derived for each of the 15 LC-MS isotope maps and all pairwise alignments between peptide maps were performed (see “Constellation Mapping and Uses Thereof” (PCT Publication No. WO 2004/049385, US Pat. Publication No. 20040172200; hereinafter referred to as “Constellation Mapping”). The reproducibility of results for the 15 injections of the same sample is shown here. The graph shows the number of peptides (Y axis) that were identified in a given number of injections (X axis) of the 15 possible injections. 90% of peptides were found in at least 14 out of the 15 injections. In addition, the median pairwise peptide matching rate between injections was 98%.

FIG. 2. Variance of peptide intensities. This figure shows the variance of the peptide intensity measurements obtained in the experiment described in the FIG. 1 legend above. These results demonstrate that the intensity values of the matched peptides showed little variance. The graph shows the number of peptides (Y axis) that had a given percentage for coefficient of variance (X axis). The median coefficient of variance (CV) was under 12%. Furthermore, each CV value was calculated over 14 to 15 peptide intensity values 90% of the time (see FIG. 1). This level of variance and high rate of matching peptide across samples allows for accurate comparison of peptide intensities across samples.

FIG. 3. Predicting differential abundance from differential intensity. This figure shows the results of a controlled experiment in which 3 proteins were spiked into a complex sample at 14 different concentrations, from 1.25 fmoles to 500 fmoles. Each of the different concentrations were analyzed in triplicate by LC-MS, for a total of 42 samples. For each of the 3 proteins, 10 peptides were identified in each sample and their intensities recorded.

All differential abundance (dA) ratios and corresponding differential intensity (dI) ratios were obtained. The figure shows a plot of all such pairs where the mean differential abundance and standard deviations are plotted. The black line is the best fit linear regression giving the equation dA=1.9311 dI-1.0523.dA is clearly predicted from dI.

FIG. 4. Hemoglobin assay for protein vs. mass spectrometry for three peptides. This figure shows the levels of three different hemoglobin tryptic peptides as determined by mass spectrometry using Constellation Mapping and “Mass Intensity Profiling System” (U.S. patent application publication number 20030129760, hereafter referred to as “MIPS”) software as compared to hemoglobin levels from the same sample as determined by colorimetric assay. Even single peptide LC-MS intensities gave a reliable picture of the behavior of the parent protein in the sample.

FIG. 5. Normal vs. Tumor MS to MS and expression confirmation for peptide #1. This figure shows a comparison of LC-MS data for peptide #1 (SEQ ID NO: 1: KTEAPAAPAAQETK) between normal and tumor samples using Constellation Mapping and MIPS software. Such data is used in manual confirmation of MS to MS matching results to exclude the possibility of peptide collision and confirm that expression levels were calculated from the correct peptide when closely migrating peptides are present. The left panel represents data from a single patient obtained from the normal tissue adjacent to the patient's tumor, and corresponds to the excised polyacrylimide gel (one-dimensional) band with the greatest intensity of peptide #1. Corresponding data from the same patient's lung tumor is presented in the panel at right. Mass-to-charge ratios (m/z) (uncorrected) are shown on the Y axes, and retention times (rt) (uncorrected) are shown on the X axes. The circles indicate the position of intensity data corresponding to peptide #1. The upper panels provide a wide m/z and rt view and the lower panels show an enlarged view of the area immediately surrounding peptide #1. Intensity, which is proportional to abundance, is depicted in gray scale with lighter shades of gray for increasing intensity on a background of white. This data indicates the overexpression of this peptide in this patient's tumor as compared to the patient's adjacent normal tissue.

FIG. 6. MS to MS/MS confirmation for peptide #1. This figure shows MS (left panel) to MS-MS (right panel) alignment of peptide #1 (SEQ ID NO: 1: KTEAPAAPAAQETK) to confirm that the peptide that was identified as being overexpressed was also the peptide that was sequenced by MS-MS. The isotopes of the peptide are expected to fall within the box present in both panels at roughly m/z 706.9, rt 10.0 to 13.0 minutes. The lower panels provide an enlarged view of the area immediately surrounding peptide #1. Constellation Mapping software is used in this confirmation. Intensity is depicted through a color scale. Increasing intensity is proportional to abundance. “X”s in the right panel indicate (m/z, rt) values for which MS/MS spectra were acquired. Note the multiple “X”s falling within the box.

FIG. 7. Spectrum for peptide #1 (SEQ ID NO 1: KTEAPAAPAAQETK). Fragment ion masses that were detected for this sequence are tabulated in the top panel. The MS/MS spectrum is shown in the bottom panel with the major b- and y-ion matches indicated. This information is generated automatically by the computer algorithm Mascot® (Matrix Science (1999) Electrophoresis 20: 3551-3567), along with a score that is a measure of the confidence that the MS/MS spectrum corresponds to the fragmentation pattern of a peptide with the given sequence. The alignment of the fragment ion masses from the sequence with the peaks in the MS/MS spectrum indicated that the raw MS/MS spectrum under study here was, in fact, the result of the fragmentation of the amino acid sequence represented by peptide SEQ ID NO 1.

FIG. 8. Peptide sequences identified and expression across patients. This table contains a summary of the proteomic data aquired for TAT-028 peptides detected in human lung tumor tissue samples. These peptides (SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17) match uniquely to the TAT-028 protein sequence in that there is a low probability that there were generated from another human protein, as indicted by the Mascot Score associated with each peptide. Based on comparisons of peptides between human tumor samples and normal tissue samples obtained from the same patients, these peptides were determined to be upregulated at a level of greater than 3-fold (differential abundance) and at the frequencies listed in the table. Frequency is expressed as a value out of 30 patient samples analyzed.

FIG. 9. Peptide expression across patients. This figure illustrates the expression profile of each of the identified peptides listed in FIG. 8 across all 30 patients of the study. Plotted is the natural logarithm of the disease/normal intensity ratio for each patient the peptide was observed in. The lines at x-values of 1.1 and −1.1 indicate disease over normal differential abundance of 5-fold, and normal over disease differential abundance of 5-fold, respectively. This data illustrates that essentially all the peptides are expressed at level of greater than 5-fold differential abundance in the majority of patient tumor samples analyzed.

FIG. 10. TAT-028 protein sequence with peptides noted. This figure shows a TAT-028 amino acid sequence (SEQ ID NO: 19). The peptide sequences shown in FIG. 8 present in lung tumor plasma membrane samples as determined from mass spectra are in boldface. The peptides were deemed to uniquely identify this protein based on an in silico tryptic digest of the July 2003 NCBI nr database of human proteins.

FIG. 11. TAT-028 coding sequence with corresponding amino acids. This figure shows an RNA/DNA coding sequence (SEQ ID NO: 20; where “t” is thymine for DNA and uracil for RNA) corresponding to the protein sequence of FIG. 10. The start codon is underlined and italicized. The stop codon is double underlined and italicized. Corresponding amino acids are noted below the appropriate codons. Peptides uniquely identifying TAT-028 from FIG. 8 and their encoding sequences are indicated in boldface.

FIG. 12. TAT-028 Proteins across species. This figure shows an approximate sequence alignment of TAT-028 polypeptide sequences from Human (GenBank gi: 30795231; SEQ ID NO: 19), Chimpanzee (GenBank gi: 55623958; SEQ ID NO: 38), Dog (gi: 73954389; SEQ ID NO: 39), Rat (gi: 11560135; SEQ ID NO: 40), Mouse (gi: 66773932; SEQ ID NO: 41) and Chicken (gi: 45383878, SEQ ID NO: 42).

FIG. 13. RNA preparation quality. This figure shows a quality control formaldehyde gel of a typical RNA preparation. The presence of distinct 28S and 18S ribosomal RNA bands as well as a 2:1 ratio of 28S:18S are indications of the integrity of the RNA species and thus may be considered a measure of the preparation's quality.

FIG. 14. Cloning process. This figure shows a flowchart of a process to clone a target. Solid boxes denote methodology with arrows directing to following tasks. The overall process is expected to be similar for every target cloned, although the specifics will vary from target to target.

FIG. 15. CD98 RACE PCR. This figure shows 5′ and 3′ RACE-PCR (rapid amplification of cDNA ends-polymerase chain reaction) products for CD98 from tumor cDNA (complementary DNA). Three different products were obtained for the 5′-RACE and one for the 3′-RACE. Sequence analysis showed the top product of the 5′ reaction mapped the CD98 start site. The middle and bottom products corresponded to RACE artifacts, possibly due to RACE primer non-specific annealing, as was revealed in the sequence analysis. The 3′ RACE reaction mapped the stop codon of CD98.

FIG. 16. SDS PAGE analysis of recombinantly produced TAT-028. This figure shows an SDS PAGE analysis of purified TAT-028. Lane 1 molecular weight markers, Lane 2 recombinantly produced and purified TAT-028. Methods for the recombinant production and subsequent purification are described in the examples.

### DETAILED DESCRIPTION OF THE INVENTION

Definitions

Unless defined otherwise, all technical and scientific terms used herein have the same meaning as commonly understood to one of ordinary skill in the art. Unless otherwise indicated, such as through context, as used herein, the following terms are intended to have the following meanings in interpreting the present invention.

“Active against” in the context of compounds, agents, or compositions having anti-cancer activity indicates that the compound exerts an effect through interaction with or modulation of a particular target or targets in a manner that is deleterious to the in vitro and/or in vivo growth, proliferation, and/or metastasis of a cancer cell or cells. In particular, a compound active against a gene exerts an action on a target which affects an expression product of that gene. This does not necessarily mean that the compound acts directly on the expression product of the gene, but instead indicates that the compound affects the expression product in a deleterious manner. Thus, the direct target of the compound may be upstream of the expression or function of a target gene in a cancer cell and be considered active against the target gene. While the term “active against” encompasses a broad range of potential activities, it also implies some degree of specificity of target. Therefore, for example, a general protease is not necessarily considered “active against” a particular gene which produces a polypeptide product. In contrast, a compound that inhibits a particular enzyme is active against that enzyme and against the gene which codes for that enzyme.

“Active agent,” “pharmacologically active agent,” “agent,” and “drug” are used interchangeably herein to refer to a compound that induces a desired phenotypic, pharmacological, or physiological effect or a desired effect on an activity. The terms also encompass pharmaceutically acceptable, pharmacologically active derivatives of those active agents specifically mentioned herein, including, but not limited to, salts, esters, amides, pro-drugs, active metabolites, analogs, and the like. When the terms “active agent,” “pharmacologically active agent”, and “drug” are used, then, it is to be understood that the applicant intends to include the active agent per se as well as pharmaceutically acceptable, pharmacologically active salts, esters, amides, pro-drugs, metabolites, analogs, etc. Anti-cancer agents are active agents that are active against one or more cancers or cellular proliferative diseases. Candidate agents are potential active agents. “Agent” may also be used in the context of “binding agent,” referring to a compound, for example a ligand, small molecule, or antibody, that exhibits specific binding with another compound, but that does not necessarily have phenotypic, pharmacological or physiological effects, or effects on an activity. TAT-028 binding agents may be identified by any of the screening methods that permit detection of specific binding provided herein, for example identified modulators of TAT-028 activity or expression that bind TAT-028 nucleic acids and/or TAT-028 polypeptides can be considered TAT-028 binding agents, or TAT-028 binding molecules.

“Activity” comprises one or more measurable properties of a protein, capable of acting or affecting a change on itself, or another molecule, or on a cell, tissue, organ, or organism. Although “activity” may often be taken to imply active function, it is meant to encompass measurable passive functions as well (e.g., maintaining structural conformation of a particular protein complex), preferably those that relate to cancer or disease phenotypes or mechanisms, and most preferably those of TAT-028, that regulate TAT-028, or that are regulated by TAT-028. Some examples, not intended to be limiting, include catalytic enzymatic activity, translocation, binding, immunological activity (including specifically immunogenicity—see for example assays under definition of “antigen” below), or participation in a biochemical, or phenotypic pathway. Those skilled in the art should be able to produce or identify appropriate assays for the activity to be assessed. The activity may be carried out indirectly, such as through functioning in a pathway, and encompasses activities that require co-factors or presence in a protein complex. A percentage activity can be determined by comparison to a control in an assay for the particular activity being examined. Methods for such comparisons are commonly known in the art. For example, the percent kinase activity of a derivative of TAT-028 can be assessed by comparison to the level of activity of underivatized TAT-028 under appropriately similar conditions in a kinase assay. Some assays may require the use of TAT-028 nucleic acids, such as for expression, or producing transgenic cell lines, or specific mutant, variant, or derivative forms of TAT-028.

Some activity assays that may be useful in carrying out the methods of the invention, including identifying functions of TAT-028 polypeptides and TAT-028 nucleic acids, not intended to be limiting, include cell proliferation assays, such as mitotic index (see, for example, Oka et al. (1994) Arch Pathol Lab Med. 118: 506-509; Weidner et al. (1994) Hum Pathol. 25: 337-342), thymidine incorporation assays (see, for example, Rodriguez et al. (1993) Am J Obstet Gynecol. 168: 228-232; Sugihara et al. (1992) Int J Cell Cloning 10: 344-351; Hayward et al. (1992) Int J Cell Cloning 10: 182-189; Sondak et al. (1988) Int J Cell Cloning 6: 378-391), bromodeoxyuridine (BrdU) incorporation assays (see, for example, Limas (1993) J Pathol. 171: 39-47), MIB-1 staining (see, for example, Spyratos et al. (2002) Cancer 94: 2151-2159), or anti-PCNA (proliferating cell nuclear antigen) staining (see, for example, Hall et al. (1990) J Pathol. 162: 285-294; Kurki et al. (1988) J Immunol Methods 109: 49-59; Kubben et al. (1994) Gut 35: 530-535; and the in situ hybridization method of Kohler et al. (Dec. 23, 2004; Epub ahead of print) Histochem Cell Biol.); growth suppression assays, such as assays of susceptibility to arrest (see, for example, Guan et al. (1994) Genes Dev. 8: 2939-2952; Gulliya et al. (1994) Cancer 74: 1725-1732), and drug resistance assays (for example, Vybrant® Multidrug Resistance Assay Kit, catalog #V13180 from Molecular Probes, Eugene, Oreg.); apoptosis assays, such as DAPI staining, TUNEL assay (e.g., Fluorescein FragEL DNA Fragmentation Detection Kit (Oncogene Research Products, Cat.#QIA39)+Tetramethyl-rhodamine-5-dUTP (Roche, Cat. #1534 378)) or APO-BrdU™ TUNEL Assay Kit, catalog #A23210 from Molecular Probes, Eugene, Oreg.) or an assay based on Protease Activity (such as caspases) (for example, EnzChek® Caspase-3 Assay Kit #1, catalog #E13183 from Molecular Probes, Eugene, Oreg.); angiogenesis assays (see, for example, Storgard et al. (2004) Methods Mol Biol. 294: 123-136; Baronikova et al. (2004) Planta Med. 70: 887-892; Hasan et al. (2004) Angiogenesis 7: 1-16; Friis et al. (2003) APMIS. 111: 658-668); cell migration assays (for example, Yarrow et al. (2004) BMC Biotechnol. 4: 21; Berens and Beaudry (2004) Methods Mol Med. 88: 219-24; Heit and Kubes (2003) Sci STKE. 2003 (170): PL5); cell adhesion assays (for example, those using enzyme substrates, such as the Vybrant® Cell Adhesion Assay Kit, catalog #V13181 from Molecular Probes, Eugene, Oregon); assays of ability to grow on soft agar or colony formation assays (see, for example, Freshney (1994) Culture of Animal Cells a Manual of Basic Technique, 3rd ed., Wiley-Liss, New York); assays for changes in contact inhibition or density limitation of growth (see, for example, Freshney (1994), supra); assays of changes in growth factor or serum dependence (see, e.g., Temin (1966) J Natl Cancer Insti. 37: 167-175; Eagle et al. (1970) J Exp Med. 131: 836-879; Freshney (1994) Culture of Animal Cells a Manual of Basic Technique, 3rd ed., Wiley-Liss, New York); assays of changes in the level of tumor specific markers (for example, Mazumdar et al. (1999) Trop Gastroenterol. 20: 107-110; Rosandic et al. (1999) Acta Med Austriaca. 26: 89-92; Clarke et al. (2003) Int J Oncol. 22: 425-30; Nowak et al. (2003) Eur J Gastroenterol Hepatol. 15: 75-80; Sarkar et al. (2002) Int J Pharm. 238: 1-9; Streckfus et al. (2001) Oral Surg Oral Med Oral Pathol Oral Radiol Endod. 91: 174-179; Werther et al. (2000) Eur J Surg Oncol. 26: 657-662; Halberg et al. (1995) In Vivo. 9: 311-314; Varela et al. (1993) Oncology 50: 430-435; Turner et al. (1990) Eur J Gynaecol Oncol. 11: 421-427; Masood (1994) J Cell Biochem Suppl. 19: 28-35; Vogel and Kalthoff(2001) Virchows Arch. 439: 109-117); assays of changes in invasiveness into Matrigel (see, for example, Freshney (1994), supra); assays of changes in cell cycle pattern (for example, as determined by flow cytometry, or mRNA or protein expression in synchronized cells (see, for example, Li et al. (1994) Oncogene 9: 2261-2268); assays of changes in tumor growth in vivo, such as in transgenic mice (for example, Huh et al. (2005) Oncogene 24: 790-800; White et al. (2004) Cancer Cell 6: 159-170; Finkle et al. (2004) Clin Cancer Res. 10: 2499-2511; Williams et al. (2004) J Biol Chem. 279: 24745-24756; Cuadros et al. (2003) Cancer Res. 63: 5895-5901; Quaglino et al. (2002) Immunol Lett. 80: 75-79; Shibata et al. (2001) Cancer Gene Ther. 8: 23-35; Nielsen et al. (2000) Cancer Res. 60: 7066-7074), or in xenografts (for example, in immune suppressed mice, such as SCID mice; see Houghton et al. (1989) Invest New Drugs. 7: 59-69; Rygaard and Spang-Thomsen (1997) Breast Cancer Res Treat. 46: 303-312; van Weerden and Romijn (2000) Prostate 2000 43: 263-271; Azzoli et al. (2002) Semin Oncol. 29: 59-65; Sliwkowski et al. (1999) Semin Oncol. 26: 60-70); binding assays; known cancer diagnostics; etc. Such assays can be used to screen for anti-cancer agents, including identification of TAT-028 nucleic acids or TAT-028 polypeptides which are capable of altering or inhibiting abnormal proliferation and transformation in host cells, and activators, inhibitors, and modulators of TAT-028 nucleic acids and TAT-028 polypeptides. Such activators, inhibitors, and modulators of TAT-028 can then be used to modulate TAT-028 expression in tumor cells or abnormal proliferative cells. Identified TAT-028 nucleic acids or TAT-028 polypeptides which are capable of inhibiting abnormal proliferation and transformation in host cells can be used in a number of diagnostic or therapeutic methods, e.g., in gene therapy to inhibit abnormal cellular proliferation and transformation.

“Administering” refers to delivering a foreign substance or a precursor thereof to one or more cells, such as a tissue or organism, for example a mouse or a human. Means of administering the foreign substance vary depending on the cell's environment. For example, a foreign substance can be delivered to a cell in culture by adding the substance to the cell culture media. Delivery of a foreign substance to a cell in a body organ or tissue might require more sophisticated means of delivery, including, but not limited to, implantation, direct injection, injection into the bloodstream or lymphatic system, encapsulated or unencapsulated oral delivery, foodstuffs, solutions, gels, ointments, and the like.

“Affinity” refers to strength of binding between substances, and/or methods based on binding. A high binding affinity is generally desired between an antibody and its antigen, or, for example, a specific and high affinity compound can generally be used to more readily purify a specific protein from a mixture than a low affinity compound. A lower affinity compound might be used, for example if broader specificity is desired, such as allowing several members of a particular protein family to be isolated. By “high binding affinity” is meant binding with an affinity constant of less than 1 micromolar, preferably, less than 100 nanomolar, and more preferably, less than 10 nanomolar. Most preferably, for TAT-028 binding molecules, especially TAT-028 antibodies, high binding affinity means a specific and/or selective TAT-028 binding molecule with greater affinity for a TAT-028 than previously demonstrated for a particular class of binding molecule (e.g., small molecule, antibody, antibody fragment, cyclic peptide, ligand, etc.) Binding and affinity assays known in the art may be used to determine such relative affinity or screen for high affinity binders.

“Affinity tag” refers to a sequence added to the coding information of an expressed protein to provide a convenient site that can be recognized by a capture reagent. The resultant protein is often referred to as a fusion protein. Affinity tags may be encoded at any point in the coding sequence, but are typically placed so as to produce an N— or C-terminal “tag.” More than one tag, possibly of more than one type, may be encoded in a coding sequence. Affinity tags may often also be used as epitope tags, but affinity tag is often used to refer to a tag commonly used in a process that involves a capture reagent other than antibodies, such as nickel beads used with a HIS-tag. Typical examples of affinity tags are the “FLAG”, “HIS” and “GST” tags.

“Altered” or “changed” refers to a detectable change or difference from a reasonably comparable state, profile, measurement, or the like. One skilled in the art should be able to determine a reasonable measurable change. Such changes may be all or none. They may be incremental and need not be linear. They may be by orders of magnitude. A change may be an increase or decrease by 1%, 5%, 10%, 20%,30%, 40%, 50%, 60%, 70%, 80%, 90%, 95%, 99%, 100%, or more, or any value in between 0% and 100%.

“Analogue” refers to a molecule, or substructure or fragment thereof, having a same or similar activity or function as another molecule (“analogous activity”). An analogue can often complement a “knockout” of the gene or protein to which it is analogous in an assay, such as a phenotypic assay. Analogous activity should generally be at least within 1 to 2 orders of magnitude for the gene or gene product to be considered an analogue, but more specific acceptable ranges may be noted and defined by context herein. Two kinases may be broadly considered to have the same activity with regard to enzymatic function, although they may or may not be considered analogous with regard to a particular substrate.

“Antibody” refers to an immunoglobulin protein (or proteins such as in the case of a polyclonal antibody) whether naturally or synthetically produced, which is capable of binding an antigen, whether the antigen is that which caused the antibodies production, one which a recombinant antibody was designed to bind, or to which the antibody's binding was identified, such as through in vitro binding assays. The term may be used to encompass the antibody, antibody fragments, a polypeptide substantially encoded by at least one immunoglobulin gene or fragments of at least one immunoglobulin gene, which can participate in specific binding with the antigen, and/or naturally-occurring forms, conjugates, and derivatives, thereof. An antibody of the invention recognizes a TAT-028 polypeptide. Preferably, an antibody of the invention specifically binds to a TAT-028 polypeptide. The immunoglobulin molecules of the invention can be of any class (e.g., IgG, IgE, IgM, IgD, and IgA) or subclass of immunoglobulin molecule. The term also covers any protein having a binding domain that is homologous to or derived from an immunoglobulin binding domain, such as a CDR region or a cyclized peptide based on a CDR amino acid sequence, though terms such as “antigen-binding region of an antibody” may also be used to encompass CDR regions and the like. An antibody can be derived from a sequence of a mammal, non-mammal (e.g., birds, chickens, fish, etc.), or fully synthetic antibody sequences. A “mammal” is a member of the class Mammalia. Examples of mammals include, without limitation, humans, primates, chimpanzees, rodents, mice, rats, rabbits, sheep, camels and cows.

Derivatives within the scope of the term include antibodies that have been modified in sequence, but remain capable of specific binding to a target molecule, including interspecies, chimeric, and humanized antibodies. An antibody may be monoclonal or polyclonal, and present in a variety of media including, but not limited to, serum or supernatant, or in purified form. As used herein, antibodies can be produced by any known technique, including harvest from cell culture of native B lymphocytes, hybridomas, recombinant expression systems, by phage display, or the like. Methods of production of polyclonal antibodies are known to those of skill in the art.

“Antibody fragment” or “antibody protein fragment” refers to a portion of an antibody (i.e., Fv) capable of binding to an antigen. Fragments within the scope of the term as used herein include those produced by digestion with various peptidases, such as Fab, Fab′ and F(ab)′2 fragments, those produced by chemical dissociation, by chemical cleavage, and by recombinant techniques, so long as the fragment remains capable of specific binding to a target molecule. Typical recombinant fragments, as are produced, e.g., by phage display, include single chain Fab and scFv (“single chain variable region”) fragments. Derivatives within the scope of the term include those that have been modified in sequence, but remain capable of specific binding to a target molecule, including interspecies, chimeric, and humanized antibodies.

“Antigen” refers to a substance that is or will be introduced or injected into a vertebrate animal such as a mammal or poultry; or presented by antigen presentation machinery; or brought into contact with a T cell, B cell, or antigen presenting cell to induce an immune response, particularly the formation of specific antibodies that can combine or bind with the antigen. An antigen may or may not be immunogenic. Antigens that can induce an immune response are often referred to as immunogenic. Antigens, such as peptides, may be tested to determine immunogenicity by an appropriate assay, which are known in the art (see, for example, Chen et al. (1994) Cancer Res. 54: 1065-1070, Coligan et al. (1998) Current Protocols in Immunology, vol. 1, Wiley Interscience (Greene 1998).

The portions of the antigen that make contact with the antibody are denominated “epitopes.” Encompassed within this term herein are haptens, small antigenic determinants capable of inducing an immune response only when coupled to a carrier. Haptens bind to antibodies but by themselves cannot induce an antibody response.

“Antigen presentation” refers to the process by which certain cells in the body (antigen presenting cells) express antigen on their cell surfaces in a form recognizable by lymphocytes.

“Antigen presentation machinery” refers to the proteins, biomolecules, and co-factors involved in the proteolysis, transport and delivery to the cell surface, and presentation of previously foreign substances as antigens on the cell surface by MHC1 and/or MHC2.

“Artificial chromosome” refers to a DNA construct that comprises a replication origin, telomere, and centromere, for replication, propagation to and maintenance in progeny human cells. In addition, they may be constructed to carry other sequences for analysis or gene transfer.

“Binding” refers to a non-covalent or a covalent interaction, preferably non-covalent, that holds two molecules together. For example, two such molecules could be an enzyme and an inhibitor of that enzyme. Another example would be an enzyme and its substrate. A third example would be an antibody and an antigen. Non-covalent interactions include, but are not limited to, hydrogen bonding, ionic interactions among charged groups, van der Waals interactions, and hydrophobic interactions among non-polar groups. One or more of these interactions can mediate the binding of two molecules to each other. Binding may exhibit discriminatory properties such as specificity or selectivity.

As used herein, “biological sample” (or “sample”) refers to any solid or fluid sample obtained from, excreted by, or secreted by any living organism, including single-celled micro-organisms (such as bacteria and yeasts) and multicellular organisms (such as plants and animals, for instance a vertebrate or a mammal, and in particular a healthy or apparently healthy human subject (e.g., a reference sample), a human patient affected by a condition or disease to be diagnosed or investigated), and those subjected to environmental or treatment conditions. A biological sample may be a biological fluid obtained from any location (such as whole blood, blood plasma, blood serum, urine, bile, cerebrospinal fluid, aqueous or vitreous humor, or any bodily secretion), an exudate (such as fluid obtained from an abscess or any other site of infection or inflammation), or fluid obtained from a joint (such as a normal joint or a joint affected by disease such as rheumatoid arthritis). Alternatively, a biological sample can be obtained from any organ or tissue (including a biopsy or autopsy specimen) or may comprise cells (whether primary cells or cultured cells) or medium conditioned by any cell, tissue, or organ. If desired, the biological sample is subjected to preliminary processing, including separation techniques. For example, cells or tissues can be extracted and subjected to subcellular fractionation for separate analysis of biomolecules in distinct subcellular fractions, e.g., proteins or drugs found in different parts of the cell. A sample may be analyzed as subsets of the sample, e.g., bands from a gel. “Sample” may also be more broadly used to encompass recombinant, synthetic, and in vitro generated compounds or collections of compounds, and/or their combination with or presence in biological samples, for example, a protein complex produced and self-assembled in reticulocyte lysate by in vitro translation (IVT, e.g., Product #L4540, Flexi® Rabbit Reticulocyte Lysate System, Promega, Madison, Wis.). Such samples may be useful as controls or in providing a desired set of experimental conditions, such as for a method of screening.

“Candidate agent” refers to a potential active agent, such as a potential anti-cancer agent. “Candidate active agent” or “candidate anti-cancer agent” may also be used herein.

A “capture reagent” is a substance that can bind to a target molecule. Generally, such binding is selective and/or specific. The affinity of such reagents may vary. Preferably the affinity is high enough to reasonably meet the aims of the method they are used to address. More preferably they are of high binding affinity. However, a collection of low affinity binders can be combined to provide a high affinity equivalent (high avidity). High avidity capture reagents are also preferable. Such reagents are often used for their selective and/or specific properties in separation or purification methods. In some cases less selective reagents may be preferable, such as those that could effectively bind and deplete a family of proteins via a similar or common epitope, but in other cases highly selective or specific reagents capable of distinguishing even small differences between similar proteins may be preferred. An example of a capture reagent is nickel, such as may be present in a column to purify histidine-tagged proteins from a bacterial cell lysate. Immunoaffinity reagents are capture reagents composed at least in part of naturally occurring or engineered antibodies, antibody fragments, including CDR peptides, and the like. Immunoaffinity reagents may recognize one or more antigens or epitopes. TAT-028 or fragments thereof may be used in the methods of the invention as capture reagents, and are preferred embodiments of such. Other preferred capture reagents include TAT-028 binding molecules and fragments thereof, of which more preferred are TAT-028 antibodies and fragments thereof.

“cDNA” means complementary deoxyribonucleic acid.

“Cellular proliferative disease” is intended to refer to any condition characterized by the undesired propagation of cells. Included are conditions such as neoplasms, cancers, myeloproliferative disorders, and solid tumors. Some non-limiting examples of cancers that may be treated by the compositions and methods of the invention include: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastom, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma [embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, lipoma, angioma, dermatofibroma, keloids; and Adrenal glands: neuroblastoma. Preferably, treatment of such cancers by the methods and compositions of the invention is in vivo in the patient of origin, however, it may occur in vitro such as treatment of derived cell lines or treatment of ex-plants or xenografts. “Cellular proliferative diseases” also include non-cancerous conditions such as benign melanomas, benign chondroma, benign prostatic hyperplasia, psoriasis, moles, dysplastic nevi, dysplasia, hyperplasias, and other cellular growths occurring within the epidermal layers, as well as angiogenesis. The term is also intended to encompass diseases that can be treated or maintained by slowing, arresting, or decreasing host cell proliferation, for example, viruses whose replication is slowed or inhibited by slowing or inhibiting host cell entry into S phase, the cell cycle phase during which host cell DNA replication occurs.

“Codes for” or “encodes” refer to a DNA or RNA sequence capable of being wholly or partially replicated, transcribed, transcribed and translated, or translated to give a particular product. Hence, DNA may be transcribed into an RNA that can be translated into a given protein and thus “encodes” the protein (likewise it encodes the RNA).

“Complementary sequence” refers to nucleic acid sequence of bases that can form a double-stranded structure by matching base pairs. For example, the complementary sequence to 5′-C-A-T-G 3′ (where each letter stands for one of the bases in DNA) is 3′-G-T-A-C -5′. A pair of complementary sequences may be RNA-RNA, RNA-DNA, DNA-RNA, or DNA-DNA. “Percent complementary” (“% complementary”) may be used to refer to the percent sequence identity to a complementary sequence of the particular type nucleic acid desired (e.g., an RNA complement to a DNA sequence, or a DNA complement thereto), generally to delimit the acceptable number of mismatches in base pairing. Such mismatches may be contiguous or discontiguous.

“Control” generally refers to an experiment or sample, condition, organism, etc., which can be used as a standard of comparison in judging, checking, or verifying experimental results. For example, an experiment in which samples are treated as in a parallel experiment except for omission of the procedure or agent under test may act as a control experiment for the parallel experiment, thereby indicating which effects may be correlated with the use of the procedure or agent. Preferably a control minimizes the number of possible differences between itself and the thing (experiment, organism, etc.) it parallels to help eliminate confounding factors. One skilled in the art may be able to determine an appropriate control when one is desired.

“Cytokine” refers to a protein or peptide that generally is a mediator of local interactions in cell-cell communication, and is often involved in signaling. Many cytokines, especially interleukins and interferons, are secreted by immune cells and are recognized by cytokine receptors on other immune cells. Cytokines cause a variety of actions, such as activation, proliferation, and maturation of the cells. The term ‘cytokine’ also encompasses any proteins or peptides referred to as a growth factor. Examples include NGF, FGF, EGF, (Nerve, Fibroblast, & Epidermal Growth Factors).

“Cytotoxic agent” refers to a compound, agent, or composition that has a toxic effect on cells. Cytotoxic agents are commonly used in chemotherapy to inhibit the proliferation of cancerous cells.

By “derivative” is meant a molecule or fragment thereof that has been chemically altered from a given state. Derivitization may occur during non-natural synthesis or during later handling or processing of a molecule or fragment thereof. Derivitization may result from a natural process, such as the steps of a cellular biochemical pathway. Recombinant nucleic acids or proteins that alter the naturally-occurring nucleic acid or amino acid sequence, respectively, may also be referred to as derivatives.

“Detect” or “detection” refers to identifying the presence, absence, or amount of the substance or state to be detected.

By “detectable label” is meant a molecule or fragment thereof that has been derivatized with an exogenous label (e.g., an isotopic label, fluoroscein, or radiolabel) that causes the molecule or fragment thereof to have different physicochemical properties compared to the naturally occurring molecule or fragment thereof.

The terms “diagnosis” and “diagnostics” also encompass the terms “prognosis” and “prognostics”, respectively, as well as the applications of such procedures over two or more time points to monitor the diagnosis and/or prognosis over time, and statistical modeling based thereupon. Furthermore the term diagnosis includes:
a. prediction (determining if a patient will likely develop a hyperproliferative disease)
b. prognosis (predicting whether a patient will likely have a better or worse outcome at a pre-selected time in the future)
c. therapy selection (some therapies, particularly those that comprise TAT-028 specific binding partners, will work better than others if TAT-028 is present; additionally, some cancers could require more aggressive treatment depending on the TAT-028 status of the tumor cells)
d. therapeutic drug monitoring (it should be possible to determine if a patient is responding well to therapy by detecting the level of TAT-028 found in patient samples taken at different times during a course of therapy)
e. relapse monitoring (if a patient has no detectable tumor or TAT-028 in a body sample over a period of time following therapy and then TAT-028 reappears in a recently obtained sample, the skilled physician should evaluate the strong possibility of a relapse)

“DNA” refers to deoxyribonucleic acid and/or modifications and/or analogs thereof.

By “effective amount” or “therapeutically effective amount” of an agent is meant a sufficient amount of the agent to provide the desired therapeutic effect, over the course of administration. An “effective amount” of an anti-cancer agent is a sufficient amount of the agent to at least partially inhibit or reverse tumor growth. Of course, undesirable effects, e.g., side effects, are sometimes manifested along with the desired therapeutic effect; hence a practitioner balances the potential benefits against the potential risks in determining what is an appropriate “effective amount” using only routine experimentation.

“ELISA” means enzyme-linked immunosorbent assay.

An “epitope” is a region on a macromolecule which is recognized by an antibody. Frequently it is in a short region of primary sequence in a protein and it is generally about 5 to 12 amino acids long (generally the size of the antigen binding site on an antibody). Carbohydrates, nucleic acids and other macromolecules may be antigens and have epitopes.

“Epitope tag” refers to an epitope added to the coding information of an expressed protein to provide a convenient antigenic site that can be recognized by a well characterized antibody. The resultant protein is often referred to as a fusion protein. Epitope tags may be encoded at any point in the coding sequence, but are typically placed so as to produce an N— or C-terminal “tag.” More than one tag, possibly of more than one type, may be encoded in a coding sequence. Typical examples of epitope tags are the “FLAG” and “myc” tags. Some affinity tags, HIS and GST tags, for example, may also be used as epitope tags as well.

“Expression” refers to the product or products of a nucleic acid sequence as mediated by transcription and/or translation, and/or the qualitative or quantitative assessment of the amount of such products. For DNA the expression products are generally the encoded RNA and/or protein. For RNA the expression product is generally protein.

“FLAG-tag” refers to one of the first epitope tag systems. The FLAG epitope is recognized, in calcium dependent binding, by commercially available M1 and M2 antibodies (Sigma-Aldrich Co., MO; U.S. Pat. Nos. 4,703,004, 4,782,137, and 4,851,341). The system can be used both for affinity purification and other immunological procedures. The most widely used hydrophilic octapeptide now is DYKDDDDK (SEQ ID NO: 23) though recent studies suggest that a shorter peptide, DYKD (SEQ ID NO: 24), can be recognized with almost the same affinity by the Ml monoclonal antibody. Also, new tag sequences have been described for other monoclonal antibodies. The peptide MDFKDDDDK (SEQ ID NO: 25) is recognized by M5 and MDYKAFDNL (SEQ ID NO: 26) recognized by M2. The binding reaction is also dependent on calcium, so proteins can frequently be eluted from an affinity matrix by an EDTA containing buffer. This system allows for the tag to be placed at either the amino-terminus (N-terminal) carboxy-terminus (C-terminal), or in association with other tags. It will not usually interfere with the fusion protein expression, proteolytic maturation, or activity. Even if the tag is placed in the MHC class I molecule, it may not interfere with either alloantibody recognition or cytotoxic T cell-MHC interactions.

“Foreign substance” refers to a substance introduced from outside a cell, collection of cells, tissue, organ or organism. Such substances include, but are not limited to, nutrients, drugs, antibodies, vaccines, pharmaceutical compositions, DNA, RNA, liposomes, microorganisms, viruses, parasites, bacteria, yeast, fungi, mycobacteria, protein plaques, protein aggregates, collagen, extracellular matrix, other cells—living or dead, and/or debris. Such substances may also be exogenously produced substances that are or could be produced in the cell, collection of cells, tissue, organ or organism—for example, a protein or antibody.

“gDNA” refers to genomic DNA.

“GST-tag” refers to a glutathione S-transferase affinity or epitope tag that may or may not have a cleavage site included. As an affinity tag, GST binds to the ligand glutathione, which is generally coupled to a Sepharose bead.

“HA-tag” refers to an epitope tag derived from haemagglutinin, generally of the amino acid sequence YPYDVPDYA (SEQ ID NO: 27).

“HIS-tag” refers to an affinity tag consisting of multiple consecutive histidine amino acids. Generally six (hexa-HIS) residues are used (SEQ ID NO: 28), or multiples thereof. His-tagged proteins have a high selective affinity for Ni²⁺ and a variety of other immobilized metal ions. Consequently a protein containing a His-tag is generally selectively bound to a metal ion charged medium while other cellular proteins bind weakly or are washed out with the binding or wash buffers.

“Homology” generally refers to the percent sequence identity, it may also be used to refer to close or equivalent structural and/or conformational homologues and/or analogues that may be reflected in direct comparisons of sequence (nucleic acid or protein), or may not, in which case the homology can be described as “cryptic”. Conformational or structural homology may be identified through structural comparisons, such as might be based on crystal structures, nuclear magnetic resonance (NMR) structures, secondary structure prediction, molecular modeling, binding assays and the like. Conformational and structural analogues may be identified through binding assays, enzymatic assays, phenotypic assays, and other methods known in the art.

“Humanized” or “humanizing” refers to methods for identifying, screening for, designing, making, and producing antibodies (e.g., methods of making recombinant antibodies from antibodies produced in an immune response in a non-human animal or fragments or sequences thereof) or the resultant antibodies themselves, which lower the chances of an undesired human immune response to the portions of the antibodies recognized as foreign, for example a HAMA (human anti-murine antibody) or HACA (human anti-chimeric antibody) response. “Humanizing” methods generally aim to convert the variable domains of non-human antibodies to a more human form by recombinant construction of an antibody variable domain having, for example, both mouse and human character. Humanizing strategies are based on several consensual understandings of antibody structure data. First, variable domains contain contiguous tracts of peptide sequence that are conserved within a species, but which differ between evolutionarily remote species, such as mice and humans. Second, other contiguous tracts are not conserved within a species, but even differ between antibody producing cells within the same individual. Third, contacts between antibody and antigen occur principally through the non-conserved regions of the variable domain. Fourth, the molecular architecture of antibody variable domains is sufficiently similar across species that correspondent amino acid residue positions between species may be identified based on position alone, without experimental data.

Humanizing strategies tend to share the premise that replacement of amino acid residues that are characteristic of murine or other non-human sequences with residues found in the correspondent positions of human antibodies will reduce the immunogenicity in humans of the resulting antibody. However, replacement of sequences between species usually results in reduced affinity for the antigen from the resultant antibody. Preferably, the humanized antibody will exhibit the same, or substantially the same, antigen-binding affinity and avidity as the parent antibody. Preferably, the affinity of the antibody will be at least about 10% that of the parent antibody. More preferably, the affinity will be at least about 25% that of the parent antibody. Even more preferably, the affinity will be at least about 50% or more that of the parent antibody. Most preferable would be improved affinity as compared to the parent antibody. Methods for assaying antigen-binding affinity are well known in the art and include half-maximal binding assays, competition assays, and Scatchard analysis. The art of humanization therefore lies in balancing replacement of the original (e.g., murine) sequence to reduce immunogenicity with the need for the humanized molecule to retain sufficient antigen binding to be therapeutically useful. This balance has previously been struck using two approaches one exemplified by U.S. Pat. No. 5,869,619 and by Padlan ((1991) Mol Immunol 28: 489-498) and a second exemplified by U.S. Pat. No. 5,225,539 to Winter and by Jones et al. ((1986) Nature 321: 522-525). To determine appropriate contiguous tracks for replacement, both Winter and Jones et al. (1986) utilized a classification of antibody variable domain sequences that had been developed previously by Wu and Kabat ((1970) J Exp Med. 132: 211-250).

U.S. Pat. No. 5,693,761 to Queen et al., discloses one refinement on Winter for humanizing antibodies using human framework sequences closely homologous in linear peptide sequence to framework sequences of the mouse antibody to be humanized.

In other approaches, criticality of particular framework amino acid residues is determined experimentally once a low-avidity humanized construct is obtained, by reversion of single residues to the mouse sequence and assaying antigen binding as described by Riechmann et al., ((1988) Nature 332: 323-327). Another example approach for identifying criticality of amino acids in framework sequences is disclosed by U.S. Pat. No. 5,821,337 to Carter et al., and by U.S. Pat. No. 5,859,205 to Adair et al. These references disclose specific Kabat residue positions in the framework, which, in a humanized antibody may require substitution with the correspondent mouse amino acid to preserve avidity.

A second type of refinement to Winter is exemplified by Padlan et al. (1995) FASEB J. 9: 133-139; and Tamura et al. ((2000) J Immunol. 164: 1432-1441), which teach that increasing the proportion of characteristically human sequence in a humanized antibody will reduce that antibody's immunogenicity, and they accordingly disclose methods for grafting partial CDR sequences.

The term “human antibodies” or “fully human antibodies” may refer to antibodies of human origin or produced having a human primary sequence to reduce chances of undesired immunogenicity in humans. For example, transgenic mice bearing human variable region sequences may be used to generate antibodies and the variable regions may be grafted to human constant regions to create fully human antibodies, or the mice may simply have fully human sequences allowing the direct generation of fully human antibodies in response to antigen. Human monoclonal antibodies can be prepared by the trioma technique; the human B-cell hybridoma technique (see Kozbor et al. (1983) Immunol Today. 4: 72-79) and the EBV hybridoma technique to produce human monoclonal antibodies (see Cole et al. (1985) in Monoclonal Antibodies and Cancer Therapy, Alan R. Liss, Inc., pp. 77-96). Human monoclonal antibodies may also be produced by using human hybridomas (see Cote et al. (1983) Proc Natl Acad Sci. U.S.A. 80: 2026-2030) or by transforming human B-cells with Epstein Barr Virus in vitro (see Cole et al. (1985) in Monoclonal Antibodies and Cancer Therapy, Alan R. Liss, Inc., pp. 77-96). Methods for producing fully human monoclonal antibodies, include phage display and transgenic methods, are known and may be used for the generation of human mAbs (for review, see Vaughan et al. (1998) Nat Biotech. 16: 535-539). For example, fully human anti-TAT-028 monoclonal antibodies may be generated using cloning technologies employing large human Ig gene combinatorial libraries (i.e., phage display) (Griffiths and Hoogenboom in: Protein Engineering of Antibody Molecules for Prophylactic and Therapeutic Applications in Man. Clark, M. (Ed.), Nottingham Academic, pp 45-64 (1993); see also, Hoogenboom and Winter (1992) J Mol Biol. 227: 381-388; Marks et al. (1991) J Mol Biol. 222: 581-597; and Burton and Barbas (1994) Adv Immunol. 57: 191-280). Along these lines, antibodies produced by the method of U.S. Pat. No. 5,840,479 are considered for the purposes of this invention “fully human” provided they provide comparable levels of anti-antibody response to other fully human antibodies as might be measured in an assay system known in the art, such as that devised by Stickler et al. ((2000) J Immunother. 23: 654-660). Fully human anti-TAT-028 monoclonal antibodies may also be produced with an antigen challenge using transgenic animals, such as mice engineered to contain human immunoglobulin gene loci as described in PCT Pat. Nos. such as WO 94/02602 and WO 98/24893 and U.S. Pat. Nos. 5,545,807; 5,545,806; 5,569,825; 5,625,126; 5,633,425; and 5,661,016 (see also, Jakobovits (1998) Exp Opin Invest Drugs. 7: 607-614; Marks et al. (1992) Biotechnology 10: 779-783; Lonberg et al. (1994) Nature 368: 856-859; Morrison (1994) Nature 368: 812-13; Fishwild et al. (1996) Nature Biotechnol. 14: 845-851; Neuberger (1996) Nature Biotechnol. 14: 826; and Lonberg and Huszar (1995) Intern Rev Immunol. 13: 65-93). Other human antibody technologies that may be of use in practicing the invention include, but are not limited to, those described in U.S. Pat. Nos. 6,657,103; 6,162,963; 6,319,690; 6,300,129; 6,673,986; 6,114,598; 6,075,181; 6,150,584; 5,770,429; 5,789,650; 5,814,318; 5,874,299; 5,877,397; 6,794,132; 6,406,863; 4,950,595; 5,286,647; 4,833,077; 4,716,111; 4,444,887; 4,594,245; 4,761,377; 4,434,230; 4,451,570; 4,464,465; and 4,529,694.

“Immune response” refers to a series of molecular, cellular, and organismal events that are induced when an antigen is encountered by the immune system. These may include the expansion of B— and T-cells and the production of antibodies. Aspects of an immune response, such as the expansion of T cell, B cell, or other antigen presenting cell populations may take place in vitro for administration to a subject. The immune response may provide a defense against foreign substances or organisms or aberrant host cells, such as cancer cells. Some tumors induce specific immune responses that suppress their growth. These often seem to be directed at peptides derived from antigens that might be mutated, inappropriately expressed, or overexpressed in the tumor cells. To determine whether an immune response has occurred and to follow its course, the immunized individual can be monitored for the appearance of immune reactants directed at the specific antigen.

“Immunoassay” refers to one of a number of techniques for the determination of the presence or quantity of a substance, especially a protein, through its properties as an antigen or antibody. The binding of antibodies to antigen is often followed by tracers, such as fluorescence or (radioactive) radioisotopes, to enable measurement of the substance. Immunoassays have a wide range of applications in clinical and diagnostic testing. An example is solid-phase immunoassay in which a specific antibody is attached to a solid supporting medium, such as a PVC sheet. The sample is added and any test antigens will bind to the antibody. A second antibody, specific for a different site on the antigen, is added. This carries a radioactive or fluorescent label, enabling its concentration, and thus that of the test antigen, to be determined by comparison with known standards.

“Immunogen” refers to an antigen capable of inducing an immune response.

“Immunogenic” refers to the ability to induce an immune response. Typically a substance capable of inducing an immune response is referred to as immunogenic.

By “immunogenically effective amount” is meant an amount of a composition that is effective in inducing an immune response (e.g., a humoral or a mucosal immune response) when administered to a patient (e.g., human patient).

“Interact” refers to binding, proteolyzing, modifying, regulating, altering, and/or the like, generally as governed by context. Often it refers simply to binding. Generally it refers to direct interaction, but it may also refer to indirect interaction such as through a biochemical or genetic pathway.

A polynucleotide may be “introduced” into a cell by any means known to those of skill in the art, including transfection, transformation or transduction, transposable element, electroporation, particle bombardment, and infection. The introduced polynucleotide may be maintained in the cell stably if it is incorporated into a non-chromosomal autonomous replicon or integrated into the fungal chromosome. Alternatively, the introduced polynucleotide may be present on an extra-chromosomal non-replicating vector and be transiently expressed or transiently active. “Introduced” may also be used in other context defined ways, such as in the recombinant “introduction” of mutations into a nucleic acid sequence.

“In vitro binding assay” refers to assays reagents and/or systems for detecting and/or measuring, qualitatively and/or quantitatively, the binding between a protein, DNA, and/or RNA and another specific substance or complex, such a protein, DNA, RNA, cyclized peptide, or small molecule in vitro. The assay may be cell-based, such as in the yeast two hybrid and variants thereupon, or, for example, as in CAT or luciferase assays in cultured cells, and may be immunologically-based, such as with the use of immunoaffinity columns, ELISA assays, and the like, but assays in a live animal or person are excluded and considered “in vivo”.

An “isolated” and/or “substantially pure” polynucleotide or nucleic acid molecule is free of genes that, in the naturally occurring genome of the organism from which the nucleic acid molecule of the invention is derived, flank the nucleic acid. The term includes, for example, a recombinant DNA that is incorporated into a vector; into an autonomously replicating plasmid or virus; or into the genomic DNA of a prokaryote or eukaryote; or that exists as a separate molecule (e.g., a cDNA, genomic, or coding fragment produced by PCR or restriction endonuclease digestion) independent of other sequences. It also includes a recombinant DNA that is part of a hybrid gene encoding additional polypeptide sequence. A polynucleotide corresponding to a polypeptide which can be identified by one skilled in the art such as through the use of Mascot (Matrix Science, Boston, Mass.) and translated mRNA databases and BLAST (Gish and States (1993) Nat Genet. 3: 266-272; Alschutl et al. (1997) Nucleic Acids Res. 25: 3389-3402; Madden et al. (1996) Methods Enzymol. 266: 131-141; Altschul et al. (1990) J. Mol. Biol. 215: 403-410) is also considered isolated. Fragments or partial sequences when considered with other data, or when they uniquely identify a full-length sequence, may be used to identify full-length sequences, which can then also be considered isolated. Such sequences may be amplified from an appropriate library through techniques such as PCR, produced by oligonucleotide synthesis, or through recombinant techniques known in the art. Alternatively, a polynucleotide is considered isolated if it has been altered by human intervention, or placed in a locus or location that is not its natural site, or if it is introduced into one or more cells. Having been isolated, a polynucleotide may readily be manipulated by molecular biological, recombinant, and other techniques and used or present in relatively pure or purified states, or be used or present in combinations, mixtures, solutions, compounds and complex isolates, such as cell lysates. The isolated polynucleotide need not be isolable, separable, or purifiable from any such compositions. The skilled person can readily employ nucleic acid isolation procedures to obtain isolated TAT-028 polynucleotides.

A polypeptide (or fragment thereof) may be said to be “isolated” when physical, mechanical or chemical methods have been employed to remove the polypeptide from cellular constituents. An “isolated polypeptide,” “substantially pure polypeptide,” or “substantially pure and isolated polypeptide” is typically considered removed from cellular constituents and substantially pure when it is at least 60% by weight, free from the proteins and naturally occurring organic molecules with which it is naturally associated. Preferably, the polypeptide is at least 75%, more preferably at least 90%, and most preferably at least 99% by weight pure. A substantially pure polypeptide may be obtained by standard techniques, for example, by extraction from a natural source (e.g., lung tissue or cell lines), by expression of a recombinant nucleic acid encoding a TAT-028 polypeptide, or by chemically synthesizing the polypeptide. Purity can be measured by any appropriate method, e.g., by column chromatography, polyacrylamide gel electrophoresis, or HPLC analysis. A polypeptide for which the encoding nucleic acid sequence has been cloned, or can be derived or identified by one skilled in the art, such as through the use of Mascot (Matrix Science, Boston, Mass.) and translated mRNA databases and BLAST (Gish and States (1993) Nat Genet. 3: 266-272; Alschutl et al. (1997) Nucleic Acids Res. 25: 3389-3402; Madden et al. (1996) Methods Enzymol. 266: 131-141; Altschul et al. (1990) J. Mol. Biol. 215: 403-410) is also considered isolated. Fragments or partial sequences when considered with other data, or when they uniquely identify a full-length sequence, may be used to identify full-length sequences, which can then also be considered isolated. Alternatively, a polypeptide is considered isolated if it has been altered by human intervention, or placed in a location that is not its natural site, or if it is introduced into one or more cells. The skilled person can readily employ protein isolation, separation, and/or purification procedures to obtain an isolated polypeptide, such as a TAT-028 polypeptide after expression by a recombinant polynucleotide encoding the polypeptide. The nature and degree of isolation and purification will depend on the intended use. Having been isolated, a polypeptide may readily be manipulated by molecular biological, recombinant, and other techniques and used or present in relatively pure or purified states, or be used or present in combinations, mixtures, solutions, compounds and complex isolates, such as cell lysates. Embodiments of a TAT-028 polypeptide include a purified TAT-028 polypeptide and a functional, soluble TAT-028 polypeptide. In one form, such functional, soluble TAT-028 polypeptides or fragments thereof retain the ability to bind antibody or other ligand.

As used herein, “lung cancer” preferably refers to cancers of the lung, but may include any disease or other disorder of the respiratory system of a human or other mammal. Respiratory neoplastic disorders include, for example, non-small cell lung cancer, including adenocarcinoma, acinar adenocarcinoma, bronchioloalveolar adenocarcinoma, papillary adenocarcinoma, solid adenocarcinoma with mucus formation, squamous cell carcinoma, undifferentiated large cell carcinoma, giant cell carcinoma, synchronous tumors, large cell neuroendocrine carcinoma, adenosquamous carcinoma, undifferentiated carcinoma; and small cell carcinoma, including oat cell cancer, mixed small cell/large cell carcinoma, and combined small cell carcinoma; as well as adenoid cystic carcinoma, hamartomas, mucoepidermoid tumors, typical carcinoid lung tumors, atypical carcinoid lung tumors, peripheral carcinoid lung tumors, central carcinoid lung tumors, pleural mesotheliomas, and dysplasia, hyperplasia, neoplasia, and metastases of respiratory system origin. Lung cancers may be of any stage or grade. Preferably the term may be used to refer collectively to any dysplasia, hyperplasia, neoplasia, or metastasis in which TAT-028 nucleic acids or TAT-028 polypeptides are expressed above normal levels as may be determined, for example, by comparison to adjacent healthy tissue.

As used herein, “lung tissue”, and “lung cancer” refer to tissue or cancer, respectively, of the lungs themselves, as well as the tissue adjacent to and/or within the strata underlying the lungs and supporting structures such as the pleura, intercostal muscles, ribs, and other elements of the respiratory system. The respiratory system itself is taken in this context as representing nasal cavity, sinuses, pharynx, larynx, trachea, bronchi, lungs, lung lobes, aveoli, aveolar ducts, aveolar sacs, aveolar capilaries, bronchioles, respiratory bronchioles, visceral pleura, parietal pleura, pleural cavity, diaphragm, epiglottis, adenoids, tonsils, mouth and tongue, and the like. The tissue or cancer may be from a mammal and is preferably from a human, although monkeys, apes, cats, dogs, cows, horses and rabbits are within the scope of the present invention.

“Mass spectrometry” refers to a method comprising employing an ionization source to generate gas phase ions from an analyte presented on a sample presenting surface of a probe and detecting the gas phase ions with a mass spectrometer.

“Method of screening” means that the method is suitable, and is typically used, for testing for a particular property or effect of a large number of compounds, including the identification and possible isolation of an individual compound or compounds based a particular property such as binding or not binding to a target molecule. Typically, more than one compound is tested simultaneously (as in a 96-well microtiter plate), and preferably significant portions of the procedure can be automated. “Method of screening” also refers to methods of determining a set of different properties or effects of one compound simultaneously. Screening may also be used to determine the properties for a complete set of compounds in a non-selective fashion, or may be used to select for a particular property or properties, such as might be desired to reduce the number of candidate compounds to be examined in later screening efforts or assays. Screening methods may be high-throughput and may be automated.

“MHC” means Major Histocompatibility Complex.

“Modulating” refers to fixing, regulating, governing, influencing, affecting, and/or adjusting one or more characteristics of a macromolecule or molecular, cellular, tissue, organ, or organismal phenotype. Modulation need not have contemporaneous effect, or be direct.

“Modulator” refers to an agent capable of modulating. Modulators are generally compounds or compositions. Compounds may be administered in a pure form, substantially pure form, and/or in mixtures, solutions, colloids, adjuvants, and/or solid mixtures containing the compound or compounds, particularly when required for delivery of the compound or compounds to the site or sites of action. Administration may be by any mode of delivery appropriate to the compound or compounds being delivered and their target cell or cells known in the art, for example, direct contact, ingestion, or injection. Modulators may be detected by screening methods known in the art, for example by treating with compounds, or modifications and analogs of substances and comparing to control samples. Such screening methods may be high-throughput.

“Myc tag” refers to an epitope tag derived from myc protein, generally of the sequence amino acid EQKLISEEDL (SEQ ID NO: 29). A number of different antibodies are known to recognize the myc epitope tag, for example 9B11 and 9E10.

“mRNA” means messenger ribonucleic acid.

“Operably linked” means incorporated into a genetic construct so that expression control sequences effectively control expression of a coding sequence of interest.

“Overexpression” is primarily used to describe the relative quantity or expression pattern of a particular peptide or protein as greater between one condition and another or between different cell or tissue types. Overexpression may also be used to refer to RNA expression, however, RNA expression is not predictive of protein expression. Generally, overexpression is measured compared to a normal or control condition. For example, a cell expressing 5 micrograms of protein X upon treatment with a compound, could be said to be overexpressing protein X compared to an untreated cell expressed 1 microgram. Due to experimental variation it is preferable for such measurements to be statistically significant and for the methods used to produce such measurements to be reasonably accurate and reproducible. Overexpression need not be a direct result of gene expression through transcription, and in some cases localization may be relevant. For example, a cell might express 5 micrograms of protein X under both treated and untreated conditions, but in the treated cells 100% of the protein might be present at the plasma membrane, as compared to 15% in the untreated cells. This might be described as overexpression relative to the plasma membrane.

Similarly, overexpression may refer to expression at the level of an individual cell, or of a population of cells, such as a tissue, organ, or organism. For example, PCNA, the proliferating cell nuclear antigen is expressed in cells undergoing DNA replication (S phase of the cell cycle). A comparison of PCNA levels in an S phase normal cell and an S phase tumor cell might show the levels to be equivalent. However, comparison of PCNA levels in the normal tissue vs. the tumor might show overexpression of PCNA in the tumor because there are more cells undergoing DNA replication in the tumor (the length of S phase is relatively constant, but the overall cell cycle tends to be shorter in tumor cells, and they divide more frequently). Measurements may be based on the relative weight or mass of samples, their relative cell numbers or volumes, or other reasonable criteria for a particular assessment. For example, whether there is a safe and effective concentration of a radiocompound as estimated by its potential number of binding sites per unit of volume might best be assessed by determining relative expression by volume, while another compound, such as an activator of apoptosis might be better assessed in terms of the expression level on a per cell basis. Potential antigens for immunotherapy would preferably be overexpressed on the plasma membrane of human lung cancer tumor cells relative to the plasma membranes of normal tissue or cells. More preferably potential antigens would also be overexpressed as compared to other normal tissue within the organism.

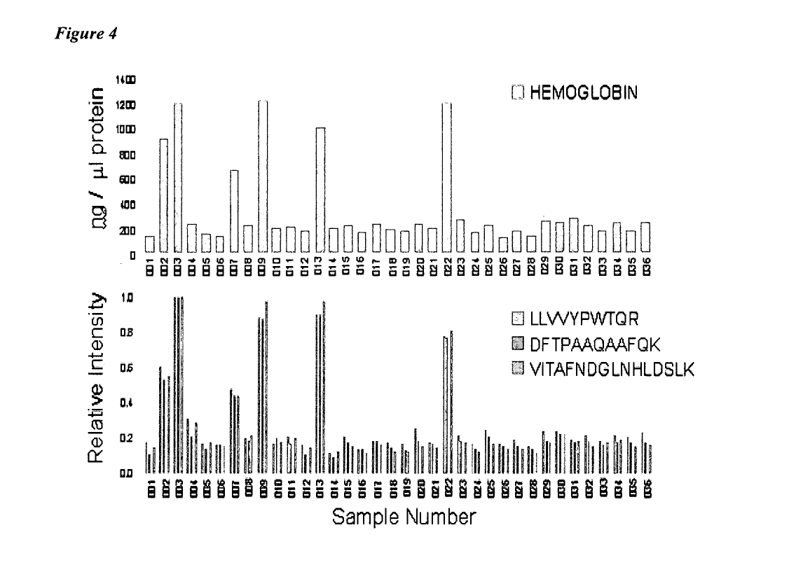

The methods initially used to identify TAT-028 expression herein (see Example 4) permit peptide quantity to be used to infer protein quantity, particularly if the peptide is a unique peptide, or if there are quantities known for multiple peptides from a particular protein. An example of the accuracy of this inference is presented in FIG. 4. One of skill in the art could also further confirm protein quantity through techniques common in the art with appropriate standards for quantitation (absolute or relative) including but not limited to western blotting, ELISA, and immunohistochemistry. Protein identity may also be further confirmed through other techniques such as, but not limited to, microsequencing and V8 protease mapping.

“Overexpression” may also be used to describe a vector used for the production of, high levels of a particular gene product or to describe the resulting gene product, generally for a particular end, such as purification of the protein or experimental assessment of the phenotype associated with overexpression. Some proteins may be difficult to overexpress given toxicity or other factors, so the “high level” of expression may vary from protein to protein, and in this context represents a goal, expression being preferably higher than in the natural state of a protein's expression under corresponding conditions.

“PCR” means polymerase chain reaction.

By “percent (%) sequence identity” is meant the identity between two or more polypeptides or nucleic acid sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7 (PMID: 7265238)); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool; (Altschul, S. F., W. Gish, et al. (1990) J Mol Biol 215: 403-10 (PMID: 2231712)), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. These programs can also be used sequentially first identifying a specific region of a protein for comparison and then performing a second alignment to that region for determination of percent sequence identity.

In general, for proteins, the length of comparison sequences will generally be at least 10 amino acids, preferably 20, 30, 40, 50, 60, 70, 80, 90, 100, 110, 120, 130, 140, 150, 200, 250 or at least 227 amino acids or more. For nucleic acids, the length of comparison sequences will generally be at least 25, 50, 100, 100, 150, 200, 250, 300, 350, 400, 450, 500, 550, 600, 650, 660, 670, 680 or at least 684 nucleotides or more. It is understood that for the purposes of determining sequence identity when comparing a DNA sequence to an RNA sequence, a thymine nucleotide is equivalent to a uracil nucleotide. One skilled in the art should be able to determine an appropriate length for comparison to the TAT-028 sequences or fragments thereof to meet particular aims, see for examples, “substantial identity” below.

Preferably, a sequence of the invention is at least about, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical to a TAT-028 sequence disclosed herein.

“Percent (%) sequence similarity” and “% similar” refer to the percentage of nucleotides or amino acids identical between two sequences or segments thereof, after aligning the sequences and introducing gaps, if necessary, to achieve the maximum percent sequence identity over the aligned portions of the sequence, plus the percentage of conservative substitutions. For purposes of classifying amino acids substitutions as conservative or nonconservative, amino acids are grouped as follows: Group I (hydrophobic side chains): norleucine, met, ala, val, leu, ile; Group II (neutral hydrophilic side chains): cys, ser, thr; Group III (acidic side chains): asp, glu; Group IV (basic side chains): asn, gin, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

By “pharmaceutically acceptable” carrier is meant a pharmaceutical vehicle comprised of a material that is not biologically or otherwise undesirable, i.e., the material may be administered to an individual along with the selected active agent without causing any undesirable biological effects or interacting in a deleterious manner with any of the other components of the pharmaceutical formulation in which it is contained. Carriers may include excipients and other additives such as diluents, detergents, coloring agents, wetting or emulsifying agents, pH buffering agents, preservatives, and the like. Similarly, a “pharmacologically acceptable” salt, ester, amide, prodrug, or derivative of a compound as provided herein is a salt, ester, amide, prodrug, or derivative that is not biologically or otherwise undesirable.

“Plasmid” refers to a small, independently-replicating, nucleic acid that can be transferred from one organism to another. Plasmids may be linear or circular. Linearized plasmids may also concatemers. ‘Stringent’ plasmids occur at low copy number in cells, ‘relaxed’ plasmids at high copy number, circa 10-50 copies per cell. Plasmids can become incorporated into the genome of the host, or can remain independent. An example is the F-factor of E. coli. Plasmids may be used to transfer genes, and plasmids carrying antibiotic-resistant genes can spread this trait rapidly through the population. Plasmids are widely used in genetic engineering as vectors, and may be recombinant.

“Post-translational modifications” or “PTMs” refers to changes that occur to proteins after peptide bond formation has occurred. Examples, not intended to be limiting, include glycosylation, acylation, limited proteolysis, phosphorylation, and isoprenylation.

“Probe” generally refers to a TAT-028 binding complex or binding molecule used in the detection, quantification, and/or qualitative assessment of a TAT-028 nucleic acid or TAT-028 polypeptide in a sample. Non-limiting examples, in addition to those discussed throughout, include a probe nucleic acid used to detect a mutant TAT-028 nucleic acid in a patient sample; a probe antibody used to quantitate the amount of TAT-028 polypeptide in a sample; a binding molecule used to determine if the native conformation of the protein is maintained, for separation from a sample, or for assessing relative purity. A probe is preferably a TAT-028 binding molecule, more preferably a TAT-028 nucleic acid, TAT-028 polypeptide, or TAT-028 antibody, but need not be, such as in the case of determining purity by probing for contaminants.

“Promoter” refers to a region of DNA, generally and preferably from a gene's genomic locus, which can be reasonably demonstrated to be involved in regulating the expression of a gene. This includes both a basal level of transcription and those elements, such as enhancer elements, repressor elements and the like which are capable of regulating gene expression under certain conditions, such as binding by a transcription factor. Generally the region includes a region of DNA to which RNA polymerase binds before initiating the transcription of DNA into RNA. The nucleotide at which transcription starts is designated+1 and nucleotides are numbered from this with negative numbers indicating upstream nucleotides and positive downstream nucleotides. Most factors that regulate gene transcription do so by binding at or near this basal promoter and affecting the initiation of transcription. Most eukaryotic promoters regulated by RNA polymerase II have a Goldberg-Hogness or “TATA box” that is centered around position-25 and has the consensus sequence 5′-TATAAAA-3′ (SEQ ID NO: 30). Several promoters have a CAAT box around -90 with the consensus sequence 5′-GGCCAATCT-3′ (SEQ ID NO: 31).

“Protein,” “peptide,” or “polypeptide” refer to any of numerous naturally occurring, recombinantly derived, or synthetic, sometimes extremely complex (such as an enzyme or antibody) substances that consist of a chain of four or more amino acid residues joined by peptide bonds. The chain may be linear, branched, circular, or combinations thereof. Intra-protein bonds also include disulfide bonds. Protein molecules contain the elements carbon, hydrogen, nitrogen, oxygen, usually sulfur, and occasionally other elements (such as phosphorus or iron). Preferably, polypeptides are from about 10 to about 1000 amino acids in length, more preferably 10-200 amino acids in length. Herein, “protein” is also considered to encompass fragments, variants and modifications (including, but not limited to, glycosylated, acylated, myristylated, and/or phosphorylated residues) thereof, including the use of amino acid analogs, as well as non-proteinacious compounds intrinsic to enzymatic function, such as co-factors, or guide templates (for example, the template RNA associated with proper telomerase function). In context, “protein” may be used to refer to a full-length (encompassing the whole of the coding sequence) or full-length post-translationally modified polypeptide as encoded by a particular nucleic acid sequence, and “peptide” may be used to refer to short amino acid sequences (roughly 4 to 50 amino acids) or non-full-length polypeptides, but this should not be taken as limiting relative to the above definition.

“Recombinant” is an adjective referring to a nucleic acid sequence produced or altered through use of recombinant DNA technology or gene splicing techniques and/or nucleic acids or proteins produced there from, such as through transcription and/or translation. As used herein, the term also encompasses nucleic acids and proteins altered from their natural state or produced through other man-made techniques, for example, oligonucleotide or protein synthesis, or PCR.

A “reference level” generally refers to a particular level of an indicator used as a benchmark for assessment, which may come from a single data point or be derived from multiple data points, such as a cut-off median, and may be measured directly, indirectly, or calculated. Typically the reference level will be used as a reference to a normal or control level allowing the identification of levels that deviate from the normal. For example, a reference level for expression of a particular protein in a patient with cancer may be used in comparison with appropriate samples from patients to determine whether their individual level of the particular protein's expression indicates the presence of cancer or not. An algorithm can be designed, such as by those with skill in the art of statistical analyses, which will allow the user to quickly calculate a reference level for use in making predictions or monitoring a particular state or condition. With additional data, generated similarly to the manner described herein, it may be possible to more accurately define appropriate reference levels. The algorithm and reference level can be used to generate a device that will allow the end user to input levels for a characteristic and quickly and easily determine the status or risk index of an individual through comparison of the level that was input and the reference level. Similarly, it is possible to provide a device that indicates the status of an individual relative to a reference level. One skilled in the art can determine an appropriate reference level when one is desired.

“Reference range” generally refers to a particular range of an indicator used as a benchmark for assessment, such as a mean deviation cut-off multiple points range within which, for example, “normal” or “disease” is expected to fall. In one example, the range of test values expected for a designated population of individuals, e.g., 95 percent of individuals that are presumed to be healthy (or normal). A reference range may be useful in minimizing variation possible with a single reference sample. Generally, all reference ranges include a set of two values with one value designated as an upper reference range limit and another designated as a lower reference range limit. A range may be sub-divided into ranges of differing significance, hence where within a range a value falls may provide additional correlates or probabilities. For example, a range for normal expression of a protein is 0.1 to 0.4 micrograms per liter of plasma, and above the reference level of 0.4 μg/l lung cancer is indicated, however, within the normal range a range of 0.3 to 0.4 μg/l may indicate an 80% probability of dysplastic or pre-cancerous tissue lining the lung. An algorithm can be designed, such as by those with skill in the art of statistical analyses, which will allow the user to quickly calculate a reference range for use in making predictions or monitoring a particular state or condition. With additional data it may be possible to more accurately define appropriate reference ranges. The algorithm and reference range can be used to generate a device that will allow the end user to input levels for a characteristic and quickly and easily determine the status or risk index of an individual through comparison of the level that was input and the reference range. Similarly, it is possible to provide a device that indicates the status of an individual relative to a reference range. One skilled in the art can determine an appropriate reference range when one is desired.

“Reference sample” generally refers to a sample used as a control, that is chosen to represent a normal, or that is designated a normal based on statistical evaluation (for example, having a value for a relevant characteristic that falls within the mean plus or minus 2 standard deviations for a given population). A reference sample may be used as a benchmark for assessment or from which such benchmarks may be derived, thus a reference sample may also be a sample chosen as representative of a particular condition or state, such as presence of a disease. Determination of appropriateness of use as a reference sample may be judged by one skilled in the art before or after measurement of the desired characteristics for which the sample will be used as a reference or as part of a population of reference samples, depending on the reasonableness to do so. For example, it may be reasonable for a group of patients to be designated as reference samples and “normal” for a mutant phenotype they do not display, and measurements of a panel of genes for gene expression may then be used as a reference range for normals relative to that phenotype. In another example, the reference level can be a level determined from a prior sample taken from the same subject. Or, for example, it may be reasonable to determine the TAT-028 concentration in blood from a random sampling of the population (the reference sample thereby being a random sample) and using statistical methods to delineate a normal range, or reference range. Or, a population of samples from untreated patients with melanoma and a population of patients with melanoma undergoing treatment might be useful in providing reference samples for comparison of the effects of a second therapy on protein expression levels. In some contexts, “reference sample” may simply refer to a sample of known quantity, of normal quantity, or readily determinable quantity for comparison. Reference samples may be used to determine reference ranges and/or reference levels for characteristics of the samples. One skilled in the art may be able to determine an appropriate reference sample when one is desired.

“Ribozyme” refers to an RNA molecule that can break or form covalent bonds in their own sequence or another molecule. i.e., it is capable of acting as an enzyme. The reactions observed include cleaving themselves or other RNA molecules, ligation, and trans-splicing. Ribozymes greatly accelerate the rate of the reaction, and can show extraordinary specificity with respect to the substrates it acts on and the products it produces. There are three common types of ribozymes: 1) self-cleaving, both of the hammerhead ribozyme and hairpin ribozyme varieties 2) self-splicing (introns) 3) ribonuclease P. Ribozymes can be generated to cleaving any desired substrate. There is a recognition complex for this enzyme consisting of oligonucleotide hybridized to external guide sequence, making it possible to synthesize a guide sequence and create a substrate for ribozyme attack. Synthetic genes for guide sequence may be transformed to a cell (e.g., a mammalian cell) through tissue-specific biological vectors or oligonucleotides encapsulated in liposomes. Thus, this technique is suitable for inactivation of any RNA inside the cell or in vitro. It may be used as the tool for inactivating genes in mammalian cells.

“RNA” refers to ribonucleic acid and/or modifications and/or analogs thereof.

“RNA equivalent” refers to an RNA sequence corresponding to a DNA or amino acid sequence. Such equivalents may correspond directly to the original sequence (in the case of a protein the “coding sequence”), or may include additional sequence, such as untranslated regions and introns. In the case of an RNA equivalent for DNA the correspondence may be complementary to the DNA strand or anti-sense, allowing for the fact that in RNA “U” replaces “T” in the genetic code.

A “solid support” is a material, essentially insoluble under the given solvent and temperature conditions, with which one or more capture reagents is retained (attached, bound, disposed thereon) and/or made more easily separable from a sample the capture reagents are brought into contact with. In a preferred embodiment, the solid support is covalently coupled to one or more capture reagents capable of directly or indirectly binding a target molecule, such as a protein. When the target molecule is a protein, the capture reagent preferably comprises an immunoaffinity reagent. The solid support is also preferably a particle such as a bead or sphere in the micron or submicron size range, referred to herein as “beads.” Preferably beads are 200 microns or less, more preferably 150 microns or less, most preferably 100 microns or less. The solid support is preferably made of materials that may include one or more of the following: silica, polyacrylate, polyacrylamide, a metal, polystyrene, latex, nitrocellulose, exocellulose, dextran, sepharose, polypropylene, and nylon. Preferably, the solid support is able to be affected by a magnetic field. In such a case, the solid support may have a magnetite core. Other preferred forms of solid supports include filters, planar surfaces, and plate wells (such as those found in high-throughput plate formats, or used for ELISA). Preferably plates are relatively rigid or self-supporting to allow for easy handling during manufacturing and easy handling during use by the end user (a human or a robot). Preferably the plate may be made of polymeric (especially thermoplastic) materials, glass, metallic materials, ceramic materials, elastomeric materials, coated cellulosic materials and combinations thereof such as epoxy impregnated glass mats. In a more preferable embodiment, the plate is formed of a polymeric material including but not limited to polyethylene, acrylic, polycarbonate and styrene. The wells can be made by injection molding, drilling, punching and any other method well known for forming holes in the material of selection. Such plates are well known and commercially available from a variety of sources in a variety of well numbers and designs. Most common are 96 and 384 well plates. Plates are typically 5 inches (127 mm) long and 3.4 inches (86.4 mm) wide. The plate thickness can vary but are generally 0.5 inches (12.7 mm) for a standard plate and 1.75 inches (44.45 mm) for a deep well plate. The well format will be determined by the end users needs, but it can have numerous configurations and the wells do not necessarily need to be all of the same shape or size. Especially with the smaller sized wells, the wells may have the same or different volumes. The wells may also have different shapes. For example, the wells of the present invention may have round, rectangular, teardrop, square, polygonal and other cross-sectional shapes or combinations of them. Virtually any shape that is required for the product may be provided. Typically, it has the wells arranged in uniformly spaced rows and columns for ease of use. Filters may be woven or non-woven, including but not limited to multilayer or composite filters. Not all layers of a multilayer filter need retain, bind, be attached to, etc. a capture reagent. Filters can be chosen with respect to their properties in a way corresponding to the requirements of the respective sample and desired purification, so that the necessary purity class for the medium to be filtered is ensured. In a preferred way, the particle retention of the filters used is >60 micrometers, preferably >100 micrometers. Columns are also preferred solid supports, but are generally a secondary support retaining another form of support such as beads and filters. Solid supports may be used in any combination. For example a column may contain multiple compartments allowing flowthrough that contain different beads with different attached capture reagents as well as filters with attached capture reagents.

“Specific binding,” “selective binding,” and “specific interaction” or “selective interaction” refer to an interaction, even briefly, between TAT-028 and one or more molecules, compounds, or complexes, wherein the interaction is dependent upon the primary amino acid sequence (or other structural elements in a non-peptidic portion of a molecule), post-translational modifications to the amino acid sequence or its modifications, and/or the conformation of TAT-028 and/or its modifications. A molecule that exhibits specific binding toward another molecule may be said to be “specific for” the other molecule. Generally specific binding provides the ability for two molecular species concurrently present in a heterogeneous (non-homogeneous) sample to bind to one another preferentially over binding to other molecular species in the sample. In other words, “specificity” refers to the potential to bind one unique chemical structure more strongly than a number of similar alternatives. Typically, a specific binding interaction will discriminate over adventitious binding interactions in the reaction by at least two-fold, more typically more than 10- to 100-fold. When used to detect an analyte, specific binding is sufficiently discriminatory when determinative of the presence of the analyte in a heterogeneous (inhomogeneous) sample. Typically, the affinity or avidity of a specific binding reaction is at least about 10⁻⁴ M, with specific binding reactions of greater specificity typically having affinity or avidity of at least 10⁻⁶ M to at least about 10⁻¹² M. It may also refer to binding to self, or other molecules of the same protein, as in the forming of dimers and other multimers. Selective binding might also be generally described as specific binding, but may also be used for example to connote a use in a discriminatory separation, diagnostic, or identification technique or a discriminatory property beyond simply recognizing the presence of the binding target in a sample—for example an antibody may be selective for different members of a closely related protein family, for specific modified forms of a protein (e.g., a phosphorylated form vs. a non-phosphorylated form), or specific conformations of a protein (e.g., PrP^{C} vs. PrP^{Sc}). Specific and/or selective binding may also be described as “recognition” or “recognizing” of a molecule by a binding molecule.

“Small molecule” typically refers to a non-peptidic molecule that has a low molecular weight, often, though not always, between 1 dalton and 5 kilodaltons (kDa). Small molecules may penetrate cell membranes and the blood brain barrier more easily than larger molecular weight compounds such as proteins, peptides and carbohydrates. Small molecules generally need to be less than 600 daltons to pass the blood brain barrier. Typically small molecules are produced through chemical reactions or synthesis, though this is not always the case, and they rarely provoke an immune response. Preferably small molecules of the invention are less than 5 kDa, more preferably they are less than 1 kDa, Most preferably they are less than 600 daltons.

The term “substantial identity” (also “substantial amino acid sequence identity”, “substantial nucleic acid sequence identity”, “substantial sequence identity”, and the like) is used herein to refer to a sequence that, when optimally aligned, for example using the methods described above, share at least 80%, 85%, 90%, 95%, 96%, 97%, 98%, 99%, or 100% sequence identity with a TAT-028 polypeptide or nucleic acid. “Substantial identity” may be used to refer to various types and lengths of sequence, such as full-length sequence, epitopes or immunogenic peptides, functional domains, coding and/or regulatory sequences, exons, introns, promoters, and genomic sequences. Some non-limiting examples and methods may be found in Bazan et al. (1989) Proc Natl Acad Sci USA. 86: 9642-9646; Simmer et al. (1990) J Biol Chem. 265: 10395-10402; Storm and Sonnhammer (2001) Bioinformatics 17: 343-348; Kong and Ranganathan (2004) Brief Bioinform. 5: 179-192; Sonnhammer and Kahn (1994) Protein Sci. 3: 482-492; and Yamaguchi et al. (2002) Plant Cell 14: 2957-2974. Substantial identity may be a more appropriate standard than percent sequence identity for short sequences, such as peptide antigens that can potentially be used to confer an immune response with specificity for TAT-028, but that, because of their short length, easily fall below the desired percent sequence identity with minor alterations, such as conservative amino acid substitutions, that may have little or no impact on the function as a TAT-028 immunogen. Substantial identity also encompasses the use of cryptic epitopes, such as for mimicking the antigenicity of TAT-028.

“TAT-028 binding protein” refers to a molecule, multimer, composition, or complex that is, at least in part, peptidic, comprising at least 4 or more amino acids, that binds a TAT-028 polypeptide. Preferably, the TAT-028 binding protein binds the TAT-028 protein (SEQ ID NO: 19), such as the denatured protein, but most preferably the native protein or its naturally modified forms. Preferably such binding is specific, and more preferably it is selective. Binding may occur anywhere on the TAT-028 molecule, including in discrete epitopes such as ones recognized in the TAT-028 peptides described herein (SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17. “TAT-028 binding protein” may also refer to a collection of binding proteins such as a polyclonal antibody. A TAT-028 binding protein may be, for non-limiting example, an antibody, antibody-related peptide, one or more CDR regions of a TAT-028 binding antibody, or TAT-028 interacting protein.

“TAT-028 binding molecule” encompasses TAT-028 binding proteins, but also includes non-peptidic molecules and compositions including, but not limited to, those generally described as small molecules.

By “therapeutically effective immune response” is meant an immune response which is effective in treating a disease, particularly a neoplasm.

“Therapeutic moiety” refers to a moiety covalently or non-covalently bound to one or more macromolecules of interest, for example an antibody. Such binding may be direct or indirect, such as through a linker region. The moiety should have a known therapeutic effect, or potentially so, at the cellular, tissue, organ, systemic, or organismal level.

“Transcriptional regulatory elements” refers to nucleic acid sequences that regulate transcription. For example, not intended to be limiting, promoters, polyadenylation signals, start codons, and stop codons.

“Translational regulatory elements” refers to nucleic acid sequences that regulate translation. Non-limiting examples of translational regulatory elements include start codons, ribosome binding regions, polyadenylation signals, and stop codons.

“Transform” refers to the introduction of a polynucleotide (e.g., single or double stranded DNA, RNA, or a combination thereof) into a living cell by any means. Transformation may be accomplished by a variety of methods, including, but not limited to, electroporation, polyethylene glycol mediated uptake, particle bombardment, agrotransformation, and the like. This process may result in transient or stable expression of the transformed polynucleotide. By “stably transformed” is meant that the sequence of interest is integrated into a replicon in the cell, such as a chromosome or episome. Transformed cells encompass not only the end product of a transformation process, but also the progeny thereof which retain the polynucleotide of interest.

“Transgenic” refers to any cell, spore, tissue or part, or higher organism such as a plant or animal (for example, a mouse) that contains all or part of at least one recombinant polynucleotide. In many cases, all or part of the recombinant polynucleotide is stably integrated into a chromosome or stable extra-chromosomal element, so that it is passed on to successive generations.

“Treating” and “treatment” refer to reduction in severity, progression, spread, and/or frequency of symptoms, elimination of symptoms and/or underlying cause, prevention of the occurrence of symptoms and/or their underlying cause, and improvement or remediation of damage. “Treatment” is meant to include therapeutic treatment as well as prophylactic, or suppressive measures for the disease or disorder. Thus, for example, “treating” a patient involves prevention of a particular disorder or adverse physiological event in a susceptible individual as well as treatment of a clinically symptomatic individual by inhibiting or causing regression of a disorder or disease. The term “treatment” includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease. Further, administration of the agent after onset and after clinical symptoms have developed where administration affects clinical parameters of the disease or disorder and perhaps amelioration of the disease, comprises “treatment” of the disease. The present method of “treating” a patient in need of anti-cancer therapy encompasses both prevention of a condition, disease, or disorder that is responsive to anti-cancer therapy and treatment of a condition, disease, or disorder that is responsive to anti-cancer therapy in a clinically symptomatic individual.

“Uniquely matching peptides” refers to peptide sequences which are contained within the amino acid sequence of proteins from the same homology cluster, where the homology cluster contains proteins which are 95% homologous over 50% of their length.

“Vaccine” refers to one or more immunogens that could be used to stimulate the production of antibodies, such as in inducing or enhancing an immune response to the immunogen that is effective in the prevention of disease, or in the treatment of disease associated with a pre-existing infection when administered to a patient. The immunogen(s) may be present in a variety of media including, but not limited to, serum or supernatant, or in purified form.

“Virus-based vector” refers to a recombinant agent for transferring genetic material, such as DNA or RNA, into a cell altered from one or more viruses or a prior altered version thereof. “Virus” generally refers to any of a large group of submicroscopic infective agents that are regarded either as extremely simple microorganisms or as extremely complex molecules, that typically contain a protein coat surrounding an RNA or DNA core of genetic material but no semi-permeable membrane, that are capable of growth and multiplication only in living cells, and that cause various diseases in humans, animals, or plants. Some, but not the only, examples are adenovirus, influenza, HIV, DNA tumor viruses, polio, and retroviruses. Exemplary vectors (not intended as limiting) may be found in Gene Transfer and Expression in Mammalian Cells Savvas C. Makrides (Ed.), Elsevier Science Ltd, 2003.

“Xenologue” refers to a homologous and/or analogous protein or amino acid sequence or a homologous and/or analogous nucleic acid sequence present in another species. Most commonly herein xenologue would refer to a non-human TAT-028 polypeptide or nucleic acid. Xenologues may be identified based on substantial sequence identity or via other methods, such as phenotypic screening for analogues. Preferably a xenologue is an analogue, related by function as may be assessable by complementation in a deficient or knockout model strain, and preferably it is homologous. Preferably it is a paralogue, one or more sequences from the other species that shares a direct common ancestor with a TAT-028 sequence, more preferably a paralogue related by both homology and function. Most preferably it is a likely orthologue, the corresponding gene in the other species sharing a direct common ancestor with a TAT-028 sequence, as may be evidenced by homology, analogy, synteny, and other models of evolutionary analysis. For some time after a speciation event this relationship is often easily inferred and cleanly defined since the two genes differ only modestly, however paralogues and orthologues can be difficult to distinguish as differences accumulate between the related sequences. Xenologues have uses in producing animal models such as transgenics and knockouts. They may also be used in screening efforts or efforts to produce binding molecules such as antibodies that take advantage of their sequence similarities, or, on occasion, their sequence differences, such as when screening for pan-species binding antibodies.

Discovery of TAT-028 and its Association with Cancer, and Uses Therefrom

The present inventors have discovered TAT-028 peptides, (SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17), that were found to be overexpressed in tumor samples. These TAT-028 peptides uniquely match the amino acid sequence encoding the TAT-028 protein, leading to the discovery that increased expression of TAT-028 protein in human patients is associated with lung tumors as compared to adjacent normal tissue and that the overexpressed protein is in plasma membrane fractions (see Example 4). Thus, the present inventors have discovered that TAT-028 is associated with abnormal development and growth, and may be useful in further studying the mechanisms of cancer, and as a target for the identification of potential anti-cancer compounds, including antibodies for use in immunotherapy. Accordingly, the present invention provides methods for the identification of compounds that modulate TAT-028 (polypeptide or nucleic acid) expression or activity. These methods include contacting a candidate compound with a TAT-028 and detecting the presence or absence of binding between the compound and the TAT-028, or detecting a change in TAT-028 expression or activity. Methods are also included for the identification of active agents, such as small molecules or antibodies, that inhibit TAT-028 expression or activity. Such methods include administering a compound to a cell or cell population, and detecting a change in TAT-028 expression or activity. The methods and compositions of the invention are also useful for the identification of anti-cancer compounds.

Although any methods, devices, and materials similar or equivalent to those described herein can be used in the practice or testing of the invention, the preferred methods, devices and materials are now described.

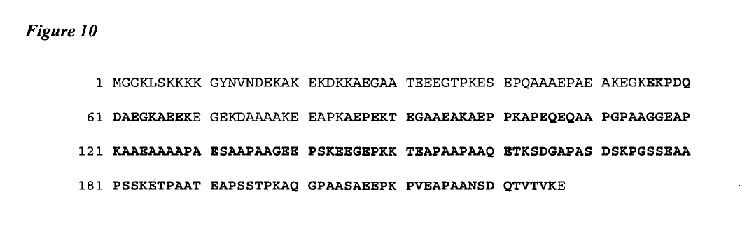

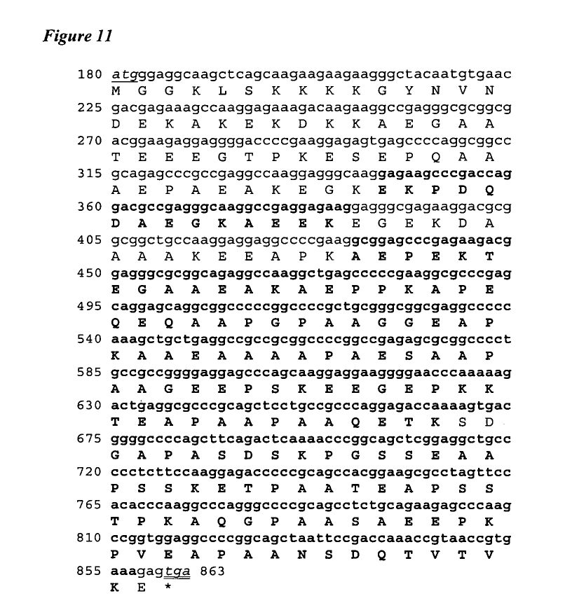

The cDNA of the TAT-028 mRNA coding sequence (SEQ ID NO: 20 and FIG. 11; full length mRNA SEQ ID NO: 21) encoding the TAT-028 protein (SEQ ID NO: 19 and FIG. 10), and a genomic DNA sequence (SEQ ID NO: 22) encoding the TAT-028 locus, can be found herein, as well as the amino acid sequences of the peptides used in the identification of TAT-028 (SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, and SEQ ID NO: 17, see also FIG. 10) and a corresponding nucleic acid sequence (SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NOS: 50-58, and SEQ ID NO: 18).

It would be obvious to one of skill in the art to use sequences in the methods of the invention that differ from the TAT-028 sequences disclosed herein (e.g., SEQ ID NO: 19, SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, and SEQ ID NO: 17), but that have substantial similarity to a TAT-028 sequence, whether naturally occurring, produced through methods of mutagenesis, such as random mutagenesis, or engineered for various reasons. Preferably such sequences have substantial sequence identity to a TAT-028 sequence and one of skill in the art should be able to determine the lengths of TAT-028 and candidate TAT-028 sequences appropriate for comparison. In general, it is preferred that the % similarity or % identity is determined over the portion relevant to the use at hand (e.g., over the kinase domain for a fragment to be used in a kinase assay), but is preferably over at least about 80% of the full length protein. Such sequences that are substantially similar or identical to a TAT-028 disclosed herein, or are derived from the same genetic locus or using a TAT-028 or fragment thereof as starting material, are considered compositions of the invention (TAT-028 polypeptides or TAT-028 polynucleotides as appropriate) and useful in the methods of the invention. Particularly preferred variant TAT-028 polypeptides or TAT-028 nucleotides are those derived from a cell with a cellular proliferative disease, such as a lung cancer.

Preferably such TAT-028 sequences also have one or more additional characteristics of a TAT-028 sequence, for example an activity analogous to that of a TAT-028 sequence. The degree of activity as compared to TAT-028 sequences disclosed herein may vary with the intended use, and appropriate degrees of activity may be determined by one skilled in the art. In general, however, null alterations, sequences lacking a given activity or having reduced activity with alterations (insertions, deletions, or substitutions) to the sequence as compared to the disclosed TAT-028 sequence, whether naturally occurring, produced through methods of mutagenesis, such as random mutagenesis, or engineered are desired. Null alterations may be useful as controls for the activity and delimiters of its likely range in TAT-028 protein. For non-null alterations, in general, it is preferable that the degree of activity be within three orders of magnitude of that of the TAT-028 sequences disclosed herein. More preferably the degree of activity non-null alterations will be within two orders of magnitude of that of the TAT-028 sequences disclosed herein. Most preferably the degree of activity non-null alterations will be within one order of magnitude of that of the TAT-028 sequences disclosed herein. It may also be preferable in some cases for a non-null alteration to be “super-active” and exceed the activity of the TAT-028 sequences disclosed herein by 4, 5, 6, or more orders of magnitude. The activity to be measured for comparison or screened for among a library of TAT-028s, such as a library of mutagenized sequences, may be any activity relevant to use in the methods of the invention, such as a characteristic of a TAT-028 that will be as a variable in, or a criterion for, assessing the outcome of a screening method. A preferred activity for comparison is immunogenicity.

Thus, point mutations, polymorphisms, splice variants, mutagenized sequences, transcription and/or translation optimized sequences, recombinant variants, modifications, derivatives, fusions, fragments, homologues, and combinations thereof that constitute TAT-028 sequences of the invention can be determined through percent sequence similarity, or preferably percent sequence identity, to a TAT-028 sequence disclosed herein (e.g., SEQ ID NO: 19), or through knowledge of the sequence's origin in a TAT-028 genetic locus or origin in a process deriving it from a TAT-028 sequence. And, preferably such sequences exhibit one or more activities of a TAT-028.

Nucleic Acids

Nucleic acids of the invention have a variety of uses, including, but not limited to, detecting and quantitating TAT-028 gene expression for diagnostic and prognostic purposes; expressing TAT-028 polypeptides; screening for modulators of TAT-028 expression, therapeutic applications such as anti-sense vectors or ribozymes; and for producing transgenic or knockout animal model systems for drug screening and testing. TAT-028 nucleic acid sequences can be initially identified by substantial nucleic acid sequence identity to the TAT-028 nucleic acid sequences described herein (e.g., SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NOS: 50-58, SEQ ID NO: 18, SEQ ID NO: 20, SEQ ID NO: 21, and SEQ ID NO: 22) or by their encoding a protein of substantial amino acid sequence identity to the TAT-028 polypeptide sequences described herein (e.g., SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 38, SEQ ID NO: 39, SEQ ID NO: 40, SEQ ID NO: 41, SEQ ID NO: 42). Such homology can be based on the overall nucleic acid or amino acid sequence, and is generally determined as outlined below, using an assessment of homology, such as, for example, may be provided by sequence alignment software, such as a BLAST program (Basic Local Alignment Search Tool; (Altschul et al. (1990) J Mol Biol 215: 403-410), NCBI BLAST2.0 software as defined by Altschul et al. (1997) Nucleic Acids Res. 25: 3389-3402, using Smith Waterman Alignment (Smith and Waterman (1981) J Mol Biol 147: 195-197) as incorporated into GeneMatcher Plus™, or, preferably bl2seq (Tatusova and Madden (1999) FEMS Microbiol Lett. 174: 247-250), or through nucleic acid hybridization conditions.

TAT-028 nucleic acids also include polynucleotides comprising TAT-028 regulatory and structural nucleic acid sequences or fragments thereof, including TAT-028 genomic sequence (e.g., SEQ ID NO: 22), introns, mRNA untranslated regions, and promoters, and nucleic acids with substantial nucleic acid sequence identity thereto. Such nucleic acid sequences are useful, for example, for generating knockout and transgenic animal models, or for screening for modulators of TAT-028 expression. TAT-028 nucleic acids also include transcription and translation optimized sequences, such as those produced through codon optimization and IRES (internal ribosomal entry site) incorporation, or tandem or concatameric sequences, which may be useful for example in expressing and purifying the protein.

TAT-028 nucleic acids may be fragments of more extensive TAT-028 nucleic acids including polynucleotides encoding fragments of TAT-028 polypeptides (e.g., SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NOS: 50-58, SEQ ID NO: 18). Encoding polynucleotides may include non-coding sequences (e.g., SEQ ID NO: 21 and SEQ ID NO: 22) and may be of as few as 10 contiguous nucleotides. They may encode TAT-028 polypeptide fragments comprising 20, 30, 40, 50, 60, 70, 80, 90, 100, 110, 120, 130, 140, 150, 200, 205, 210, 220 or at least 227 amino acids or more contiguous amino acids of a TAT-028 polypeptide. Such fragments may be used, for example, as primers for PCR, as probes in hybridization, in screening for binders to the nucleic acid or modulators of its expression, or in expressing peptidic fragments of TAT-028, etc.

The invention further provides for TAT-028 nucleic acids comprising polynucleotides substantially complementary to all or part of the TAT-028 nucleic acids, for example an anti-sense fragment complementary to bases 26-78 of the TAT-028 mRNA coding sequence (SEQ ID NO: 20). Thus, for example, both strands of a double stranded nucleic acid molecule are included in the present invention (whether or not they are associated with one another), such as dual strands of DNA, but also including double-stranded RNA, and DNA/RNA hybrids. Also included are mRNA molecules and complementary DNA molecules (e.g., cDNA molecules). Substantially complementary sequences should be complementary enough to hybridize to the corresponding TAT-028 nucleic acid under normal reaction conditions, particularly high, or moderate stringency hybridization conditions. A variety of hybridization conditions may be used in the present invention, including high, moderate and low stringency conditions. High stringency conditions are known in the art; see for example Maniatis et al. Molecular Cloning: A Laboratory Manual, 2nd Edition (1989), and Short Protocols in Molecular Biology, ed. Ausubel, et al., (1989) both of which are hereby incorporated by reference. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993).Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Moderate or low stringency conditions may also be used, as are known in the art; see Maniatis and Ausubel, supra, and Tijssen, supra. Complementary nucleic acids may be useful as probes in hybridization, in vectors comprising double-stranded DNA molecules, or in modulating TAT-028 expression through use of anti-sense, RNAi, or ribozymes, etc.

As used herein, “highly stringent conditions” means hybridization to filter-bound DNA in 0.5 M NaHP0₄, 7% sodium dodecyl sulphate (SDS), 1 mM EDTA at 65° C., and washing in 0.1× SSC/0.1% SDS at 68° C. For some applications, less stringent conditions for duplex formation are required. As used herein “moderately stringent conditions” means washing in 0.2 x SSC/0.1% SDS at 42° C. (Ausubel et al. (1989), supra). Hybridization conditions can also be rendered more stringent by the addition of increasing amounts of formamide, to destabilize the hybrid duplex. Thus, particular hybridization conditions can be readily manipulated, and will generally be chosen depending on the desired results. In general, convenient hybridization temperatures in the presence of 50% formamide are: 42° C. for a probe which is 95 to 100% identical to the fragment of a TAT-028 nucleic acid molecule or a nucleic acid molecule encoding a TAT-028 polypeptide as defined herein, 37° C. for 90 to 95% identity and 32° C. for 70 to 90% identity.

Additional TAT-028 nucleic acids, including homologues, paralogues, and orthologues from species other than human, may be obtained using standard cloning techniques, screening techniques, or homology search techniques. For example, a cDNA library derived from mRNA in murine cells, using expressed sequence tag (EST) analysis (Adams et al. (1991) Science 252: 1651-1656; Adams et al. (1992) Nature 355: 632-634; Adams et al. (1995) Nature 377: (6547 Suppl): 3-174) could be probed by BLAST homology search ((Altschul et al. (1997) Nucleic Acids Res. 25: 3389-3402; Altschul et al. (1990) J Mol Biol. 215: 403-410)) to identify TAT-028 homologues. Alternatively, a murine cDNA library might be screened using a human TAT-028 cDNA under low stringency conditions. Additional TAT-028 nucleic acids may also be obtained from natural sources such as genomic DNA libraries or can be synthesized using well known and commercially available techniques.

One skilled in the art will understand that, in many cases, an isolated cDNA sequence will be incomplete, in that the region coding for the polypeptide is cut short at the 5′ end of the cDNA. This is often a consequence of reverse transcriptase, an enzyme with inherently low processivity (a measure of the ability of the enzyme to remain attached to the template during the polymerization reaction), failing to complete a DNA copy of the mRNA template during 1^{st} strand cDNA synthesis. Using the sequences provided herein, additional TAT-028 nucleic acid sequences may be obtained by using techniques well known in the art for either extending sequences or obtaining full length sequences (see Maniatis et al., and Ausubel, et al., supra), for example, RACE (Rapid amplification of cDNA ends; e.g., Frohman et al. (1988) Proc Natl Acad Sci U.S.A. 85: 8998-9002) and modifications to RACE (exemplified by the MarathonT Technology of Clontech Laboratories Inc.). Indeed, PCR techniques may be used to amplify any desired TAT-028 nucleic acid sequence. Thus the sequence data for TAT-028 nucleic acids, such as is provided herein, can be used to design primers for use in PCR so that a desired TAT-028 sequence can be targeted and then amplified to a high degree. Typically, primers will be at least five nucleotides long and will generally be at least ten nucleotides long (e.g., fifteen to twenty-five nucleotides long). In some cases, primers of at least thirty or at least thirty-five nucleotides in length may be used. As a further alternative, chemical synthesis which may be automated may be used. Relatively short sequences may be chemically synthesized and ligated together to provide a longer sequence.

Unless the context indicates otherwise, TAT-028 nucleic acid molecules may have one or more of the following characteristics: 1) they may be DNA or RNA; 2) they may be single or double stranded; 3) they may be provided in recombinant form, e.g., covalently linked to a 5′ and/or a 3′ flanking sequence to provide a molecule which does not occur in nature; 4) they may be provided without 5′ and/or 3′ flanking sequences which normally occur in nature; 5) they may be provided in substantially pure form. Thus, they may be provided in a form which is substantially free from contaminating proteins or other nucleic acids; and 6) they may be provided with or without introns (e.g., as cDNA). The nucleic acid molecule may be in recombinant or chemically synthetic form. Preferably, the nucleic acid is in isolated form.

Manipulation of the nucleic acid encoding a TAT-028 polypeptide can be used to produce both modified proteins and for generating large quantities of protein for purification purposes. TAT-028 polypeptide derivatives can be created by introducing one or more nucleotide substitutions, additions or deletions into the nucleotide sequence of a TAT-028 nucleic acid such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues. Random mutagenesis may even be used to produce a library of modified TAT-028 proteins (see for example Xu et al. (1999) Biotechniques 27: 1102-4, 1106, 1108; Lin-Goerke et al. (1997) Biotechniques 23: 409-412; Fromant et al. (1995) Anal Biochem. 224: 347-53; Fujii et al. (2004) Nucleic Acids Res. 32(19): e145; Chusacultanachai and Yuthavong (2004) Methods Mol Biol. 270: 319-34).

Vectors

The invention also relates to recombinant vectors, such as recombinant vectors, which include one or more TAT-028 nucleic acids, as well as host cells containing the vectors or which are otherwise engineered to contain or express TAT-028 nucleic acids or polypeptides, and methods of making such vectors and host cells and their use in production of TAT-028 polypeptides by recombinant or synthetic techniques.

In one embodiment, the polynucleotides of the invention are joined to a vector (e.g., a cloning or expression vector. The vector may be, for example, a phage, plasmid, or viral vector. Viral vectors may be replication competent or replication defective. In the latter case, viral propagation generally will occur only in complementing host cells. The polynucleotides may be joined to a vector containing a selectable marker for propagation in a host. Introduction of the vector construct into the host cell can be effected by techniques known in the art which include, but are not limited to, calcium phosphate transfection, DEAE-dextran mediated transfection, cationic lipid-mediated transfection, electroporation, transduction, infection or other methods. Such methods are described in many standard laboratory manuals, such as Davis et al. (1986) Basic Methods in Molecular Biology.

i.) Expression Vectors

TAT-028 nucleic acids that include sequences encoding TAT-028 polypeptides can be used for the recombinant production of the TAT-028 polypeptides. The TAT-028 nucleic acids may include the coding sequence for the mature polypeptide alone, or the coding sequence for the mature polypeptide in reading frame with other coding sequences, such as those encoding a leader or secretory sequence, a pre-, pro- or prepro-protein sequence, a cleavable sequence (e.g., a cleavable GST fusion protein) or other fusion peptide portions, such as an affinity tag or an additional sequence conferring stability during production of the polypeptide. Preferred affinity tags include, but are not limited to, multiple histidine residues (for example see Gentz et al. (1989) Proc Natl Acad Sci U.S.A. 86: 821-824), a FLAG tag, HA tag, or myc tag. The TAT-028 nucleic acids may also contain non-coding 5′ and 3′ sequences, such as transcribed, non-translated sequences, splicing and polyadenylation signals, ribosome binding sites and sequences that stabilize mRNA. The TAT-028 polypeptides may be produced by culturing a host cell transformed with an expression vector containing a TAT-028 nucleic acid encoding a TAT-028 polypeptide under the appropriate conditions to induce or cause expression of the TAT-028 polypeptide. The conditions appropriate for TAT-028 polypeptide expression will vary with the choice of the expression vector and the host cell and may be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest of the polypeptide from the host cell is important (e.g., the baculoviral systems used in insect cell expression are lytic viruses, and thus harvest time selection can be crucial for product yield).

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), α-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide (tag) imparting desired characteristics, for example, stabilization or simplified purification of expressed recombinant product. In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

In addition, the expression vector may comprise additional elements. For example, the expression vector may have two replication systems, thus allowing it to be maintained in two organisms, for example in mammalian or insect cells for expression and in a procaryotic host for cloning and amplification. In another example, the vector is an integrating expression vector in which the expression vector contains at least one sequence homologous to the host cell genome, and preferably two homologous sequences which flank the expression construct. The integrating expression vector may be directed to a specific locus in the host cell by selecting the appropriate homologous sequence for inclusion in the vector. Constructs for integrating expression vectors are well known in the art.

In one embodiment, the DNA of the invention is operatively associated with an appropriate heterologous regulatory element (e.g., promoter or enhancer), such as, the phage lambda PL promoter, the E. coli lac, trp, phoA, and tac promoters, the SV40 early and late promoters and promoters of retroviral LTRs. Promoter sequences generally encode either constitutive or inducible promoters. The promoters may be either naturally occurring promoters or hybrid promoters, with a combination of elements from more than one promoter. Other suitable promoters will be known to the skilled artisan.

Useful expression vectors for bacterial use can comprise a selectable marker and bacterial origin of replication derived from commercially available plasmids comprising genetic elements of the well-known cloning vector pBR322 (ATCC 37017). Such commercial vectors include, for example, pKK223-3 (Pharmacia Fine Chemicals, Uppsala, Sweden) and GEM1 (Promega Biotec, Madison, Wis., USA). These pBR322 “backbone” sections are combined with an appropriate promoter and the structural sequence to be expressed. Among vectors preferred for use in bacteria include pHE4-5 (ATCC Accession No. 209311; and variations thereof), pQE70, pQE60 and pQE-9, available from QIAGEN, Inc., supra; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Preferred expression vectors for use in yeast systems include, but are not limited to, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, pPIC9K, and PAO815 (all available from Invitrogen, Carlsbad, Calif.). Among preferred eukaryotic vectors are pWLNEO, pSV2CAT, pOG44, pXT1 and pSG available from Stratagene, and pSVK3, pBPV, pMSG and pSVL (available from Pharmacia). Other suitable vectors will be readily apparent to the skilled artisan.

Additional expression vectors useful in any of the methods of the invention include retrovirus vectors (e.g., as described in WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), parvovirus based vectors such as adeno-associated virus (AAV) vectors, and adenoviral vectors (e.g., those described in WO 94/12649, WO 93/03769; WO 93/19191; WO 94/28938; WO 95/11984 and WO 95/00655). Administration of DNA linked to kill adenovirus as described in Curiel (1992) Hum Gene Ther. 3: 147-154 may be employed.

Other gene delivery vehicles and methods may be employed including, liposomes; polycationic condensed DNA linked or unlinked to killed adenovirus alone; eukaryotic cell delivery vehicle cells; deposition of photopolymerized hydrogel materials; hand-held gene transfer particle gun; ionizing radiation; mechanical delivery systems such as the approach described in Woffendin et al. (1994) Proc Natl Acad Sci. U.S.A. 91: 11581-11585; naked DNA; biodegradable latex beads to improve uptake efficiency; and/or nucleic charge neutralization or fusion with cell membranes. Additional approaches are described in Philip (1994) Mol Cell Biol. 14: 2411-2418, and in Woffendin (1994) Proc Natl Acad Sci. U.S.A. 91: 1581-1585.

ii.) Other Vectors

TAT-028 nucleic acids may also be used in other vectors known in the art including but not limited to vectors for producing gene disruptions (“knockouts”), other transgenic modifications (“knockins”), anti-sense vectors, RNAi vectors, gene therapy vectors, and vectors for assessing or utilizing TAT-028 promoter activity.

TAT-028 nucleic acids and vectors comprising TAT-028 may also be used for screening compounds for candidate agents that can modulate TAT-028 expression. For example, a library of mammalian transcription factors can be screened against a vector containing the TAT-028 promoter operably linked to a reporter gene sequence to determine transcription factors capable of modulating expression from the TAT-028 promoter. For example, a yeast one-hybrid system (Clontech, Palo Alto, Calif.) (Wang and Reed (1993) Nature 364: 121-126; Strubin et al. (1995) Cell 80: 497-506; Lehming et al. (1994) Nature 371: 175-179; Li et al. (1993) Science 262: 1870-1873; Luo et al. (1996) Biotechniques. 20: 564-568; Gstaiger et al. (1995) Nature 373: 360-362) or variations thereupon may be used to isolate transcription factors binding the TAT-028 promoter, or, for example, a CAT reporter system may be used to assess small molecule impact on expression from the TAT-028 promoter.

iii.) Host Cells

Host cells useful for the expression of TAT-028 nucleic acids can be a higher eukaryotic cell, such as a mammalian cell (e.g., a human derived cell), or a lower eukaryotic cell, such as a yeast cell, or the host cell can be a prokaryotic cell, such as a bacterial cell. Examples of appropriate hosts include, but are not limited to, bacterial cells, such as E. coli, Bacillis subtilis, Salmonella typhimurium, and various species within the genera Pseudomonas, Streptomyces, and Staphylococcus); archaebacteria; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, C129 cells, Neurospora, BHK, HeLa cells, THP1 cell line (a macrophage cell line), Bowes melanoma cells, and human cells and cell lines; and plant cells. Appropriate culture mediums and conditions for the above-described host cells are known in the art.

The host strain may be one which modulates the expression of the inserted gene sequences, or modifies and processes the gene product in the specific fashion desired. Expression from certain promoters can be elevated in the presence of certain inducers; thus, expression of the genetically engineered polypeptide may be controlled. Furthermore, different host cells have characteristics and specific mechanisms for the translational and post-translational processing and modification (e.g., phosphorsylation and cleavage) of proteins. Appropriate cell lines can be chosen to ensure the desired modifications and processing of the foreign protein expressed. Selection of appropriate vectors and promoters for expression in a host cell is a well-known procedure and the requisite techniques for expression vector construction, introduction of the vector into the host, and expression in the host are routine skills in the art.

Following transformation of a suitable host strain and growth of the host strain to an appropriate cell density, the selected promoter is induced by appropriate means (e.g., temperature shift or chemical induction) if necessary or desired, and cells are cultured for an additional period. Cells are typically harvested by centrifugation, disrupted by physical or chemical means, and the resulting crude extract retained for further purification.

Host cells employed in expression of proteins can be disrupted by any convenient method, including freeze-thaw cycling, sonication, mechanical disruption, or use of cell lysing agents. Such methods are well known to those skilled in the art.

Therapeutic Nucleic Acids

Symptoms of cancer may be ameliorated by decreasing the level or activity of a TAT-028 polypeptide or nucleic acid by using TAT-028 nucleic acid sequences as defined herein in conjunction with well-known gene “knock-out,” anti-sense, RNAi, ribozyme, or triple helix methods to decrease gene expression. In this approach, ribozyme or triple helix molecules are used to modulate the activity, expression or synthesis of the gene, and thus to ameliorate the symptoms of cancer. Such molecules may be designed to reduce or inhibit expression of a mutant or non-mutant target gene. Such techniques are well known to those of skill in the art.

i.) Anti-Sense and RNAi

The invention also provides for the use of at least one TAT-028 nucleic acid in the preparation of a pharmaceutical composition for use in the treatment of cancer, preferably a lung cancer or metastases therefrom. In a specific embodiment, TAT-028 nucleic acid molecules are used as anti-sense molecules or as molecules for RNA interference (RNAi), to alter the expression of TAT-028 polypeptides by binding to and/or triggering the destruction of TAT-028 nucleic acids and thus may be used in the treatment or prevention of cancer. Anti-sense nucleic acids of the invention include TAT-028 nucleic acids capable of hybridizing through sequence complementary to a portion of a TAT-028 RNA, preferably a TAT-028 mRNA encoding a TAT-028 polypeptide. The anti-sense nucleic acid can be complementary to a coding and/or non-coding region of an mRNA encoding such a polypeptide. Most preferably, expression of a TAT-028 nucleic acid or polypeptide or both is inhibited by use of anti-sense nucleic acids. Complementary to a nucleotide sequence in the context of antisense oligonucleotides and methods therefore means sufficiently complementary to such a sequence as to allow hybridization to that sequence in a cell, i.e., under physiological conditions. Preferably such sequences are at least 40% complementary to a TAT-028 nucleic acid, or at least 50%, or at least 60%, more preferably the percent complementarity is at least 70%, most preferably the percent complementarity is at least 80% or 90 or 95 or 99% complementary to a TAT-028 nucleic acid, or any integer value from 40-100% complementarity in ascending order. Antisense oligonucleotides preferably comprise a sequence containing from about 8 to about 100 nucleotides, more preferably the antisense oligonucleotides comprise from about 15 to about 30 nucleotides. Antisense oligonucleotides can also contain a variety of modifications for example, modified internucleoside lineages (Uhlmann and Peyman (1990) Chemical Reviews 90: 543-548; Schneider and Banner (1990) Tetrahedron Lett. 31: 335); modified nucleic acid bases as disclosed in U.S. Pat. No. 5,958,773 and patents disclosed therein; and/or sugars and the like. Preferred modifications are those that confer resistance to nucleolytic degradation.

Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050, and 5,958,773. Modifications can include natural and non-natural oligonucleotides, both modified (e.g., phosphorothiates, phosphorodithiates, and phosphotriesters) and unmodified, oligonucleotides with modified (e.g., morpholino linkages and heteroatom backbones) or unmodified backbones, as well as oligonucleotide mimetics such as Protein Nucleic Acids, locked nucleic acids, and arabinonucleic acids. Numerous nucleobases and linkage groups may be employed in the nucleobase oligomers of the invention, including those described in U.S. Patent Application Nos. 20030114412 and 20030114407, incorporated herein by reference.

The antisense compounds of the invention can include modified bases. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758; 5,565,552; 5,567,810; 5,574,142; 5,585,481; 5,587,371; 5,597,696 and 5,958,773.

Chimeric antisense oligonucleotides are also within the scope of the invention, and can be prepared from the present inventive oligonucleotides using the methods described in, for example, U.S. Pat. Nos. 5,013,830; 5,149,797; 5,403,711; 5,491,133; 5,565,350; 5,652,355; 5,700,922 and 5,958,773.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. mRNA levels in the cell can be measured routinely in treated and control cells by reverse transcription of the mRNA and assaying the cDNA levels. The biological effect can be determined routinely by measuring cell growth or viability as is known in the art. Measuring the specificity of antisense activity by assaying and analyzing cDNA levels is an art-recognized method of validating antisense results. It has been suggested that RNA from treated and control cells should be reverse-transcribed and the resulting cDNA populations analyzed (Branch (1998) Trends Biochem Sci. 23: 45-50).

The invention further embraces the use of interfering RNA (RNAi) to disrupt TAT-028 expression. This can be accomplished by various means. For example, in one method all or a portion of the targeted gene can be incorporated into a vector and used to target desired cells, e.g., lung cancer cells. RNAi can be used to collectively refer to several gene silencing techniques, including the use of siRNA (short interfering RNAs), shRNA (short hairpin RNA—an RNA bearing a fold-back stem-loop structure), dsRNA (double-stranded RNA, itself on occasion used to encompass any double-stranded RNA, but also used in this section to discuss double-stranded RNAs of greater length than, for instance, siRNAs as a class, in particular because longer double-stranded RNAs are more likely to activate non-specific host responses to double-stranded RNA (see, for example, Williams (1997) Biochem Soc Trans. 25: 509-513; Gil and Esteban (2000) Apoptosis 5: 107-114; Clarke and Mathews (1995) RNA. 1: 7-20; Baglioni and Nilsen (1983) Interferon. 5: 23-42)), miRNA (micro RNAs), stRNAs (short (or “small”) temporal RNAs), and the like. RNA interference is a mechanism to suppress gene expression in a sequence specific manner. See, for example, Brumelkamp et al. (2002) Sciencexpress (Mar. 21, 2002); Sharp (1999) Genes Dev. 13: 139-141; and Cathew (2001) Curr Op Cell Biol. 13: 244-248; Zamore et al. (2000) Cell 101: 25-33; Bass (2001) Nature 411: 428-429; Elbashir et al. (2001) Nature 411: 494-498; PCT Publication Nos. WO 00/44895; WO 01/36646; WO 99/32619; WO 00/01846; WO 01/29058; WO 99/07409; and WO 00/44914; Allshire (2002) Science 297: 1818-1819; Volpe et al. (2002) Science 297: 1833-1837; Jenuwein (2002) Science 297: 2215-2218; and Hall et al. (2002) Science 297: 2232-2237; Hutvagner and Zamore (2002) Science 297: 2056-60; McManus et al. (2002) RNA. 8: 842-850; Reinhart et al. (2002) Genes Dev. 16: 1616-1626; and Reinhart and Bartel (2002) Science 297:1831.e

In certain embodiments of the invention, TAT-028 nucleic acids can be, or will be used as guide sequences to produce, RNAi molecules of the invention which comprise sense and antisense sequences or regions, wherein the sense and antisense regions are generally covalently linked by nucleotide or non-nucleotide linker molecules as is known in the art, or are alternately non-covalently linked by ionic interactions, hydrogen bonding, van der waals interactions, hydrophobic intercations, and/or stacking interactions. In mammalian cells, short, e.g., 21 nt, double stranded small interfering RNAs (siRNA) have been shown to be effective at inducing an RNAi response. See, e.g., Elbashir et al. (2001) Nature 411: 494-498. The mechanism may be used to downregulate expression levels of identified genes, e.g., treatment of or validation of relevance to disease. siRNAs are preferably between 19 and 29 nucleotides in length, most preferably between 21 and 25 nucleotides in length. By comparison dsRNAs can be considered to be at least 30 nucleotides in length, at least 50 nucleotides in length, at least 100 nucleotides in length, at least 500 nucleotides in length. shRNAs preferably form double-stranded regions of 19 to 29 nucleotides in length, preferably 22 to 29 nucleotides in length, more preferably 25 to 29 nucleotides in length, most preferably 29 nucleotides in length (see Paddison et al. (2002) Genes Dev. 16: 948-58). Exemplary requirements for siRNA length, structure, chemical composition, cleavage site position, and sequences essential to mediate efficient RNAi activity are described in (Elbashir et al. (2001) EMBO J. 20: 6877-6888) and (Nykanen et al. (2001) Cell 107: 309-321).

RNAi has been studied in a variety of systems, and a number of methods for producing and selecting RNAi molecules, such as shRNAs, siRNAs, and dsRNAs. Some methods for this embodiment of the invention are reviewed or documented in Paddison et al. (2004) Methods Mol Biol. 265: 85-100; Kakare et al. (2004) Appl Biochem Biotechnol. 119: 1-12; Paddison et al. (2004) Nature 428: 427-31; Paddison and Hannon (2002) Cancer Cell 2: 17-23; Paddison et al. (2002) Genes Dev. 16: 948-958; Hannon and Conklin (2004) Methods Mol Biol. 257: 255-266; Katoh et al. (2003) Nucleic Acids Res Suppl. (3): 249-250; Koper-Emde et al. (2004) Biol Chem. 385: 791-794; Gupta et al. (2004) Proc Natl Acad Sci U.S.A. 101: 1927-1932; Paddison et al. (2002) Proc Natl Acad Sci U.S.A. 99: 1443-1448 and the references thereto, and kits for some vectors are available (e.g. GeneEraser™ (catalog #240090) from Stratagene, La Jolla, Calif.). Fire et al. ((1998) Nature 391: 806-811) were the first to observe RNAi in C. elegans. Wianny and Goetz ((1999) Nature Cell Biol. 2: 70-75) describe RNAi mediated by dsRNA in mouse embryos. Hammond et al. ((2000) Nature 404: 293-296) describe RNAi in Drosophila cells transfected with dsRNA. Elbashir et al. ((2001) Nature 411: 494-498) describe RNAi induced by introduction of duplexes of synthetic 21-nucleotide RNAs in cultured mammalian cells including human embryonic kidney and HeLa cells. (Elbashir et al. (2001) EMBO J. 20: 6877-6888)(Nykanen et al. (2001) Cell 107: 309-321)

RNAi molecules include any form of RNA such as partially purified RNA, essentially pure RNA, synthetic RNA, recombinantly produced RNA, as well as altered RNA that differs from naturally occurring RNA by the addition, deletion, substitution, and/or alteration of one or more nucleotides. Such alterations can include the addition of non-nucleotide material, such as to the end(s) of the 21 to 23 nucleotide RNA or internally (at one or more nucleotides of the RNA). In a preferred embodiment, the RNA molecule contains a 3′hydroxyl group. Nucleotides in the RNAi molecules of the present invention can also comprise non-standard nucleotides, including non-naturally occurring nucleotides or deoxyribonucleotides. Additional modifications of the RNAi molecules (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incoporation) can be found in US Pat. Publication No. 20040019001.

ii.) Ribozymes

In addition to antisense polynucleotides, ribozymes can be used to target and inhibit transcription of cancer-associated nucleotide sequences such as TAT-028 nucleotides. Different kinds of ribozymes have been described, including group I ribozymes, hammerhead ribozymes, hairpin ribozymes, RNase P, and axhead ribozymes (see, e.g., Castanotto et al. (1994) Adv Pharmacol. 25: 289-317). The general features of hairpin ribozymes are described, e.g., in Hampel et al. (1990) Nucleic Acids Res. 18: 299-304; European Patent Publication No. 0 360 257; and U.S. Pat. No. 5,254,678. Methods of preparation are described in, e.g., WO 94/26877; Ojwang et al. (1993) Proc Natl Acad Sci. U.S.A. 90: 6340-6344; Yamada et al. (1994) Human Gene Ther. 1: 39-45; Leavitt et al. (1995) Proc Natl Acad Sci U.S.A. 92: 699-703; Leavitt et al. (1994) Human Gene Ther. 5: 1151-1120; and Yamada et al. (1994) Virology 205: 121-126.

TAT-028 nucleic acids such as ribozymes, RNAi constructs, and anti-sense molecules—collectively TAT-028 therapeutic nucleic acids—may be introduced into a cell containing the target nucleotide sequence using any techniques known in the art. In one example, the therapeutic nucleic acid is introduced by formation of a conjugate with a ligand binding molecule (e.g., cell surface receptors, growth factors, and other cytokines) as described in PCT Publication No. WO 91/04753. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. In another embodiment, a TAT-028 therapeutic nucleic acid may be introduced into a cell containing the target nucleic acid sequence, e.g., by formation of a polynucleotide-lipid complex, as described in WO 90/10448. It is understood that the use of antisense molecules or knock-out and knock-in models may also be used in screening assays as discussed above, in addition to methods of treatment. Delivery may also be per gene therapy methods described below.

Thus, the present invention provides for the therapeutic or prophylactic use of TAT-028 nucleic acids that are complementary to at least eight consecutive nucleotides of a gene or cDNA encoding a TAT-028 polypeptide. The nucleic acids can be antisense molecules, dsRNA or siRNA molecules, or vectors to produce such in the case of RNAi. TAT-028 nucleic acids may also be used directly as immunogens, or in vectors to provide immunogens through protein expression, for vaccination, or to design guide sequences for therapeutic and prophylactic ribozymes.

iii.) Gene Therapy

In a specific embodiment, TAT-028 nucleic acid molecules are used for gene therapy (see for example Hoshida et al. (2002) Pancreas. 25: 111-121; Ikuno (2002) Invest Ophthalmol Vis Sci. 43: 2406-2411; Bollard (2002) Blood. 99: 3179-3187; Lee (2001) Mol Med. 7: 773-782), such as in the treatment or prevention of cancer. Gene therapy refers to administration to a subject of an expressed or expressible nucleic acid. Any of the methods for gene therapy available in the art can be used according to the present invention. In one example, the TAT-028 nucleic acid can be administered as a pharmaceutical composition, for example as part of an expression vector that expresses a TAT-028 polypeptide or chimeric protein thereof in a suitable host. In particular, such a nucleic acid has a promoter (e.g., inducible or constitutive, and, optionally, tissue-specific) operably linked to the polypeptide coding region. In another example, a TAT-028 nucleic acid molecule is used in which the coding sequences and any other desired sequences are flanked by regions that promote homologous recombination at a desired site in the genome, thus providing for intrachromosomal expression of the nucleic acid (Koller and Smithies (1989) Proc Natl Acad Sci. U.S.A. 86: 8932-8935; Zijistra et al. (1989) Nature 342: 435-438).

Delivery of the TAT-028 nucleic acid into a patient may be direct (i.e. in vivo gene therapy), the patient is directly exposed to the nucleic acid or nucleic acid-carrying vector. Alternatively, delivery of the nucleic acid into the patient may be indirect (i.e. ex vivo gene therapy), cells are first transformed with the nucleic acid in vitro and then transplanted into the patient.

TAT-028 nucleic acids, TAT-028 polypeptides (for example, to target the therapy to cells which bind the polypeptide), or both may be utilized in gene delivery vehicles. The gene delivery vehicle may be of viral or non-viral origin (see Jolly (1994) Cancer Gene Ther. 1: 51-64; Kimura (1994) Human Gene Ther. 5: 845-852; Connelly (1995) Human Gene Ther. 1: 185-193; and Kaplitt (1994) Nat Gen. 6: 148-153). Exemplary gene delivery vehicles include those described above under “Expression vectors.” Gene therapy vehicles for delivery of constructs can be administered either locally or systemically. These constructs can utilize viral or non-viral vector approaches. Expression of such coding sequences can be induced using endogenous mammalian or heterologous promoters. Expression of the coding sequence can be either constitutive or regulated.

iv.) Aptamers

The invention also contemplates TAT-028 binding molecules and TAT-028 modulators that are aptamers. Methods are known in the art for designing, screening for, isolating, and selecting aptamers and using them as binding molecules and modulators. For example, see U.S. Pat. Nos. 5,582,981; 6,001,570; 6,180,348; 6,369,208; 6,458,559; and 6,949,379. Therapeutic use of such aptamers, alone and in combinatorial therapies, is also contemplated, and examples of such are known in the art, e.g., Lee et al. (Dec. 15, 2005) Proc Natl Acad Sci USA. Epub; Proske et al. (2005) Appl Microbiol Biotechnol. 69: 367-374; Siddiqui and Keating (2005) Drugs 65: 1571-1577; Bourgouts et al. (2005) Expert Opin Biol Ther. 5: 783-797; and Nimjee et al. (2005) Annu Rev Med. 56: 555-583. Analytical applications such as detecting TAT-028 are also contemplated Tombelli et al. (2005) Biosens Bioelectron. 20: 2424-2434.

Polypeptides

The invention also provides TAT-028 polypeptides. Polypeptides of the invention have a variety of uses, including, but not limited to: immunogenic compositions, screening for modulators of TAT-028 expression, screening for molecules that bind to TAT-028, and use as reagents and controls in assays of TAT-028 protein, such as diagnostic or prognostic assays. The TAT-028 protein preferably has the amino acid sequence of a naturally occurring TAT-028 found in a human, fungus, animal, plant, or microorganism, or a sequence derived therefrom. Preferably the TAT-028 is a human TAT-028. It will be apparent to one skilled in the art that peptides for use in the invention include TAT-028 and TAT-028 fragments, derivatives, and modified forms (e.g., analogues) thereof.

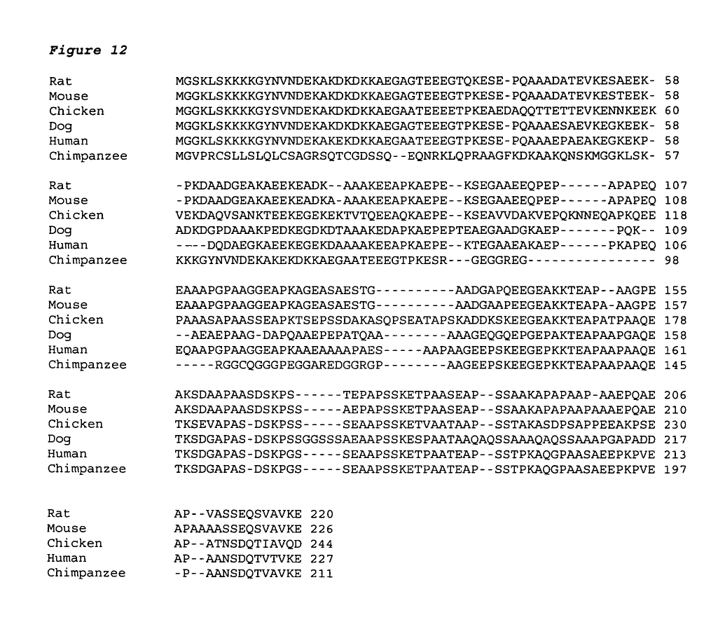

TAT-028 polypeptide sequences can be initially identified by substantial amino acid sequence similarity and/or identity to the TAT-028 polypeptide sequences described herein (e.g., SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 or SEQ ID NO: 19). Such similarity or identity can be based on the overall amino acid sequence, and is generally determined as described above. TAT-028 polypeptide sequences may alternatively be initially identified through structural homology or analogy, as determined by the functional or binding assays described herein and their results as compared to those of the TAT-028 polypeptide sequences described herein in the same assay. Activity as measured in such assays of a TAT-028 polypeptide is preferred to be at least 0.1%, at least 1%, at least 5%, or at least 10% that of a TAT-028 polypeptide sequence described herein. More preferably, the polypeptide has at least 25%, at least 50%, at least 75%,or at least 90% of the activity of a TAT-028 polypeptide sequence described herein. Most preferably, the polypeptide has at least 95%, at least 96%, at least 97%, at least 98%, or at least 99% of the activity of a TAT-028 polypeptide sequence described herein. Preferred TAT-028 polypeptides of the invention retain one or more activities of TAT-028, however, substantially homologous TAT-028 polypeptides need not be active to be useful, and as such may be useful, for example, as controls for functional TAT-028 polypeptides. Specific functional residues or combinations thereof may also be delineated in part through comparative assays, such as comparing the activity of the native sequence in a binding assay to that of a mutagenized sequence that lacks functional activity, as might be produced by techniques including but not limited to alanine scanning (see for example Chatellier et al. (1995) Analytical Biochemistry 229: 282-290), site-directed mutagenesis (Near et al. (1993) Mol Immunol. 30: 369-377), or saturation mutagenesis (Jeffrey et al. (1995) Nat Struct Biol. 2: 466-471). Additional TAT-028 polypeptides, including homologues, paralogues, and orthologues from species other than human, may be obtained using standard cloning techniques, screening techniques, or homology search techniques. For example, a phage display library derived from mRNA from murine cells may be screened with anti-TAT-028 antibodies to identify TAT-028 homologues or xenologues. Alternatively, a library may be screened using a yeast two-hybrid system and a TAT-028 binding protein as bait. Additional TAT-028 polypeptides may also be obtained from natural sources such as cell lysates via purification or can be synthesized using well known and commercially available techniques. TAT-028 polypeptides identified as xenologues include sequences from Chimpanzee (GenBank gi: 55623958; SEQ ID NO: 38), Dog (gi: 73954389; SEQ ID NO: 39), Rat (gi: 11560135; SEQ ID NO: 40), Mouse (gi: 66773932; SEQ ID NO: 41) and Chicken (gi: 45383878, SEQ ID NO: 42). An alignment of these sequences is provided in FIG. 12.

Fragments of a TAT-028 polypeptide may be used in the methods of the invention; preferably the fragments include an intact epitope that occurs in the biologically active wildtype TAT-028. The fragments comprise at least 4 consecutive amino acids of a TAT-028 polypeptide. Preferably, the fragment comprises at least 10, 15, 20, 25, 30, 35, 40, 50, 60, 70, 80, 90, 100, 110, 120, 130, 140, 150, 160, 170, 180, 190, 200, 210, 220, or at least 227 consecutive amino acids. In one embodiment, the fragment is from a human TAT-028 polypeptide. Preferably, the fragment contains an amino acid sequence conserved among mammalian TAT-028s, more preferably among primate TAT-028s. The skilled person can determine whether or not a particular fragment has activity using the techniques known in the art or disclosed herein for assessing the appropriate activity. Any given fragment of a polypeptide may or may not possess a functional activity of the parent polypeptide. Preferably the fragment has substantial sequence identity over the length of the corresponding TAT-028 sequence.

Fragments may be part of fusion proteins comprising or consisting of one or more TAT-028 fragments. Such fusion proteins may alter the order of the normal TAT-028 amino acid sequence or repeat certain elements or structures therein. Multiple fragments may be linked by non-TAT-028 fragments. Such non-TAT-028 fragments may or may not be considered immunogenic, and may or may not induce the included fragments to maintain a particular structural conformation or conformations. Fusion proteins comprising or consisting of one or more TAT-028 fragments are contemplated as encompassed in the definition of TAT-028 fragments (fragments of a TAT-028 polypeptide).

Alterations in the amino acid sequence of a protein can occur which do not affect the function of a protein. These include amino acid deletions, insertions, and substitutions, and can result from alternative splicing and/or the presence of multiple translational start sites or stop sites. Polymorphisms may arise from infidelity of the translational process. Thus, changes in amino acid sequence which do not affect biological or immunological function may be tolerated while maintaining substantially the same activity.

A “derivative” of a polypeptide includes a polypeptide comprising an amino acid sequence of a parent polypeptide that has been altered by the introduction of amino acid residue substitutions, deletions, or additions, and/or amino acid modifications, e.g., phosphorylation and glycosylation. Such introductions may be engineered for a polypeptide or an encoding nucleic acid or produced naturally. A derivative may also encompass homologues, analogues and orthologues of a parent polypeptide. The derivative polypeptide may possess a similar or identical function to the parent polypeptide. TAT-028 derivatives also preferably possess at least a degree of the antigenicity and/or immunogenicity of the protein or polypeptide from which they are derived.

An example of a derivative or variant of a TAT-028 polypeptide for use in the present invention is a TAT-028 polypeptide as defined by SEQ ID NO: 19, apart from the substitution of one or more amino acids with one or more other amino acids. Amino acid substitutions may be conservative or semi-conservative as known in the art and preferably do not significantly affect the desired activity of the polypeptide. Substitutions may be naturally occurring or may be introduced, for example, using mutagenesis (e.g., Hutchinson et al. (1978) J Biol Chem. 253: 6551-6560). Typically “variant” is used to describe a naturally occurring difference in sequence, while “derivative” typically describes a difference produced recombinantly or through other synthetic means, but may be used interchangeably or indiscriminately. Thus, the amino acids glycine, alanine, valine, leucine and isoleucine can often be substituted for one another (amino acids having aliphatic side chains). Of these possible substitutions, it is preferred that glycine and alanine are used to substitute for one another (since they have relatively short side chains) and that valine, leucine and isoleucine are used to substitute for one another (since they have larger hydrophobic aliphatic side chains). Other amino acids which can often be substituted for one another include: phenylalanine, tyrosine, and tryptophan (amino acids having aromatic side chains); lysine, arginine, and histidine (amino acids having basic side chains); aspartate and glutamate (amino acids having acidic side chains); asparagine and glutamine (amino acids having amide side chains); cysteine and methionine (amino acids having sulphur-containing side chains); and aspartic acid and glutamic acid can substitute for phospho-serine and phospho-threonine, respectively (amino acids with acidic side chains).

In a particular embodiment, the substituted amino acid(s) significantly affect the activity of the TAT-028 polypeptide and may be selected specifically to render dominant negative activity upon the peptide. In another embodiment, the substituted amino acid(s) may be selected to render the polypeptide constitutively active. Such alterations may be useful in screens or assays, such as phenotypic screens or enzymatic assays, or in the use of a TAT-028 polypeptide or fusion or conjugate thereof as a therapeutic molecule. Alterations that impact immunogenicity typically will be used to increase immunogenicity of particular sequences, such as increasing accessibility of the desired epitope, or altering loop stability (see, for example, Dai et al. (2002) J Biol Chem. 277: 161-168; Srivastava et al. (2003) J Virol. 77: 2310-2320; Yang et al. (2004) J Virol. 78: 4029-4036; Oomen et al. (2003) J Mol Biol. 328: 1083-1089), but may also be used to reduce immunogenicity of particular epitopes, such as when a heterogenous sequence is used to produce antibodies for use in humans, e.g., when murine peptides are used for immunization which contain an undesirable epitope not present in the human sequence, such as one that might produce undesirable cross-reactivity with other human proteins (see, for a related example, Vanderschueren et al. (1994) Thromb Haemost. 72: 297-301; Collen et al. (2000) Circulation. 102: 1766-1772; Su et al. (2004) Acta Biochim Biophys Sin (Shanghai) 36: 336-342). Techniques are known to the skilled artisan for making and measuring the impact of such alterations.

Amino acid deletions or insertions may also be made relative to a TAT-028 polypeptide sequence. Thus, for example, amino acids which do not have a substantial effect on the biological and/or immunological activity of the polypeptide, or at least which do not eliminate such activity, may be deleted. Such deletions can be advantageous since the overall length and the molecular weight of a polypeptide can be reduced while still retaining activity. Similarly, deletions may be made to produce an inactive form of a TAT-028 polypeptide.

Polypeptides comprising amino acid insertions relative to a TAT-028 polypeptide sequence are also within the scope of the invention. Such changes may alter the properties of a polypeptide used in the present invention (e.g., to assist in identification, purification or expression, as explained above in relation to fusion proteins). For example, insertion of an IL-1 beta peptide sequence may be used to enhance immunogenicity (see Beckers et al. (1993) J Immunol. 151: 1757-1764). Such amino acid changes can be made using any suitable technique, for example, site-directed mutagenesis (Hutchinson et al. (1 978) supra). It should be appreciated that amino acid substitutions or insertions to the polypeptide for use in the present invention can be made using naturally occurring or non-naturally occurring amino acids. Whether or not natural or synthetic amino acids are used, it is preferred that only L-amino acids are present.

Epitopes

It is well known that is possible to screen an antigenic protein or polypeptide to identify epitopic regions, i.e., those regions responsible for antigenicity or immunogenicity. Amino acid and peptide characteristics well known to the skilled person can be used to predict the antigenic index (a measure of the probability that a region is antigenic) of a TAT-028 polypeptide. For example, the PeptideStructure program (Jameson and Wolf (1988) Comput Appl Biosci. 4: 181-186) and/or a technique referred to as threading (Altuvia et al. (1995) J Mol Biol. 249: 244-250) may be used. Thus, the TAT-028 polypeptides may include one or more such epitopes or be sufficiently similar to such regions as to retain antigenic or immunogenic properties. Methods well known to the skilled person can be used to test fragments, and/or homologues and/or derivatives of a polypeptide for immunogenicity. Thus, the fragments for use in the present invention may include one or more such epitopic regions or be sufficiently similar to such regions to retain their antigenic or immunogenic properties. Isolated TAT-028 polypeptides of the invention (and their encoding nucleic acids) may therefore be screened for use in inducing an immune response based on known and/or predicted immunogenicity, or judged individually. Such immunogenic polypeptides may be referred to as “immunogenic isolated polypeptides” of the invention.

Polypeptide Expression

In another aspect, the invention provides for isolated or recombinant TAT-028 polypeptides or fragments. The isolated or recombinant TAT-028 polypeptides or fragments may also be fused to other moieties. Such moieties or amino acid sequences may be optionally removed as required by incorporating a cleavable sequence or moiety as an additional sequence or part thereof. In particular, fusions of the polypeptides or fragments thereof with localization-reporter proteins such as the Green Fluorescent Protein (U.S. Pat. Nos. 5,625,048; 5,777,079; 6,054,321 and 5,804,387) or the DsRed fluorescent protein (Matz et al. (1999) Nat Biotech. 17: 969-973) are specifically contemplated. Also contemplated are affinity tag and epitope tag fusions, for example, HIS-tag, HA-tag, FLAG-tag, and Myc-tag fusions, respectively. Fusions can be useful in improving recombinant expression, improving purification, or regulation of expression in particular expression systems. For example, an additional sequence may provide some protection against proteolytic cleavage. Additional N-terminal or C-terminal amino acid sequences may, however, be present simply as a result of a particular technique used to obtain a polypeptide and need not provide any particular advantageous characteristic to the polypeptide. Such polypeptides are within the scope of the present invention.

The polypeptides or fragments thereof may be provided in substantially pure form, that is to say free, to a substantial extent, from other proteins. Thus, a polypeptide may be provided in a composition in which it is the predominant component present (i.e., it is present at a level of at least 50%; preferably at least 75%, at least 90%, or at least 95%; when determined on a weight/weight basis excluding solvents, carriers, or coupling agents).

The skilled person will appreciate that for the preparation of one or more such polypeptides, the preferred approach will be based on recombinant DNA techniques (some of which may be represented in “Nucleic Acids” above). Recombinant TAT-028 polypeptides may be prepared by processes well known in the art from genetically engineered host cells comprising expression systems. Accordingly, the present invention also relates to expression systems which comprise a TAT-028 polypeptide and/or TAT-028 nucleic acid, to host cells which are genetically engineered to incorporate such expression systems or portions thereof, and to the production of TAT-028 polypeptides by recombinant techniques. Cell-free translation systems can also be employed to produce recombinant polypeptides (e.g., rabbit reticulocyte lysate, wheat germ lysate, SP6/T7 in vitro T&T and RTS 100 E. coli Hy transcription and translation kits from Roche Diagnostics Ltd., Lewes, UK; and the TNT Quick coupled Transcription/Translation System from Promega UK, Southampton, UK).

A wide variety of expression systems (a term inclusive of expression constructs) can be used, such as and without limitation, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. Generally, any system or vector which is able to maintain, propagate or express a nucleic acid to produce a polypeptide in a host may be used. The appropriate TAT-028 nucleic acid sequence may be inserted into an expression system by any variety of well-known and routine techniques, such as those set forth in Sambrook et al., supra.

An expression system or construct can be introduced into a host cell. The host cell comprising the expression construct can be any suitable prokaryotic or eukaryotic cell. Expression systems in bacteria include those described in Chang et al. (1978) Nature 275: 617-624; Goeddel et al. (1979) Nature 281: 544-548; Goeddel et al. (1980) Nucleic Acids Res. 8: 4057-4074; EP 36,776; U.S. Pat. No. 4,551,433; deBoer et al. (1983) Proc Natl Acad Sci. U.S.A. 80: 21-25; and Siebenlist et al. (1980) Cell 20: 269-281.

Representative examples of host cells include bacterial cells (e.g., E. coli, Streptococci, Staphylococci, Streptomyces and Bacillus subtilis cells); fungal cells (e.g., yeast cells and Aspergillus cells); insect cells (e.g., Drosophila S2 and Spodoptera Sf9 cells); animal cells (e.g., CHO, COS, HeLa, C127, 3T3, HEK 293, BHK, and Bowes melanoma cells); and plant cells.

Expression systems in yeast include those described in Hinnen et al. (1978) Proc Natl Acad Sci. U.S.A. 75: 1929-1933; Ito et al. (1983) J Bacteriol. 153: 163-168; Kurtz et al. (1986) Mol Cell Biol. 6: 142-149; Kunze et al. (1985) J Basic Microbiol. 25: 141-144; Gleeson et al. (1986) J Gen Microbiol. 132: 3459-3465; Roggenkamp et al. (1986) Mol Gen Genet. 202: 302-308; Das et al. (1984) J Bacteriol. 158: 1165-1167; De Louvencourt et al. (1983) J Bacteriol. 154: 737-742; Van den Berg et al. (1990) Biotechnology. 8: 135-139; Kunze et al. (1985) J Basic Microbiol. 25: 141-144; Cregg et al. (1985) Mol Cell Biol. 5: 3376-3385; U.S. Pat. Nos. 4,837,148; 4,929,555; Beach et al. (1982) Nature 300: 706-709; Davidow et al. (1985) Curr Genet. 10: 39-48; Gaillardin et al. (1985) Curr Genet. 10: 49-58; Ballance et al. (1983) Biochem Biophys Res Commun. 112: 284-289; Tilburn et al. (1983) Gene. 26: 205-22; Yelton et al. (1984) Proc Natl Acad Sci. U.S.A. 81: 1470-1474; Kelly and Hynes (1985) EMBO J. 4: 475-479; U.S. Pat. No. 4,937,189; EP 244,234; and WO 91/00357.

Expression of heterologous genes in insects may be accomplished as described in U.S. Pat. No. 4,745,051; Friesen et al. (1986) “The Regulation of Baculovirus Gene Expression” in: The Molecular Biology of Baculoviruses (W. Doerfier, ed.); EP 127,839; EP 155,476; Vlak et al. (1988) J Gen Virol. 69: 765-776; Miller et al. (1988) Ann Rev Microbiol. 42: 177-199; Carbonell et al. (1988) Gene. 73: 409-418; Maeda et al. (1985) Nature 315: 592-594; Lebacq-Verheyden et al. (1988) Mol Cell Biol. 8: 3129-3135; Smith et al. (1985) Proc Natl Acad Sci. U.S.A. 82: 8404-8408; Miyajima et al. (1987) Gene. 58: 273-281; and Martin et al. (1988) DNA. 7: 99-106. Numerous baculoviral strains and variants and corresponding permissive insect host cells from hosts are described in Luckow and Summers (1988) Biotechnology. 6: 47-55; Luckow (1993) Curr Opin Biotechnol. 4: 564-572; Miller et al. in Genetic Engineering (Setlow, J. K. et al. eds.), Vol. 8, pp. 277-279 (Plenum Publishing, 1986); and Maeda et al. (1985) Nature 315: 592-594.

Mammalian expression can be accomplished as described in Dijkema et al. (1985) EMBO J. 4: 761-767; Gorman et al. (1982b) Proc Natl Acad Sci. U.S.A. 79: 6777-6781; Boshart et al. (1985) Cell 41: 521-530; and U.S. Pat. No. 4,399,216. Other features of mammalian expression can be facilitated as described in Ham and McKeehan (1979) Meth Enz. 58: 44-93; Barnes and Sato (1980) Anal Biochem. 102: 255-270; U.S. Pat. Nos. 4,767,704; 4,657,866; 4,927,762; 4,560,655; WO 90/103430; WO 87/00195; and U.S. Pat. No. RE 30,985.

Expression systems or constructs, in whole or in part, can be introduced into host cells using any technique known in the art (see e.g., Davis et al. (1986) Basic Methods in Molecular Biology and Sambrook et al. (1989) Molecular Cloning: A Laboratory Manual, 2nd Ed., Cold Spring Harbour laboratory Press, Cold Spring Harbour, N.Y.).

The expression systems may contain control regions that regulate as well as engender expression. For example, expression of an endogenous gene encoding a protein of the invention can also be manipulated by introducing, by homologous recombination, a DNA construct comprising a transcription unit in frame with the endogenous gene, to form a homologously recombinant cell comprising the transcription unit. This method of affecting endogenous gene expression is taught, for example, in U.S. Pat. No. 5,641,670.

Appropriate secretion signals may be incorporated into the TAT-028 polypeptide to allow secretion of the translated protein into the lumen of the endoplasmic reticulum, the periplasmic space or the extracellular environment. These signals may be endogenous to the TAT-028 polypeptide or they may be heterologous signals.

If a TAT-028 polypeptide is to be expressed for use in cell-based screening assays, it is preferred that the polypeptide be produced at the cell surface. In this event, the cells may be harvested prior to use in the screening assay. If the TAT-028 polypeptide is secreted into the medium, the medium can be recovered in order to isolate the polypeptide. If produced intracellularly, the cells must first be lysed before the TAT-028 polypeptide is recovered.

TAT-028 polypeptides can be recovered and purified from recombinant cell cultures by well-known methods including, ammonium sulphate or ethanol precipitation, acid extraction, anion or cation exchange chromatography, phosphocellulose chromatography, affinity chromatography, hydrophobic interaction chromatography, hydroxylapatite chromatography, molecular sieving chromatography, centrifugation methods, electrophoresis methods and lectin chromatography. In one embodiment, a combination of these methods is used. In another embodiment, high performance liquid chromatography is used. In a further embodiment, an antibody which specifically binds to a TAT-028 polypeptide can be used to deplete a sample comprising a TAT-028 polypeptide of the polypeptide or to purify the polypeptide. Techniques well-known in the art, may be used for refolding to regenerate native or active conformations of the TAT-028 polypeptides when the polypeptides have been denatured during isolation and or purification, should such be desired.

Transgenics and Knockouts

The polypeptides of the invention can also be expressed, or otherwise have their expression altered (for example, a “knockout”), in transgenic animals. Animals may be of any species, including, but not limited to, mice, rats, rabbits, hamsters, guinea pigs, pigs, micro-pigs, goats, sheep, cows and non-human primates (e.g., baboons, monkeys, and chimpanzees) may be used to generate transgenic animals. Preferably, transgenic animals of the invention are mammals. Mammalian TAT-028 xenologue genomic sequences, in particular rodent, can be determined using the methods of Example 5 and standard DNA sequencing methods or by assessment of homology and sequence identity using the methods and TAT-028 sequences described herein (Human (GenBank gi: 30795231; SEQ ID NO: 19) Chimpanzee (GenBank gi: 55623958; SEQ ID NO: 38), Dog (gi: 73954389; SEQ ID NO: 39), Rat (gi: 11560135; SEQ ID NO: 40), Mouse (gi: 66773932; SEQ ID NO: 41) and Chicken (gi: 45383878, SEQ ID NO: 42)).

Any technique known in the art may be used to introduce the transgene (i.e., polynucleotides of the invention) into animals to produce the founder lines of transgenic animals. Such techniques include, but are not limited to, pronuclear microinjection (Paterson et al. (1994) Appl Microbiol Biotechnol. 40: 691-698; Carver et al. (1993) Biotechnology (NY) 11: 1263-1270; Wright et al. (1991) Biotechnology (NY) 9: 830-834; and U.S. Pat. No. 4,873,191); retrovirus mediated gene transfer into germ lines (Van der Putten et al. (1985) Proc Natl Acad Sci. U.S.A. 82: 6148-6152), blastocysts or embryos; gene targeting in embryonic stem cells (Thompson et al. (1989) Cell 56: 313-321); electroporation of cells or embryos (Lo (1983) Mol Cell Biol. 3: 1803-1814); introduction of the polynucleotides of the invention using a gene gun (see, e.g., Ulmer et al. (1993) Science 259: 1745-1749; introducing nucleic acid constructs into embryonic pleuripotent stem cells and transferring the stem cells back into the blastocyst; and sperm-mediated gene transfer (Lavitrano et al. (1989) Cell 57: 717-723). For a review of such techniques, see Gordon (1989) Intl Rev Cytol. 115: 171-229, which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al. (1996) Nature 380: 64-66; Wilmut et al. (1997) Nature 385: 810-813).

The present invention provides for transgenic animals that carry the transgene in all their cells, as well as animals which carry the transgene in some, but not all their cells (i.e., mosaic or chimeric animals). The transgene may be integrated as a single transgene or as multiple copies such as in concatamers (e.g., head-to-head tandems or head-to-tail tandems). Thus, animal models of TAT-028 overproduction can be generated by integrating one or more TAT-028 sequences into the genome of an animal, according to standard transgenic techniques. Moreover, the effect of TAT-028 gene mutations (e.g., dominant gene mutations) can be studied using transgenic mice carrying mutated TAT-028 transgenes or by introducing such mutations into the endogenous TAT-028 gene, using standard homologous recombination techniques. The transgene may also be selectively introduced into and activated in a particular cell type by following, for example, the teaching of Lasko et al. ((1992) Proc Natl Acad Sci. U.S.A. 89: 6232-6236). The regulatory sequences required for such a cell-type specific activation will depend upon the particular cell type of interest, and will be apparent to those of skill in the art. The transgene may also be selectively introduced into a particular cell type, thus inactivating the endogenous gene in only that cell type (see e.g., Gu et al. (1994) Science 265: 103-106). The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques including Southern blot anaysis, PCR techniques, northern blot analysis, in situ hybridization analysis, reverse transcriptase PCR (rtPCR), immunocytochemistry, and immunohistochemistry. Once the founder animals are produced, they may be bred, inbred, outbred, or crossbred to produce colonies of the particular animal.

Endogenous gene expression may also be reduced by inactivating or “knocking out” the TAT-028 gene and/or its promoter using targeted homologous recombination in animals. (e.g., see Smithies et al. (1985) Nature 317: 230-234; Thomas and Capecchi (1987) Cell 51: 503-512; Thompson et al. (1989) Cell 5: 313-321; and Zijistra et al. (1989) Nature 342: 435-438; each of which is incorporated by reference herein in its entirety). Characterization of TAT-028 genes provides information that allows TAT-028 knockout animal models to be developed by homologous recombination. A “knockout animal” is preferably a mammal, and more preferably a mouse, containing a knockout mutation, as defined below. By a “knockout mutation” is meant an artificially-induced alteration in a nucleic acid molecule (created by recombinant DNA technology or deliberate exposure to a mutagen) that reduces the biological activity of the polypeptide normally encoded therefrom by at least 80% relative to the unmutated gene. The mutation can be, without limitation, an insertion, deletion, frameshift mutation, or a missense mutation. In a specific embodiment, techniques described herein or otherwise known in the art, are used to effect a “knockout” of the invention in humans, as part of a gene therapy protocol.

A replacement-type targeting vector, which can be used to create a knockout model, can be constructed using an isogenic genomic clone, for example, from a mouse strain such as 129/Sv (Stratagene Inc., LaJolla, Calif.). The targeting vector can be introduced into a suitably-derived line of embryonic stem (ES) cells by electroporation to generate ES cell lines that carry a profoundly truncated form of a TAT-028 gene. To generate chimeric founder mice, the targeted cell lines are injected into a mouse blastula-stage embryo. Heterozygous offspring can be interbred to homozygosity. TAT-028 knockout mice provide a tool for studying the role of TAT-028 in disease such as cancer. Moreover, such mice provide the means, in vivo, for testing therapeutic compounds for amelioration of diseases or conditions involving a TAT-028-dependent or TAT-028-affected pathway.

Cell lines for use under cell culture conditions may be derived from transgenic and knockout animal models by methods commonly known in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g.,—lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention. Transgenic and “knock-out” animals of the invention and tissues, organs, cell lines, and the like derived therefrom have uses which include, but are not limited to, animal model systems useful in elaborating the biological function of polypeptides of the present invention, studying diseases, disorders, and/or conditions associated with aberrant expression of TAT-028. Animal model systems are also useful for screening for compounds effective in ameliorating such diseases, disorders, and/or conditions.

Immunotherapy

As discussed below, TAT-028 nucleic acids and TAT-028 polypeptides are of use in an immunotherapeutic approach to proliferative discorders (e.g., cancer). In some embodiments, immunotherapy may be active immunotherapy (e.g., vaccines), in which treatment relies on the in vivo stimulation of the endogenous host immune system to react against tumors with the administration of immune response-modifying agents (such as TAT-028 polypeptides, TAT-028 nucleic acids, or effector cells). In other embodiments, immunotherapy may be passive immunotherapy, in which treatment involves the delivery of agents with established tumor-immune reactivity (e.g., effector cells or antibodies) that can directly or indirectly mediate antitumor effects and do not necessarily depend on an intact host immune system.

Examples of effector cells include T cells, T lymphocytes (e.g., CD8⁺ cytotoxic T lymphocytes and CD4⁺ T-helper tumor-infiltrating lymphocytes), killer cells (e.g., natural killer cells and lymphokine-activated killer cells), B cells, and other antigen-presenting cells (e.g., dendritic cells and macrophages (in various parts of the body, the macrophage may be referred to as alveolar cells (lungs); mesangial cells (kidneys); microglial cells (brain); Kupffer cells (liver); and dendritic Langerhans cells (skin))), expressing, presenting, or contacted with a TAT-028 polypeptide provided herein.

Effector cells may generally be obtained in sufficient quantities for adoptive immunotherapy by growth in vitro. Culture conditions for expanding single antigen-specific effector cells to several billion in number with retention of antigen recognition in vivo are well known in the art. In particular, antigen-presenting cells, such as dendritic, macrophage, monocyte, fibroblast and/or B cells, may be pulsed with immunogenic polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al. (1997) Immunol Rev. 157: 177-194).

In one embodiment, autologous dendritic cells are pulsed with TAT-028 polypeptides capable of binding to MHC molecules (as may be determined using methods known in the art (see for example, Rammensee et al. (1999) Immunogenetics. 50: 213-219). In another embodiment, dendritic cells are pulsed with the complete TAT-028 protein. Yet another embodiment involves engineering the overexpression of the TAT-028 gene in dendritic cells using various implementing vectors known in the art, such as adenovirus (Arthur et al. (1997) Cancer Gene Ther. 4: 17-25), retrovirus (Henderson et al. (1996) Cancer Res. 56: 3763-3770), lentivirus, adeno-associated virus, DNA transfection (Ribas et al. (1997) Cancer Res. 57: 2865-2869), and tumor-derived RNA transfection (Ashley et al. (1997) J Exp Med. 186: 1177-1182).

Particularly, the invention also encompasses the use of an antigen encoded by a TAT-028 nucleic acid. It is anticipated that these antigens may be used as therapeutic or prophylactic anti-cancer vaccines, and thus as anti-cancer agents. For example, a particular contemplated application of these antigens involves their administration with adjuvants that induce a cytotoxic T lymphocyte response. An especially preferred adjuvant is disclosed in U.S. Pat. Nos. 5,709,860; 5,695,770; and 5,585,103, incorporated herein by reference. Also, administration of the subject novel antigens in combination with an adjuvant may result in a humoral immune response against such antigens, thereby delaying or preventing the development of a cancer, such as lung cancer.

Alternatively, a vector expressing a TAT-028 polypeptide may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

T cell receptors and antibody receptors specific for TAT-028 polypeptides may be cloned, expressed and transferred into other vectors or effector cells for adoptive immunotherapy. TAT-028 polypeptides provided herein may also be used to generate antibodies or anti-idiotypic antibodies (as herein and in U.S. Pat. No. 4,918,164) for passive immunotherapy.

Thus, the invention also provides a method of inducing an immune response to a TAT-028 polypeptide that includes providing a TAT-028 polypeptide that comprises at least one T cell antigen or at least one B cell antigen or at least one antigen presenting cell antigen; and, contacting the antigen with an immune system T cell or B cell or antigen presenting cell respectively, whereby an immune response is induced. Within the scope of this method, the polypeptide may be accompanied by an adjuvant, and within the scope of “contacting” the antigen may be made available to antigen presenting cells by the embodiments described above.

Vaccines

As already noted, a further aspect of the invention relates to a vaccine composition of use in the treatment of cancer. Thus, a TAT-028 polypeptide or TAT-028 nucleic acid may be useful as antigenic material, and may be used in the production of vaccines for treatment or prophylaxis of cancer. Such material can be “antigenic” and/or “immunogenic”. Generally, “antigenic” is taken to mean that the protein or nucleic acid is capable of being used to raise antibodies or indeed is capable of inducing an antibody response in a subject. “Immunogenic” is taken to mean that the protein or nucleic acid is capable of inducing a protective immune response in a subject. Thus, in the latter case, the TAT-028 polypeptide or TAT-028 nucleic acid may be capable of not only generating an antibody response but also non-antibody-based immune responses.

The invention further involves the identification of human patients for administration of a TAT-028 vaccine. A TAT-028 vaccine of the invention may be administered to healthy individuals as a prophylactic therapy or to individuals diagnosed with a neoplasm (e.g., lung cancer). Individuals selected for prophylactic administration of recombinant TAT-028 include any individual at risk of developing a neoplasm as based upon age, sex, geographical location, family history, or the presence of a condition (e.g., the presence of precancerous lesions or cells) which renders the individual susceptible to a neoplasm (e.g., lung cancer). Individuals who may receive the recombinant TAT-028 vaccine as a therapeutic include those individuals with symptoms of lung cancer, a family history of lung cancer, or a predisposition to developing lung cancer.

Individuals who have a neoplasm such as lung cancer may also be treated by administration of a vaccine of the invention, preferably in an immunogenically effective amount. Lung cancer disorders include any disease or other disorder of the respiratory system of a human or other mammal. Lung neoplastic disorders include, for example, non-small cell lung cancer, including adenocarcinoma, acinar adenocarcinoma, bronchioloalveolar adenocarcinoma, papillary adenocarcinoma, solid adenocarcinoma with mucus formation, squamous cell carcinoma, undifferentiated large cell carcinoma, giant cell carcinoma, synchronous tumors, large cell neuroendocrine carcinoma, adenosquamous carcinoma, undifferentiated carcinoma; and small cell carcinoma, including oat cell cancer, mixed small cell/large cell carcinoma, and combined small cell carcinoma; as well as adenoid cystic carcinoma, hamartomas, mucoepidermoid tumors, typical carcinoid lung tumors, atypical carcinoid lung tumors, peripheral carcinoid lung tumors, central carcinoid lung tumors, pleural mesotheliomas, and dysplasia, hyperplasia, neoplasia, and metastases of respiratory system origin. Alternatively, it may be desirable to administer the vaccine to asymptomatic individuals, particularly where the individual may be susceptible to a neoplasm.

TAT-028 polypeptides of the invention and mixtures and combinations thereof may be useful as active components of vaccines capable of inducing a prophylactic or therapeutically effective immune response against cancer. Routes of administration, antigen doses, number and frequency of injections will vary from species to species and may parallel those currently being used in the clinic and/or experimentally to provide immunity or therapy against other diseases or cancer. For example, the vaccines are pharmaceutically acceptable compositions containing one or more of the TAT-028 polypeptides of this invention, its analogues or mixtures or combinations thereof, in an amount effective in the mammal, including a human, treated with that composition to raise immunity sufficient to protect the treated mammal from cancer for a period of time.

Different types of vaccines can be developed according to standard procedures known in the art. For example, a vaccine may be peptide-based, nucleic acid-based, bacterial- or viral-based vaccines. A vaccine formulation containing at least one TAT-028 polypeptide or nucleic acid may contain a variety of other components, including stabilizers, flavor enhancers (e.g., sugar). The vaccine also optionally comprises or is co-administered with one or more suitable adjuvants, such as a mucosal adjuvant. The mucosal adjuvant may be any known in the art appropriate for human use (e.g., cholera toxin (CT), enterotoxigenic E. coli heat-labile toxin (LT), or a derivative, subunit, or fragment of CT or LT which retains adjuvanticity). The mucosal adjuvant is co-administered with TAT-028 vaccine in an amount effective to induce or enhance a mucosal immune response, particularly a humoral and/or a mucosal immune response. The ratio of adjuvant to TAT-028 vaccine may be determined by standard methods by one skilled in the art. Preferably, the adjuvant is present at a ratio of 1 part adjuvant to 10 parts TAT-028 vaccine.

In another embodiment, peptide vaccines may utilize peptides corresponding to a TAT-028-specific epitope or functional derivatives thereof can be utilized as a prophylactic or therapeutic vaccine in a number of ways, including: 1) as monomers or multimers of the same sequence, 2) combined contiguously or non-contiguously with additional sequences that may facilitate aggregation, promote presentation or processing of the epitope (e.g., class I/II targeting sequences) and/or additional antibody, T helper or CTL epitopes to increase the immunogenicity of the TAT-028-specific epitope as a means to enhance efficacy of the vaccine, 3) chemically modified or conjugated to agents that would increase the immunogenicity or delivery of the vaccine (e.g., fatty acid or acyl chains, KLH, tetanus toxoid, or cholera toxin), 4) any combination of the above, 5) any of the above in combination with adjuvants, including but not limited to inorganic gels such as aluminium hydroxide, and water-in-oil emulsions such as incomplete Freund's adjuvant, aluminum salts, saponins or triterpenes, MPL, cholera toxin, ISCOM'S®, PROVAX®, DETOX®, SAF, Freund's adjuvant, Alum®, Saponin®, among others, and particularly those described in in U.S. Pat. Nos. 5,709,860; 5,695,770; and 5,585,103; and/or delivery vehicles, including but not limited to liposomes, VPLs or virus-like particles, microemulsions, attenuated or killed bacterial and viral vectors, and degradable microspheres (see e.g., Kersten and Hirschberg (2004) Expert Rev of Vaccines. 3: 453-462; Sheikh et al. (2000) Curr Opin Mol Ther. 2: 37-54), and 6) administered by any route or as a means to load cells with antigen ex vivo.

Examples of these nucleic-acid based vaccines as a prophylactic or a therapeutic include: 1) any nucleic acid encoding the expression (transcription and/or translation) of TAT-028-specific epitope, 2) additional nucleic acid sequences that facilitate processing and presentation, aggregation, secretion, targeting (to a particular cell type) of a TAT-028-specific epitope, either translational fusions or independent transcriptional units, 3) additional nucleic acid sequences that function as adjuvants/immunomodulators, either translational fusions or independent transcriptional units, 4) additional antibody, T helper or CTL epitopes that increase the immunogenicity of a TAT-028-specific epitope or efficacy of the vaccine, either translational fusions or independent, and 5) any combination of the above, 6) the above administered in saline (‘naked’ DNA) or in combination with an adjuvant(s), (e.g., aluminum salts, QS-21, MPL), immunomodulatory agent(s) (e.g., rIL-2, rGM-CSF, rIL-12), and/or nucleic acid delivery agents (e.g., polymer-, lipid-, peptide-based, degradable particles, microemulsions, VPLs, attenuated bacterial or viral vectors) using any route or ex vivo loading.

The process for formulation of a TAT-028 vaccine involves standard methods known in the art, for example see Kersten and Hirschberg (2004) supra for review and U.S. Pat. Nos. 6,126,938 and 6,630,455).

Thus, in a further aspect, the present invention provides the use of a TAT-028 polypeptide or a TAT-028 nucleic acid in the production of a pharmaceutical composition for the treatment or prophylaxis of cancer, wherein the composition is a vaccine. For prophylactic therapy, a vaccine containing at least one TAT-028 polypeptide may be administered at any time prior to contact with, or establishment of, a lung carcinoma.

Dosages of a TAT-028 vaccine administered to the individual as either a prophylactic therapy or an antineoplastic therapy can be determined by one skilled in the art. Generally, dosages will contain between about 10 μg to 1,000 mg, preferably between about 10 mg and 500 mg, more preferably between about 30 mg and 120 mg, more preferably between about 40 mg and 70 mg, most preferably about 60 mg of a TAT-028 vaccine.

At least one dose of a TAT-028 vaccine will be administered to the patient, preferably at least two doses, more preferably four doses, with up to six or more total doses administered. It may be desirable to administer booster doses of a TAT-028 vaccine at one or two week intervals after the last immunization, generally one booster dose containing less than, or the same amount of, a TAT-028 vaccine as the initial dose administered. Most preferably, the vaccine regimen will be administered in four doses at one week intervals. Since a polypeptide or a nucleic acid may be broken down in the stomach, the vaccine composition is preferably administered parenterally (e.g., subcutaneous, intramuscular, intravenous, or intradermal injection). The progress of immunized patients may be followed by general medical evaluation, screening for infection by serology and/or gastroscopic examination.

Antibodies

The invention preferably includes the preparation and use of anti-TAT-028 antibodies and fragments for use as diagnostics and therapeutics. The unique ability of antibodies to recognize and specifically bind to target proteins provides approaches for both diagnosing and treating a cancer characterized by overexpression of one or more TAT-028 polypeptides. Thus, another aspect of the present invention provides for a method for preventing or treating diseases (e.g., cancer) involving overexpression of TAT-028 by treatment of a patient with antibodies that specifically bind to TAT-028 protein. To this end, the invention provides antibodies that bind to TAT-028 polypeptides and fragments thereof, including, but not limited to, polyclonal and monoclonal antibodies, anti-idiotypic antibodies, murine and other mammalian antibodies, antibody fragments, bispecific antibodies, antibody dimers or tetramers, single chain antibodies (e.g., scFv's and antigen-binding antibody fragments such as Fabs, 2 Fabs, and Fab′ fragments), recombinant binding regions based on antibody binding regions, chimeric antibodies, primatized antibodies, humanized and fully human antibodies, domain deleted antibodies, and antibodies labeled with a detectable marker, or coupled with a toxin or radionucleide. Such antibodies can be produced by conventional methods. However, the preferred embodiment of the invention will comprise the preparation of monoclonal antibodies or antibody fragments against the antigens encoded by TAT-028 nucleic acids, preferably those encoded by SEQ ID NO: 20. Accordingly, a TAT-028 polypeptide may be used as an immunogen to generate antibodies.

Thus, if an antibody molecule that specifically binds a particular TAT-028 antigen is desired, particularly should one not be otherwise available (or a source for a cDNA library for cloning a nucleic acid encoding such an antibody), antibodies specific for the particular antigen may be generated by any suitable method known in the art, examples of which are discussed below. In one example, murine or human monoclonal antibodies can be produced through recombinant methods by hybridoma technology, preferably in eukaryotic cells. In another example, the protein, or an immunologically active fragment thereof, or an anti-idiotypic antibody, or fragment thereof can be administered to an animal to induce the production of antibodies capable of recognizing and binding to the protein. Genetic immunization can be carried out by injecting the animals with cDNA encoding the target protein, obviating the need to prepare a protein or peptide immunogen. In general, antibody generation will comprise immunization of an appropriate (generally non-homologous) host with the desired TAT-028 polypeptide(s) or TAT-028 nucleic acid(s) (collectively TAT-028 antigens, though preferentially this term refers to TAT-028 polypeptides, most preferably the peptides of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17 and/or the protein of SEQ ID NO: 19). Specific antibodies or fluids, tissues, organs or cells containing them may be isolated from the host for purification or use in unpurified form, such as rabbit sera. Or, in a preferred embodiment, the isolation of immune cells therefrom, use of such immune cells to make hybridomas, and screening for monoclonal antibodies that specifically bind to a TAT-028 polypeptide will be carried out. Such antibodies can be from any class of antibodies including, but not limited to IgG, IgA, IgM, IgD, and IgE or in the case of avian species, IgY and from any subclass of antibodies.

Most preferred are antibodies that bind specifically to one or more TAT-028 polypeptides. In one embodiment, antibodies may be used to inhibit the activity of the TAT-028 polypeptides, and/or to target therapeutic agents (e.g., radionucleides or an immune response) to a tumor. Preferably, such antibodies will bind TAT-028 antigens with high affinity, e.g., possess a binding affinity (Kd) on the order of 10⁻⁶ to 10⁻¹² M or greater, preferably at least 10⁻⁶, at least 10⁻⁷, more preferably at least 10⁻⁸, at least 10⁻⁹, at least 10⁻¹⁰, most preferably at least 10⁻¹¹, at least 10⁻¹², or greater.

i.) Polyclonals

Polyclonal antibodies can be prepared by immunizing rabbits or other animals by injecting antigen followed by subsequent boosts at appropriate intervals. The animals are bled and sera assayed against purified protein usually by ELISA or by bioassay based upon the ability to block the action of the corresponding gene. When using avian species, e.g., chicken, turkey and the like, the antibody can be isolated from the yolk of the egg.

Polyclonal antibodies to TAT-028 antigens can be raised in animals by multiple subcutaneous (sc) or intraperitoneal (ip) injections of the antigen and an adjuvant. It may be useful to conjugate the antigen or a fragment containing the target amino acid sequence to a protein that is immunogenic in the species to be immunized (e.g., keyhole limpet hemocyanin, serum albumin, bovine thyroglobulin, or soybean trypsin inhibitor) using a bifunctional or derivatizing agent (e.g., maleimidobenzoyl sulfosuccinimide ester (conjugation through cysteine residues), N-hydroxysuccinimide (through lysine residues), glutaraldehyde, or succinic anhydride).

For example, animals can be immunized against the TAT-028 polypeptide or fragment thereof, immunogenic conjugates, or derivatives by combining 1 μg to 1 mg of the peptide or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer to the antigen or a fragment thereof. Animals are boosted until the titer plateaus. Preferably, the animal is boosted with the conjugate of the same polypeptide or another TAT-028 polypeptide or fragment thereof, but conjugated to a different protein and/or through a different cross-linking reagent. Conjugates also can be made in recombinant cell culture as protein fusions. Also, aggregating agents such as alum are suitably used to enhance the immune response.

Chimeric, humanized, or fully human polyclonals may be produced in animals transgenic for human immunoglobulin genes, or by isolating two or more TAT-028 reactive B-lymphocytes from a patient for starting material.

Polyclonals may also be purified and selected for (such as through affinity for a conformationally constrained antigen peptide), iteratively if necessary, to provide a monoclonal antibody. Alternatively or additionally, cloning out the nucleic acid encoding a single antibody from a lymphocyte may be employed.

ii.) Monoclonals

In a preferred embodiment of the invention, monoclonal antibodies are obtained from a population of substantially homogeneous antibodies (i.e., the individual antibodies comprising the population are identical except for possible naturally occurring mutations that may be present in minor amounts). Thus, the modifier “monoclonal” indicates the character of the antibody as not being a mixture of discrete antibodies.

Monoclonal antibodies can be prepared by methods known in the art, such as the hybridoma method of Kohler and Milstein by fusing splenocytes from immunized mice with continuously replicating tumor cells such as myeloma or lymphoma cells. (Kohler and Milstein (1975) Nature 256: 495-497; Gulfre and Milstein (1981) Methods in Enzymology: Immunochemical Techniques 73: 1-46, Langone and Banatis eds., Academic Press). The hybridoma cells are then cloned by limiting dilution methods and supernates assayed for antibody production by ELISA, RIA or bioassay. In another embodiment, monoclonals may be made by recombinant DNA methods.

For preparation of monoclonal antibodies (mAbs) directed toward a TAT-028 polypeptide, any technique which provides for the production of antibody molecules by continuous cell lines in culture may be used. For example, the hybridoma technique originally developed by Kohler and Milstein ((1975) supra, as well as in Kohler and Milstein (1976) Eur J Immunol. 6: 511-519; Kohler et al. (1976) Eur J Immunol. 6: 292-295; Hammerling et al. (1981) in: Monoclonal Antibodies and T-Cell Hybridomas, Elsevier, N.Y., pp. 563-681), and the trioma technique, the human B-cell hybridoma technique (Kozbor et al. (1983) Immunol Today. 4: 72-79), and the EBV-hybridoma technique to produce human monoclonal antibodies (Cole et al. (1985) in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs in the invention may be cultivated in vitro or in vivo. In an additional embodiment of the invention, monoclonal antibodies can be produced in germ-free animals utilizing technology known in the art.

In general, a mouse or other appropriate host animal, such as a hamster, is immunized with a TAT-028 polypeptide(s), or, more preferably, with a secreted TAT-028 polypeptide-expressing cell to induce lymphocytes that produce or are capable of producing antibodies that will specifically bind to the antigen or fragment thereof used for immunization. Alternatively, lymphocytes are immunized in vitro. TAT-028 polypeptide-expressing cells may be cultured in any suitable tissue culture medium, preferably in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of the immunized host animal (e.g., a mouse) are extracted and fused with a suitable myeloma cell line using a suitable fusing agent, such as polyethylene glycol, to form a hybridoma cell (Goding (1986) Monoclonal Antibodies: Principles and Practice, pp. 59-103, Academic Press). Any suitable myeloma cell line may be employed in accordance with the present invention; however, preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

The hybridoma cells thus prepared may be seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which prevent the growth of HGPRT-deficient cells. The hybridoma cells may be cloned by limiting dilution as described by Wands et al. ((1981) Gastroenterology 80: 225-232). The hybridoma cells obtained through such a selection and/or culture medium in which the hybridoma cells are being maintained can then be assayed to identify production of monoclonal antibodies directed against a TAT-028 antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by the Scatchard analysis of Munson and Rodbard (1980) Anal Biochem. 107: 220-239.

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxyapatite chromatography, gel electrophoresis, dialysis, or affinity chromatography.

DNA encoding the monoclonal antibodies of the invention is readily isolated and sequenced using conventional procedures (e.g., using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as E. coli cells, COS cells, Chinese hamster ovary (CHO) cells, or myeloma cells that do not otherwise produce immunoglobulin protein, to obtain the synthesis of monoclonal antibodies in the recombinant host cells (see e.g., Skerra et al. (1993) Curr Opin Immunol. 5: 256-262 and Pluckthun (1992) Immunol Rev. 130: 151-188).

The DNA also may be modified, for example, by substituting all or part of the coding sequence for human heavy- and light-chain constant domains in place of the homologous murine sequences (Morrison et al. (1984) Proc Natl Acad Sci. U.S.A. 81: 6851-6855), or by covalently joining to the immunoglobulin coding sequence all or part of the coding sequence for a non-immunoglobulin polypeptide. In that manner, “chimeric” or “hybrid” antibodies are prepared that have the binding specificity of an anti-TAT-028 antigen monoclonal antibody. Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for a TAT-028 antigen according to the invention and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

The antibodies in the present invention can also be generated using various phage display methods known in the art where functional antibody domains are displayed on the surface of phage particles carrying the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, for example, using labelled antigen or antigen bound or captured to a solid surface or bead. Phage display methods that can be used to make the antibodies of the present invention include those disclosed in Brinkman et al. (1995) J Immunol Meth. 182: 41-50; Ames et al. (1995) J Immunol Meth. 184: 177-186; Kettleborough et al. (1994) Eur J Immunol. 24: 952-958; Persic et al. (1997) Gene. 187: 9-18 ; Burton et al. (1994) Adv Immunol. 57: 191-280; European Publication No. EP 0589877; PCT Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, for example, as described in detail below. For example, techniques to recombinantly produce Fab, Fab′ and F(ab′)2 fragments can also be employed using methods known in the art such as those disclosed in WO 92/22324; Mullinax et al. (1992) Biotechniques. 12: 864-869; and Sawai et al. (1995) AJRI 34: 26-34; and Better et al. (1988) Science 240: 1041-1043.

Alternatively, additional antibodies capable of binding polypeptide(s) of the invention can be produced in a two-step procedure using anti-idiotypic antibodies. Exemplary methods for making anti-idiotypic antibodies may be found in, Asai (Ed.) (1993) Antibodies in Cell Biology. Methods in Cell Biology, Vol. 37, Academic Press, and U.S. Pat. No. 5,270,202, incorporated herein by reference. This method uses the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the polypeptide(s) of the invention protein-specific antibody can be blocked by polypeptide(s) of the invention. Such antibodies comprise anti-idiotypic antibodies to the polypeptide(s) of the invention protein-specific antibody and are used to immunize an animal to induce formation of further polypeptide(s) of the invention protein-specific antibodies.

iii.) Chimeric, Humanized, Primatized, Fully Human

Monoclonal antibodies of the invention include, but are not limited to, human monoclonal antibodies, primatized monoclonal antibodies, and chimeric monoclonal antibodies (for example, human-mouse chimeras). A chimeric antibody is a molecule in which different portions are derived from different animal species, such as those having a human immunoglobulin constant region and a variable region derived from a murine mAb (see e.g., U.S. Pat. Nos. 4,816,567 and 4,816,397). Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin, such as one or more complementarity determining regions (CDRs) from the non-human species and a framework region from a human immunoglobulin molecule (see e.g., U.S. Pat. No. 5,585,089).

Humanized antibodies include human immunoglobulins (recipient antibody) in which residues from a complementary-determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin, and all or substantially all of the FR regions are those of a human immunoglobulin consensus sequence. The humanized antibody optimally also will comprise at least a portion of an immunoglobulin constant region (Fc), typically that of a human immunoglobulin.

Chimeric and humanized monoclonal antibodies can be produced by recombinant DNA techniques known in the art, for example using methods described in WO 87/02671; EP 184,187; EP 171,496; EP 173,494; WO 86/01533; U.S. Pat. No. 4,816,567; EP 125,023; Better et al. (1988) Science 240: 1041-1043; Liu et al. (1987) Proc Natl Acad Sci. U.S.A. 84: 3439-3443; Liu et al. (1987) J Immunol. 139: 3521-3526; Sun et al. (1987) Proc Natl Acad Sci. U.S.A. 84: 214-218; Nishimura et al. (1987) Cancer Res. 47: 999-1005; Wood et al. (1985) Nature 314: 446-449; Shaw et al. (1988) J Natl Cancer Inst. 80: 1553-1559; Morrison (1985) Science 229: 1202-1207; Oi et al. (1986) Biotechniques. 4: 214; U.S. Pat. No. 5,225,539; Jones et al. (1986) Nature 321: 552-525; Verhoeyan et al. (1988) Science 239: 1534; and Beidler et al. (1988) J Immunol. 141: 4053-4060. See, below for a further discussion of humanized antibodies and methods related thereto.

Another highly efficient means for generating recombinant antibodies is disclosed by Newman ((1992) Biotechnology. 10: 1455-1460) incorporated herein by reference; see also U.S. Pat. Nos. 5,756,096; 5,750,105; 5,693,780; 5,681,722; and 5,658,570. Antibodies generated in this manner have previously been reported to display human effector function, have reduced immunogenicity, and long serum half-life.

Methods for humanizing non-human antibodies are well known in the art. Humanization may be essentially performed following the method of Winter and co-workers as described above (including Jones et al. (1986) Nature 321: 522-525; Riechmann et al. (1988) Nature 332: 323-327; Verhoeyen et al. (1988) Science 239: 1534-1536), by substituting rodent CDRs or CDR sequences for the corresponding sequences of a human antibody. Accordingly, such “humanized” antibodies are chimeric antibodies (U.S. Pat. Nos. 4,816,567 and 6,331,415). In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al. (1993) J Immunol. 151: 2296-2308; Chothia and Lesk (1987) J Mol Biol. 196: 901-917). Another method uses a particular framework derived from the consensus sequence of all human antibodies of a particular subgroup of light or heavy chains. The same framework may be used for several different humanized antibodies (Carter et al. (1992) Proc Natl Acad Sci. U.S.A. 89: 4285-4289; Presta et al. (1993) J Immunol. 151: 2623-2632). Another method may be found in US Pat. Publication No. 20030190705.

It is also desired that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues may be selected and combined from the consensus and import sequences so that the desired antibody characteristic, such as increased affinity for the target antigen(s), is achieved. In general, the CDR residues are directly and most substantially involved in influencing antigen binding.

Completely human antibodies are particularly desirable for therapeutic treatment of human patients. Such antibodies may be produced, for example, using transgenic mice which are incapable of expressing endogenous immunoglobulin heavy and light chain genes, but which can express human heavy and light chain genes. The transgenic mice may be immunized in the normal fashion with a selected antigen, e.g., all or a portion of a TAT-028 polypeptide. See for examples, PCT Publication Nos. WO 94/02602, WO 00/76310; U.S. Pat. Nos. 5,545,806; 5,545,807; 5,569,825; 6,150,584; 6,512,097; and 6,657,103; Jakobovits et al. (1993) Proc Natl Acad Sci. U.S.A. 90: 2551; Jakobovits et al. (1993) Nature 362: 255-258; Bruggemann et al. (1993) Year in Immunol. 7: 33-40; Mendez et al. (1997) Nat Gene. 15: 146-156, and Green and Jakobovits (1998) J Exp Med. 188: 483-495.

Human monoclonal antibodies can also be made by the hybridoma method. Human myeloma and mouse-human heteromyeloma cell lines for the production of human monoclonal antibodies have been described, for example, by Kozbor (1984) J Immunol. 133: 3001-3005; Brodeur et al. (1987) Monoclonal Antibody Production Techniques and Applications, pp.51-63, Marcel Dekker, Inc., New York; and Boerner et al. (1991) J Immunol. 147: 86-95.

Completely human antibodies which recognize a selected epitope can also be generated using a technique referred to as “guided selection.” In this approach, a selected non-human monoclonal antibody, e.g. a mouse antibody, is used to guide the selection of a completely human antibody recognizing the same epitope (Jespers et al. (1994) Biotechnology. 12: 899-903).

Alternatively, the phage display technology (McCafferty et al. (1990) Nature 348: 552-553) can be used to produce human antibodies and antibody fragments in vitro, from immunoglobulin variable (V) domain gene repertoires from non-immunized donors. Phage display can be performed in a variety of formats; for their review see, e.g., Johnson and Chiswell (1993) Curr Opin Struct Biol. 3: 564-571. Several sources of V-gene segments can be used for phage display. Clackson et al. (1991) Nature 352: 624-628 isolated a diverse array of anti-oxazolone antibodies from a small random combinatorial library of V genes derived from the spleens of immunized mice. A repertoire of V genes from non-immunized human donors can be constructed and antibodies to a diverse array of antigens (including self-antigens) can be isolated essentially following the techniques described by Marks et al. (1991) J Mol Biol. 222: 581-597, or Griffith et al. (1993) EMBO J. 12: 725-734.

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al. (1992) Biotechnology 10: 779-783). In this method, the affinity of “primary” human antibodies obtained by phage display can be improved by sequentially replacing the heavy and light chain V region genes with repertoires of naturally occurring variants (repertoires) of V domain genes obtained from non-immunized donors. This technique allows the production of antibodies and antibody fragments with affinities in the nM range. A strategy for making very large phage antibody repertoires has been described by Waterhouse et al. (1993) Nucleic Acids Res. 21: 2265-2266.

Gene shuffling can also be used to derive human antibodies from rodent antibodies, where the human antibody has similar affinities and specificities to the starting rodent antibody. According to this method, also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e., the epitope governs (imprints) the choice of partner. When the process is repeated in order to replace the remaining rodent V domain, a human antibody is obtained (see WO 93/06213). Unlike traditional humanization of rodent antibodies by CDR grafting, this technique provides completely human antibodies, which have no framework or CDR residues of rodent origin.

Examples of techniques which can be used to produce single-chain Fvs and antibodies include those described in U.S. Pat. Nos. 4,946,778 and 5,258,498; Huston et al. (1991) Meth Enzymol. 203: 46-88; Shu et al. (1993) Proc Natl Acad Sci. U.S.A. 90: 7995-7999; and Skerra et al. (1988) Science 240: 1038-1040.

iv.) Bispecific

The invention further provides bispecific antibodies, which can be made by methods known in the art. Bispecific antibodies are monoclonal, preferably human or humanized, antibodies that have binding specificities for at least two different antigens. Traditional production of full length bispecific antibodies is based on the coexpression of two immunoglobulin heavy chain-light chain pairs, where the two chains have different specificities (Milstein and Cuello (1983) Nature 305: 537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al. (1991) EMBO J. 10: 3655-3659.

In another, more preferred, approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, C_{H}2, and C_{H}3 regions. It is preferred to have the first heavy-chain constant region (C_{H}1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This approach is disclosed in WO 94/04690. For further details for generating bispecific antibodies see, for example, Suresh et al. ((1 986) Meth Enzymol. 121: 210-228).

v.) Other

Heteroconjugate antibodies are also within the scope of the present invention. Heteroconjugate antibodies are composed of two covalently joined antibodies. Such antibodies can be, for example, diabodies, triabodies or tetrabodies. Such antibodies have, for example, been proposed to target immune system cells to unwanted cells (U.S. Pat. No. 4,676,980), and for treatment of HIV infection (WO 91/00360; WO 92/00373; and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

In another preferred embodiment, multi-specific antibodies, fragments, and fusion proteins of the present invention, such as heteroconjugate antibodies, can be targeted against an antigens selected from the group of known immunotherapy targets consisting of CD2 (GenBank GI #115975), CD3 (GenBank GI #1345708 (epsilon subunit)), CD4 (GenBank GI #116013), CD5 (GenBank GI #116024), CD8 (GenBank GI #116035), CD11c (GenBank GI #386831), CD14 (GenBank GI #29741), CD15 (GenBank GI #4503811), CD19 (GenBank GI #178667), CD20 (GenBank GI #115968), CD21 (GenBank GI #117315), CD22 (GenBank GI #29779), CD23 (GenBank GI #119862), CD25 (GenBank GI #124317), CD30 (GenBank GI #115978), CD33 (GenBank GI #115979), CD37 (GenBank GI #115983), CD38 (GenBank GI #180119), CD40 (GenBank GI #116000), CD44 (GenBank GI #950417), CD44v6 (CD44 isoforms containing variant exon 6, e.g., GenBank GI #48255937, GenBank GI #48255935), CD45 (GenBank GI #34281), CD46 (GenBank GI #262938), CD48 (GenBank GI #114871), CD51 (integrin αv) (GenBank GI #124959), CD52 (GenBank GI #3182945), CD54 (GenBank GI #124098), CD56 (GenBank GI #3334473), CD61 (integrin β3) (GenBank GI #124968), CD66e (CEA) (GenBank GI #115940), CD70 (GenBank GI #545773), CD71 (transferrin receptor) (GenBank GI #136378), CD72 (GenBank GI #116029), CD74 (GenBank GI #10835071), CD80 (GenBank GI #461606), CD87 (uPAR) (GenBank GI #465003), CD95 (Apo-1, Fas) (GenBank GI #119833), CD97 (GenBank GI #42560541), CD98 (4F2) (GenBank GI #112803), CD105 (GenBank GI #182091), CD122 (GenBank GI #124321), CD126 (GenBank GI #124343), CD135 (Flt3) (GenBank GI #544320), CD144 (vascular endothelial cadherin) (GenBank GI #13432109), CD146 (MUC18) (GenBank GI #1171064), CD152 (CTLA4) (GenBank GI #27735177), CD154 (CD40L) (GenBank GI #38412), CD155 (GenBank GI #1346922), CD178 (CD95L) (GenBank GI #1345957), CD221 (insulin-like growth factor receptor 1) (GenBank GI #124240), CD224 (gamma glutamyl transferase) (GenBank GI #121148), CD227 (MUC1) (GenBank GI #547937), CD243 (MDRI) (GenBank GI #2506118), BAFF (GenBank GI #13124573), BAFF receptor (GenBank GI #21264093), BST2 (GenBank GI #1705508), endosialin (GenBank GI #9966885), HLA-DR beta (GenBank GI #188241), tenascin (GenBank GI #3915888), her2/neu (GenBank GI #119533), Muc16 (GenBank GI #34501467), G250 (GenBank GI #5915865), TweakR (GenBank GI #21263626), PSMA (GenBank GI #548615), TRAIL-R1 (DR4) (GenBank GI #21264525), TRAIL-R2 (DR5) (GenBank GI #17380321), TP-1 antigen (Bruland et al. (1988) Cancer Res. 48: 5302-5309), 8H9 glycoprotein (Modak et al. (2001) Cancer Res. 61: 4048-4054), EGP-1 (TACSTD-2) (GenBank GI #1346075), KGF-2 (FGF-10) (GenBank GI #6015141), A33 antigen (GenBank GI #2842765), MCSP (GenBank GI #20141463), lactadherin (GenBank GI #2506380), EphA2 (GenBank GI #125333), EphA4 (GenBank GI #1711371), EphB2 (GenBank GI #12644190), CCR4 (GenBank GI #1705894), E48 (GenBank GI #2501524), 5T4 fetal protein trophoblast (GenBank GI #435655), Muc5AC (GenBank GI #46397621), FAPA (GenBank GI #20140021), LTBR (GenBank GI #549090), CFR-1 (GenBank GI #17376711), PGRN (GenBank GI #121617), VEGFR-2 (GenBank GI #9087218), MOv18 (GenBank GI #544337), Cripto (GenBank GI #117473), Wnt-1 (GenBank GI #139743), Wnt-2 (GenBank GI #4507927), parathyroid hormone-related peptide (GenBank GI #131542), scatter factor (GenBank GI #123116), EGF receptor (GenBank GI #2811086), TAG72 (Muraro et al. (1988) Cancer Res. 48: 4588-4596), CanAg (Baeckstrom et al. (1991) J Biol Chem. 266: 21537-21547 and GenBank GI #547937), C30.6 (Mount et al. (1994) Cancer Res. 54: 6160-6166), GD2 ganglioside (Nagata et al. (1992) J Biol Chem. 267: 12082-12089), GD3 ganglioside (Zou et al. (2004) J Biol Chem. 279: 25390-25399), adenocarcinoma Lewis Y antigen (Nudelman et al. (1986) J Biol Chem. 261: 11247-11253; Kim et al. (1986) Cancer Res. 46: 5985-5992), Human carcinoma L6 carbohydrate (Hellstrom et al. (1986) Cancer Res. 46: 3917-3923; Fell et al. (1992) J Biol Chem. 267:15552-15558), IL-8 (GenBank GI #124359), EpCam (TACSTD-1, EGP-2) (GenBank GI #120749), LI-CAM splice variant (Meli et al. (1999) Int J Cancer. 83: 401-408; GenBank GI #4557707, 13435353), vitronectin (GenBank GI #139653), placental alkaline phosphatase (GenBank GI #130737), neuropilin (GenBank GI #9297107), and B-cell-tumor-associated antigens, including vascular endothelial antigens, such as vascular endothelial growth factor (VEGF) (GenBank GI #30172564) and placenta growth factor (PLGF) (GenBank GI #17380553). For brevity, the GenBank GI #s provided are intended as representative and may be considered a preferred sequence, however they are meant to encompass splice variants, variants, isoforms, polymorphisms, mutations, modifications, and the like, preferably those associated with cancer. Preferably such variant sequences have at least 90% sequence identity to the representative sequence, more preferably at least 95% sequence identity, or at least 96%, 97%, 98%, or 99% sequence identity. Proteins presented in their precursor form, are also preferred in their mature form. Proteins present in hetero- or homo-multimers may be targeted as individual proteins or as part of their multimeric complex (e.g., integrin αvβ3). Multimer subunits presented (e.g. CD3 epsilon subunit) may be taken as more preferable subunits, but the other subunits and multimeric forms are also preferred. In a related vein, additional specificities of the antibodies and the like can be the same or different. Methods for producing tetrameric antibodies and domain-deleted antibodies, in particular CH₂ domain-deleted antibodies, are disclosed in WO 02/060955 and WO 02/096948.

As discussed above, because humanized and human antibodies are far less immunogenic in humans than other species monoclonal antibodies, e.g., murine antibodies, they can be used for the treatment of humans with far less risk of anaphylaxis. Thus, these antibodies may be preferred in therapeutic applications that involve in vivo administration to a human such as the use of such antibodies as radiation sensitizers for the treatment of neoplastic disease or in methods to reduce the side effects of additional therapies such as cancer therapy.

The invention provides functionally-active fragments, derivatives or analogues of the anti-TAT-028 polypeptide immunoglobulin molecules. “Functionally-active” in this context means that the fragment, derivative or analogue is able to induce anti-anti-idiotype antibodies (i.e. tertiary antibodies) that recognize the same antigen that is recognized by the antibody from which the fragment, derivative or analogue is derived. Specifically, in a preferred embodiment, the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

The present invention provides antibody fragments such as, but not limited to, F(ab′)₂, F(ab)₂, Fab′, Fab, scFvs. Antibody fragments which recognize specific epitopes may be generated by known techniques, e.g., by pepsin or papain-mediated cleavage.

The invention also provides heavy chain and light chain dimers of the antibodies of the invention, or any minimal fragment thereof such as Fvs or single chain antibodies (SCAs) (e.g., as described in U.S. Pat. No. 4,946,778; Bird (1988) Science 242: 423-42; Huston et al. (1988) Proc Natl Acad Sci. U.S.A. 85: 5879-5883; and Ward et al. (1989) Nature 334: 544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al. (1988) Science 242: 1038-1041).

Alternatively, a clone encoding at least the Fab portion of the antibody may be obtained by screening Fab expression libraries (e.g., as described in Huse et al. (1989) Science 246: 1275-1281) for clones of Fab fragments that bind the specific antigen or by screening antibody libraries (see, e.g., Clackson et al. (1991) Nature 352: 624-628; Hanes and Pluckthun (1997) Proc Natl Acad Sci. U.S.A. 94: 4937-4942).

In other embodiments, the invention provides fusion proteins of the immunoglobulins of the invention, or functionally active fragments thereof. In one example, the immunoglobulin is fused via a covalent bond (e.g., a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

Intrabodies—intracellular antibodies or fragments thereof—are also contemplated. See for example: Bonnin et al. (2004) Methods 34: 225-232; Auf der Maur et al. (2004) Methods 34: 215-224; Kontermann (2004) Methods 34: 163-170; Visintin et al. (2004) Methods 34: 200-214; Colby et al. (2004) J Mol Biol. 342: 901-912; Ewert et al. (2004) Methods 34: 184-199; Strube and Chen (2004) Methods 34: 179-183; Blazek and Celer (2003) Folia Microbiol (Praha). 48: 687-698; Tanaka et al. (2003) J Mol Biol. 331: 1109-1120; Donini et al. (2003) J Mol Biol. 330: 323-332; Tanaka et al. (2003) Nucleic Acids Res. 31: e23; Nam et al. (2002) Methods Mol Biol. 193: 301-327; Auf der Maur et al. (2002) J Biol Chem. 277: 45075-45085; Auf der Maur et al. (2001) FEBS Lett. 508: 407-412; Cohen (2002) Methods Mol Biol. 178: 367-378; Strube and Chen (2002) J Immunol Methods. 263: 149-167; Rajpal and Turi (2001) J Biol Chem. 276: 33139-33146; Ohage and Steipe (1999) J Mol Biol. 291: 1119-1128; Ohage et al. (1999) J Mol Biol. 291: 1129-1134; Wirtz and Steipe (1999) Protein Sci. 8: 2245-2250; Proba et al. (1998) J Mol Biol. 275: 245-253; Steipe (2004) Methods Enzymol. 388: 176-186.

In another embodiment, the invention provides for the compositions and use of pooled antibodies, antibody fragments, and the other antibody variants described herein. For example, two or more monoclonals may be pooled for use.

In the production of antibodies, screening for the desired antibody, fragment, or modification thereof can be accomplished by techniques known in the art, e.g., ELISA (enzyme-linked immunosorbent assay), or panels of hybridomas or purified monoclonal antibodies may be screened using antigen displayed on the surface of filamentous bacteriophage as described in Lijnen et al. (1997) Anal Biochem. 248: 211-215. For example, to select antibodies which recognize a specific domain of a TAT-028 polypeptide, one may assay generated hybridomas for a product which binds to a polypeptide fragment containing such domain. For selection of an antibody that specifically binds a first polypeptide homologue but which does not specifically bind to (or binds less avidly to) a second polypeptide homologue, one can select on the basis of positive binding to the first polypeptide homologue and a lack of binding to (or reduced binding to) the second polypeptide homologue. Antibodies can also be evaluated by flow cytometry on cells transfected with the target protein. Antibodies that contain appropriate reactivity can then be tested for their specificity in transfected cells and tissue sections, if applicable.

vi) Antibody Nucleic Acids

The nucleic acid encoding an antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridizable to the 3′ and 5′ ends of the sequence or by cloning using an oligonucleotide probe specific for the particular gene sequence.

The nucleic acid encoding the antibody may be used to introduce the nucleotide substitution(s) or deletion(s) necessary to substitute (or delete) one or more variable region cysteine residues participating in an intrachain disulphide bond with an amino acid residue that does not contain a sulphydryl group. Such modifications can be carried out by any method known in the art for the introduction of specific mutations or deletions in a nucleotide sequence, including, for example, but not limited to, chemical mutagenesis, in vitro site directed mutagenesis (Hutchinson et al. (1978) J Biol Chem. 253: 6551-6560) and PCR based methods. In addition, techniques developed for the production of “chimeric antibodies” (Morrison et al. (1984) Proc Natl Acad Sci. U.S.A. 81: 851-855; Neuberger et al. (1984) Nature 312: 604-608; Takeda et al. (1985) Nature 314: 452-454) by splicing genes from a mouse antibody molecule of appropriate antigen specificity together with genes from a human antibody molecule of appropriate biological activity can also be used. As described supra, a chimeric antibody is a molecule in which different portions are derived from different animal species, such as those having a variable region derived from a murine mAb and a human antibody constant region, e.g., humanized antibodies.

vii) Antibody Production

The antibodies of the invention can be produced by any method known in the art for the synthesis of antibodies (e.g., chemical synthesis), and are preferably produced by a recombinant expression technique. Recombinant expression of antibodies, or fragments, derivatives or analogues thereof, requires construction of a nucleic acid that encodes the antibody. If the nucleotide sequence of the antibody is known, a nucleic acid encoding the antibody may be assembled from chemically synthesized oligonucleotides (e.g., as described in Kutemeier et al. (1994) Biotechniques 1 7: 242-246).

Immunoglobulins (Ig) and certain variants thereof are known and many have been prepared in recombinant cell culture. For example, see U.S. Pat. Nos. 4,745,055 and 5,116,964; EP 256,654; EP 120,694; EP 125,023; EP 255,694; EP 266,663; WO 88/03559; Falkner and Zachau (1982) Nature, 298: 286-288; Morrison (1979) J Immun. 123: 793-800; Koehler et al. (1980) Proc Natl Acad Sci. U.S.A. 77: 2197-2199; Raso and Griffin (1981) Cancer Res. 41: 2073-2078; Morrison and Oi (1984) Ann Rev Immunol. 2: 239-256; Morrison (1985) Science 229: 1202-1207; and Morrison et al. (1984) Proc Natl Acad Sci. U.S.A. 81: 6851-6855. Reassorted immunoglobulin chains are also known. See, for example, U.S. Pat. No. 4,444,878; WO 88/03565; and EP 68,763 and references cited therein. The immunoglobulin moiety in the chimeras of the present invention may be obtained from IgG-1, IgG-2, IgG-3, or IgG-4 subtypes, IgA, IgE, IgD, or IgM, preferably from IgG-1 or IgG-3.

Once a nucleic acid encoding at least the variable domain of the antibody molecule is obtained, it may be introduced into a vector containing the nucleotide sequence encoding the constant region of the antibody molecule (see, e.g., WO 86/05807; WO 89/01036; and U.S. Pat. No. 5,122,464). Vectors containing the complete light or heavy chain for co-expression with the nucleic acid to allow the expression of a complete antibody molecule are also available.

The expression vector may be transferred to a host cell by conventional techniques and the transfected cells can then be cultured by conventional techniques to produce an antibody of the invention (see e.g., Ramirez-Solis et al. (1990) Gene. 87: 291-4; Foecking and Hofstetter (1986) Gene. 45: 101-105; Cockett et al. (1990) Biotechnology. 8: 662-667).

A variety of host-expression vector systems, inclusive of those described herein for TAT-028 polypeptides, may be utilized to express an antibody molecule of the invention. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CAMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

For long-term, high-yield production of recombinant antibodies, stable expression is preferred. For example, cells lines that stably express an antibody of interest can be produced by transfecting the cells with an expression vector comprising the nucleotide sequence of the antibody and the nucleotide sequence of a selectable marker (e.g., neomycin or hygromycin), and selecting for expression of the selectable marker. Such engineered cell lines may be particularly useful in screening and evaluation of compounds that interact directly or indirectly with the antibody molecule. The expression levels of the antibody molecule can be increased by vector amplification (for a review, see Bebbington and Hentschel (1987) “The use of vectors based on gene amplification for the expression of cloned genes in mammalian cells” in DNA cloning, Vol. 3, Academic Press, New York). When a marker in the vector system expressing antibody is amplifiable, an increase in the level of inhibitor present in culture of host cell will increase the number of copies of the marker gene. Since the amplified region is associated with the antibody gene, production of the antibody will also increase (Crouse et al. (1983) Mol Cell Biol. 3: 257-266).

The host cell may be co-transfected with two expression vectors for use within the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides (see Proudfoot (1986) Nature 322: 562-565; Kohler (1980) Proc Natl Acad Sci. U.S.A. 77: 2197-2199). The coding sequences for the heavy and light chains may comprise cDNA or genomic DNA. Once the antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any antibody fusion protein may be readily purified by utilizing an antibody specific for the fusion protein being expressed. For example, a system described by Janknecht et al. ((1991) Proc Natl Acad Sci. U.S.A. 88: 8972-8976)

The immunoglobulins of the invention include analogues and derivatives that are either modified, i.e. by the covalent attachment of any type of molecule as long as such covalent attachment that does not impair immunospecific binding beyond the preferred binding affinity range discussed above. For example, the derivatives and analogues of the immunoglobulins include those that have been further modified, e.g., by glycosylation, acetylation, pegylation, phosphylation, amidation, derivatisation by protecting/blocking groups, proteolytic cleavage, and linkage to a cellular ligand or other protein, etc. Any of numerous chemical modifications may be carried out by known techniques, for example specific chemical cleavage, acetylation, formylation, etc. Additionally, the analogue or derivative may contain one or more non-natural amino acids.

Antibodies of the invention and fragments thereof, e.g., domain-deleted antibody fragments, will be useful for purifying TAT-028 antigens, and for passive anti-cancer immunotherapy, or may be attached to therapeutic effector moieties, e.g., radiolabels, cytotoxins, therapeutic enzymes, agents that induce apoptosis, in order to provide for targeted cytotoxicity, i.e., killing of human lung tumor cells.

Anti-TAT-028 antibodies or fragments thereof may be administered in labeled or unlabeled form, alone or in combination with other therapeutics, e.g., chemotherapeutics such as cisplatin, methotrexate, adriamycin, and other chemotherapies suitable for lung cancer therapy, therapeutic proteins such as lymphokines and cytokines, diagnostic and therapeutic enzymes, radionuclides, prodrugs, cytotoxins, and the like. Antibodies of the invention or fragments thereof can thus be conjugated to a therapeutic agent or drug moiety to modify a given biological response. The therapeutic agent or drug moiety can include classical chemical therapeutic agents (e.g., adriamycin, methotrexate, cisplatin, daunorubicin, doxorubicin, methopterin, caminomycin, mitheramycin, streptnigrin, chlorambucil, and ifosfimide).For example, the drug moiety may be a protein or polypeptide possessing a desired biological activity. Such proteins may include toxins, e.g., abrin, ricin A, calicheamicin, euperamicin, dynemicin, pseudomonas exotoxin, cholera toxin, diphtheria toxin and variants thereof; therapeutic proteins (tumor necrosis factor, α-interferon, γ-interferon, nerve growth factor, platelet derived growth factor, and tissue plasminogen activator); a thrombotic agent; an anti-angiogenic agent;and other growth factors; hormones and hormone antagonists, e.g., corticosteroids (e.g., prednisone), progestins, antiestrogens (e.g., tamoxifin), androgens (e.g., testosterone), and aromatase inhibitors. Other therapeutic moieties may include radionuclides such as ⁹⁰Y, ¹²⁵I, ¹³¹I, ¹¹¹In, ¹⁰⁵Rh, ¹⁵³Sm, ⁶⁷Cu, ⁶⁷Ga, ¹⁶⁶Ho, ¹⁷⁷Lo, ¹⁸⁶Re, ²¹³Bi, ²¹¹At, ¹⁰⁹Pd, ²¹²Bi, and ¹⁸⁸Re; antibiotics, e.g., calicheamicin; pro-drugs such as phosphate-containing prodrugs, thiophosphate-containing prodrugs, sulfate containing prodrugs peptide containing prodrugs, and beta lactam containing prodrugs; and drugs such as but not limited to, alkylphosphocholines, topoisomerase I inhibitors, taxoids and suramin.

Techniques for conjugating such therapeutic moieties to antibodies are well known; see, e.g., Arnon et al. (1985) “Monoclonal Antibodies for Immunotargeting of Drugs in Cancer Therapy” in Monoclonal Antibodies and Cancer Therapy, Reisfeld et al. (Eds.), pp. 243-256, Alan R. Liss, Inc.; Hellstrom et al. (1987) “Antibodies for Drug Delivery” in Controlled Drug Delivery, 2nd Edit. Robinson et al. (Eds.) pp. 623-653, Marcel Dekker, Inc.; Thorpe (1985) “Antibody Carriers of Cytotoxic Agents in Cancer Therapy: A Review” in Monoclonal Antibodies: Biological and Clinical Applications Pinchera et al. (Eds.) pp. 475-506; (1985) “Analysis, Results, and Future Prospective of the Therapeutic Use of Radiolabelled Antibody in Cancer Therapy” in Monoclonal Antibodies For Cancer Detection And Therapy, Baldwin et al. (Eds.) pp. 303-316, Academic Press; Thorpe et al. (1982) Immunol Rev. 62: 119-158; and Dubowchik et al. (1999) Pharmacol Ther. 83: 67-123. In another embodiment, an antibody may be conjugated to a second antibody to form an antibody heteroconjugate as described in U.S. Pat. No. 4,676,980. An antibody, with or without a therapeutic moiety conjugated to it, can be used as a therapeutic agent that is administered alone or in combination with cytotoxic factor(s) and/or cytokine(s).

The administered composition may include a pharmaceutically acceptable carrier, and optionally adjuvants and/or stabilizers used in antibody compositions for therapeutic use. Administration may be local or systemic.

Screening Methods

The invention provides methods for identifying candidate compounds that bind to a TAT-028 polypeptide or have a stimulatory or inhibitory effect on the expression or activity of a TAT-028 polypeptide. Examples of compounds, include, but are not limited to, nucleic acids (e.g., DNA and RNA), carbohydrates, lipids, proteins, peptides, peptidomimetics, hormones, cytokines, antibodies, agonists, antagonists, small molecules, aptamers (see U.S. Pat. Nos. 5,756,291 and 5,792,613), nucleic acid-protein fusions (see U.S. Pat. No. 6,489,116), other drugs, and combinations and variations thereupon. These methods, whether cell-based or cell-free, can be used to screen a plurality (e.g., a library) of candidate compounds.

Compounds can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12: 145-167; U.S. Pat. Nos. 5,738,996; and 5,807,683).

Examples of methods for the synthesis of molecular libraries may be found in the art, for example in: DeWitt et al. (1993) Proc Natl Acad Sci. U.S.A. 90: 6909-6913; Erb et al. (1994) Proc Natl Acad Sci. U.S.A. 91: 11422-11426; Zuckermann et al. (1994) J Med Chem. 37: 2678-2685; Cho et al. (1993) Science 261: 1303-1305; Carell et al. (1994) Angew Chem Int Ed Engl. 33: 2059-2061; Carell et al. (1994) Angew Chem Int Ed Engl. 33: 2061-2064; and Gallop et al. (1994) J Med Chem. 37: 1233-1251. Libraries of compounds may be presented, e.g., presented in solution (e.g., Houghten (1992) Biotechniques. 13: 412-421), or on beads (Lam (1991) Nature 354: 82-84), chips (Fodor (1993) Nature 364: 555-556), bacteria (U.S. Pat. No. 5,223,409), spores (U.S. Pat. Nos. 5,571,698; 5,403,484; and 5,223,409), plasmids (Cull et al. (1992) Proc Natl Acad Sci. U.S.A. 89: 1865-1869) or phage (Scott and Smith (1990) Science 249: 386-390; Devlin (1990) Science 249: 404-406; Cwirla et al. (1990) Proc Natl Acad Sci. U.S.A. 87: 6378-6382; and Felici (1991) J Mol Biol. 222: 301-310).

In a preferred embodiment, the invention provides methods for the identification of compounds that modulate (e.g., upregulate or downregulate) TAT-028 polypeptide and/or polynucleotide expression or activity, that includes contacting a candidate compound with a TAT-028 and detecting the presence or absence of binding between the compound and the TAT-028, or detecting an alteration or modulation in TAT-028 expression or activity. Further, methods are also included for the identification of compounds that modulate (e.g., upregulate or downregulate) TAT-028 expression or activity that include administering a compound to a cell or cell population, and detecting an alteration in TAT-028 expression or activity. Preferably, such compounds inhibit TAT-028 binding, expression, or activity by at least 0.1%, at least 1%, at least 5%, or at least 10% of the activity of a TAT-028 polypeptide or TAT-028 nucleic acid sequence described herein. More preferably, such compounds inhibit at least 25%, at least 50%, at least 75%, or at least 90% of the activity of a TAT-028 polypeptide or TAT-028 nucleic acid sequence described herein. Most preferably, such compounds inhibit at least 95%, at least 96%, at least 97%, at least 98%, or at least 99% of the activity of a TAT-028 polypeptide or TAT-028 nucleic acid sequence described herein. Such compounds can be identified in a cell based or cell free assay. Inhibition or modulation of TAT-028 expression or biological activity by a compound in a sample treated with the compound can be determined by comparison to an untreated sample, a sample treated with a second compound, a control or a reference sample or value.

Candidate compounds can be identified as a modulator of the expression of the TAT-028 polypeptide or nucleic acid based on a comparison to a control or referenced sample, preferably one that is not treated with the candidate compound. For example, when expression of the TAT-028 polypeptide or mRNA encoding said polypeptide is significantly greater in the presence of the candidate compound than in its absence, the candidate compound is identified as a stimulator of expression of the TAT-028 polypeptide or mRNA encoding said polypeptide.

Alternatively, when expression of the TAT-028 polypeptide or mRNA encoding the polypeptide is significantly less in the presence of the candidate compound than in its absence, the candidate compound is identified as an inhibitor of the expression of the TAT-028 polypeptide or mRNA encoding the polypeptide. The level of expression of a TAT-028 polypeptide, or the mRNA that encodes it, can be determined by methods known to those of skill in the art based on the present description. For example, TAT-028 mRNA expression can be assessed by Northern blot analysis or RT-PCR, and protein levels can be assessed by Western blot analysis or other means known in the art.

In another embodiment, compounds that modulate an activity or characteristic of a TAT-028 polypeptide are identified by contacting a preparation containing the TAT-028 polypeptide, or cells expressing the TAT-028 polypeptide with a candidate compound or a control and determining the ability of the candidate compound to modulate (e.g., stimulate or inhibit) an activity of the TAT-028 polypeptide. An activity of a TAT-028 polypeptide can be assessed by detecting its effect on a “downstream effector” for example, induction of a cellular signal transduction pathway of the polypeptide (e.g., intracellular Ca2+, diacylglycerol, IP3, cAMP, or other intermediate), detecting catalytic or enzymatic activity of the TAT-028 polypeptide on a suitable substrate, detecting the induction of a reporter gene (e.g., a regulatory element that is responsive to a TAT-028 polypeptide and is operably linked to a nucleic acid encoding a detectable marker, e.g., luciferase), or detecting a cellular response, for example, cellular differentiation, or cell proliferation as the case may be, based on the present description, techniques known to those of skill in the art can be used for measuring these activities (see, e.g., U.S. Pat. No. 5,401,639).

Methods are also provided for selecting TAT-028 binding molecules, such as antibodies, antibody-related proteins, or small molecules are provided. Such methods include a method for selecting an antibody that binds with high binding affinity to a mammalian TAT-028 that includes the steps of: (a) providing a peptide comprising a TAT-028 polypeptide, optionally coupled to an immunogenic carrier and (b) contacting the TAT-028 polypeptide with a TAT-028 binding molecule, wherein the TAT-028 binding molecule is an antibody, under conditions that allow for complex formation between the TAT-028 polypeptide and the antibody, thereby selecting a TAT-028 binding molecule that binds with high binding affinity to a mammalian TAT-028. Preferably such compounds bind one or more TAT-028 polypeptides specifically. Such compounds may also include, but are not limited to, nucleic acids (e.g., DNA and RNA), carbohydrates, lipids, proteins, peptides, peptidomimetics, hormones, cytokines, antibodies, agonists, antagonists, small molecules, aptamers (see U.S. Pat. Nos. 5,756,291 and 5,792,613), nucleic acid-protein fusions (see U.S. Pat. No. 6,489,116), other drugs, and combinations and variations thereupon. Such compounds may have uses in diagnosis of cancer, such as lung cancer. Such compounds may also have uses in treatment of cancer, such as lung cancer, even in the absence of a measurable alteration in TAT-028 expression or activity, for example, such as might be expected in a non-activity based binding assay.

The ability of the candidate compound to interact directly or indirectly with the TAT-028 polypeptide can be determined by methods known to those of skill in the art (e.g., by flow cytometry, a scintillation assay, immunoprecipitation or Western blot analysis).

In one embodiment, a TAT-028 polypeptide is used as a “bait protein” in a two-hybrid assay or three-hybrid assay to identify other proteins that bind to or interact with the TAT-028 polypeptide (see e.g., U.S. Pat. No. 5,283,317; Zervos et al. (1993) Cell 72: 223-232; Madura et al. (1993) J Biol Chem. 268: 12046-12054; Bartel et al. (1993) Biotechniques. 14: 920-924; Iwabuchi et al. (1993) Oncogene. 8: 1693-1696; and WO 94/10300). As those skilled in the art will appreciate, such binding proteins are also likely to be involved in the propagation of signals by a TAT-028 polypeptide. For example, they may be upstream or downstream elements of a signaling pathway involving a TAT-028 polypeptide. Alternatively, polypeptides that interact with a TAT-028 polypeptide can be identified by isolating a protein complex comprising a TAT-028 polypeptide (i.e. a TAT-028 polypeptide which interacts directly or indirectly with one or more other polypeptides) and identifying the associated proteins using methods known in the art such as mass spectrometry or Western blotting (for examples see Blackstock and Weir (1999) Trends in Biotechnology 17: 121-127; Rigaut (1999) Nat Biotechnol. 17: 1030-1032; Husi (2000) Nat Neurosci. 3: 661-669; Ho et al. (2002) Nature 415: 180-183; Gavin et al. (2002) Nature 415: 141-147).

In all cases, the ability of the candidate compound to interact directly or indirectly with the TAT-028 polypeptide can be determined by methods known to those of skill in the art including, for example, flow cytometry, a scintillation assay, an activity assay, mass spectrometry, microscopy, immunoprecipitation, and Western blot analysis. Panels of hybridomas or purified monoclonal antibodies may be screened, for example, using antigen displayed on the surface of filamentous bacteriophage as described in Lijnen et al. (1997) Anal Biochem. 248: 211-215.

Also provided are comparative methods for identifying a candidate compound for the treatment of cancer, that include: (a) measuring the binding of a TAT-028 binding molecule to a TAT-028 polypeptide in the presence of a test compound; and (b) measuring the binding of the TAT-028 binding molecule to a TAT-028 polypeptide in the absence of the test compound; wherein a level of binding of the TAT-028 binding molecule to a TAT-028 polypeptide in the presence of the test compound that is less than the level of binding of the TAT-028 binding molecule to a TAT-028 polypeptide in the absence of the test compound is an indication that the test compound is a potential therapeutic compound for the treatment of a cancer. Also provided are methods for identifying a compound for diagnosing a cancer that include: (a) measuring the binding of a TAT-028 binding molecule to a TAT-028 polypeptide in the presence of a test compound; and (b) measuring the binding of the TAT-028 binding molecule to a TAT-028 polypeptide in the absence of the test compound; wherein a level of binding of the TAT-028 binding molecule to a TAT-028 polypeptide in the presence of the test compound that is less than the level of binding of the TAT-028 binding molecule to a TAT-028 polypeptide in the absence of the test compound is an indication that the test compound is a potential compound for diagnosing a cancer.

In another embodiment, the availability of isolated TAT-028 polypeptides also allows for the identification of small molecules and low molecular weight compounds that inhibit the binding of TAT-028 polypeptides to binding partners (such as antibodies, CDR regions, substrates, or interacting cellular biomolecules) through routine application of high-throughput screening methods (HTS) (Gonzalez et al. (1998) Curr Opin Biotech. 9: 624-631; Sarubbi et al. (1996) Anal Biochem. 237: 70-75; Martens et al. (1999) Anal Biochem. 273: 20-31).

In a preferred embodiment for therapeutic applications, identified compounds (preferably antibodies) that bind TAT-028 and/or modulate TAT-028 expression or activity also inhibit cell and/or tumor growth, proliferation, and/or metastasis, for example, such as might be present in a cellular proliferative disease; or contribute to cell death, such as through apoptosis. For example, an anti-TAT-028 antibody may inhibit cell proliferation or promote cell death in lung tumor xenografts in mice via an immune response. Such properties may be assayed by methods known in the art, for example, cell death can be measured by determining cellular ATP levels, wherein a cell that is undergoing cell death has a decreased level of cellular ATP compared to a control cell. Cell death may also be measured by staining with a vital dye, for example, trypan blue, wherein a cell that is dying will be stained with the vital dye, and a cell that is not dying will not be stained with the dye. Inhibition of cell proliferation can be measured, for example, by determining by standard means the number of cells in a population contacted with the compound compared to the number of cells in a population not contacted with the compound. If the number of cells in the population contacted with the compound does not increase over time or increases at a reduced rate compared to cells not contacted with the compound, the candidate compound inhibits the proliferation of the cells. Common proliferation assays include incorporating a radiolabelled substance such as ³H-thymidine in the DNA, and the assay for incorporating bromodeoxyuridine developed by the Boehringer Mannheim GmbH. Cell growth can be measured, for example, by determining the relative size of individual cells or the relative mass of a population of cells between cells or populations of cells treated with the compound and untreated cells. Metastasis may be measured by, for example, by the methods described in U.S. Pat. Nos. 6,245,898 or 6,767,700, using appropriate tumor samples. Assays may be performed in cell culture, animal models, or in human clinical trials.

Compounds or agents identified as modulators of TAT-028 polypeptide or TAT-028 nucleic acid expression and/or activity, and/or identified as TAT-028 binding compounds by any of the methods herein may be used in further testing, or in therapeutic or prophylactic use as an anti-cancer agent. Thus, the present invention also provides assays for use in drug discovery or target validation in order to identify or verify the modulators of TAT-028, preferably for treatment or prevention of cancer. Test compounds can be assayed for their ability to modulate levels of a TAT-028 polypeptide in a subject having cancer. Compounds able to modulate levels of a TAT-028 polypeptide in a subject having cancer towards levels found in subjects free from cancer or to produce similar changes in experimental animal models of cancer can be used as lead compounds for further drug discovery, or used therapeutically. Such assays can also be used to screen candidate drugs, in clinical monitoring or in drug development, where an abundance of a TAT-028 polypeptide can serve as a surrogate marker for clinical disease.

Diagnostics

The invention provides methods for detecting the presence and status of TAT-028 polypeptides in various biological samples, as well as methods for identifying cells that express TAT-028 polypeptides. A typical embodiment of this invention provides methods for monitoring TAT-028 protein in a tissue or bodily fluid sample having or suspected of having some form of growth dysregulation such as cancer.

In general, a cancer may be detected in a patient based on the presence of one or more lung cancer proteins and/or polynucleotides encoding such proteins in a biological sample (for example, blood, sera, sputum, urine and/or tumor biopsies) obtained from the patient. In other words, such proteins may be used as markers to indicate the presence or absence of a cancer such as lung cancer. In addition, such proteins may be useful for the detection of other cancers. The binding agents provided herein may generally permit detection of the level of TAT-028 antigen that binds to the agent in the biological sample. Binding agents may be compared or screened for based on their strength of binding, selectivity, and/or other properties to find preferrable binding agents for assays.

There are a variety of assay formats known to those of ordinary skill in the art for using a binding agent to detect polypeptide markers in a sample, for example, immunoprecipitation followed by sodium dodecyl sulfate polyacrylamide gel electrophoresis, 2-dimensional gel electrophoresis, competitive and non-competitive assay systems using techniques such as Western blots, immunocytochemistry, immunohistochemistry, immunoassays, e.g., radioimmunoassays, ELISA (enzyme linked immunosorbent assay), “sandwich” immunoassays, immunoprecipitation assays, precipitation reactions, gel diffusion precipitation reactions, immunodiffusion assays, agglutination assays, complement-fixation assays, immunoradiometric assays, fluorescent immunoassays and protein A immunoassays (See also, e.g., Harlow and Lane (1988) Antibodies: A Laboratory Manual, Cold Spring Harbor Laboratory). In general, the presence or absence of a cancer in a patient may be determined by (a) contacting a biological sample obtained from a patient with a binding agent; (b) detecting in the sample a level of TAT-028 polypeptide that binds to the binding agent; and (c) comparing the level of TAT-028 polypeptide with a cut-off value, preferably a predetermined cut-off value. Cut-off values may be determined by methods known in the art, such as by establishing ranges of expression that give degrees of confidence in distinguishing a tumor sample from a normal sample.

In a preferred embodiment, the assay involves the use of a binding agent immobilized on a solid support to bind to the TAT-028 polypeptide(s) in a sample. The bound polypeptide may then be detected using a detection reagent that contains a reporter group and specifically binds to the binding agent/TAT-028 polypeptide complex. Such detection reagents may comprise, for example, a binding agent that specifically binds to the TAT-028 polypeptide or an antibody or other agent that specifically binds to the binding agent, such as an anti-immunoglobulin, protein G, protein A, or a lectin. Alternatively, a competitive assay may be utilized, in which a TAT-028 polypeptide is labeled with a reporter group and allowed to bind to the immobilized binding agent after incubation of the binding agent with the sample. The extent to which components of the sample inhibit the binding of the labeled TAT-028 polypeptide to the binding agent is indicative of the reactivity of the sample with the immobilized binding agent. Suitable polypeptides for use within such assays include full length TAT-028 proteins and polypeptide portions thereof to which the binding agent binds, as described above.

The solid support may be any material known to those of ordinary skill in the art to which a TAT-028 polypeptide may be attached. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment. Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.(see, e.g., Pierce Immunotechnology Catalog and Handbook (1991) at A12-A13).

In one embodiment, an antibody is used in the methods of screening and diagnosis to detect and quantify a TAT-028 polypeptide. Preferably, the antibody is used for detecting and/or quantifying the amount of a polypeptide, as defined in the first aspect of the invention, in a biological sample obtained from said subject.

In one example, binding of antibody in tissue sections can be used to detect aberrant TAT-028 polypeptide localization or an aberrant level of a TAT-028 polypeptide. In a specific embodiment, an antibody recognizing a TAT-028 polypeptide can be used to assay a patient tissue (e.g., a lung biopsy) for the level of the TAT-028 polypeptide where an aberrant level of the TAT-028 polypeptide is indicative of carcinoma. An “aberrant level” includes a level that is increased or decreased compared with the level in a subject free from cancer or a reference level.

In a further aspect, the method of detecting/quantifying the presence of a TAT-028 polypeptide comprises detecting the captured polypeptide using a directly or indirectly labelled detection reagent, e.g., a detectable marker such as, without limitation, a chemiluminescent, enzymatic, fluorescent, or radioactive moiety. If no labelled binding partner to the capture reagent is provided, the anti-TAT-028 polypeptide capture reagent itself can be labelled with a detectable marker (see above).

In a preferred embodiment, antibodies of the invention or fragments thereof are conjugated to a diagnostic or therapeutic moiety. The antibodies can be used for diagnosis or to determine the efficacy of a given treatment regimen. Detection can be facilitated by coupling the antibody to a detectable substance.

Examples of detectable substances include various enzymes, prosthetic groups, fluorescent materials, luminescent materials, bioluminescent materials, radioactive nuclides, positron emitting metals (for use in positron emission tomography), and non-radioactive paramagnetic metal ions (see U.S. Pat. No. 4,741,900 for metal ions which can be conjugated to antibodies for use as diagnostics according to the present invention). Suitable enzymes include horseradish peroxidase, alkaline phosphatase, beta-galactosidase, and acetylcholinesterase. Suitable prosthetic groups include streptavidin, avidin and biotin. Suitable fluorescent materials include umbelliferone, fluorescein, fluorescein isothiocyanate, rhodamine, dichlorotriazinylamine fluorescein, dansyl chloride and phycoerythrin. Suitable luminescent materials include luminol. Suitable bioluminescent materials include luciferase, luciferin, and aequorin. Suitable radioactive nuclides include I¹²⁵, I¹³¹, IN¹¹¹ and Tc⁹⁹.

The foregoing antibodies can be used in methods known in the art relating to the localization and activity of the TAT-028 polypeptides of the invention, e.g., for imaging or radio-imaging these proteins, measuring levels thereof in appropriate physiological samples, in diagnostic methods, etc. and for radiotherapy.

In certain embodiments, the assay is a two-antibody sandwich assay, where antibodies are immobilized on a solid support and exposed to the sample, allowing polypeptides in the sample a to bind to the immobilized antibody. Once the antibody is immobilized on the support the non-specific protein binding sites on the support are typically blocked using blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as lung cancer, the signal detected from the reporter group that remains bound to the solid support is generally compared to a signal that corresponds to a cut-off value, preferably a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the cut-off value is considered positive for the cancer. In an alternate preferred embodiment, the cut-off value is determined using a Receiver Operator Curve, according to the method of Sackett et al. (1985) Clinical Epidemiology: A Basic Science for Clinical Medicine, Little Brown and Co., p. 106-7. Briefly, in this embodiment, the cut-off value may be determined from a plot of pairs of true positive rates (i.e., sensitivity) and false positive rates (100% specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of TAT-028 polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the TAT-028 polypeptides or binding agents of the present invention. The above descriptions are intended to be exemplary only. For example, it will be apparent to those of ordinary skill in the art that the above protocols may be readily modified to use TAT-028 polypeptides to detect antibodies that bind to such polypeptides in a biological sample. The detection of such TAT-028 specific antibodies may correlate with the presence of a cancer.

A cancer may also be detected based on the presence of T cells that specifically react with a TAT-028 polypeptide in a biological sample. Using known methods, a biological sample comprising CD4⁺ and/or CD8⁺ T cells isolated from a patient is incubated with a TAT-028 polypeptide, a polynucleotide encoding such a polypeptide and/or an antigen presentation complex (APC) that expresses at least an immunogenic portion of such a polypeptide, and the presence or absence of specific activation of the T cells is detected. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a cancer in the patient.

In another embodiment, the compositions described herein may be used as markers for the progression of cancer. In this embodiment, assays as described above for the diagnosis of a cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is advancing in those patients in whom the level of TAT-028 polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, TAT-028 polynucleotide probes may be used within such applications.

As noted above, to improve sensitivity, multiple tumor protein markers in addition to TAT-028 may be assayed within a given sample. It will be apparent that binding agents specific for different proteins may be combined within a single assay. For example, such proteins may include any of the antigens listed above as known immunotherapy targets (see “Antibodies, v.) other”). For brevity, the GenBank GI #s provided are intended as representative and may be considered a preferred sequence, however they are meant to encompass splice variants, variants, isoforms, polymorphisms, mutations, modifications, and the like, preferably those associated with cancer. Preferably such variant sequences have at least 90% sequence identity to the representative sequence, more preferably at least 95% sequence identity, or at least 96%, 97%, 98%, or 99% sequence identity. Proteins presented in their precursor form, are also preferred in their mature form. Proteins present in hetero- or homo-multimers may be probed for as individual proteins or as part of their multimeric complex (e.g., integrin αvβ3). Multimer subunits presented may be taken as more preferable subunits, but the other subunits and multimeric forms are also preferred. Further, multiple primers or probes may be used concurrently. The selection of tumor protein markers may be based on routine experiments to determine combinations that result in optimal sensitivity. In addition, or alternatively, assays for TAT-028 polypeptides and/or nucleic acids provided herein may be combined with assays for other known tumor antigens.

In addition, nucleic acid molecules encoding the polypeptides or fragments thereof may be used for diagnostic assays of the invention. The use of nucleic acid molecules which may hybridize to any of the TAT-028 nucleic acid molecules is included in the present invention. Such nucleic acid molecules are referred to herein as “hybridizing” nucleic acid molecules. Hybridizing nucleic acid molecules can be useful as probes or primers, for example, or in hybridization assays. Desirably such hybridizing molecules are at least 8 nucleotides in length and preferably are at least 25 or at least 50 nucleotides in length.

Hybridization assays can be used for detection, prognosis, diagnosis, or monitoring of conditions, disorders, or disease states, associated with aberrant expression of genes encoding a TAT-028 polypeptide, or for differential diagnosis of patients with signs or symptoms suggestive of cancer.

Desirably the hybridizing molecules will hybridize to TAT-028 nucleic acids under stringent hybridization conditions as known in the art and described above.

Nucleic acid molecules encoding the TAT-028 polypeptides or fragments thereof can also be used to identify subjects having a genetic variation, mutation, or polymorphism in a TAT-028 nucleic acid molecule that is indicative of a cancer or a predisposition to develop cancer. These polymorphisms may affect TAT-028 nucleic acid or polypeptide expression levels or biological activity. Such genetic alterations may be present in the promoter sequence, an open reading frame, intronic sequence, or untranslated 3′ region of a TAT-028 gene. As noted throughout, specific alterations in the biological activity of TAT-028 can be correlated with the likelihood of cancer, e.g., lung cancer, or a predisposition to develop the same. As a result, one skilled in the art, having detected a given mutation, can then assay one or more metrics of the biological activity of the TAT-028 protein to determine if the mutation causes or or correlates with an increase in the likelihood of developing cancer.

Diagnostic Kits

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment that specifically binds a TAT-028 protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a tumor protein in a biological sample. Such kits generally comprise at least one oligonucleotide probe or primer, as described above, that hybridizes to a polynucleotide encoding a tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a tumor protein.

The invention also provides diagnostic kits, comprising a capture reagent (e.g., an antibody) against a TAT-028 polypeptide as defined above. In addition, such a kit may optionally comprise one or more of the following: (1) instructions for using the capture reagent for diagnosis, prognosis, therapeutic monitoring or any combination of these applications; (2) a labelled binding partner to the capture reagent; (3) a solid phase (such as a reagent strip) upon which the capture reagent is immobilized; and (4) a label or insert indicating regulatory approval for diagnostic, prognostic or therapeutic use or any combination thereof.

Pharmaceutical Compositions and Therapies

The invention also provides various immunogenic or therapeutic compositions and strategies for the prophylaxis and/or treatment of cancers that express TAT-028 such as lung cancers in a subject, including therapies aimed at inhibiting the transcription, translation, processing or function of TAT-028 as well as cancer vaccines.

In another aspect, the present invention provides a method treatment of cancer in a subject, which comprises administering to said subject a therapeutically effective amount of at least one TAT-028 polypeptide.

In a yet another aspect, the present invention provides the use of at least one TAT-028 polypeptide in the preparation of a pharmaceutical composition for use in the prophylaxis and/or treatment of cancer. The subject may be a mammal and is preferably a human.

In a particular embodiment, a TAT-028 polypeptide is fused to another polypeptide, such as the protein transduction domain of the HIV/TAT protein, which facilitates the entry of the fusion protein into a cell (Asoh et al. (2002) Proc Natl Acad Sci. U.S.A. 99: 17107-17112), is provided for use in the manufacture of a pharmaceutical composition for the treatment of cancer.

In another aspect, the present invention provides a method for the prophylaxis and/or treatment of cancer in a subject, which comprises administering to said subject a therapeutically effective amount of at least one TAT-028 nucleic acid.

In a yet another aspect, the present invention provides the use of at least one TAT-028 nucleic acid in the preparation of a pharmaceutical composition for use in the prophylaxis and/or treatment of cancer. The subject may be a mammal and is preferably a human.

The present invention provides a method for the treatment and/or prophylaxis of cancer in a subject comprising administering to said subject, a therapeutically effective amount of at least one antibody that binds to a TAT-028 polypeptide. In another aspect, the present invention provides the use of an antibody which binds to at least one TAT-028 polypeptide in the preparation of a pharmaceutical composition for use in the prophylaxis and/or treatment of cancer. In particular, the preparation of vaccines and/or compositions comprising or consisting of antibodies is a preferred embodiment of this aspect of the invention.

Any of the compounds described herein, when used for therapeutic or prophylactic methods (human or veterinary) will normally be formulated into a pharmaceutical composition in accordance with standard pharmaceutical practice, e.g., by admixing the active agent and a pharmaceutical acceptable carrier. Thus, according to a further aspect of the invention there is provided a pharmaceutical composition comprising at least one active agent of the invention and a pharmaceutical acceptable carrier. Pharmaceutical acceptable carriers for use in the invention may take a wide variety of forms depending, e.g., on the route of administration.

Thus, the pharmaceutical compositions described herein may be used for the treatment of cancer, particularly for the immunotherapy of lung cancer. Within such methods, the pharmaceutical compositions described herein are administered to a patient. A patient may or may not be afflicted with cancer. Accordingly, the pharmaceutical compositions herein may be used to prevent the development of a cancer or to treat a patient afflicted with a cancer. Pharmaceutical compositions and vaccines may be administered either prior to or following surgical removal of primary tumors and/or treatment such as administration of radiotherapy or conventional chemotherapeutic drugs. Administration of the pharmaceutical compositions may be by any suitable method, including administration to a subject by any of the routes conventionally used for drug administration, for example they may be administered parenterally, orally, topically (including buccal, sublingual or transdermal), intravenously, intraperitoneally, intramuscularly, subcutaneously, intranasally, intradermally, anally, vaginally, topically, and by oral routes or by inhalation. The most suitable route for administration in any given case will depend on the particular active agent, the cancer involved, the subject, and the nature and severity of the disease and the physical condition of the subject.

Compositions for oral administration may be liquid or solid. Oral liquid preparations may be in the form of, for example, aqueous or oily suspensions, solutions, emulsions, syrups or elixirs, or may be presented as a dry product for reconstitution with water or other suitable vehicle before use. Oral liquid preparations may contain suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; water; non-aqueous vehicles (which may include edible oils), for example almond oil, oily esters such as glycerine, propylene glycol, or ethyl alcohol; preservatives, for example methyl or propyl p-hydroxybenzoate or sorbic acid; flavoring agents, preservatives, coloring agents and the like may also be used.

In the case of oral solid preparations such as powders, capsules and tablets, carriers such as starches, sugars, microcrystalline cellulose, diluents, granulating agents, lubricants, binders, disintegrating agents, and the like may be included.

Such compositions may be prepared by any of the methods of pharmacy but all methods include the step of bringing into association the active agent with the carrier, which constitutes one or more necessary ingredients. Desirably, each composition for oral administration contains from about 1 mg to about 500 mg of the active agent.

Compositions comprising an anti-cancer agent of the invention may also be prepared in powder or liquid concentrate form. Thus, particularly suitable powders of this invention comprise 50 to 100% w/w, and preferably 60 to 80% w/w of the combination and 0 to 50% w/w and preferably 20 to 40% w/w of conventional excipients. When used in a veterinary setting such powders may be added to animal feedstuffs, for example by way of an intermediate premix, or diluted in animal drinking water.

Liquid concentrates of this invention for oral administration suitably contain a water-soluble compound combination and may optionally include a pharmaceutically acceptable water miscible solvent, for example polyethylene glycol, propylene glycol, glycerol, glycerol formal or such a solvent mixed with up to 30% v/v of ethanol. Pharmaceutical compositions suitable for parenteral administration may be prepared as solutions or suspensions of the active agents of the invention in water suitably mixed with a surfactant such as hydroxypropylcellulose. Dispersions can also be prepared in glycerol, liquid polyethylene glycols, and mixtures thereof in oils.

The pharmaceutical forms suitable for injectable use include aqueous or non-aqueous sterile injection solutions which may contain anti-oxidants, buffers, bacteriostats and solutes which render the composition isotonic with the blood of the intended recipient, and aqueous and non-aqueous sterile suspensions which may include suspending agents and thickening agents. Extemporaneous injection solutions, dispersions and suspensions may be prepared from sterile powders, granules and tablets.

Exemplary targeting moieties include folate or biotin (see, e.g., U.S. Pat. No. 5,416,016); mannosides (Umezawa and Eto (1988) Biochem Biophys Res Comm. 153: 1038-1044); antibodies (Bloemen et al. (1995) FEBS Lett. 357: 140-144; Owais et al. (1995) Antimicrob Agents Chemother. 39: 180-184); surfactant protein A receptor (Briscoe et al. (1995) Am J Physio. 268: 374-380), different species of which may comprise the compositions of the inventions, as well as components of the invented molecules; psi 20 (Schreier et al. (1994) J Biol Chem. 269: 9090-9098); see also Keinanen and Laukkanen (1994) FEBS Lett. 346: 123-126; and Killion and Fidler (1994) Immunomethods 4: 273-279. In one embodiment of the invention, the anti-cancer agents of the invention are formulated in liposomes; in a more preferred embodiment, the liposomes include a targeting moiety. For methods of manufacturing liposomes; see, for example, U.S. Pat. Nos. 4,522,811; 5,374,548; and 5,399,331. The liposomes may comprise one or more moieties which are selectively transported into specific cells or organs, thus enhancing targeted drug delivery (see, e.g., Ranade (1989) J Clin Pharmacol. 29: 685-694). In a most preferred embodiment, the therapeutic compounds in the liposomes are delivered by bolus injection to a site proximal to the tumor.

Pharmaceutical compositions suitable for rectal administration wherein the carrier is a solid are most preferably presented as unit dose suppositories. Suitable carriers include cocoa butter or other glyceride or materials commonly used in the art, and the suppositories may be conveniently formed by admixture of the combination with the softened or melted carrier(s) followed by chilling and shaping moulds. They may also be administered as enemas.

Pharmaceutical compositions adapted for vaginal administration may be presented as pessaries, tampons, creams, gels, pastes, foams or spray compositions. These may comprise emollient or bases as commonly used in the art.

Pharmaceutical compositions may be conveniently presented in unit dose forms containing a predetermined amount of an active agent of the invention per dose. For example, the compositions may contain from 0. 1% by weight, preferably from 10-60% by weight, of the active agent of the invention, depending on the method of administration. The dosage to be administered of an active agent may vary according to several factors, including, but not limited to, the particular active agent, the cancer involved, the subject, the nature and severity of the disease and the physical condition of the subject, and the selected route of administration; the appropriate dosage can be readily determined by a person skilled in the art. For prophylactic or therapeutic use in humans and animals, a dosage unit may contain, for example, but without limitation, 0.001 mg/kg to 750 mg/kg of active agent, depending on factors such as those aforementioned. Preferred unit dosage compositions are those containing a daily dose or sub-dose, as recited above, or an appropriate fraction thereof, of the anti-cancer agent.

It will be recognized by one of skill in the art that the optimal quantity and spacing of individual dosages of an anti-cancer agent of the invention will be determined by the nature and extent of the condition being treated, the form, route and site of administration, and the particular subject being treated, and that such optimums can be determined by conventional techniques. It will also be appreciated by one of skill in the art that the optimal course of treatment, i.e. the number of doses of an active agent of the invention given per day for a defined number of days, can be ascertained by those skilled in the art using conventional course of treatment determination tests.

In a particular embodiment, a therapeutically effective amount of an agent can be determined by monitoring an amelioration or improvement in disease symptoms, to delay onset or slow progression of the disease, for example but without limitation, a reduction in tumor size. Preferably such improvements in disease symptoms are by at least 0.1%, at least 1%, at least 5%, or at least 10%. More preferably, such improvements are by at least 25%, at least 50%, at least 75%, or at least 90%. Most preferably, such improvements are by at least 95%, at least 96%, at least 97%, at least 98%, or at least 99%. Dosage regimens can be adjusted to provide the optimum desired response (for example, see Hardman and Limbird (eds.) (2001) Goodman & Gilman's The Pharmacological Basis of Therapeutics, 10th edition, McGraw Hill, New York; Beers and Berkow (eds.) (1999) The Merck Manual, 17^{th} edition, Merck Research Laboratories, Whitehouse Station, N.J.). In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a tumor protein generally correlate with an improved clinical outcome. Such immune responses may generally be evaluated using standard proliferation, cytotoxicity or cytokine assays, which may be performed using samples obtained from a patient before and after treatment. Such response can also be monitored by measuring the anti-TAT-028 antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients.

The present invention also features a combination therapy involving the use of a TAT-028 antibody or a TAT-028 vaccine, and that further includes administration to the patient an additional treatment for cancer, with the additional treatment administered within six months of administering the TAT-028 antibody or TAT-028 vaccine. In one embodiment, one or more anti-cancer agents are administered alone or in combination (e.g., simultaneously, sequentially or separately) with one or more additional treatments or therapeutic compounds for cancer and/or symptoms or conditions related to the treatment thereof, wherein at least one of the therapies involves TAT-028 peptides, TAT-028 nucleic acids, TAT-028 antibodies, TAT-028 binding molecules, or TAT-028 vaccines. The additional treatment can be, but is not limited to, surgery, radiation therapy, chemotherapy, immunotherapy, anti-angiogenesis therapy, or gene therapy. Examples of other preferable contemplated treatments for use in combination with TAT-028-based treatments (see, for additional examples, Goodman & Gilman's The Pharmacological Basis of Therapeutics, supra, Chapter 52). Drug administration may be performed at different intervals (e.g., daily, weekly, or monthly) and the administration of each agent can be determined individually. Combination therapy may be given in on-and-off cycles that include rest periods so that the patient's body has a chance to build healthy new cells and regain its strength.

### EXAMPLES

### Example 1

### Reproducibility of Peptide Matching and Variance of Peptide Intensities

An experiment was conducted using a complex human tissue sample and the sample was processed (solubilized and fractionated by 1D SDS polyacrylamide gel electrophoresis (PAGE)). The gels were cut into 24 equal bands and each band was digested with trypsin to obtain peptides for analysis by nano-liquid chromatography-mass spectrometry (LC-MS)) to provide a total of 15 injections into the mass spectrometer after pooling. Each peptide fraction was injected onto a reverse phase capillary nano-liquid chromatography C₁₈ column, coupled by electrospray to a QTOF (quadrapole time of flight) mass spectrometer. Peptide maps were derived for each of the 15 LC-MS isotope maps and all pairwise alignments between peptide maps were performed according to methods found in “Constellation Mapping and Uses Thereof” (PCT publication number WO 2004/049385, U.S. patent application publication number 20040172200; hereinafter “Constellation Mapping”).

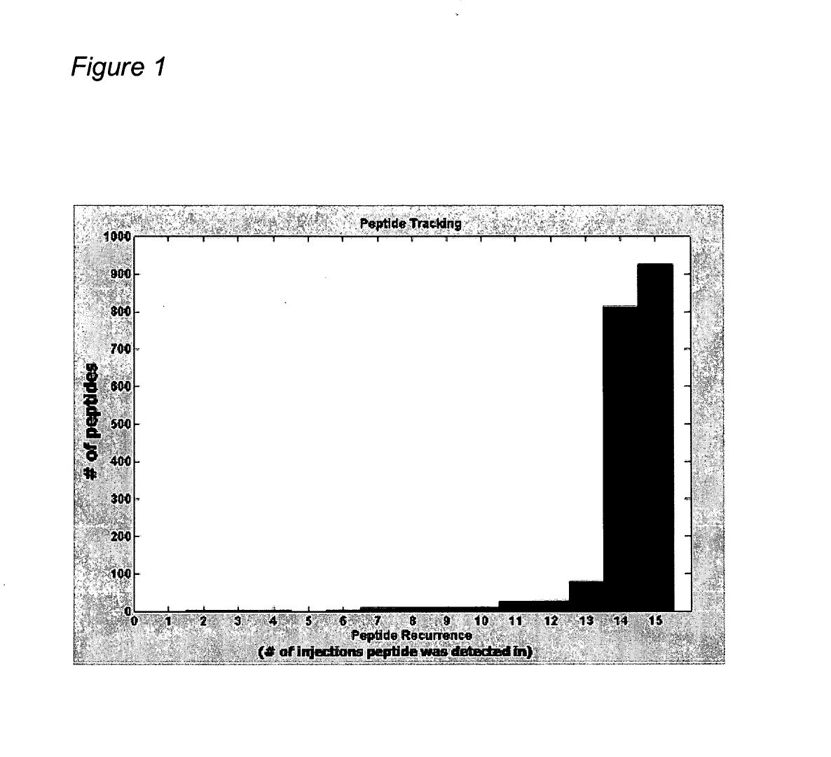

The reproducibility of peptide matching results for the 15 injections of the same sample are summarized in FIG. 1 demonstrating that 90% of peptides were found in at least 14 out of the 15 injections. In addition, the median pairwise peptide-matching rate was 98%.

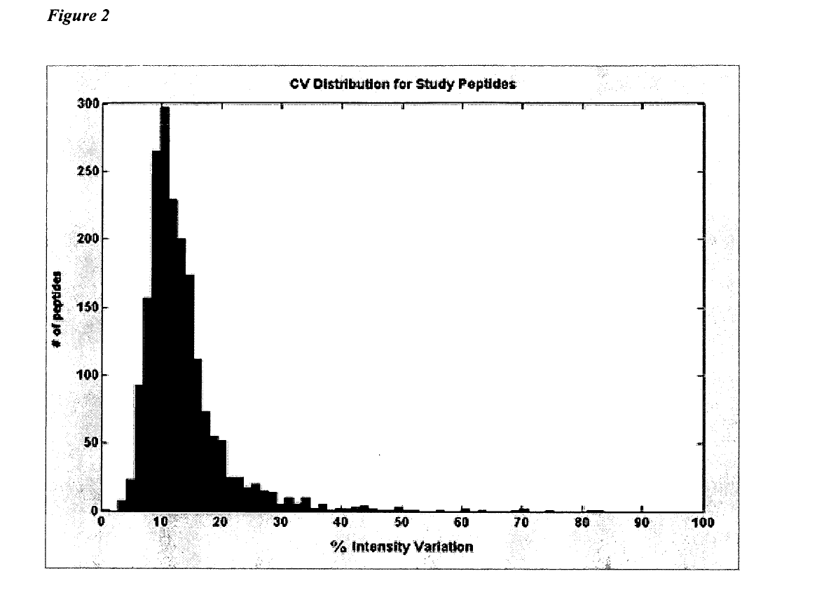

The variance in peptide intensity results are summarized in FIG. 2 where it is demonstrated that the intensity values of the matched peptides showed little variance. The median coefficient of variance (CV) was under 12%. Furthermore, each CV value was calculated over 14 to 15 peptide intensity values, 90% of the time (see FIG. 1).

### Example 2

### Predicting Differential Abundance from Differential Intensity

A controlled experiment was conducted where 3 proteins were spiked into a complex sample at 14 different concentrations, from 1.25 fmoles to 500 fmoles, each in triplicate yielding 42 samples that were analyzed by LC-MS. For each of the 3 proteins, 10 peptides were identified in each sample and their intensities recorded. Peptide intensity was derived from the height of the peptide peak within the LC-MS data.

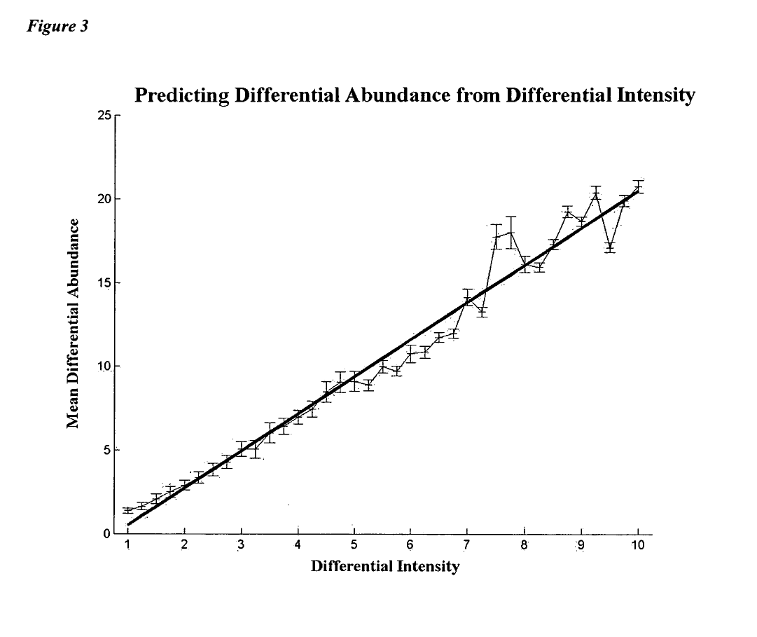

All differential protein abundance (dA) ratios and corresponding differential peptide intensity (dI) ratios were obtained. FIG. 3 shows a plot of all such pairs where the mean differential abundance (black line) and standard deviations were plotted. Protein differential abundance (dA) was clearly predicted from peptide differential intensity (dI).

### Example 3

### Predicting Protein Abundance from Peptide Abundance

Intensities were acquired from mouse plasma samples for three different hemoglobin tryptic peptides by mass spectrometry using Constellation Mapping and Mass Intensity Profiling System (PCT Publication No. WO 03/042774 and US Publication No. 20030129760; hereinafter “MIPS”) software. Briefly, proteins from the plasma samples were solubilized and fractionated by 1D SDS-PAGE. Gels were cut into 24 equal bands and each band was digested by trypsin to obtain peptides for analysis by nano-LC-MS. Each peptide fraction was injected onto a reverse phase capillary nano-liquid chromatography C₁₈ column, coupled by electrospray to a QTOF mass spectrometer.

Plasma samples were subjected, in parallel, to proteomics analysis through a pair-wise comparison of the samples using MIPS and Constellation Mapping softwares. The analyses yielded isotope maps (see Constellation Mapping) in which thousands of peptide ions were visible, separated by retention time and a mass/charge ratio. Each isotope map was converted to a peptide map with each complex peptide isotope signature replaced by a single point, represented by the mass, charge, retention time, and intensity of that peptide. A nonlinear and dynamic retention time correction procedure was devised (see Constellation Mapping) to match the retention time when comparing two or more samples. The retention time correction procedure was based on pattern matching at each time point, resulting in the ability to accommodate even highly erratic behavior. Also identified in this process were those peptides unique to one sample or the other.

Peptide matching between samples was followed by a determination of relative intensity for each peptide, the automated calculation of which involved a form of the MIPS technology. (While each peptide has a unique ionization potential, making determination of absolute abundance difficult, the relative abundance of a peptide is directly related to its concentration in samples of similar complexity.) Peptide data was also later subjected to manual validation to correct potential errors in peptide matching. (Failures in peptide matching are largely due to peptide collision or heavily populated regions of the peptide maps.)

LC-MS/MS analysis of the samples was used in peptide sequence determination. Parent protein identification proceeded through Mascot (Matrix Science, Boston, Mass.) and BLAST (Altschul et al. (1997) Nucleic Acids Res. 25: 3389-3402; Altschul et al. (1990) J Mol Biol. 215: 403-410), and identified hemoglobin spectra were manually validated to confirm correct sequence assignment to the spectra. The three peptides represented in FIG. 4 were identified with m/z ratios of 637.8, 647.8, and 586.3. Manual validation of the peptide-matching between the LC-MS run and the LC-MS/MS run was also performed to ensure that the sequenced peptide corresponded to the desired hemoglobin peptide. Intensities of validated hemoglobin peptides were normalized by dividing the intensity of a peptide in each sample by the maximum intensity of that peptide.

Hemoglobin levels for the same samples were also determined for comparison by an independent assay based on the catalytic activity of hemoglobin in the oxidation of TMB (tetramethyl benzidine) in the presence of peroxide (Standefer and Vanderjagt (1977) Clin Chem. 23: 749). Briefly, 50 ml tubes were labeled for each sample and placed on ice. Two additional 50 ml tubes were also prepared and placed on ice: one a blank, one a control. The control was a pooled rat plasma (Pel-Freez Biologicals, Rogers, Ark.; catalog number 36142) of known hemoglobin content, used as a standard to calculate the hemoglobin content of the unknown samples: (Control concentration X OD₆₀₀)/unknown sample OD₆₀₀. Two ml of TMB 1-Component Microwell Peroxidase Substrate solution (KPL, Gaithersburg, Md.; catalog number 52-00-02) was added to all the labeled tubes, followed by addition of 10 μl of control plasma sample or plasma samples sequentially to their respective labeled tube(s). The tube labeled ‘Blank’ did not contain any plasma. Note that the time interval between additions of two consecutive plasma samples was one minute. Samples were vortexed for 2 seconds at maximum speed, then left at ambient temperature for 10 minutes. A BeckmanCoulter DU640B spectrophotometer was zeroed with the Blank sample at 600 nm wavelength, after ensuring that the lamp was turned on at least 20 minutes prior to reading. Samples were transferred into disposable cuvettes after 10 minutes, and the absorbance read at 600 nm. As seen in these results (FIG. 4), even a single peptide result as determined by mass spectrometry was able to give a reliable picture of the behavior of the parent protein in the sample.

### Example 4

### Identification of TAT-028 Overexpression in Lung Tumors

Tumor and normal epithelial cells were obtained from fresh lung resections from 30 individuals. Purified plasma membrane (20 μg) was obtained from each matching sample through the use of magnetic beads, coated with antibodies specific for epithelial cell plasma membrane proteins. Procedures were essentially as described in Sircar et al., Clin. Cancer Res. 2006; 12:4178-4184 with some modifications.

Benign and tumor tissues were cut into small pieces (about 3 mm size cube) and homogenization buffer [250 mmol/L sucrose, 10 mmol/L Tris-HCl (pH 7.4)], 100 units/ml of DNase 1 (Roche, Laval, Canada), 5 mmol/L MgCl₂, Complete protease inhibitor EDTA-free cocktail (Roche, Laval, Canada) ) was added at a concentration of 10 mL per gram of tissue. Normal and Tumor tissues were homogenized 3×20 seconds and 3×10 seconds respectively using a polytron (Kinematica, Newark, N.J.) set at speed 8 (˜20000 rpm). Three blocks of tissue from each matched normal and tumor specimen were also kept for RNA extraction. Each block weighed approximately 50 mg, and was archived in RNA later (Sigma-Aldrich; Product code R0901) at −80° C. Homogenates were filtered through a 180 μm nylon mesh and centrifuged at 900 g (2000 rpm) for 10 min, at 4° C. Supernatants were collected, brought to 12.5 ml with the homogenization buffer and transferred into 12.5 ml/Ultra-clear centrifuge tube (Beckman Coulter, Mississauga, Canada). For each tube, a cushion made of 100 μL of 50% w/v sucrose was placed at the bottom. Samples were then centrifuged at 35,000 rpm (100,000 g) for 60 min at 4° C. to pellet the membranes. Membrane pellets were resuspended at 1 ml of homogenization buffer per g of tissue and incubated with 500 units/ml of Micrococcal Nuclease (US Biologicals, Swampscott, Mass.) and 1 mM CaCl₂ for 15 min at 4° C. To the resuspended membranes, 2.55 M sucrose solution was added to obtain a final sucrose concentration of 1.7 M. To isolate crude plasma membranes, isopycnic centrifugation using discontinuous sucrose gradients was performed as follow: On top of the 1.7 M sucrose fraction containing membranes, the 1.5 M, 1.3 M and 0.5 M sucrose layers were overlaid and samples were then centrifuged at 35,000 rpm (100,000 g) for 18 hours at 4° C. After centrifugation, the crude plasma membrane fraction located at 0.5M/1.3M sucrose interface was collected. Amount of protein was determined using the BCA assay according to manufacturer's instructions (Pierce, Rockford Ill.). Following BCA assay, the crude plasma membrane fractions were snap-frozen in liquid nitrogen and stored at −80° C. Crude plasma membranes were thawed and incubated with mouse anti-epithelial plasma membrane antibody cocktail for 60 min at 4° C. For 1 mg of crude plasma membranes, 20 μg of CEA (Neomarkers, Freemont, Calif., Catalog Number MS-613-P), 20 μg of ESA (Neomarkers, Freemont, Calif., Catalog Number MS-181-P), 20 μg of EMA (Serotec, Oxford UK Catalog Number MCA1742) and 20 μg of CD66c (InnoGenex, San Ramon, Calif., Catalog Number AM-1410-11) antibodies were added and the incubation was performed in 10 ml of isolation buffer [PBS, 0.5 mg/ml PVP-40T (Sigma, St-Louis Mo.), 0.5 mg/ml skim milk, Complete protease inhibitor EDTA-free cocktail]. Samples were then transferred into 12.5 mL ultra-centrifuge tubes. Cushion of 100 μl of 50% sucrose was placed at the bottom of the tubes and samples were centrifuged at 40,000 rpm for 60 min at 4° C. to pellet membranes. Membranes were resuspended in 2 ml of isolation buffer/mg of crude plasma membrane and incubated 30 min at 4° C. with goat anti-mouse MACS immunomagnetic beads (Miltenyi Biotech, Auburn, Calif.) at a ratio of 1 μl of beads/μg of crude plasma membrane in a total volume of 10 ml isolation buffer. To reduce cytoskeletal protein content associated with plasma membrane, potassium iodine (KI) was added to the samples to obtain a final concentration of 600 mmol/L and then incubated for 30 min at 4° C. In a cold room, samples were applied on magnetic LS columns according to manufacturer instructions (Miltenyi Biotech, Auburn, Calif.). Columns were washed 3 times with 8 ml of isolation buffer containing 600 mmol/L of KI and once with 8 ml of 250 mmol/L sucrose, 10 mmol/L Tris-HCl (pH 7.4), Complete protease inhibitor EDTA-free cocktail buffer. Columns were then removed from magnet and purified epithelial plasma membranes were eluted with 3.5 ml of 250 mmol/L sucrose, 10 mmol/L Tris-HCl (pH 7.4), Complete protease inhibitor EDTA-free cocktail buffer into 15 ml tubes. To determine the amount of protein, 350 μL of eluted plasma membranes fraction was centrifuged at 55,000 rpm (190,000 g) for 60 min at 4° C. using a TLA 55 rotor and the Optima MAX Ultracentrifuge (Beckman Coulter, Mississauga, Canada). Membrane pellets were solubilized in 250 mmol/L sucrose, 10 mmol/L Tris-HCl (pH 7.4), Complete protease inhibitor EDTA-free cocktail buffer containing 1% SDS and protein concentration was determined using the micro-BCA assay. The remaining eluate was transferred in a 4 ml ultra-clear centrifuge tube. A cushion of 50 μL 33% sucrose was placed at the bottom of the tube and samples were spun at 50,000 rpm (337 000 g) for 30 min at 4° C. to pellet the plasma membranes. According to the protein concentration obtained by the micro-BCA assay, add Leammli buffer containing 5.3 mol/L of Urea to the pellets to obtain a final concentration of 1.32 ug/ul. Samples were vortexed at ambient temperature for 15 minutes. Solubilized proteins were then snap-frozen in liquid nitrogen and stored at −80° C.

Solubilized proteins from plasma membrane fractions from normal and tumor tissues were fractionated by 1D SDS polyacrylamide gel electrophoresis (PAGE). Gels were cut into 24 equal bands and each band was digested by trypsin to obtain peptides for analysis by nano-liquid chromatography-mass spectrometry (LC-MS). Each peptide fraction was injected onto a reverse phase capillary nano-liquid chromatography C₁₈ column, coupled by electrospray to a QTOF (quadrapole time of flight) mass spectrometer

In addition, tumor and normal purified plasma membrane was subjected, in parallel, to proteomics analysis through a pair-wise comparison of samples from a single individual using MIPS and Constellation Mapping softwares. The analyses yielded isotope maps in which thousands of peptide ions were visible, separated by retention time and a mass/charge ratio. Each isotope map was converted to a peptide map with each complex peptide isotope signature replaced by a single point, represented by the mass, charge, retention time, and intensity of that peptide. A nonlinear and dynamic retention time correction procedure was devised to match the retention time when comparing two or more samples. The retention time correction procedure was based on pattern matching at each time point, resulting in the ability to accommodate even highly erratic behavior. Also identified in this process were those peptides unique to one sample or the other.

Peptide matching between samples was followed by a determination of relative intensity for each peptide and its automated calculation involved a form of the MIPS technology. (While each peptide has a unique ionization potential, making determination of absolute abundance difficult, the relative abundance of a peptide is directly related to its concentration in samples of similar complexity.) Peptide data was also later subjected to manual validation to correct potential errors in peptide matching. (Failures in peptide matching are largely due to peptide collision or heavily populated regions of the peptide maps.)

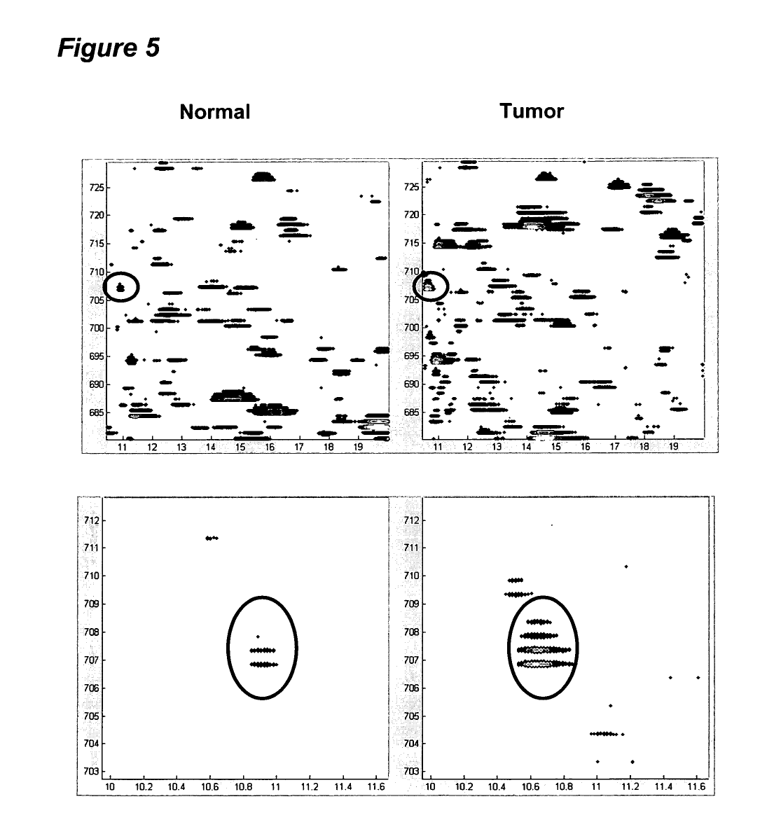

Of the peptides detected across all of the samples of the sample set the relative abundance of the majority of peptides varied with a standard deviation of the mean of only 14%. Such tightly reproducible results allowed for the reliable detection of only slight differences between healthy and diseased lung samples. Intensity differences of two-fold were readily and accurately detected across many patient samples. One of the peptides which was identified as being differentially expressed was subjected to manual MS to MS peptide-matching validation to ensure that the target peptide was matched correctly and expressed at the expected levels (see FIG. 5).

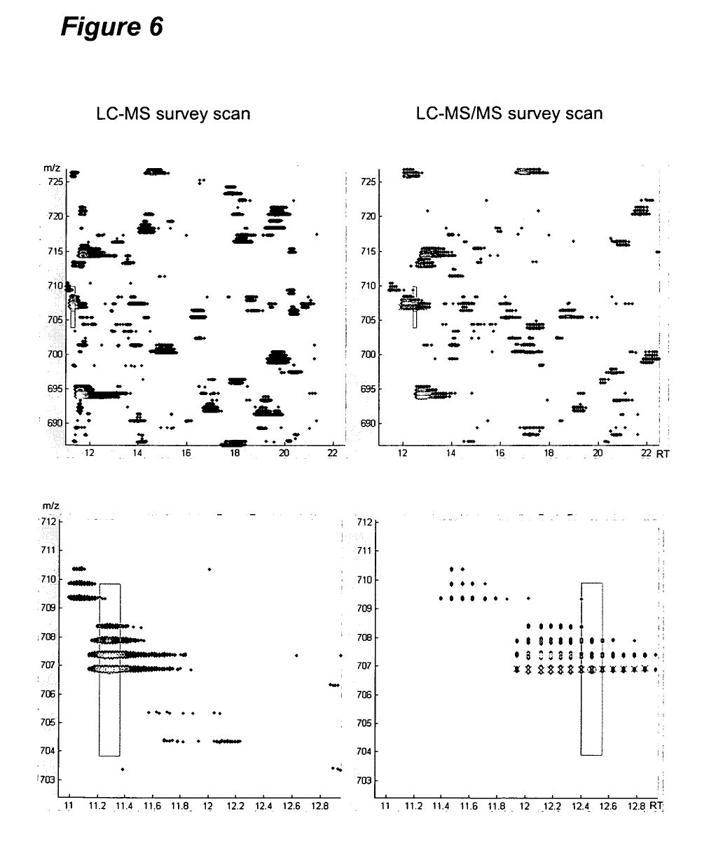

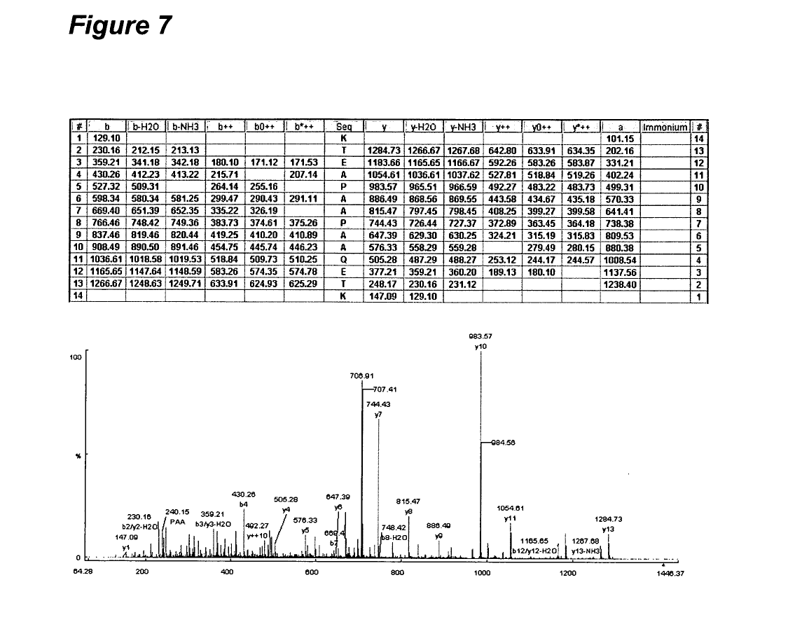

Once all patient samples were processed, a cross-study analysis was performed to identify those peptides determined to be over-expressed at a minimum pre-determined threshold level in a minimum pre-determined percentage of patients. For example, in the analysis of the thirty patient matched lung tumor and normal samples, 224,380 peptides were observed, 39,722 of which were reproducibly observed in 30% or more of the study patients. Of these, 1344 were seen to be at least ten-fold up-regulated in over 30% of the patients, 4309 at least five-fold, and 6649 at least three-fold. Peptides identified as over-expressed under these criteria were subjected to targeted LC-MS/MS analysis for sequence determination. Manual validation of the peptide-matching between the LC-MS run and the LC-MS/MS run was performed on selected peptides to ensure that the sequenced peptide corresponded to the desired differentially expressed peptide (see FIGS. 6 and 7) for peptide #1 (SEQ ID NO: 1)) Parent protein identification proceeded through Mascot (Matrix Science, Boston, Mass.) and BLAST (Altschul et al. (1997) Nucleic Acids Res. 25: 3389-3402; Altschul et al. (1990) J Mol Biol. 215: 403-410). Peptides and proteins identified by these methods are potential immunotherapy targets.

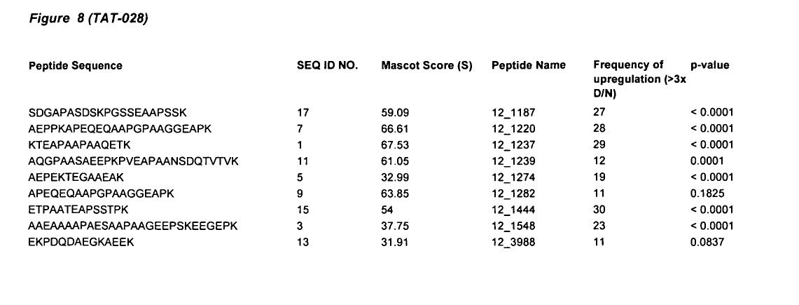

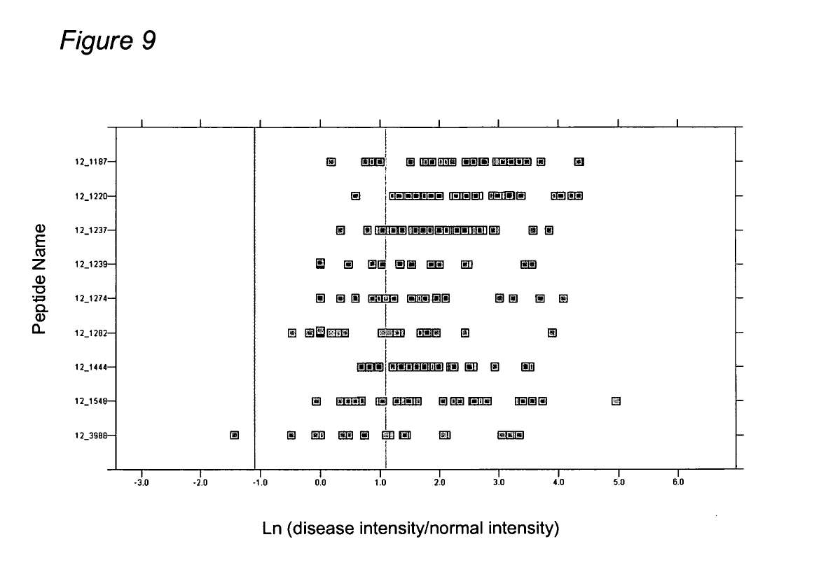

TAT-028 peptides (SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17) were determined to be differentially expressed by at least 3-fold (2.1-fold differential intensity corresponding to 3-fold differential abundance) between normal and tumor lung samples in 30% or more of the patient samples examined (FIGS. 8, and 9). TAT-028 peptides (SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17) were found to be uniquely matching to the TAT-028 sequence (SEQ ID NO: 19), representative GenBank gi: 30795231. The Mascot Scores (FIG. 8) is given as S=−10*log(P) where P represents the probability that the observed match between experimental data and a protein sequence, present in the database searched, is a random event. The significance depends on the size of the database being searched. Based on the size of the database and on experimental evidence obtained in house, 90% of peptides with a score >35 passed manual inspection to validate the match between the peptide sequence obtained and the MS-MS spectral data used in the search. These sequenced peptides were found to be overexpressed at a level of greater than 3-fold differential abundance in at least 30% of the 30 lung tumor samples relative to normal tissue obtained from the same patient (FIG. 8). P-values listed in FIG. 8 were calculated from the raw peptide intensities measured in each sample using a paired t-test and represent the probability that the overexpression of a peptide observed occurred by chance alone.

The position of the TAT-028 peptide sequences identified in the TAT-028 protein sequence is illustrated in FIG. 10.

As a plasma membrane protein differentially expressed at a higher level in tumor cells as compared to adjacent normal cells, TAT-028 (SEQ ID NO: 19, see FIG. 10) and the sequenced peptides (SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17) (see FIG. 10) were identified as targets for immunotherapy of lung cancer.

### Example 5

### TAT-028 cDNAs

TAT-028 encoding nucleic acids (e.g., SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NOS: 50-58, SEQ ID NO:18, SEQ ID NO: 20, SEQ ID NO: 21) maybe obtained by methods known in the art and from other readily available sources. For example, I.M.A.G.E. Consortium clones (ATCC, Manassas, Va.) containing a TAT-028 nucleic acid sequence for example, ATCC® Number: 5497589, IMAGE:3284010 (GenBank No: BE670129) may be ordered and sequenced using appropriate primers and methods known in the art (see, for example, Glover and Hames, DNA Cloning 1: Core Techniques, New York 1995, Roe et al., DNA Isolation and Sequencing, New York, 1996 or Sambrook et al., Molecular Cloning: A Laboratory Manual Vols. 1, 2, and 3, Cold Spring Harbor Laboratory Press, NY, 1989). A coding sequence is illustrated in FIG. 11 (SEQ ID NO: 20).

Alternatively, primers may be designed based on the ends or any facilitating intervening sequences of a TAT-028 GenBank sequence (with or without flanking sequences such as introduced restriction sites) to amplify TAT-028 nucleic acids by PCR from a human cDNA library using appropriate temperatures and cycle times for the nucleic acid sequences. Primers may also be comprised of or contain regions of the protein sequence that correspond to the peptides that were observed to be over-expressed in human tissues.

A cDNA library and 5′-RACE and/or 3′-RACE can be used to obtain clones encoding portions of previously uncloned regions. RACE (Rapid Amplification of cDNA Ends; see, e.g., M. A. Frohman, “RACE: Rapid Amplification of cDNA Ends,” in Innis et al. (eds) (1990) PCR Protocols: A Guide to Methods and Applications, pp. 28-38; and Frohman et al. (1988) Proc Natl Acad Sci. U.S.A. 85: 8998-9002) is used to generate material for sequence analysis and subcloning if necessary.

Genomic and cDNA libraries may also be screened to identify any libraries that contain the TAT-028 gene (e.g., SEQ ID NO: 22) or closely related genes or sequences such as those corresponding to the polypeptide xenologues sequences disclosed herein: Chimpanzee (GenBank gi: 55623958; SEQ ID NO: 38), Dog (gi: 73954389; SEQ ID NO: 39), Rat (gi: 11560135; SEQ ID NO: 40), Mouse (gi: 66773932; SEQ ID NO: 41) and Chicken (gi: 45383878, SEQ ID NO: 42)). Zenologue genomic or nucleotide sequences can be obtained from the Entrez Nucleotide or Entrez Gene entries corresponding to the NCBI Entrez Protein entries listed above. In the preparation of genomic libraries, for example, DNA fragments are generated, some of which will encode parts or the whole of a polypeptide as defined herein. The DNA may be cleaved at specific sites using various restriction enzymes. For example, one may use DNAse in the presence of manganese to fragment the DNA, or the DNA may be physically sheared, as for example, by sonication. The DNA fragments may then be separated according to size by standard techniques, including but not limited to agarose and polyacrylamide gel electrophoresis, column chromatography and sucrose gradient centrifugation. The DNA fragments may then be inserted into suitable vectors, including but not limited to plasmids, cosmids, bacteriophages lambda or T4, and yeast artificial chromosomes (Yacs) (see, for example, Sambrook et al. (1989) Molecular Cloning, a Laboratory Manual, 1st Ed., Cold Spring Harbor Laboratory Press, Cold Spring Harbor, N.Y.; Glover, D. M. (Ed.) (1985) DNA Cloning: A Practical Approach, MRL Press, Ltd., Oxford, U.K. Vol. L, Li; Ausubel F. M. et al. (Eds.) (1989) Current Protocols in Molecular Biology, Vol. I, Green Publishing Associates, Inc., and John Wiley & Sons, Inc., New York). The genomic library may be screened by nucleic acid hybridization to labelled probe (Benton and Davis (1977) Science 196: 180-182; Grunstein and Hogness (1975) Proc Natl Acad Sci. U.S.A. 72: 3961-3965).

Dot blot hybridization (Leary et al. (1983) Proc Natl Acad Sci. U.S.A. 80: 4045-4049; Grunstein and Hogness (1975) Proc Natl Acad Sci. U.S.A. 72: 3961-3965; Benton and Davis (1977) Science 196: 180-182) may be performed to pre-screen libraries. Positive libraries may then be screened by colony or plaque hybridization to obtain genomic and/or cDNA versions of the TAT-028 gene (see, for example, Glover and Hames (1995) DNA Cloning 1: Core Techniques, New York; Roe et al. (1996) DNA Isolation and Sequencing, New York; or Sambrook et al. (1989) Molecular Cloning: A Laboratory Manual, Vols. 1, 2, and 3, Cold Spring Harbor Laboratory Press, NY).

### Example 6

### TAT-028 Vectors

TAT-028 nucleic acid sequences may be used as linearized DNA for direct in vitro translation, or may be subcloned into vectors such as plasmids or viral vectors. Such vectors have use in producing TAT-028 proteins and nucleic acids as well as phenotypes associated with their expression, or inhibition thereof such as in a transgenic “knockout” mouse, but are not limited to these uses. PCR, incorporation of restriction sites, and the like for use in subcloning into vectors may be found for example in Sambrook et al., Molecular Cloning: A Laboratory Manual, Vols. 1, 2, and 3, Cold Spring Harbor Laboratory Press, NY, 1989.

An expression vector, in this embodiment utilizing pGEX-6P-1 (Product #27-4597-01, Amersham Biosciences, San Francisco) as a backbone, comprising the sequence of FIG. 11, is useful for producing a purified GST-TAT-028 fusion protein, and the GST peptide portion may be removed by protease cleavage, according to manufacturer's instructions.

Briefly, in one working example, a pGEX-6P-1 vector is produced utilizing a PCR product of the TAT-028 coding sequence (obtainable per Example 5 or Examples 7 and 8). Primers are designed for both the 5′ and 3′ ends of the TAT-028 coding sequences to incorporate one or more restriction enzyme sites found in the pGEX-6P-1 vector multiple cloning site (e.g., BamHI, EcoRI, SmaI, SalI, XhoI, and NotI sites) and remain in-frame with the GST peptide. Temperatures and cycle times are calculated for the primers chosen. After digestion with the appropriate restriction enzyme(s) and gel purification, the PCR fragment is ligated into dephosphorylated (with calf intestine alkaline phosphatase, see for example Seeburg et al. (1977) Nature 220: 486; Ullrich et al. (1977) Science 196: 1313) pGEX-6P-1 digested with the same restriction enzyme(s). Expression of recombinant protein is evaluated by SDS-PAGE and Western blot analysis. A pGEX-6P-1 vector as described herein can be used to produce a readily purifiable GST-TAT-028 fusion protein, and the GST peptide portion may be removed by cleavage.

Similarly a HIS-tag expression vector, such as pET-45b from Novagen (San Diego) is produced using primers to incorporate a Kpn1 flanking the TAT-028 coding sequence and keeping it in-frame with the HIS-tag. Baculovirus (Pharmingen) and Yeast (Invitrogen) expression vectors containing His/fusion protein tags are also made in this way and the expression of recombinant protein is evaluated by SDS-PAGE and Western blot analysis.

Similar subcloning strategies are used with the desired TAT-028 nucleic acid sequences to produce other vectors, such as knock-out and knock-in vectors, expression vectors for mammalian cells, adenoviral vectors, vaccinia virus vectors, other tag or fusion vectors, and the like.

### Example 7

### Extraction of RNA from Tumors

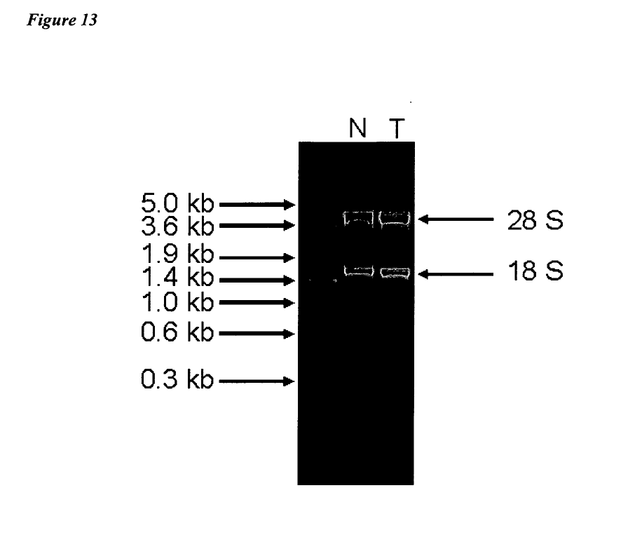

Three blocks of tissue from each matched normal and tumor specimen were kept for RNA extraction. Each block weighed approximately 50 mg, and was archived in RNAlater (Sigma-Aldrich; Product code R0901) at −80° C. (see Example 4). High quality RNA may later be obtained from most tissues using an RNeasy Mini Kit from QIAGEN (Valencia, Calif.). Each RNA preparation quality may be assessed by formaldehyde-agarose gel electrophoresis (see FIG. 13). Generally, at least 5 mg of RNAlater-stored material was used for target cloning. Approximately 35 μg of RNA was typically recovered from a 50 mg piece of archived tissue. The RNA was converted to cDNA using standard reverse transcription with oligo-dT and random primers (Invitrogen, Carlsbad, Calif.).

### Example 8

### Cloning TAT-028 Nucleic Acids from Tumors

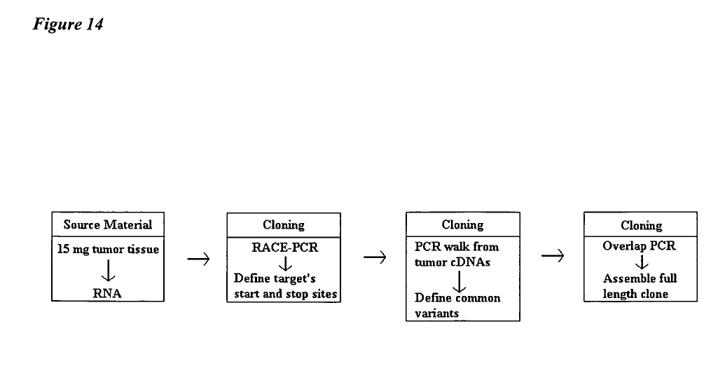

The TAT-028 nucleic acid sequences may be confirmed by cloning from the lung tumor tissues used. This process may also identify polymorphisms, mutations, and/or variants including those particular to, or common to, the tumors used. One method that may be used for cloning TAT-028 nucleic acids is taken from the general cloning methodology used for cloning CD44 and CD98 from tumor cDNA. This method includes: 1) defining the start and stop sites of the target clone by RACE-PCR (Rapid Amplification of cDNA Ends-Polymerase Chain Reaction), preferably using the peptide sequence information obtained through the proteomics analysis for primer generation; 2) discovering variants by PCR walk from one end of the target to the other; and 3) assembly of full length clones by overlap PCR (see FIG. 14).

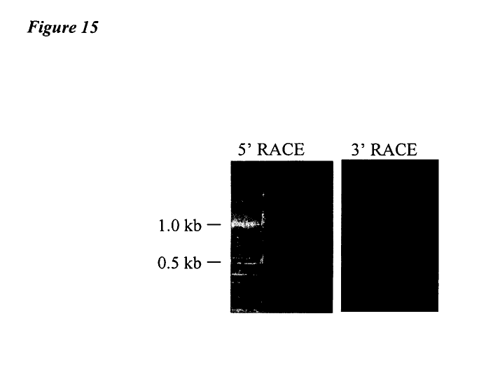

In step 1 (see FIG. 14), RACE-PCR is performed to define the 5′ and 3′ ends of the target nucleic acid, and to confirm the open reading frame of TAT-028. The GeneRacer kit from Invitrogen (Carlsbad, Calif.) may be used for the 5′ and 3′ RACE-PCR reactions. The primers are derived from identified TAT-028 peptides (e.g., SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17), with fallback to any TAT-028 GenBank or isolated sequence. Both 5′ and 3′ RACE-PCR reactions are subcloned and sequenced. The sequences obtained are checked for the presence of the identified peptides. The sequences are then used to define the PCR primers for the next step in the process. A typical RACE-PCR reaction from a tumor is shown as an example in FIG. 15. RACE-PCR may be foregone should the genomic organization of the gene be considered to have been reliably described previously.

In step 2 (see FIG. 14), PCR walking may be performed from both the 5′- and 3′-ends, using primers designed from the sequence confirmed by RACE-PCR, with the primers usually defined at about 400-500 base pair intervals along the length of the target. With that size amplimer, standard agarose gels may generally be used to distinguish PCR products with even small differences in length (i.e., potential variants). The walk may be done in single or multiple exon-sized steps. One primer at the 5′ end of the target is paired to primers that are progressively more distant. The same process is repeated from the 3′ end. The PCR products obtained are cloned and sequenced to define the variants and allow further primer definition. PCR walks will be conducted using cDNA from patients that demonstrated a differential expression for the particular target. The amplimer patterns will be compared. If there are no differences, amplimers from 1 patient will be subcloned and sequenced to confirm the gene identity and the location of identified target peptides (e.g., peptide #1 (SEQ ID NO: 1)). Amplimers that do not match in size across the patients or are not found in all patients will be individually subcloned and sequenced. Once the identity of the target and the presence of the target peptides are confirmed, a full-length clone per target or target variant will be generated. The approach may depend on the length of the target gene. Targets greater than 5 or 6 kb may require multiple PCRs and assembly via restriction digest and subcloning. For targets without variants and up to ˜6 kb long, full length cDNAs may be recovered by PCR, using primers specific to the 5′ and 3′ ends. For targets with variants, full length clones may be recovered by Overlap PCR.

In step 3, Overlap PCR, (see FIG. 14), full length target clones may be retrieved by a series of overlapping PCR reactions. The following strategy is typically used: the first reaction is used to amplify the variant-specific region. Then, other amplifications using primers defined within the variant-specific region and adjoining 5′ and 3′ areas would be done. These amplification products would be used as templates with primers specific for the 3′ and 5′ ends, to generate amplification products that span the entire cDNA. The full length cDNA would then be subcloned, and sequenced to confirm its correctness. The tumor cell origin of full length clones could then be further confirmed through antibody generation and use in immunostaining (see, for example, Examples 10, 11, 14, 15, and 19).

The following case study further exemplifies the use of this method, cloning of CD98 based on the peptide information obtained by mass spectrometry using the methods described in Example 4. CD98, a protein of 529 amino acids with a single transmembrane domain was cloned using primers corresponding to the following 5 peptide sequences (IGDLQAFQGHGAGNLAGLK (SEQ ID NO: 32), VILDLTPNYR (SEQ ID NO: 33), LLTSFLPAQLLR (SEQ ID NO: 34), GQSEDPGSLLSLFR (SEQ ID NO: 35) and ADLLLSTQPGREEGSPLELER (SEQ ID NO: 36)). Cloning of CD98 was done from cDNA of tumor RNA from a patient in which the peptides were identified. A single CD98 variant, containing the overexpressed peptides detected by mass spectrometry was successfully cloned. Exemplary RACE-PCR reactions for CD98 are shown in FIG. 15.

### Example 9

### Expression and Purification of a TAT-028 Polypeptide

A number of protocols may be used to purify TAT-028 polypeptides, such as immunoaffinity purification with available antibodies. Alternatively, tagged or fusion proteins such as those produced by vectors described in Example 6 may be expressed and purified with appropriate methodologies.

GST-TAT-028 fusion polypeptides, such as may be produced with the GST-fusion expression vector of Example 6 may be purified as follows, or alternately by following Amersham protocols (GST Gene Fusion System Handbook, product number 18-1157-58). pGEX-TAT-028 is transformed using Top 10 (Invitrogen, Inc) competent cells. A 5 ml culture of cells containing the pGEX-TAT-028 vector is grown in LB (containing 100 mg/litre ampicillin) at 37° C. This culture is used inoculate and expand the culture, eventually inoculating 1 litre of LB broth (containing 100 mg/1 liter ampicillin) with 100 ml of cell culture (1:10 culture and LB dilution). The cells are grown until the OD (optical density) reaches 0.6-1.0 at 600 nm fixed wavelength. Cells are induced with IPTG to a final concentration of 1 mM for several hours (as best maximizes expression per pre-testing with several different time points). Cells are pelleted in a centrifuge over 15 minutes at 2000 RPM and washed three times with 1× PBS, keeping the cells on ice at all times. 10 ml of lysis solution (1× PBS, 100 mM EDTA, 1% 1000× apropotin, 1 mM AEBSF, 0.5 mM DTT) are then added to the pellet and the cells are sonicated three times for 45 seconds each. Triton X is added to a 1% final concentration. The solutions containing the cells are then placed on a rotary shaker at 4° C. for 15 minutes, followed by spinning the cells for 15 minutes at 7000 RPM, and collect the supernatant into Beckman centrifuge tubes. The supernatant is spun again for 30 minutes at 45 K and the supernatant is separated. 2 ml of 50% gluthione sepharose beads (Pharmacia) is added to the lysed cells, and the samples are incubated at 4° C. for 5 hours or overnight on a rotator. The beads are spun and the supernatant is separated. The beads are then washed 3 times with 50 volumes of 1× PBS (containing 1% triton) and one time with 50 volumes of 50 mM Tris (pH 7.5) and 150 mM NaCl. The protein is then eluted from the beads using 3-4 mls of 10 mM reduced gluthione in 50 mM Tris (pH 8.0) and again with 1-2 mls of the 10 mM gluthione. The eluted protein is dialyzed in dialysis buffer (20 mM Hepes, 150 mM KCL, 0.2 mM EDTA, 1 mM AEBSF, 20% glycerol) for 5-8 hours, but preferably overnight. The dialysed protein is analyzed by SDS-PAGE to verify the protein size and the purification procedure.

To remove the GST portion of the fusion protein, follow manufacturer instructions for pGEX-6P-1. Alternatively a GST-fusion may be designed that relies on other proteases, such as thrombin for cleavage.

His-tagged TAT-028 polypeptides may be expressed (see Example 6 for a potential vector description) in E. coli, and then extracted. Recombinant protein from a 250 ml cell pellet is extracted in 3 ml of extraction buffer by sonicating 6 times, with 6 second pulses at 4° C. The extract is then centrifuged at 15000×g for 10 minutes and the supernatant collected. The recombinant protein may be assayed for biological activity at this time.

The recombinant protein is purified by Ni—NTA affinity chromatography (Qiagen) according to the following protocol, performing all steps at 4° C. (refer to Qiagen protocols for more detail): use 3 ml Ni-beads (Qiagen), equilibrate column with equilibration buffer, load protein extract, wash with the equilibration buffer, elute bound protein with 0.5 M imidazole.

Recombinant TAT-028 proteins may also be purified using other routine protein purification methods, such as ammonium sulfate precipitation, affinity columns (e.g., immunoaffinity), size-exclusion, anion and cation exchange chromatography, gel electrophoresis and the like (see, generally, R. Scopes (1982) Protein Purification, Springer-Verlag, N.Y. and Deutscher (ed.) (1990) Methods in Enzymology Vol. 182: Guide to Protein Purification, Academic Press, Inc. N.Y.).

The purified TAT-028 polypeptides, and TAT-028 complexes provided by the present invention are, in one embodiment, highly purified (i.e., at least about 90% homogeneous, more often at least about 95% homogeneous). Homogeneity may be determined by standard means such as SDS-polyacrylamide gel electrophoresis and other means known in the art (see, e.g., Ausubel et al., supra). It will be understood that, although highly purified TAT-028 polypeptides, or TAT-028 complexes are sometimes desired, substantially purified (e.g., at least about 75% homogeneous) or partially purified (e.g., at least about 20% homogeneous) TAT-028 polypeptides, or TAT-028 complexes are useful in many applications, and are also provided by the present invention. For example, partially purified TAT-028 may be useful for screening test compounds for TAT-028 modulatory activity, and other uses.

### Example 10

### Antibody Generation

Monoclonal antibodies in humanized or chimeric forms are useful for treating a variety of neoplastic diseases. TAT-028 antibodies are produced as follows. A TAT-028 polypeptide or modification thereof may be coupled to a carrier, such as keyhole limpet hemocyanin (KLH). Coupling of TAT-028 to KLH is performed as follows. 10 mg of the TAT-028 polypeptide is dissolved in 2 ml of phosphate buffered solution (PBS 1×). 1 ml of KLH (Pierce products #77100) is added to the peptide solution and stirred (1 mole of peptide/50 amino acids). The KLH concentration is 10 mg/ml. 20 μl of glutaraldehyde (25% aqueous solution) is added to the peptide/carrier solution with constant stirring, incubated for 1 hour, and then a glycine stop solution is added. The peptide/carrier conjugate is separated from the peptide by dialysis against PBS.

Polyclonal antibodies may be prepared according to standard methods, and an immune response enhanced with repeated booster injections, at intervals of 3 to 8 weeks. The success of the immunization may be verified by determining the concentration of antibodies in a western blot or ELISA or both. More specifically, to generate polyclonal antibodies to TAT-028, the TAT-028 polypeptide conjugated to KLH is injected into rabbits in accordance with an 164 day immunization regimen, after which the animals that produce specific antibodies are bled.

In order to sample the serum prior to immunization, 10 ml of blood per rabbit may be taken as a pre-immune control. TAT-028 polypeptides may also be used in competing peptide controls. Primary immunizations may be carried out with Freund's complete adjuvant and subsequent boosts with incomplete Freund's adjuvant (IFA) (1 ml per rabbit, 0.5 ml per thigh muscle). Each injection consists of approximately 200 μg of the purified peptide. At days 21, 42, and 70, a booster injection is given with IFA. At days 31, 42 and 80, 10 ml of blood is collected from the central ear artery for titer determination (6 ml/kg/rabbit). At day 80, the titer of the sera is checked, and 3 more injections are given (IFA) at 4 week intervals, followed by blood sampling 10 days later. 10 days after the last boost, anesthetized rabbits are exsanguinated via cardiac puncture, and antisera are collected.

Goat polyclonal antibodies can also be generated according to standard methods. Goats can be immunized as follows. On day 1, all goats receive a primary immunization of 1 mg of TAT-028 polypeptide-KLH conjugates in complete Freund's adjuvant. Boosts are done by injection of 1 mg TAT-028 polypeptide-KLH in incomplete Freund's adjuvant for the goats. Serum samples from bleeds are tested for reactivity by ELISA against TAT-028-BSA conjugates. From the third set of bleeds, total IgG can be purified by ammonium sulfate precipitation and TAT-028 polypeptide-reactive IgG can be purified using a TAT-028 polypeptide affinity column. IgG fractions are tested for reactivity to TAT-028 polypeptide as described herein. The exact immunization schedule was as follows: Day 1, primary immunization; Day 21, first boost immunization; Day 30, first bleed; Day 46, second boost immunization; Day 53, second boost immunization; Day 60, second bleed; Day 76, third boost immunization; Day 83, third boost immunization; and Day 90, third bleed.

Monoclonal antibodies may be prepared using TAT-028 polypeptides and standard hybridoma technology (see, e.g., Kohler et al. (1975) Nature 256: 495-497; Kohler et al. (1976) Eur J Immunol. 6: 511-519; Kohler et al. (1976) Eur J Immunol. 6: 292-295; Hammerling et al. (1981) in Monoclonal Antibodies and T Cell Hydridomas, Elsevier, NY; Ausubel et al. (1999) Current Protocols in Molecular Biology, Wiley Interscience, New York). Once produced, monoclonal antibodies are also tested for specific TAT-028 polypeptide recognition by immunoprecipitation and western blot analysis (e.g., by using the methods described in Ausubel et al., supra).

The generation of monoclonal antibodies can be carried out as follows. Mice are immunized initially with a TAT-028 polypeptide in complete Freund's adjuvant. All subsequent immunizations are made with a TAT-028 polypeptide in Freund's incomplete adjuvant or PBS (in a final volume of 0.5 ml; 1:1 with adjuvant) as a vehicle. The following booster immunizations are made at 2-6 week intervals: Boost 1, TAT-028 polypeptide; Boost 2, PBS and 100 μg of 8-map KTEAPAAPAAQETK peptide (SEQ ID NO: 1); Boost 3, purified TAT-028 (SEQ ID NO: 19) and 100 μg of 8-map KTEAPAAPAAQETK peptide (SEQ ID NO: 1); Boost 4, purified TAT-028 (SEQ ID NO: 19) and 200 μg CKTEAPAAPAAQETK -KLH conjugate (SEQ ID NO: 37 and KLH conjugate); Boost 5, purified TAT-028 and 100 μg CKTEAPAAPAAQETK-KLH conjugate (SEQ ID NO: 37 and KLH conjugate). Splenocytes from these mice are fused to the FO murine B cell line (ATCC CRL-1646) to generate specific hybridoma clones. Hybridoma supernantants are screened by ELISA.

Monoclonal antibodies can also be made in mice by genetic immunization. Plasmids containing a TAT-028 coding sequence, along with a restriction map, can be provided to Genovac (Aldevron LLC, Fargo, N.D.). Genovac subclones the TAT-028 or a portion thereof into their immunization vector, and mice are be immunized. Transfections of the same construct will are used to screen by flow cytometry the resulting hybridomas. Antibody reactivity can be confirmed by immunohistochemistry on cells transiently transfected or mock transfected cells with an expression vector containing TAT-028 coding sequence.

### Example 11

### Screening for Antibodies

The antibodies of the invention may be selected by immobilizing a TAT-028 peptide and then panning a library of human antibody chains as described herein using the immobilized TAT-028 domain to bind antibody. The specificity and activity of specific clones can be assessed using assays known in the art. After a first panning step, a library of phage containing a plurality of different single chain antibodies displayed on phage having improved binding to the TAT-028 peptide is obtained. Subsequent panning steps provide additional libraries with higher binding affinities.

### Example 12

### Cloning of Antibody Sequences

For recombinant production of the antibody, the nucleic acid encoding it may be isolated and inserted into a replicable vector for further cloning (amplification of the DNA) or for expression. DNA encoding the monoclonal antibody is isolated and sequenced using conventional procedures (e.g., by using oligonucleotide probes that are capable of binding specifically to genes encoding the heavy and light chains of the antibody). Many vectors, as described herein, are available. The vector components generally include, but are not limited to, one or more of the following: a signal sequence, an origin of replication, one or more marker genes, an enhancer element, a promoter, and a transcription termination sequence.

### Example 13

### Antibody Production

Suitable host cells for cloning or expressing the DNA in the vectors herein are prokaryote, yeast, or higher eukaryote cells including animal and plant cell cultures. In general, host cells are transformed with the expression or cloning vectors for anti-TAT-028 antibody production and cultured in conventional nutrient media modified as appropriate for inducing promoters, selecting transformants, or amplifying the genes encoding the desired sequences. The antibody composition prepared from the cells can be purified according to standard methods well known in the art.

Amino acid sequence variants of the antibody are prepared by introducing appropriate nucleotide changes into the antibody DNA, or by peptide synthesis. Such variants include, for example, deletions from, and/or insertions into and/or substitutions of, residues within the amino acid sequences of the antibodies of the examples herein. Any combination of deletion, insertion, and substitution is made to arrive at the final construct, provided that the final construct possesses the desired binding characteristics. A useful method for identification of certain residues or regions of the antibody that are preferred locations for mutagenesis is called alanine scanning mutagenesis.

### Example 14

### Antibody Purification

Total rabbit IgG can be purified from serum using a Pharmacia protein A HiTrap column according to the manufacturer's recommendations. Briefly, the HiTrap column is equilibrated with 3 column volumes of start buffer (0.2 M sodium phosphate buffer, pH 7.0). Serum is applied, using a syringe through a luer adaptor, onto the column. The column is subsequently washed with 5 ml of start buffer. Bound protein is eluted with 0.1 M glycine, pH 3.0, and collected in eppendorf tubes containing 1M Tris pH 8.0 (50 μl/500 μl sample). Fractions are analyzed on SDS-PAGE.

Goat polyclonal antibodies can be purified from serum samples as is described above.

Mouse monoclonal antibodies can be produced as ascites, and purified using a protein A column kit (Pierce) according to the manufacturer's instructions. Briefly, a sample of ascites is diluted with binding buffer at a 1:1 final ratio. The sample is then added to the top of the column, which has been previously equilibrated with binding buffer, and allowed to flow through the matrix. The pass-through material is collected and the column washed with 5 volumes of binding buffer. Mild elution buffer is added to the column to release the bound IgG antibody from the matrix. Other antibody isotypes are collected by switching to the IgG elution buffer. All the antibodies are collected in 1 ml fractions, which are analyzed by BCA to determine total protein content and SDS-PAGE electrophoresis to establish the degree of antibody purity. The fraction containing the most yield of IgG is desalted by passing it through a D-salt column (Pierce). The antibody fraction is allocated and stored at −80° C. in PBS.

### Example 15

### Antibody Fragments

Antigen-binding fragments of anti-TAT-028 antibodies, which may be produced by conventional techniques, are also encompassed by the present invention. Examples of such fragments include, but are not limited to, Fab and F(ab′)₂ fragments. Antibody fragments and derivatives produced by genetic engineering techniques are also provided.

In one working example, pepsin digestion may be used to cleave the intact TAT-028 antibody into antibody fragments as follows. A buffer exchange with 100 mM sodium citrate (pH 3.5) using NAP™-10 columns (Amersham Pharmacia Biotech) can be used. Pepsin digestion can also be done with an unrelated human antibody (for example, Chrompure IgM, Dianova, Hamburg, Germany) to obtain a suitable negative control. For each milligram of antibody, 5 μg pepsin (Sigma Aldrich, Taufkirchen, Germany) is added, followed by incubation for 10-15 minutes in a 37° C. water bath. The reaction is stopped by adding 1/10 volume of 3.0 M Tris (pH 8.8) followed by centrifuging at 10,000 g for 30 minutes. Prior to use in experiments, the fragmented TAT-028 antibody and the fragmented human control antibody can be dialyzed against PBS.

Following cleavage, the success of pepsin digestion may be analyzed by SDS-PAGE and Western blotting under non-reducing conditions. After blotting, the intact antibody may show the characteristic bands corresponding to intact antibody, monomeric forms, and light chains. By SDS-PAGE, the intact antibody may be unable to migrate into the stacking gel. However, following 10-15 minutes of treatment with pepsin, intact antibodies are completely digested into monomeric, F(ab)₂, Fab, and light chain fragments which may be identified by molecular weight. The fragmented TAT-028 antibody may be tested for tumor-binding on paraffin sections of human lung carcinomas and compared to the intact TAT-028. Both antibody forms may possess similar binding patterns on tumor cells.

### Example 16

### CDR Consensus Sequences as Immunogens and Antigens

Cloning of the complementary-determining regions (CDRs) of anti-TAT-028 antibodies may be performed as follows. Total RNA from hybridomas which secrete a TAT-028-specific monoclonal antibody can be prepared according to a standard extraction procedure, and DNA fragments encoding the variable regions of the heavy and light chains can be amplified from poly(A)+RNA. The PCR products are then cloned into a vector such as pCR4-TOPO, pCR2.1-TOPO, or pBADThio-TOPO (Invitrogen) according to the manufacturer's instructions. The resulting clones are amplified in E. coli TOP10 cells (Invitrogen) with ampicillin (Roche) as a selective marker. Plasmid DNA is isolated from amplified clones using QIAGEN maxiprep kits, and nucleic acid sequencing is performed according to standard methods. Predicted amino acid sequences are then derived from the DNA sequences using Vector NTI (Informax).

On the basis of determining the predicted amino acid sequences, and according to the Chothia CDR definitions (Chothia et al. (1989) Nature 342: 877-83), CDRs of each variable region of mouse monoclonal antibodies to TAT-028 can be determined.

Several algorithms are available, such as the Dayhoff and Genetiq symbol comparison tables (Corpet (1988) Nucleic Acids Res. 16: 10881-10890), for aligning CDR3 sequences in order to derive a consensus sequence if multiple CDR sequences are available. These algorithms seek the minimum common elements in a collection of sequences. Immunizing antigens can be derived from determined CDR sequences and/or from deduced consensus sequences. Such sequences may also be used as antibody fragments, for example in TAT-028 binding assays, or as the basis for constrained peptides.

### Example 17

### TAT-028 Localization

To further characterize the cell surface expression of TAT-028, cell lines can be transfected with expression vectors containing full-length TAT-028 as well as a negative control and stained with anti-TAT-028 antibodies post-transfection (generally about 24 to 72 hours later). Antibodies should be directed to an external portion of TAT-028, and a panel of peptide directed antibodies may be used to map external epitopes. Control antibodies, such as pre-immune serum for rabbit polyclonals, or antibody pre-incubated with antigen peptide to compete the specific binding. Surface expression can be visualized with the aid of microscopy, or analyzed by FACS. Tumor samples and normal tissues may also be stained to further confirm disease specific expression.

### Example 18

### Protein Body Atlas

A determination of the distribution of TAT-028 in diseased and normal by tissue can be made by immunostaining of archived tissue sections, such as lung, lung, heart, liver and kidney, using anti-TAT-028 antibodies. Paraffin embedded formalin-fixed tissue can be sliced into 4 micron sections. Steam heat induced epitope retrieval (SHIER) in 0.1M sodium citrate buffer (pH 6.0) may be used for optimal staining conditions. Sections are incubated with 10% serum/PBS for 5 minutes. Primary antibody is added to each section for 25 minutes at varying concentrations, followed by a 25 minute incubation with a species-appropriate biotinylated secondary antibody. A negative control, such as pre-immune IgG in the case of rabbit antibodies should be used. Endogenous peroxidase activity is blocked by three 1.5 minute incubations with hydrogen peroxidase. The avidin biotin complex/horse radish peroxidase (ABC/HRP) system is used along with DAB chromogen to visualize antigen expression, and slides are counterstained with hematoxylin. SHIER and ABC/HRP may be used per Ventana Medical Systems, Tucson, Ariz.

### Example 19

### Animal Models (Transgenics and Knockouts)

A replacement-type targeting vector, which can be used to create a knockout model, can be constructed using an isogenic genomic clone, for example, from a mouse strain such as 129/Sv (Stratagene Inc., LaJolla, Calif.). Rat and mouse genomic sequences can be obtained from the NCBI Entrez Gene entries corresponding to the rat and mouse zenologues protein sequences provided (Rat (gi: 11560135; SEQ ID NO: 42), Mouse (gi: 66773932; SEQ ID NO: 41)). Additional rodent TAT-028 zenologue sequences can be determined using the methods of Example 5 and standard DNA sequencing methods. The targeting vector can be introduced into a suitably-derived line of embryonic stem (ES) cells by electroporation to generate ES cell lines that carry a profoundly truncated form of a TAT-028 gene. To generate chimeric founder animals, for example, mice, the targeted cell lines are injected into a blastula-stage embryo. Heterozygous offspring can be interbred to homozygosity.

### Example 20

### Antibody-Based Therapeutics

A patient diagnosed with a neoplasm (e.g., a lung carcinoma) may be treated with TAT-028 antibodies or fragments thereof as follows. Lugol's solution may be administered, e.g., 7 drops 3 times daily, to the patient. Subsequently, a therapeutic dose of ¹³¹I-TAT-028 antibody may be administered to the patient. For example, a ¹³¹I dose of 50 mCi may be given weekly for 3 weeks, and then repeated at intervals adjusted on an individual basis, e.g., every three months, until hematological toxicity interrupts the therapy. The exact treatment regimen is generally determined by the attending physician or person supervising the treatment. The radioiodinated antibodies may be administered as slow intravenous infusions in 50 ml of sterile physiological saline. After the third injection dose, a reduction in the size of the primary tumor and metastases may be noted, particularly after the second therapy cycle, or 10 weeks after onset of therapy.

### Example 21

### Vaccines

In one working example, human administration of a TAT-028 polypeptide is performed as follows. A vaccine composed of 60 mg of a recombinant TAT-028 polypeptide in a total volume of 15 ml of water containing 2% w/v sucrose, pH 7.5 is orally administered to the patient. Administration of the vaccine is repeated at weekly intervals for a total of 4 doses. Symptoms are recorded daily by the patient. To determine adverse effects, physician interviews are performed weekly during the period of vaccine administration, as well as 1 week and 1 month after the last immunization. Anti-TAT-028 antibodies are measured in serum and saliva, and antibody-secreting cells are monitored in peripheral blood collected 7 days after the last immunization.

### Example 22

### cDNA Cloning and Expression Vector Construction

Human brain total RNA (Catalogue #636643, Clontech Laboratories, Inc., Mountain View, Calif.) was used in a reverse transcription PCR reaction to amplify TAT-028 utilizing oligodeoxynucleotide primers 1-2 (SEQ ID NO: 43 and SEQ ID NO: 44) and the SuperScript™ one-step RT-PCR system (Catalogue #10928-034, Invitrogen, Carlsbad, Calif.) following the manufacturer's instructions.

The PCR reaction was separated on a 1% agarose gel and the amplified DNA was purified using Qiagen QIAquick Gel Extraction Kit (Catalogue #28704, Qiagen, Hilden, Germany) in accordance with the manufacturer's instructions. The purified PCR product was cloned into the pCR2.1-TOPO vector using an TOPO TA cloning Kit (Catalogue #45-0641, Invitrogen, Carlsbad, Calif.) following the manufacturer's instructions. Basically five μl of PCR product were incubated with 1 μl of pCR2.1-TOPO vector and 1 μl of salt solution at room temperature for 5 minutes. The mixtures were transformed into E. coli TOP10 cells (Invitrogen Catalogue #44012) and grown on LBamp^{r} agar plates, at 37° C., in the presence of 5-bromo-4-chloro-3-indolyl-β-D-galactoside. White colonies were selected, grown in liquid culture and plasmid DNA was isolated using Qiaprep Spin Miniprep Kit in accordance with the manufacturer's instructions (Qiagen, Catalogue #27106) The identity of the cloned region (SEQ ID NO: 47) was verified by DNA sequencing using standard M13 forward and reverse primers (SEQ ID NO: 45 & SEQ ID NO: 46, respectively) and ABI BigDye chemistry. Reactions were analyzed on ABI automated DNA sequencers (Applied Biosystems Inc. Foster City, Calif.). The protein sequence (SEQ ID NO: 19) coded by the DNA identically matched the known sequence of TAT-028 (Accession No.: NP_{—}006308 (National Center for Biotechnology Information)).

A modified version of bacterial expression vector pET15b (Catalogue #69661-3, EMD Bioscience, Darmstadt, Germany) was used for bacterial expression. PET15b encodes ampicillin antibiotic resistance and contains a bacterial origin of replication, an IPTG inducible promoter, a ribosome binding site, six codons encoding histidine residues that allow affinity purification using nickel-affinity resin and single restriction enzyme cleavage sites. The modification was made to add two BsaI restriction sites in the multiple cloning sites to generate NcoI and XhoI sticky end, and to change the 6-histidine tag to the C-terminus. These elements are arranged such that a DNA fragment encoding a polypeptide may be inserted in such as way as to produce that polypeptide with the 6-hisitdine tag covalently linked to the carboxyl terminus of that polypeptide.

The TAT-028-pCR2.1-TOPO plasmid was cleaved at 55° C. for 2 hours with restriction enzyme BsmBI (Catalogue #R0580S, New England Biolabs, MA) to generate a TAT-028 fragment with 5′NcoI and 3′XhoI compatible ligation sites. The cleaved TAT-028 insert was separated from the vector by 1% agarose gel electrophoresis and purified using Qiagen QIAquick Gel Extraction Kit (Catalogue #28704, Qiagen, Hilden, Germany) in accordance with the manufacturer's instructions. Six μl of purified insert was then ligated to 1 μl of modified pET15b vector linearized by restriction enzyme BsaI (Catalogue #R0535S, New England Biolabs, MA) using T4 DNA ligase (Catalogue #15224-017 Invitrogen, Carlsbad, Calif.). The mixtures were transformed into E. coli strain DH5α and grown on LB agar plates containing carbenicillin (Cat #L1010, Teknova, Hollister, Calif.) at 37° C. Colonies with inserts were identified through colony PCR utilizing T7 promoter primer and T7 terminator primer (SEQ ID NO: 48 & SEQ ID NO: 49, respectively) and a Qiagen master mix PCR kit (Catalogue #201443, Qiagen, Hilden, Germany). The positive colony was picked to grow in LB liquid culture at 37° C. and plasmid DNA was isolated using Qiaprep Spin Miniprep Kit in accordance with the manufacturer's instructions (Catalogue #27106, Qiagen).

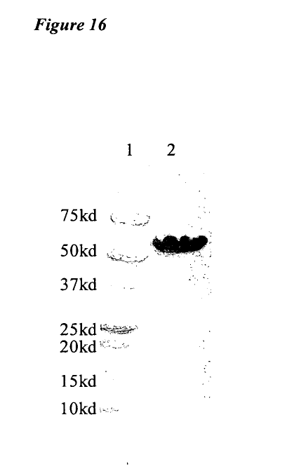

Bacteria strain BL21DE3 cells harboring the TAT-028 expression plasmid were grown in shaken flasks at 37° C. until OD600 reached 0.8. Protein expression was induced with 1 mM IPTG at 25° C. for 4 hours. The cells were collected by centrifugation at 4,000×g for 20 minutes. Two grams of cell paste were resuspended in 100 ml of cell lysis buffer (20 mM Tris-HCl, pH 8, 200 mM NaCl, and 10 mM Immidazole) and lysed by microfluidizer. The cell lysate was centrifuged at 27,000×g for 30 minutes. TAT-028 recombinant protein was purified from the supernatant of the cell lysate using Nickel affinity ProBond Resin (Catalogue #46-0019, Invitrogen, Carlsbad, Calif.) in accordance to the manufacturer's protocol. The purified protein showed one single band on 4-20% SDS PAGE gel (FIG. 16). The protein was confirmed by protease digestion and Mass spectroscopy sequencing. The molecular weight determined by mass spectroscopy was 24,626 daltons (see FIG. 16).

| | |
|---|---|
| SEQ ID NO:43 | acgacgacgtctcccatgggaggcaagctcagcaag |
| SEQ ID NO:44 | cgagagacgtctcctcgagctctttcacggttacggt |
| | ttg |
| SEQ ID NO:45 | gtaaaacgacggccag |
| SEQ ID NO:46 | caggaaacagctatgac |
| SEQ ID NO:47 | atgggaggcaagctcagcaagaagaagaagggctaca |
| | atgtgaacgacgagaaagccaaggagaaagacaagaa |
| | ggccgagggcgcggcgacggaagaggaggggaccccg |
| | aaggagagtgagccccaggcggccgcagagcccgccg |
| | aggccaaggagggcaaggagaagcccgaccaggacgc |
| | cgagggcaaggccgaggagaaggagggcgagaaggac |
| | gcggcggctgccaaggaggaggccccgaaggcggagc |
| | ccgagaagacggagggcgcggcagaggccaaggctga |
| | gcccccgaaggcgcccgagcaggagcaggcggccccc |
| | ggccccgctgcgggcggcgaggcccccaaagctgctg |
| | aggccgccgcggccccggccgagagcgcggcccctgc |
| | cgccggggaggagcccagcaaggaggaaggggaaccc |
| | aaaaagactgaggcgcccgcagctcctgccgcccagg |
| | agaccaaaagtgacggggccccagcttcagactcaaa |
| | acccggcagctcggaggctgccccctcttccaaggag |
| | acccccgcagccacggaagcgcctagttccacaccca |
| | aggcccagggccccgcagcctctgcagaagagcccaa |
| | gccggtggaggccccggcagctaattccgaccaaacc |
| | gtaaccgtgaaagag |
| SEQ ID NO:19 | Mggklskkkkgynvndekakekdkkaegaateeegtp |
| | kesepqaaaepaeakegkekpdqdaegkaeekegekd |
| | aaaakeeapkaepektegaaeakaeppkapeqeqaap |
| | gpaaggeapkaaeaaaapaesaapaageepskeegep |
| | kkteapaapaaqetksdgapasdskpgsseaapsske |
| | tpaateapsstpkaqgpaasaeepkpveapaansdqt |
| | vtvke |
| SEQ ID NO:48 | taatacgactcactataggg |
| SEQ ID NO:49 | ctagttattgctcagcgg |

### Example 23

### Generation of TAT-028 Monoclonal Antibodies

Recombinant TAT-028 (100 μg) was used to immunize Balb/c mice (Jackson Labs, Bar Harbor, Me.) subcutaneously using AdjuLite® Complete Freunds Adjuvant (Catalogue #A5001, Pacific Immunology, Ramona, Calif.). Subsequent boosts were given at 3-4 week intervals, at 10-50 μg/mouse in Freunds Incomplete Adjuvant (Cat #A5002, Pacific Immunology). Beginning 7-10 days after the first boosts, antibody titers were monitored by standard enzyme-linked immunosorbent assay (ELISA) methodology described below. Once satisfactory titers were achieved (˜1:100,000), a final IV prefusion boost was given, without adjuvant three to four days prior to fusion. For fusions, isolated lymphocytes from immunized mice spleens were fused with SP2/0 myeloma cells (ATCC #CRL1646, American Type Culture Collection, Manassas, Va.) at a 1:1 ratio. Positive hybrids and clones were identified by testing the tissue culture supernatants by antibody capture ELISA. Specifically, 50 μl of a 1 μg/mL soluble TAT-028 antigen was directly absorbed onto microtiter assay plates for 30 minutes at room temperature, and unbound sites were blocked (2% bovine serum albumin in 1× PBS, pH 7.4) for 30 minutes at room temperature. After three washes using 1× PBS, 0.05% Tween-20, 50 μl of tissue culture supernatant from hybrids or clones were added to the wells, and incubated for two hours at room temperature. Unbound antibody was washed from the assay plates (1× PBS, 0.05% Tween-20), followed by the addition of 50 μl of HRP-labeled anti-mouse Ig conjugate diluted 1:5000 in 2% BSA-PBS. Unbound conjugate was washed from the assay plates (1× PBS, 0.05% Tween-20), and 50 μl of OPD (O-phenylenediamine-dihydrochloride) enzyme substrate (Sigma #P9187) was added in each well for 5 minutes at room temperature for color development. Optical densities (OD) were read at 490 nm and readings greater than 2-fold over background were considered positive. Positive clones were expanded and monoclonal antibodies were purified from tissue culture supernatants using protein A chromatography. A summary of the TAT-028 monoclonal antibodies generated and their signal to noise optical density values by ELISA is shown in Table 1.

TABLE 1
Summary of monoclonal antibodies generated to TAT-028.

| TAT-028 mAb | Signal to Noise Ratio |
|---|---|
| Clone | (OD at 490 nm) |
| H36C8 | 10.1 |
| H36C9 | 10.6 |
| H19C2 | 2.1 |

### Example 24

### Binding of TAT-028 Monoclonal Antibodies to the Surface of Human Tumor Cell Lines as Assessed by Flow Cytometry

Flow cytometry was performed to determine whether TAT-028 monoclonal antibodies bind to the surface of human tumor cell lines. Specifically, 1×10⁶ logarithmically growing human tumor cell lines were dislodged from a T75 flask using Versene (Catalogue #15040066, Invitrogen, Carlsbad, Calif.), resuspended in 100 μl of FACS buffer (1× PBS, 1% FBS), and cells stained with 2 μg of TAT-028 monoclonal antibody on ice for 1 hour. Cells were washed 1× with FACS buffer, resuspended in 100 μl of FACS buffer containing 1 μg of PE-conjugated anti-mouse IgG, and incubated for 30 minutes on ice. Cells were washed 1× with FACS buffer, fixed in 300 μl of 1% formaldehyde and analyzed using a Becton Dickinson LSR II (San Jose, Calif.). To determine the monoclonal antibody binding specificity to TAT-028 protein on the cell surface, a 600-fold molar excess of TAT-028 recombinant protein was pre-incubated with the TAT-028 monoclonal antibodies for 15 minutes prior to addition to the tumor cells. These studies revealed that TAT-028 monoclonal antibodies bind to the surface of human tumor cell lines relative to PE-conjugated anti-mouse IgG staining only as shown by the geometric mean fluorescence units (Table 2). This binding is competed away using TAT-028 recombinant protein.

TABLE 2
Summary of TAT-028 monoclonal antibody binding activity −/+ TAT-028
protein as assessed by flow cytometry.

| | | NCI-H460 + | | HEK293 + | | HepG2 + |
|---|---|---|---|---|---|---|
| mAb | | TAT-028 | | TAT-028 | | TAT-028 |
| Clone | NCI-H460 | ECD | HEK293 | ECD | HepG2 | ECD |
| H36C8 | 314* | 175 | 473 | 308 | 535 | 196 |
| H36C9 | 365 | 170 | 457 | 201 | 479 | 201 |
| H19C2 | 456 | 221 | 452 | 357 | 585 | 164 |
| PE only | 37 | ND** | 124 | ND | 178 | ND |

*Units as geometric mean fluorescence.
**ND, not determined.

### Example 25

### Gene Amplification of TAT-028 in Human Tumors

Genetic heterogeneity of cancer is a factor complicating the development of efficacious cancer drugs. Cancers that are considered to be a single disease entity according to classical histopathological classification often reveal multiple genomic subtypes when subjected to molecular profiling. In some cases, molecular classification proved to be more accurate than the classical pathology. The efficacy of targeted cancer drugs may correlate with the presence of a genomic feature, such as a gene amplification (Cobleigh, M. A., et al., “Multinational study of the efficacy and safety of humanized anti-HER2 monoclonal antibody in women who have HER2-overexpressing metastatic breast cancer that has progressed after chemotherapy for metastatic disease”, J. Clin. Oncol., 17: 2639-2648, 1999) or a mutation (Lynch, T. J., et al., “Activating mutations in the epidermal growth factor receptor underlying responsiveness of non-small-cell lung cancer to gefitinib”, N. Engl. J. Med., 350: 2129-2139, 2004). For HER-2 in breast cancer, it has been demonstrated that detection of gene amplification provides superior prognostic and treatment selection information as compared with the detection by immunohistochemistry (IHC) of the protein overexpression (Pauletti, G., et al., “Assessment of Methods for Tissue-Based Detection of the HER-2/neu Alteration in Human Breast Cancer: A Direct Comparison of Fluorescence In Situ Hybridization and Immunohistochemistry”, J. Clin. Oncol., 18: 3651-3664, 2000). A need therefore exists for genomic classification markers that may improve the response rate of patients to targeted cancer therapy.

Genomic DNA from 62 human non-small cell lung cancer tumor samples was run on 100K SNP genotyping array sets. Each 100K set consists of two 50K arrays, HindIII (GeneChip® Human Mapping 50K Array Hind 240, Catalog#900523, Affymetrix, Santa Clara, Calif.) and XbaI (GeneChip® Human Mapping 50K Array Xba 240, Catalog#900518, Affymetrix, Santa Clara, Calif.). Briefly, total genomic DNA was isolated from human tumor tissue using a DNeasy Tissue Kit (Catalog#69504, Qiagen, Valencia, Calif.) according to manufacturer's instructions. 250 ng of genomic DNA from each tumor sample was digested with the corresponding restriction enzyme (HindIII or XbaI, New England Biolabs, Boston, Mass.). HindIII Adapters (Catalog#900521, Affymetrix, Santa Clara, Calif.) or XbaI Adapters (Catalog#900520, Affymetrix, Santa Clara, Calif.) and ligated to the HindIII-digested or XbaI-digested DNA, respectively, followed by PCR amplification with Pfx DNA polymerase (Invitrogen, Carlsbad, Calif.). The PCR products were purified, fragmented, labeled, and hybridized to the SNP microarray. Starting with the 250 ng of genomic DNA through to the beginning of the hybridization all procedures strictly followed the Affymetrix GeneChip® Mapping 100K Assay Manual. After a 16-hour hybridization at 48° C. at 60 rpm, the arrays were washed using the GeneChip® Fluidics Station 450(Affymetrix, Santa Clara, Calif.) and scanned in Affymetrix GeneChip® Scanner 7G following manufacturer's instructions. The data were processed using the Affymetrix GTYPE software to create copy number (.cnt) files containing information on the inferred copy number for each probeset (SNP). The GTYPE software generates an inferred copy number for each SNP by comparing the signal intensity for the sample with an internal data set from a healthy population, which is included in the GTYPE software. The .cnt files contained combined information from both arrays in the set. These files were converted into .txt files and loaded into an internally developed software program for further analysis.

The software program was used for the graphical display and analysis of multiple .txt files. The data were displayed chromosome by chromosome as a histogram of copy number versus SNP's ordered sequentially along the chromosome. For each SNP, the predicted cytogenetic band as well as any genes between this and the next adjacent SNP were reported. The gene coordinates and cytogenetic band positions were inferred from the Build 35 of the Human Genome. From a selected region of the histogram, for example, 5p15.1-p14, a summary file can be produced that contains the coordinates of all probesets on the microarray for that region (individual SNP's) with the corresponding copy numbers, cytogenetic bands, gene IDs, names, and the coordinates of all the genes residing in the region (regardless of whether a gene is actually represented by SNP's on the array). Any region with a copy number greater than 2.75 was considered to be significantly amplified. By scoring each SNP probeset (genomic region) across all of the human tumor samples a value for the percentage of the samples that were amplified at any particular region (Amplification Frequency) was determined. Three probes (SNP_A_{—}1724129, SNP_A_{—}1668550, and SNP_A_{—}1702153) lie within the TAT-028 gene. Table 3 shows the Amplification Frequency for probes in this region. The TAT-028 gene was amplified in 21-23% of the human tumor samples evaluated.

TABLE 3
Amplification of TAT-028 gene in human non-small cell lung cancer
tumor samples. Physical location is measured in base pairs.

| | | Physical | Amplification |
|---|---|---|---|
| Probeset | Chromosome | Location | Frequency |
| SNP_A-1737853 | 5 | 16954752 | 8% |
| SNP_A-1738059 | 5 | 16956718 | 8% |
| SNP_A-1739678 | 5 | 16963605 | 24% |
| SNP_A-1728550 | 5 | 17165996 | 47% |
| SNP_A-1713212 | 5 | 17246121 | 23% |
| SNP_A-1713461 | 5 | 17246633 | 23% |
| SNP_A-1724129 | 5 | 17288998 | 23% |
| SNP_A-1668550 | 5 | 17302420 | 21% |
| SNP_A-1702153 | 5 | 17308364 | 21% |
| SNP_A-1663501 | 5 | 17380540 | 21% |
| SNP_A-1707324 | 5 | 17421172 | 18% |
| SNP_A-1714315 | 5 | 17435703 | 15% |
| SNP_A-1669745 | 5 | 17445029 | 15% |
| SNP_A-1722826 | 5 | 17446190 | 15% |
| SNP_A-1717894 | 5 | 17452882 | 15% |
| SNP_A-1717998 | 5 | 17452899 | 15% |
| SNP_A-1719806 | 5 | 17456662 | 11% |
| SNP_A-1719972 | 5 | 17457483 | 11% |

### Example 26

### Example: Tumoricidal Effect of a Monoclonal Antibody In Vitro

Monoclonal antibodies diluted in D-PBS (Dulbecco's phosphate buffered saline) are added to human tumor cells at final concentrations of 0.05 μg/mL-50 μg/mL. The plates are incubated at 37° C. in a humidified, 5% CO₂ atmosphere for 3 to 6 days. The number of live cells in each well are quantified using the ATPlite™ assay according to the manufacturer's instructions (PerkinElmer Life Sciences, Downers Grove, Ill.) to determine the percent of tumor growth inhibition. Wells without treatment are used as controls of 0% inhibition whereas wells without cells are considered to show 100% inhibition. Cell death is measured using the ToxiLight® assay (Cambrex Corporation, Rockland, Me.). The ToxiLight® BioAssay Kit is a bioluminescent assay designed to measure the release of adenylate kinase, which is released into the culture medium when cells die after monoclonal antibody treatments. The enzyme actively phosphorylates ADP to form ATP and the resultant ATP is then measured using firefly luciferase. As the level of cell rupture increases, the amount of light generated also increases. Cells treated with monoclonal antibody show an increase in the amount of light generated, indicating increased cell death. Raw data values are recorded as luciferase units on a 1420 VICTOR Multilabel Counter (PerkinElmer Life Sciences, Downers Grove, Ill.).

For assessment of apoptosis, caspase-3 activation is determined by the following protocol: antibody-treated cells in 96 well plates are lysed in 120 μl of 1× lysis buffer (1.67 mM HEPES, pH 7.4, 7 mM KCl, 0.83 mM MgCl₂, 0.11 mM EDTA, 0.11 mM EGTA, 0.57% CHAPS, 1 mM DTT, 1× protease inhibitor cocktail tablet; EDTA-free; Roche Pharmaceuticals, Nutley, N.J.) at room temperature with shaking for 20 minutes. After cell lysis, 80 μl of a caspase-3 reaction buffer (48 mM HEPES, pH 7.5, 252 mM sucrose, 0.1% CHAPS, 4 mM DTT, and 20 μM Ac-DEVD-AMC substrate; Biomol Research Labs, Inc., Plymouth Meeting, Pa.) is added and the plates are incubated for 2 hours at 37° C. The plates are read on a 1420 VICTOR Multilabel Counter (Perkin Elmer Life Sciences, Downers Grove, Ill.) using the following settings: excitation=360/40, emission=460/40. An increase of fluorescence units from antibody-treated cells relative to the isotype antibody control-treated cells is seen, which is indicative of apoptosis.

### Example 27

### Efficacy of Monoclonal Antibody by Itself or in Combination with Chemotherapy on the Growth of Human Carcinoma Xenografts (Subcutaneous Flank, Orthotopic or Spontaneous Metastases)

Human cancer cells are grown in vitro to 99% viability, 85% confluence in tissue culture flasks. SCID female or male mice (Charles Rivers Labs) at 19-25 grams, are ear tagged and shaved. Mice are then inoculated subcutaneously into the right flank with 0.2 ml of 2×10⁶ human tumor cells (1:1 Matrigel™) on study day 0. Administration (IP, Q3D/week) of vehicle (PBS), antibody, and/or chemotherapy is initiated after mice are size matched into separate cages of mice with mean tumor volumes of approximately 150 to 200 mm³. The tumors are measured by a pair of calipers twice a week starting on approximately day 10 post inoculation and the tumor volumes calculated according to the formula V=L×W²/2 (V: volume, mm³; L: length, mm. W: width, mm). Reduction in tumor volume is seen in animals treated with monoclonal antibody alone or in combination with chemotherapy relative to tumors in animals that received only vehicle or an isotype control monoclonal antibody. The mice are also weighed once a week to monitor for weight loss due to toxicity or excessive tumor burden. The mice are humanely euthanized when the tumor volumes reach a predetermined size.

### Other Embodiments

It will be clear that the invention may be practiced other than as particularly described in the foregoing description and examples. Numerous modifications and variations of the present invention are possible in light of the above teachings and, therefore, are within the scope of the claims.

Preferred features of each aspect of the invention are as for each of the other aspects mutatis mutandis. The documents including patents, patent applications, journal articles, abstracts, laboratory manuals, books, or other disclosures mentioned herein are hereby incorporated by reference to the fullest extent permitted by law. Further, the hard copy of the sequence listing submitted herewith and the corresponding computer readable form are both incorporated herein by reference in their entireties.

What is claimed is:

1. An antibody or fragment thereof that specifically binds to a TAT-028 polypeptide.

2. The antibody of claim 1, wherein said polypeptide comprises the amino acid sequence of any of SEQ ID NOS: 1, 3, 5, 7, 9, 11, 13, 15, 17, 19, 38, 39, 40, 41, and 42.

3. The antibody of claim 1, wherein said antibody is a monoclonal antibody, a polyclonal antibody, a single-chain antibody, a chimeric antibody, a humanized antibody, a fully-humanized antibody, a human antibody, or a bispecific antibody.

4. The antibody fragment of claim 1, wherein said antibody fragment is a Fab fragment, an F(ab)′₂ fragment, or an Fv fragment.

5. The antibody of claim 1, wherein said antibody is conjugated to a therapeutic moiety, a detectable label, a second antibody or a fragment thereof, a cytotoxic agent or a cytokine.

6. A method of diagnosing an increased likelihood of developing a TAT-028-related disease or condition in a test subject, said method comprising analyzing nucleic acid molecules of the test subject to determine whether said test subject contains a mutation in a TAT-028 gene, wherein the presence of said mutation is an indication that said test subject has an increased likelihood of developing a TAT-028-related disease.

7. The method of claim 6, further comprising the step of using nucleic acid molecule primers specific for the TAT-028 gene for nucleic acid molecule amplification by the polymerase chain reaction.

8. The method of claim 7, further comprising the step of sequencing TAT-028 nucleic acid molecules from said test subject.

9. The method of claim 6, wherein said test subject is a mammal.

10. The method of claim 9, wherein said test subject is human.

11. The method of claim 6, wherein said analyzing is carried out by restriction fragment length polymorphism (RFLP) analysis.

12. The method of claim 6, wherein said disease or condition is a cellular proliferative disease.

13. The method of claim 12, wherein said cellular proliferative disease is cancer.

14. The method of claim 13, wherein said cancer is lung cancer.

15. A probe for analyzing the TAT-028 nucleic acid molecules of an animal, said probe having at least 60% nucleic acid sequence identity to a sequence encoding a TAT-028 polypeptide or a fragment thereof, wherein said fragment encodes at least six contiguous amino acids and said probe hybridizes under high stringency conditions to at least a portion of a TAT-028 nucleic acid molecule.

16. A method of detecting the presence of a TAT-028 nucleic acid in a sample, said method comprising contacting said sample with a probe of claim 15.

17. A kit for the analysis of a TAT-028 nucleic acid molecule, said kit comprising a nucleic acid molecule probe of claim 15 for analyzing the nucleic acid molecules of a test subject.

18. A method of detecting the presence of a TAT-028 polypeptide in a sample, said method comprising contacting said sample with a TAT-028 binding molecule that specifically binds to a TAT-028 polypeptide and assaying for binding of said molecule to said polypeptide.

19. A method of detecting the presence of a mutant TAT-028 polypeptide in a sample, said method comprising contacting said sample with an antibody that specifically binds to a mutant TAT-028 polypeptide and assaying for binding of said antibody to said mutant polypeptide.

20. A kit for the analysis of a TAT-028 polypeptide, said kit comprising an antibody for analyzing the TAT-028 polypeptide of a test subject.

21. A method for preventing or ameliorating the effect of a TAT-028 deficiency, said method comprising administering to a subject having a TAT-028 deficiency a therapeutically-effective amount of a compound to prevent or ameliorate said effect of said TAT-028 deficiency.

22. The method of claim 21, wherein said compound comprises a functional TAT-028 polypeptide.

23. A method for preventing or ameliorating the effect of a TAT-028 polypeptide excess, said method comprising administering a therapeutically-effective amount of a compound to a subject having a TAT-028 excess, wherein said compound is sufficient to prevent or ameliorate said effect of said TAT-028 polypeptide excess.

24. The method of claim 23, wherein said TAT-028 excess is caused by a cellular proliferative disorder.

25. The method of claim 24, wherein said cellular proliferative disorder is cancer.

26. The method of claim 25, wherein said cancer is lung cancer.

27. The method of claim 23, wherein said compound is an antibody or fragment thereof which binds to a TAT-028 polypeptide.

28. A substantially pure TAT-028 polypeptide or fragment thereof.

29. A substantially pure nucleic acid molecule comprising a sequence encoding a TAT-028 polypeptide, or fragment thereof.

30. A vector comprising the nucleic acid molecule of claim 29.

31. A cell comprising the vector of claim 30.

32. A non-human transgenic animal comprising the nucleic acid molecule of claim 29.

33. A composition for inducing an immune response in a subject, said composition comprising a substantially pure TAT-028 polypeptide or fragment thereof in a pharmaceutically-acceptable carrier.

34. A composition for inducing an immune response in a subject, said composition comprising the nucleic acid molecule of claim 29 and a pharmaceutically-acceptable carrier.

35. A method of inducing an immune response to a TAT-028 polypeptide, said method comprising the steps of:
(a) providing a TAT-028 polypeptide; and
(b) contacting said polypeptide with an immune system cell, thereby inducing an immune response to said polypeptide.

36. A method of inducing an immune response in a subject comprising administering to said subject a composition comprising a TAT-028 polypeptide.

37. A method of inducing an immune response in a subject comprising administering to said subject a composition comprising the nucleic acid molecule of claim 29.

38. A pharmaceutical composition comprising (i) a TAT-028 polypeptide and (ii) a pharmaceutically acceptable carrier.

39. A method of preventing or treating a cellular proliferative disease in a subject comprising administering to said subject the pharmaceutical composition of claim 38.

40. A pharmaceutical composition comprising (i) a compound that binds to a TAT-028 polypeptide and (ii) a pharmaceutically acceptable carrier.

41. The composition of claim 40, wherein said compound is an antibody or fragment thereof that binds to said TAT-028 polypeptide.

42. A method of preventing or treating a cellular proliferative disease in a subject patient, said method comprising administering to said subject the pharmaceutical composition of claim 40.

43. A pharmaceutical composition comprising (i) a TAT-028 nucleic acid molecule and (ii) a pharmaceutically acceptable carrier.

44. A method of preventing or treating a cellular proliferative disease in a subject patient, said method comprising administering to said subject the pharmaceutical composition of claim 43.


SEQUENCE LISTING (58 sequences)

SEQ ID NO: 1 - protein, 14 aa, Homo sapiens
KTEAPAAPAAQETK

SEQ ID NO: 2 - DNA, 42 bp, Homo sapiens
aaaactgaagctccagccgcacctgcagcgcaagagacaaag

SEQ ID NO: 3 - protein, 28 aa, Homo Sapiens
AAEAAAAPAESAAPAAGEEPSKEEGEPK

SEQ ID NO: 4 - DNA, 84 bp, Homo Sapiens
gccgcagaggctgcagcggcacctgccgaaagtgcggctcctgcagcaggagaagagccaagcaaagaagagggtgaaccaaag

SEQ ID NO: 5 - protein, 13 aa, Homo Sapiens
AEPEKTEGAAEAK

SEQ ID NO: 6 - DNA, 39 bp, Homo Sapiens
gctgaaccagagaagacagagggagcggcagaagccaaa

SEQ ID NO: 7 - protein, 24 aa, Homo Sapiens
AEPPKAPEQEQAAPGPAAGGEAPK

SEQ ID NO: 8 - DNA, 72 bp, Homo Sapiens
gctgaacctccaaaggccccagagcaagaacaagcagcgcctggtcctgcagctggaggggaggcaccaaaa

SEQ ID NO: 9 - protein, 19 aa, Homo Sapiens
APEQEQAAPGPAAGGEAPK

SEQ ID NO: 10 - DNA, 57 bp, Homo Sapiens
gccccagagcaagaacaagctgcgccaggacctgcagcaggtggggaagcacctaaa

SEQ ID NO: 11 - protein, 28 aa, Homo Sapiens
AQGPAASAEEPKPVEAPAANSDQTVTVK

SEQ ID NO: 12 - DNA, 84 bp, Homo Sapiens
gcgcaaggacctgctgccagcgctgaagagccaaaacctgttgaagcaccagcagcaaatagtgatcaaacagtaactgtgaag

SEQ ID NO: 13 - protein, 14 aa, Homo Sapiens
EKPDQDAEGKAEEK

SEQ ID NO: 14 - DNA, 42 bp, Homo Sapiens
gagaaaccagatcaagacgctgaaggaaaggcagaggaaaaa

SEQ ID NO: 15 - protein, 14 aa, Homo Sapiens
ETPAATEAPSSTPK

SEQ ID NO: 16 - DNA, 42 bp, Homo Sapiens
gagacacctgctgcaacagaagcgccaagcagtactccaaaa

SEQ ID NO: 17 - protein, 21 aa, Homo Sapiens
SDGAPASDSKPGSSEAAPSSK

SEQ ID NO: 18 - DNA, 63 bp, Homo Sapiens
agtgatggtgcaccagcgagtgactccaaaccaggaagctctgaagccgctccttcatcgaag

SEQ ID NO: 19 - protein, 227 aa, Homo sapiens
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 20 - DNA, 684 bp, Homo sapiens
atgggaggcaagctcagcaagaagaagaagggctacaatgtgaacgacgagaaagccaaggagaaagacaagaaggccgagggcgcggcgacggaagaggaggggaccccgaaggagagtgagccccaggcggccgcagagcccgccgaggccaaggagggcaaggagaagcccgaccaggacgccgagggcaaggccgaggagaaggagggcgagaaggacgcggcggctgccaaggaggaggccccgaaggcggagcccgagaagacggagggcgcggcagaggccaaggctgagcccccgaaggcgcccgagcaggagcaggcggcccccggccccgctgcgggcggcgaggcccccaaagctgctgaggccgccgcggccccggccgagagcgcggcccctgccgccggggaggagcccagcaaggaggaaggggaacccaaaaagactgaggcgcccgcagctcctgccgcccaggagaccaaaagtgacggggccccagcttcagactcaaaacccggcagctcggaggctgccccctcttccaaggagacccccgcagccacggaagcgcctagttccacacccaaggcccagggccccgcagcctctgcagaagagcccaagccggtggaggccccggcagctaattccgaccaaaccgtaaccgtgaaagagtga

SEQ ID NO: 21 - DNA, 1820 bp, Homo sapiens
gcggcgacgacggcggcggcagcgctccaactggctcctcgctccgggctccgccgtcgagccgggagagagcctccgccagcggccaggcaccagccagacgacgccagcgaccccggcctctcggcggcaccgcgctaactcaggggctgcataggcacccagagccgaactccaagatgggaggcaagctcagcaagaagaagaagggctacaatgtgaacgacgagaaagccaaggagaaagacaagaaggccgagggcgcggcgacggaagaggaggggaccccgaaggagagtgagccccaggcggccgcagagcccgccgaggccaaggagggcaaggagaagcccgaccaggacgccgagggcaaggccgaggagaaggagggcgagaaggacgcggcggctgccaaggaggaggccccgaaggcggagcccgagaagacggagggcgcggcagaggccaaggctgagcccccgaaggcgcccgagcaggagcaggcggcccccggccccgctgcgggcggcgaggcccccaaagctgctgaggccgccgcggccccggccgagagcgcggcccctgccgccggggaggagcccagcaaggaggaaggggaacccaaaaagactgaggcgcccgcagctcctgccgcccaggagaccaaaagtgacggggccccagcttcagactcaaaacccggcagctcggaggctgccccctcttccaaggagacccccgcagccacggaagcgcctagttccacacccaaggcccagggccccgcagcctctgcagaagagcccaagccggtggaggccccggcagctaattccgaccaaaccgtaaccgtgaaagagtgacaaggacagcctataggaaaaacaataccacttaaaacaatctcctctctctctctctctctctctctctatctctctctctatctcctctctctctctcctctcctatctctcctctctctctctcctatactaacttgtttcaaattggaagtaatgatatgtattgcccaaggaaaaatacaggatgttgtcccatcaagggagggagggggtgggagaatccaaatagtatttttgtggggaaatatctaatataccttcagtcaactttaccaagaagtcctggatttccaagatccgcgtctgaaagtgcagtacatcgtttgtacctgaaactgccgccacatgcactcctccaccgctgagagttgaatagcttttcttctgcaatgggagttgggagtgatgcgtttgattctgcccacagggcctgtgccaaggcaatcagatctttatgagagcagtattttctgtgttttctttttaatttacagcctttcttattttgatatttttttaatgttgtggatgaatgccagctttcagacagagcccacttagcttgtccacatggatctcaatgccaatcctccattcttcctctccagatatttttgggagtgacaaacattctctcatcctacttagcctacctagatttctcatgacgagttaatgcatgtccgtggttgggtgcacctgtagttctgtttattggtcagtggaaatgaaaaaaaaaaaaaaaaaaagtctgcgttcattgcagttccagtttctcttccattctgtgtcacagacaccaacacaccactcattggaaaatggaaaaaaaaaacaaaaaaaaaacaaaaaaatgtacaatggatgcattgaaattatatgtaattgtataaatggtgcaacagtaataaagttaaacaattaaaaagaaaaaaaaaaaaaaaaaaaaaaaaa

SEQ ID NO: 22 - DNA, 59194 bp, Homo sapiens
  CODING_SEQUENCE (1)..(170)
  CODING_SEQUENCE (57568)..(59194)
gcggcgacgacggcggcggcagcgctccaactggctcctcgctccgggctccgccgtcgagccgggagagagcctccgccagcggccaggcaccagccagacgacgccagcgaccccggcctctcggcggcaccgcgctaactcaggggctgcataggcacccagagccggtaaggaggcgcgcccggtggccccgcgccgccgcggacccgggggcctccgtcccccaccgcgccgcgccccgccccgcagcccccgggctctcccttcatgcagcgggcagcagggatgtgggcccggattggacggcaagggcctggggtccctctgtgagggctccgtcgcacttcggggatcccgggtgatggggggcccagaagtgggaggcttcagccgctcgcatggtggaggggtgaggggggcggcgaggttggtaaacggagctggggggagggggctgtcggggagaggagctgcgggtgacgggaggaattgccgaatgcgaaaccccctcagtgcgttgagtcccggggagacggttctgtgggctgaagggagcgcttccgtcatcggagcccttaccagcctgggagaaagcgctcggggcgaggggcgcagcagccaaagatggagggtgggggtggggtggggcgggagtccggggagaaagaagatggggaatctggcccgatcagaaggaagtgggtggctttttgctttggcaaacgggttttctcgacccatgggactcctggatcttggttctggggccaaggaatacgaggttcacacggcgacgtgcactcagaagcacctcccgagcagatcggagccccagcctcgtcagtggcggaattgccttcctacgaacccgtgggctgcctcggagcagtttgttttttcgagcctttggttccaagaatgcgccaggcgcctccgccctgggtcccggccccggcccgggcccggggggtgtgggcttgaacctgacagcgcgcggccctaactgggctcagccccgctgggagcgtagggttggggttgagcagcttttgcccaaagcgctaaagctgaaacccggcgcccggttcctgctccccccgcccccgtcccccctccatttccagaaaaataaaaattgcggaatcctggatccgatcgcacgcacaatgcgcggctgcaaaccgcattcttttcatgaatattcatcgaacgcttggccaggctccttatctgcgtccccagagacatttccgccttgaaacgaactcggtggacagcccttgcccatgtaattctcactctgctgaaattagcacagcacacatgggtgcattttttaggaccttgtacaggttgacaacacctctgtctcccccgcggccactcgcactgtggggcgggctaaaacccaacactagcgtcgaaatggccttctgctatagggttgtggaccgaccccccacccccacctcccgggattacagcccgggccaacagacccccagtatactgcaggcaaaatgaatgaatcgggactgtaattacctattcctttgtcttggcctcgcgcatgcgccggagagcagggtgtggagggcccggcagccccatcccatctccatcatccagccacccactcccctcccgagccgaggaacaaaggcgtttgccctaaatgttgcgggaaacccttggacaccaatgacagtttttcccgcctgtgtgtgtgtttcttcttttccaggctgtctctcatcttttgctttcttgctgatttcaattgaccaaaattaagatttgccttgatgaggatgaaggcaaattgtggattaaatattttgtacccgcacactgttattttcctttctagtactgttcattgttctttttcctgtaacatatcttactgcagtgagggtggctttcttaatttgcatcaattaaccgtttgacagatcttttttggtcatttgattaagggaaggtggcctcaaaactgtcagtgggcgttgcctccagtgacaccacctggggaaaattaagacatcttggcgtttattcgaacaagatggcctacaaaaaccggagagaagaaaacgattaccttgtcgggatcctaaccctttctgttttctctggactccgaactcattgtttcattaaggattccaaaaggcgaatggcgtccagatggaaatctgatgggggcagaaagaaggcagtcgttggggtgaagctggttgattaggttcgcaatgattcctttttccccattacctaacagaagccatgatatacctttgatggctgattttctttcagatgatctattttccaggttatggccatttagatttattttcaggtagtttgtttttaccacacaaaaagggacttattgtgtatattaaataggagccaaatatgatattttttgcagtggtaggttatgtttaatagggttttatgcttaagtactgtcagtacatcctggtggaataacgcgtggcagcaatggaaatcatctcttttgtcctttttgatgagaacttgatattgaccacgtttaatatatcagggcccatctactggctacggtggctgtctagctgtcttctaggtggcatggattgtaccctctgtactccttagtattgagtaggttattatgatttagagagctacgttctttcttttttcttttcctttaggttgtgtttttggcgaggggtatgttggaatatagaacaaattcaatagcaaatatataccagcatgtccccaattcagtaaatcttaggtttttgagtgagataatatttaaatcgatttttaaaaattattacagaattaaaatatttctatcagttcgcttattcttttagccagctagctttttgccaagctttagcagatagagaaattgagccttttgataaaaagcaccaacaatgacagtggcatttctgttctttaactggtactcagtctgccattgtgacgtgtagaagagaaccttatctccccacatcaccctagtttaaaggtccaatgggagagccctaaacatttaggctgtctatccttggaggcctcttcataatcctggatttactaggatccataatcctcctggatcttatggtgcaagaggtgatgcgttctgtctctttattttttaatttcacaattaagaacagtttgaagccattttcatttcatggtcaagcggagtatgacatcagagagaaactttcatttggaggagatttgatcactctaaaatggatgtctgtgttatttacggtagttttgtaaaaattgcaaaatccacacccatttttaaaaacaaggtgtcatgtatatacacatgtacacatacacatacttgtttatacacactctaaaatcttatgaataacttgaaatcaaactggttggtatagaatattattttatagtgctttgatactggtctttggaacataatcaatttctgtctttctaaatatgtgtttgaaagggaaagcctagcaagtctattaataagctcacttcccatttatcccagtgtacctggagcattaagctaagacgttcatccacaggcttaaaaacttacatcaagcactactgaactttacaagctggaataaacaatgcctactaaataaaagatttataaaattgttctgtcttatttttgtgatctcttgtaaatgttttttttttttttttttttaaatatccaaagaagacctgtgaactattatttgtcagaagcaattgcccttggtatctgattctgttgaaagaaatgcgttgatttgtccagacctttatcttaatgtagtttgtacctacttatttcacatcttctttctcttcttaatgatggtcttagctggtaccctggaaaccaggtaactgctggggaaaaacactgagtagatatatagacaacttgaatattctctgaaaatgggcatggaatctttattgttatatcaaattttctctaattatatctaatcctttaaatttattgtatctcttaagacattagataagccagtgaacatgagcttttttctttggcttaattatagtggacaactattaggaaggaactgtgaggttgcaaatagaatattttgaaagataaagtactctttatgtgttttggggtaatgtttttggagtggtttctcaggaaatcgtataaagactggatttgaagaatctaataattcctattttggggttgtaataagaactacatatgatatctaaaaatgacttaagtgtggaatggctggctctgaagggctcctcaacctctcagtctcaattagtgatatcaattacactgaaagtgttttcaacaactgaaagtgtcaatcaacactttcagtgtaattgacatcactaactaatttcagtatattcagctgtatttttgttccagcttgattaatgaagcccgggggaccccaggagtattaagttagaagtaggatgtctcaatcctaaaggggaccaaatatcatggttcctattttctgtccttgtgtttgtagatatttgaaattctgtattatatgggaattagcctgaaagtggatttcatgatatcaagatggccaccttccttgaattccccaggtctgcttatacttatacttatgtaacttaagaagtggcatttaagttctcttgagaaaatacaagggtagtcttgaaaccacagcagtgcccagcctgccaaaggaaggcttttgtgaaacgcctttccagccacagcctttgaggcagacgtctaggtaagtgtcacatgagaacagatgtctggtaaatgctctcaagagtaaaaatcaaccatacagagtccatttctcaccagaatatagttcaattgtaattttgaggagccacaagcctaagaaggaagcagtgtgattaaaacagcattctccttttccctctagaagtcagaaatgggatgaaatgggaggcagagagaatgaaatagtgtgtttttcttctgcaccctaaggatttaaataacataaaaaattatttcttaatgttttacagactttccatactcagtacatttttgaacaaaaatagggaaagctgagattttttgactcaacaaggccgtcagtgctgagaggtggtgattgagcaggcttggaagtctaggaatacattcctgaaagctttagagatttagaaatagaaaccatctgcaagcaccacaggggcccagggcataaggaaacacttcaggataatataggaaaatggtgtgcagtggagaaagcaaagcatttacacaatattttccccttatcacttaagcattttataaactctttttttttttggttcgttttgaaggaggcaggactggacttcattggtttgtgttttgaacttctgtttgaagtagatatttcagcaatttgtacattgtgtagggagtataatatttagaaagcatgatttggagggttaggcatggtggggatggactttggagccacagcaccaggttcgaatcttctctcagccatttactggttgtatgatctttggtaaattgcttaccttctctgtgcctcaattttatttagaaaatgggaattgtgatacatggttactgtaagtattaaacaaaattgtcatgcacttaggacaatgcctccttatattcagtgcatctttttaaaggagtgtgaatatcagttttctttactgcatccttcatctatagctactcatatgtttgtttcaaagagaccattattgcaagacaggccaatggtggtgccttgctcagtaggtagaatatagaagtctgcggtttagaaaactcctccaggtgtggttaagaatacgctaaatacgtcagtagttcatgtggactaagtttcaaaagtgggtaggttaatacaacccagcctccatactgaatttgtactttttttaattgaaggaaaaaaatgaaagtccttggaacttcagagacaggattactgctttgatatgttgagacctggactcaccctgattagctgtatgaccttgggtgtagatgtaactcattcagattcctgaaaatccttagctttgttgtgtcttgaatcagatgtatcattagcatttgaaagaagtctaagtcattagtactaaggttgataagccaacttaggtattttacattccatattattcccactacctccaccctgcccctgccaaacaggcatcagatgcccaccgcctccatacctggagtaaaactgaaagttgtattatgtaggaatgtaaagatgttgtgatctattgacaaatctttggaaggaattatccccgtggggaccttagaaatcaggaactctgaacaacaaaacaaaaaagtctttggaatttcagctatttggggtggatcaattctggaactggtcttaacatttcagacatttacttcagggatatgcccaaggatttgcttttgtccttctggacactttgttgttgagcaaaggtgaattcactctcattttgggtaaattgttgtttaacagcttccttaaacaggtttttgtatcttacggattctggagggaagtcatatattttactctatctgtcaggtgttcactgtaaggaaggggattttagatatagtggttatgtcttttaaaaagtttcctaggatttcaatccgtgtagatggagctacactgtacttctcctaagccttggccattttatcttaggtaatatggactcttcagggggggaaaaaaggggaaggggggctcttgaataaaatgggtagccttttcacatattgacatcttcagtattaatttagagagagcgatactaacttcttcaatgaaaaattatttatggaagagatccagtgtatacttctaatatttcatcagtattacgttaaattgcttgtagattagaggtgcttcttgtcatagtgaatggagactaataccttaagtcattcctttctagtatgatgattgtggttgctcttactgaggactataaatgtggatttggttgatactttcacgtttctccttggacattgcagatggaagtcataaccccaaatgtcttacttatctgcaattttgctctgagatatggaccaggctctagcagaccgtctgccactgtgtagttagtgttgcctgattagaaataaaccaagtcctgcctaaattggtcaatctgttttgttccgcgtccacgcaggatccacttacagaaagccagtggctgcttttaaccactatcgaaacctcagtcctttgtaagtactttccttggagcagaaggtttaaaagctcatgactaaacactcattttggaactcaaaataatcttagagaagttagtgggttgctcagagtcccacgagctgcttatgggctgcgtggaaacccaggttatttccccataaagcctcactcctgctgcccctctcttctgagagagaagcagagaatgtctaattgagtcactcttactcactgtctgcaacgtaaaacttttttttctaccatttttttttgtaccctagaaccattaaaaaaaaaaccagtctggatcctatatccgtctcatgatctcccccagaggagacctcttcggccttttaatacaccacacatgccccagggggacggtgcagtcactacctgctgccacttcatccacaggccatcttcccctcaacttactcctttctgcttcttacattatgtgcacgtggtttcctttatatgccaaaaagtatatggcatgcactgaaataactttcttcataaaactaaactttagtttttctaaaatttaagttaaatccttgtttttctcccaaaatgcacagtggttgggggctgggaagctggcgatggtgggtatgtgtttgaaaagaacgccagctggctttagacttatttgggggctatagagcaagcggagcagtcaggcatcccacccacccctgaaatgctcggggccctcttggcagatccagtgtgcacgttgtcgtggcaggtggtggatggagagcttttatgccgatttctagcgttatctggcttattggtttgagagctgcatacaacgggatgtttaatcttttttatttaaaattaccatttatagtatttggggtgatttagaggatttaaaaaatctgttagattctgaagtagtccttaaaatatggaacataagtgcttaagatgctaaaacactttctaaagcatactattgggatgtttgaaactgatgttggtaaatgtaaactgtgtgcatttgtgtttggataatctgtgataagtttagtttttgatgagacacaattttaatttcacatttaaaatgacatattgaggtcaataagagcaggtttgaatttacattttccagagaaacccacaattaataggtaacttttattttcataatgcttttaattattacacttacgtagattcagaaatggatatattcctaaaataaaccaaaattaaaacttctttgtctttttaaagaaaagctgtggttctattagagaaaaatacttcttttttaagtttatgaagaaatagctgttagataaaaacttttcctggttgagctttctaaattacaaatttcctgaattgcatggcaaagtgactttgaaaagacccacttcatttttcctttgacagtctattcaaaacaccattaccacgtagactaatacaataaccaaggtaaatagtaacaagatctaatgggggaaatagtgtatcaaaagggggaaaacagtatccactgacagagagaactagctgtttcttaaagagacagagaagaaaggtttcctcggagttattgggtcttcataggatggtctcctgtccttctctgagcttgccttggaaactgtagaggctgtaggaatatttgctgtctttaacccttactaatgttctcagctatttcattagaggatcacagtgtctgtatttgccttaggtgtaagatgattggagaaaaggaaaatgtagagaaatcagattgactttagatggacaggcagagagttctggagtgacttcccactccttaattctgcaatgcacccagaaggcagggtgaggttagatgatcacctgggtatttactatagccattattactgtgattagataaagtgtcctggaatcttctctgaataactcatttttcttttttttttttttctttttttcgttttgctctgtcgcccaggctggagtgcagtggcgcaatctcagctcactgcaacctcagcctcctgggttcaagcgattctcctgcctcagcctcccgagtagctgggattactggcacccgccaccatgcccagctaattttttcgtatttttagtagagacaaggtttcaccatgttggtcaggctggtcttgaactcctgacttcgtgatccacctgtcttggcctcccaaagtgctaggattacaggtgtgagccaccgcacccgtccttttttctttttagacagtctcactctgtcgcccaggctggagtgcagtggcgcaatttctgctcaccacaacctctgcctcctgggttcaagcaattctcgtgcctcagcctccctagtagctgggactacaggtgtacgccaccaagcttgactaatttttatatatatatatatatacaatattttttttgacagagtctcgccgtgtcgcccaggctggagtgcagtggcacgatctcggctcactgcaacctccgcctcccaggttcaagcaattcttttgccccagcctccaaagtagctgggcctacaggtgccgccactatgcccggctaatttttttgtattttttagtagagatggggtttcactatattggccaggctggtcacaaactcctaacctcaagtgatctgctctccttggcctcccaaagtgctgggattacaggcgtgagccaccatgcccggccaataactcatttttctaaggaagaaagaatctcgattaacaattggtgatgacagtgcctccaaaggcttctcataatcacccaacctcaggtaaacagggtgaatgccaccctattgacaggaggtttattgtggcactgggttttgctcagtagccctggtgatctgttgttgagaacccaagctcatatttcccaaggctcacccaactggactttagaaacctggttccagaattttcaaatgtttggttttcaggcagatagtaacaagtgtttgtaccaatgttctccagaaggttgttaagacaagattgataatgcttcccaggtatgtgtttctttttctgactcacagctgcatgttactagttttcctcatctgacccgatgattgactgatggaaagtctcattgaaggtttggttatcctttcattggtagaagagacaaaaacaggacataagtggcttattccattcacctctacttttctattgtaactggatttatttctgccatccagacagagagcctgggctttgatttcatctgtaccaaagttttctgggtggacaggtacagggggtcatcgtaagagaactcaactgaccacaatgcagagagaggtaactatccaatggaatggtaacatggttccactacggtaagaactgtagtgttaaatgaaatgaattcttaatgggaaaggagtcctatgaatatttctaagttgttatgtttaacttaactaactcttaaatgcagggaatttaacttaaaagtgaatttaacaaagaagtgaatttaaaagccaacaagcttcatatctctccacctcaccccaaacccgtgttaattagcccttcttgcttaaatcaaatcaccttgttgaaatttaatgctgtgactagcaatgtattctttgaccatatccttgtttccagcaacaaatactgccacgttaaggggagaaggaagtgatggatagagggaccagttggcagattgggacacacgtacaaacactattcctttaacttgctttctttagatccgtagttcctacctgtgagtctcatttctgtattgatccgcattggctaagcatacgactaaagcgggattctttgtcttttcttcagtgagctagccaggaatctcagaaaaagggcaaagaagtccagaatgatgtaatgctgcaaaaaatgttactgctgtttctaaggggagtggaagaaacccaagccagttttgagttgagtgtgcttcctgttggaagggggtgagaattcagagatgtatacctattaaaaaaaaaacaaaactcttaggatatcacacagcgtggcaaaccagcaagccctgacctgttagcagttgtttgcatagtttaccaggtagagaaagcatccacagatagaatcaaatgtttgaggacactaacgtgcactgtggttagtgtgaggtttaactggctggggacacatttccacttaaatctgattctgataacacatttaactacttccctagctaattacacagttccaggcttgagagaggaaaaggcacctgtgagcagtggcatgtcttgttaaataaggtttgaaaacccttccgaaaaggaagaaatggctgtacttggggacgtttgagcatagtaattgtgcagccttgctcctggctgaggggaggggaaagaaagcccttggtttctgctccaagaagtgttcaaattccccttatgtgggcagaatttttggtaagtacaatacaggaagcaaaactgcattgttctaggtcagcagtcaatgacctattgtttctgagcccctctgttccctcctcactttgtgttcaggggacgaaagcccagagcaggagaagaggaagggaagtctgggagaggctgggaagatgcacccgcattaacacaagttggagaggcaccaatgctctcctcctgtgtctactcaggctgccataacaaaataacagatgggataacttaacaaaaatatattttttcatagttctgcaggctggaagtccaagatcaacatgttggtaggattgggtttttttttttttttttttgatggagtcttgctctgtcacccaggctggagtgcagtggcactatcttggctcactgcaacctctgcctcccgggttcaagcagttctctgcctcagcctcccaagtagctgggattataggcgtgtgccaccacgcccagctaatttttttgtatttttagtagagatagggtttcaccatcttggccaggctggtcttgaactccaaagtgctgggattacaggcatgagctaccgcacctggcccagacttgagttttctaaatcagaatggaatgatcctggggaagctcattttaaaatctcctacccagagtggcaatggtagagagcccttcttgttctagacagaagagttgaccagatgcctaggccactttaaaatcctattctagcatctgtgctttcttttagggattcattagacagactcttaacggtggaacagattttactggatagagacttggggttgaagacagaaggcaggggcttgaatagaagcaagtgcaggaagagtggtgggggttttgacagaaagtttatgaatcagtgggacaccgaggaaagggaagtgtgtgtcgggaatcatggaaatcctccatgactatagaaaccaaggctaatgaattttggggcctttccaagcaataaaacaagtagtgatacttacagggattcttttgaggggcattctcaacaagtgtgggtgtgggtaagtctacacttaacccttttgagagggtcctttttattattatttcattttattttattttttgagacaggggctcgctctgtgatgcaggctgcagtggtgtgatctcatctcactgcaacctccgcctcccgagttcaagcgattctcctgcctcagcctcccaagtaggtgggattacaggtgtgtgccaccatgcctggctaatttttgtcttttgttttaggggagatggggtttcgtcatgttggccaggctggtctccaactcctgacctcaaatgatccacccgccttggcctcccaaagtgctgggattacaggccactgcgcccagcccgagagggtctttttaaaacagtttacgacttttaggcgtaccctgtcaagtaattcagttattgctttcctggctcaaatttaaggtcacattacgttaactctggcaatacaaatattgcctcatgaatccccagtgaaattgagaattaacaatttaattttactacactagataaacaaaccactggtcttaaaaataattgaaacgatgctgtttaatgtcttttataacaactttcactgaattgttttctacataaaacataggatgtaagttattgtagaatttaaaaattttaactcttaaaatttatgcagtgtctattttcaatgcaatgtcagtgaggacctttagcactcactttcctggtagatagcaccttcttccaatcatgccagaaaactaggcttgtctcctggaattccccatatggcctccttcctgggtagtgaaactcacatatgtccatttccctctacttgaagccattaatctagtggaagcttctattctctcctggatcactgccctaatactctcctaaatccaagcatgacattcctctgcttaagattttaggcctaactctccattaaacctggaataaaacaccggctctcagcatgttcaccttctattttcgtctcttacctccgttcctacacactctctcacccaccctcttcagccataccaacaggctcccaggtctcccagggctccaagctcatgcctactgctttttctgcctggaatattcttttccccactgttgaagttactcctagttcagatctcagcttaaatgtcactcttgcaaagatgttcccacacctcctggcactgggcatttttgtccctgcatttgccagaattgatgagattatgtaacgtctctgctcctctagattaaaacatccaggaggcagggatgtgtctgttttgtggacccataacagtgccctataacaatttctgatccataccaagcaccccatgtctgttcagtgttgaaggttgtctacagtacattatgttcatggagagcacgtgtcttctgaacatcgcctttggcagctgagatgacttccataaatgggccaaaatttcacctcatcagttgtgcttactatgaagatgaatagctgatgaactccaattacagtaattattctgtttccaggcaataaccttagcagtagttgttctaaagccctgagacattacaagtaatgagtactgatggctcatgcatctgagtgtcctcccaaggtagagtccgaaatgaaaaatgaagctgtgtattatttgagggagacaaatagtattacattgtgggaacctttcagaaacacaacacgatgagtgaaagctgaggatgattgtggttacactcctcgtctgtccttccaggttattgaatgcttttggtctgctttggatcaatcattattgaacaacttaactgaatgcattttagtaacagcctttgtgaagctgcgaacatcaggtccctagaaaagcaggtggcttgaaatctttacacattagtcaattataaacctaatgaagtaaaaacgaatttagctctaattctgcgctgtgactacttactctgcatcgtgagttctacgaggttaggaccttgctttgttcatctttggatgtcagtttctagcacctaaccgaacctgtggtccagtaaagggctggatacgtggaagggcatgtttcatggtcatgtgacatcatgctgtgcaaataaggcacatttcagaacgctgtcctttactttcacatccaggaaccagcttgtaacgaacctttggcaaacatcaccgtggatttttctttttcttttttttttggaggaggaggtggggcacaactaagacatcatttgtgatttactccccctcctcttccatgtcacatacctcttagatggtcaacattcaccttctgctgtctcatttctgagacatgagacagccatcggacttagttttaactaggattattagaaatatttttgtgtccatcttgagatctctcatcagatcaatgtctagttttcttttgctgattcttattgatggaattcttattaaataaaaaagtttgtagcttttcacaatggtatgaaattgatttctaaagtaatctgtagttaaataataatgcaagattccagtaagcatgtctagttctagcaatacaatttactcattccaaaacatttctggatgttgatcctttatttgcaaaatttttattttcataggcttctggtttgcatattaaaactatagttgtagttgaggtgatggtaggcgtctttattgtctatacctgtgagctgggttgaaatataagatattctcataccatctctataatctatgccataaacacttggcactttattaaatcaatacttggtctgaagattgtctcattttaaatgaattcagctcatgaattggccagtggcatcctcttcagctgaaactcctatatctccttccccttctttgtgttcctcccctaacatacagctgatactccctgggtaagatctgaaggagctggctgtgggtcatctagtccagaaggccatgtcagtgttatttaaatcaggtagtagaatgggaagggcagtttgcaattaattttttatcttaatgtttcgagtctctagtgcaccatactgcctcctggtttctcacagacctactgtatgttttgtttggaagccttggctcatcttgtgggttgtgtacttagctggaatgacatggtatcgatggttgaggagcaatttatgacacagatgagtaattaggtgactgatacgccttttgatgtgtgtttccaagtagatgatattttcttttctttgttttttttttttcaacattctgatatgatttgctagtgatggtgatggtgtcattttggtttcaaaactggaaaatacaggccgggcgcagtggctcacgcctgtaatgccagcactttgggaggccgaggcggacagattgcctgagttcaggagttctataccagcctggctaacatagtgaaaccccatctctactaaaaatacaaaaaaattagctgggcgtggtggcacacgcctgttgtctcagctactggggaggctgaggcagtagaattgcttcaacccaggaggtggaggttgtagtgagttgagatcgcgccactgcactccagcctgcacgacagagtgagactcagtctcaaaaacaaacaaacaaacaaaacaaacaaaaaacaatactggaaaatacaaacaagcccaaggagtgattggtagatgtttagagtctaccatttctcgacttcaggaaatgtctgtgtgtatctcagtcttctgctattgtaggcacacatcatacttcaactgtatgaagattattctattcctgctctataaatatgttattggattggatgagcaagatatacacttacacatttgaagccgtaagtaaaaaaaaaaattatatagttaaaaggtacaaataattgcttttgtgatgaatttgttctttaaatgaaacatcagtggcatttgttcatgtagttctcagataaaacttcctaaaagtggttcaagtgatttcaaaatgccaagaatatgttgcaaacttgtttttttgtatacaaatgaacagctgacatgaggtttttctaccatgtgaatgtgacgtgggaacatacaaaccacactaaaatattgcagaattgatgtttgtttattgatggagttgggagacgcatctcctttaagtagcttacagtgggtgaaggcaaacaaggaaatcagtaattacagcgaagcaaatgttggtccgtagatacatggggtagagatataaacagcagatagtcattaagtttactactccgatcaattatattctttttcttcaatctgtccctagatccacctcttttgcctgactaagccatgtgagatattccagtgagatattcaattggctattatttaaagagttttatgtgaatatcactgtagagaatatggaagaattcagcatcgattacatttagtttaaaggtcttaatcactagatagaaaagagatttacacatcttaaatttccattaaaaagcaggcatttggggattagaaactgaaggaaaagatttccaagagtagctagttcttaaaacacagtaagaatatgctttagctcttgaacacagtgtttgtagttactgtcagactccttggggaagtgtcttctgctggggctctcacctgacaggctaactgcattaagatgggccactcagcttgagaaacaggaagaattgctgttgctcccgtctgctatttatgtttttcttttttttttttttgagactgagtctcactctgtcacccaggctggagtgcagtggtgcgatcttggctcactgaaagcttcatctcccaggttcacgccattctcctgcctcagcctcccgagtagctgggattacaggtgcccgccaccaggccgggctaattttttttgtatttttagtagagacggggtttcaccgtgttagccaggatggtctcgatctcatgacctcatgatcagcccacctcggcctcccaaagtgctaggattacaggcgtgagccaccacgcctggcctgctctttatgttttttcatatgaattacatttccttacatccctattcagatgtcgacgttacttttgctcaaaagtgctcctttctcttgactcctgagttgcaaatagcttaccaaggtagctgggactcttgaccatgatgttttttaaaatgaaatctgtcaacaaatgaaagacaggattggtaactagtattttatcattcctccagccttcatcctccagcttgatttatactaaccaccatctgaaattgatgattgtttcccatgaccccacagggagattccttggatgtaggaactctgcctgatttttgttttcctagaccttcagacactacttggcccagagtaattgctctttatgaatcactgctttagtccaagcttgtccagttctctgcccaggactgctttgaatgtggcccaacaccagtttgcaaactttcttaaaacatgagacttttttttttcttttttagctcattagctattgttagcgtattttatgtgtggcccaagacaattcttccagtgtggcccagggaagccaaaagattggacacccccacccttgcgtttagtctttaagactattctaccttgggggagaaaagcaaaaatgatttcttattacacgtatttaaaccagtatttacatttgtagggatcttgggcaggttatccagagagcgagtttcttttcttttttttgggatggagtttcactcttgttgcccaggctggagtgtaatggcgtgatctcggctcactgtggcctctgcctcccgggttcaagcaattctcctgcctcagcctctggagtagctgggattacaggcacctgccaccatgcctggctaattttttgtatttttagtagagacggggtttcgccttgttggtcaggctggtctcaaactcctgaccttaggtgatccgcctgcctctgcctcccaaagtgctgggattacaggcgtgagccaccgcgctcggccgagagctagtttcctgattgataaaatgttcactgtttgttcatatcctctcctgtgtagttagatggctactgaatatatcctgagagcaggtcctaagagtttatttttcatgttattgttggaaataaaaacccagaaatattttaataataggaattaaagatatgaagaccctgatatctgaaagctgagtggttcaaatatggtgagcaatgcactcttaactagacccatatcttaagggaaaaaaatgtttttagtacttttcttggtatcttctaatcttcagaaaaaaattcttaataggcagtcgatgtaaataaaacaaaattctgtatatttaaaaagctgcttgcgctactgtgtcattcctctggaattttcaaaagctgagatttttctgtatactttgaaagctaatgaaatctaccagcccaccgggtagtaaacaaccaagaattcttgatggaggtattgtaaagaaaggtgataaggtggctgggcatggtggctcacgcctgtaattgcagcactttgggaggtcgaggtgggcggatcacgaggttaggagatccagaccatcctggctaacacgtgaaaacctgtctctactaaaaatacaaaaaattagctgggcgtggtggtgggcgcctgtagtcccagctacttgggaggctgaggcaggagaatggcgtgaacccgggaggcggagcttgcagtgagccgagatcgcaccactgcactccagcctgggcgacacagcgagactccgtctcaaaacaaaacaaaacaaaaaaaagaaagaaaggtgataaggctatctaatgaaggacccaaagaactctcactgagctcacccagaatgcttttctggattaaagcacagactgagaacctgttcttaacattagtgacttcggtatggctcattttgaagaaagaaaattgcctaaatctatagttgagcttgcacacattacaggtctctggaaataagatgcttaaacaggttctataatctggttcttgctaggtgagtgtcccctgacattgcttttttttttttttttccgtgatggagtctcgctctgccactcaggctgtagtgcagtggctcactgtaacctccacctcttgggttcaagtgaatctcctgcctccacctcccgagtagctgggactacaggcatgcaccaccatgcctggctaatttttgaatttttagtagagacagtgtttcaccatgttggccaggcttgtcttgaattcctcacctcaagtgatccacccacgtcggcctcccaaagtgctgggattacaggcatgagccactgtgtccggccccctgacattgctttctaaagtaagattacctgtagttctcagtgttcgccaagcatgtatgaaacacaacgtatgttaagaggctgtttaattacataaatgcttatttgggggattcttgttctcagtggccttctctcttttggatggatatacacagtttgattggcactcctattgacactttatttggtttgtgacttgtttgtctcggtgcctttctattaatatattttttctatttaaactaccctctttttttcagtacagcactggacattttctttgaaatcttccagattcatttggttgtcttcctcatctcagctgtcttatgcaggaaaagtggtcactgttcagtcaattcattctacgtagctggctatctgagttttttttttatcgaaaccaaaataaaacttaaaagaaacagaagaagctgagtctccctcccctccctttcaactaacacctactgatgtttatcttggaaataatttgacgtctatttgatgaaacctgtagttaagtgttaatggagactaacgtcaagcttcagaacagtgaggagaaacctttagtattgtgtgtctagcatttgatgcaagtgctctgtggaaaccatggaagctgggataaaaatgctcgcctgatccccagatagtctcatctgaatgtactgaggcatcaccgcattcattccaaatctgaacctgacctacttttccccttcatctcctgccatctcttccctagtttgtcatcttcttttccagcaccacgaatggttcatgctttctgataaatatctgatgtatggcatcaattatatgactatttaaacatgactcatagtgaactgtatttaaatattgatatgactccatgcgttttcaaagaatgtgtggaatgttctttttgagtgtcagttattaatcatctgttttccgttcaactttggttatcttcagtaaattttaatatctttcccttcaagaggacttcccagctgtctgatcaacgggtaggaaaggacagttggttcagtgcttgctcatgttagccctgtaagaagtcagctcagtttagttcatccaatagctggggaataaaaccacacaattcccttccttacttgatatcaatcaacacttgactaaatcttacatcctgtcttcagctgttttttttttcttttttgagatggagtctcgctctgtcacccaggctggagtgcagtggcatgatctcagctcactgcaacctctgcctcccgggttcaagtgattctcctgcctcagcctcctgagtagctgggactacaggtgcccgccaccatatctggctaattttttgtattttcagtagagacggggtttcaccatgttagccaggatggtctcgatctcctgaccttgtgatccgcccgcctcagcctcccaaagtgcggggattacaggtgtgagccactgcacccggcccagctctttcgtaaagaaagccttggcaaatgtggaaattctccctattcactacggtaaataattcttgaggttactaaactcagtatctaaataaaacagatcagtgtgttagtacatggaacagaaggccatatttttggttataaccgttgaccccacactcaccctggggtcttggtgtcaatgtcaggatgtgtttaaataccctaggctgtcaatcttgtaatacaactttggttatctttgttctagagccttgcaaagtaaagctgatcctagttaggacccagttaggaatttggatatatgtgttgtatttgcatgtgatactgcccttcatttgaaaacacaggcttaaattagtgaagtagaactcttttagtccagattacccccaaaatctgtgaatttgtactaattcaaaatcctctttgctggtaaccgttgtaatgaggtctttggaaattttatcctggtagcatggaggtgttgcttcttttcatttgtctagttttgtagtcagaactgtttatagagatgtaattttaaagtgaccctatgctatggtttgaatatatgtgttccaccaaaattcctacgttggaacctaatgcccagtgtgatggtattaagtaagagctggggccttttgggaaatgtcgaggtcatgattattccaccttcactaatgggattagtgctcttatagaacatgttgggaaaaaaataaaaataaagataaataaataaataaataaataaataaataaataacaggttggagcccaggagtttgaagctggggtgagccgtgatcatgccactgcactccatcctgggcaacagagtgaggctctggccgggcatggtggctcacgcctgtaatcccagcactttggtaggccgatgtaggcagatcacctgaggtcagaagtttgaaaccagcctggccaacatggtgaaatcctgtctctactgaaaaatacaaaaattagctgggcgtggtggcgggcacctgtgattgcagctcctcaggaggctgaggcaggagaatcacttgaatctgggaggcagaggttgaagtgagctgagattgcgccattgcacttcagcctgggtgacagagtgagactcctctcaataaaataaagtaaaataaaataataaaataaaaaacagagtgaggttctgactctttaaaaaaagagaaataatgtgcccacttcatcagcacatatactaaaattggaaccatatagagatgaacatggctgctatgcaagaatgacagtgcaaatttgtgaagcattccatatattttggaaaataaaaaagttgaagggagtgccctagtccccttttgtctttgatcttggacttcccaacctacagaactgtgagaagtaagtattgattctttataaattacccaatctaaggtattttgttagggtacctactaaacaaaaagataaaactcttgtgattttgatcttaagttggaaacataaaaccatcgttacagagaaggcaaaactaggcaaagatttatttttgcaatggcatttaaaaatatttaatagtttcttttaattataacattacaaaataacaccttgtagtctggtgagcacaggcttgcctagtgcttagcatgtgccaggcaggccacattctaagctttacacagattgacattttccttttttttttttttttcttgaggccgagttttgctctgttgcccaggctggagtgcagtgatgtgatctcgcctcactgcaactcccacctctcagattcaagcgattcttgtgcctcagcctcctgagtagctgggattatagacatgtaccaccagtcccggctaatttttgtgtttttagtagagatggggtttcgccatgttggccaggatggtcttgaactccaggcctcaagtgatctgcctgcctcagcttcccaaagtgttgtgattacaggagtgaaccactgcgcctggcccaaagattgacattttaaaatacagcagctaggtggggtggattctattattatcccattgtacaaatgaagaaactgtggcacagagaaattaataataggttacgtagccagtgtaacccagaacatcttagttcatgttgcatgtctctcatttgctggaagcatatcagtcaacaagaaaattattgttttgcttccagtttgagtaatgttcctgtgacttctttaagtgctgtatatttgctgcatttgctgggagtgaacgaggccctgaagctttcgtagatctgtgcaggaactggagtgagccagtgtgtgccaccatgggtgtccctcgatgttctctcctgtcccttcaactgtgctctgcaggagtcagacctgtggggacagttcacaggaacaaaacagaagctccaaccaagagctgcaggatttaaggacaaggctgcaaaacaagtacgtggagctggcacgcaaactcagggctttctgaaacacaaacttcagcatttaattaaatgggggaaggaaggaactctcaaccgagatactgaactgcactgtgtctctacaaaatgcgagtgtcccacattttaagggtgaatggatatctctaaaagaaaacacctctctcattcacgtctattctgtatgctttcttgattggctgaggaaggggtgaaatcatgctctcctttcattcccctccttcgactaggaaggcgttctttgatgcaccattaatagttttagcaattactctcaaccatagtgaatgtgtaactcctgttttcatttaaatttgatcaatgactcagttgttattaaaatgcaagaaatagtagaagtttttgtatatatatacctgtgatgagtgcgggtgagaatggtcctgtggttttatttctcttttttgttcgactttatttgttagatcagtttcatgttcacacgaaattgagtggaaggtacggaggtttcccatataagtcttgcgccgccccccaccccatgcatagtttccctcattatcaacatcccccagcagaatacgtctgtcacaattgataaacttgcattgacacatcattgtcacccaaagtcctttgtttacattagggttccctcttggtgttgtaccttctgtgagtttggacaaatttataatgacacgtatctccaattagagtagcataattttatctctttataaatgctcaagtcagggcttcttattttaatttaagggggattaaatgatcgtaagtgcatttgttacatcattgtgcttattaataatctgtttaaagtatctgtaatgctttgtgttataactttaaacaacagaaaatgatgttatttgtaacggtacatccttgctctgcgtgctaatgtccttgaggagtcgaagtaagtctaggaaaaaaaaagtcaactcagtttaaaaaaaaaaaaaggtaatcgtaagcatttttttttccttctaaaaggatttctttgtgtttgggggcagggaggacatgtcttgaaataagtgaacatgtgcttgtaaaatgttttttaagtgttgtcgtccctgtaagtctgtgcctttctgaaaatctgggtatgaaaccagatcatgtgtgtaaagggaaatggaacattctggaacacctgtagtctgttgcccccagcaagttgacatgctaacatgctaggtgacaaaagggcatcatgataaagccactgagacttaaattcttgcttttttttttttttaagagggagtctcattctgtcgcccaggctggagtgcagggccgcgatcttgactcactgcaacctccactttctgggttcaagcaattcttgtgccttaaccccccgagtagctgggactatgggcgtggtgcgtaccaccacgtccagctgatttttgtatttttagtagagacagggtttcgccacgttggccaggctagtctcgaagtcctgacctcagatgatttgcctgccttggcctcccaaagtgctggggttacaggcatgagccaccacgcccggctccctccaaccttcttaacttcagtttccggtgtctaacttgacactattgatgtctgcctcttgctatgaattagcaaaaggtgaggattggccaggcacagagtaatgtctccataagggttggctgttcctgccattctcctgcctgttctaatctagatctctgtttcctttatagcattcacaacacatggtcattattttatttgtgaatttgtcttacttgtttttggattgtgtctcaccatatgatcttgttgttggtgaacttgtcttatttatgtgtgactctcaccatttcccttcctagtctgtcatggaagctctatgcctccagaccctgggtctgtctccctaacaccctgcaccttggttctcaccgaatgcttgtccactgccagcagaaaacaaaacactgctgtgtccctgcaaagcactgcagctcttgccagaatgttcatcgtgtgtgacttttcaagtgcaataagtcacgggtactatgtgaaattggataaagataatttttttgcaaatatatccttacattgtaaactatcagctagggttgaagttttctaattgatattttaggttcttaaatattggaagataacttttaatttggtgagattctatacatgaacatatttccaatgtataccttcttgataagattatacgtattgtacattctcggaatctggaggacacaataattaagtgaaaggtggctttaacaccatgtgtgctttttcagacaagtaaattagtccatgccagtgtaatgggatctgcttataagtgatctactttaacatcagacttgattctctatggtgtcactttttgtttgatttgttttgtgtatttaggtaacaatgatatcaaacctcaaacataaaacataatagtgttttccagtatcaggaaaacagattccttaaggatccctaatgcatgagtgtctaggggcctaggttctagttagtaaactccactaagctgaggatctgagactgggcacggccagaggaatcctgggagtcatcgtttacaactctgcattttaggggtgagggagctcgtgacagagggtctgggtgtgacccacaagcacactgccattgtttacatcgtcagacttaagagttctgttttggccttcgtgggacttgataggagggaaggcattgaatatatatttgtgtttatgtatatcaaagatactataattgtagtgttttttttttttttttgagacggagtctcgctctgttgccaggctgtagtgcagtggtgcgatcttggctaactgcaacctccacctcctgggttcaagcggttctcctgcctcagcctcctgagtagctgggactacagatgtgcgccaccatgcccagctaattttgtagttttagtagagatggggtttcaccatgttggccaggctggtctcatgatccacccgcctcggcctcccaaagtgctgggattacaggcgtgagccaccatgcccagccgatactataattatagttttaaaaaatgtacttaaatatcagtgttgagccgggtgcagtggctcacgcctgtaatcccagcactttgggaggccgaggtgggtagatcttgaggtcaggagttcaagaccagcctgaccaatatgatgaaaccccgtctctactaaaaatacaaaaattagctgggtgtggtggtgtgcatctgtaatcccagctactcaggaggctgaggcaggagaattgcttgcagctgggagatggaggttgccgtgagctgagctcacaccattacactctagcctgggcaacagagtgaaagtgagactccgtctcaaaaaaaaaaaaaaaaaatcagtgttgaaatgtgacatttttttccctgtagtggctgtcaaataattcatatttaaaattatttttgctttctcccattttgaagatggagttttatgttataattgttgatattacaacagacatcttacaaatgctaaatctaaatgccaaatatatgatattattgtattcaggagaccaaatctatcaataccactttgtggttaatttatttttaaaaataaacactcttacctcagcaatttaatatgagattaatgataataaattttataggcctttaaaaattagtaggctctgtgtagaaagttacttttttttttttctctaggcagaaaatctcagtttttactgtttggcttacttttttaaaaaacaaaaacttacagagaaaaattgctggagtggctgaaggaaaaatggagaaagggataaaagaaagatggaagaagaaattgagatcacagtgaataaatacattgctcaggataactcagggggtagcagaagccctgggattcagaaccaagtctgtataatgcctgagcttgtgtgctggctttcatggagtcaaaatgtaaaactaggggacagtagggcattactactaataagtcgaataataccaacgcgtggatagagaaaaaaaaatcaagagaattccaggaaaaagaaaaaaacaccacggttagatgttttctagaattttctataattattggagaaggtttctctggggtatttggaggagttctttcagccagaaacaatagccacaacttgtcccagctgtgggatgggcttggcctctttaaaggacagttgagactctcttgacaagaacccaacactgtaaaataaaagacctggccgggcgcggtggctcacgcctgtaatcccagcactttgagaggccaaggcgggtggatcataaggtcaggagttcgaggcctgcctggccaacatggtgaaaccctgtctctactaaagataaaaaaaaaaaaaaattacctgggagtggtggtgcgcacctgtcatcccagctacttgggatgctgaggcaggagaatcgcttgaacctgggaggcagaggttgcggtgagccgagatggcaccattgcactccatcctgggtgacagggtgagactgtgtctcaataaataaataaaagacttttggaaaggtggagactctagactgcaggagatgtggccgccccgaaaactggctgaactttgttaaatacaactcatgattagaggaatgaatagtgctgtgttttaactttgtaaaatacaactcatgattagaggaatgaataatgtcaactcacatctctgtgactagatgcaggcttcttaggatatttgtaacttgaagaaatctggaaggacctcattctctaattgcccattttcccaatttgctttacaaatccttctgtggaaagtctttttgaaaatagcatttttgctccctcttagcctctggggttctgaacgctttggcagtgaccattgagaatcaccctgccttccatcagttaccttgtatcaccttataaaagtcatttggccgttgcagtggctcacacctgtaatcccagcactttgtgaggctgaggccagcggattgcctgagtacagaagttgaagaccagtctgggtaacatggcgaaaccctgtgtctacaaaaaatacaaaaaatagctgggcatggtggtgcacctgtagtcccagctactccggaagctgaggtgggaggattgcttgtgtccaggaggcagaggctgcagtgagccgagattgcgttgttgcactccagcctgggcgacagagccagaccctgtctctaaataaataaataaataagtagatagataaatgccatttggatttctgaaagcaaggtttgtcttttaaatgtcagttggatttccgaatgttggattcttaaagttctgaacatgtaaagtataggccactggatctcagagctttttatccaggtgcccagaggctgagaaaatgccccataggttctataaaggacatggatcctgacttcatacaggtcaacaactatgttactgtgtttttatctggtttagaggatgggaaacccatcagtgttattatgccttcattcaattatggaagaaatcggttgaataccagactgcccaatgaatgtagtttctgtatattttcaggcttttagactgatctgaagttgtgcctctatgcacgaatgctgcttcttcaaaaggtgccagtggctgggggtcaactttgatgtggcctataaagttcctggcccagcacttaggcctgcctttcaggacataacctctcagctggatttaagaaatagcatctctttcagtgtgaggcagcggcaggaatgaagtgtgttcgtgtccgtgtgctggcgcttcgtatgtacatttttgttgcgtctttaacttagaaaatcagtagactttatgagtatcagaggataaatgtactgcagaggatgagaaaatatttagcgtttttgtggtctctcttcagcagtgcctccctctttcctggaaaagggcattggtgaccagagaaattgtgattctgtttgcatcctaggccaccatggagttgacttatgtgtagactggccatagcccagggtcatccctcccctctctgtcttgttctcagttgcttgtcatcatttccattcaaacacttgaatattccagatctgggatagtgggcagatggtttcatgctattcaatattctagtaaatatcttttcaatggttacctgtagtcagtcacattgtcagaattctcacttcagctatacgtgtgacaatcccttggctcacacttaaatttaacccagtgcttgtaaactgtggaaattaaaggacctagttgataccaaatgtccgttatctggagtagcctctgtcggtagctggatgccttcatttgatgtgaaaacaattgtgtcatagttgaaaataaaagaatgtttttcagcatgtgctgttgctgtgtgctggataagacttttataattaaaaatttcccctgaatgtcaatacatatatggtattcattttatagtggactatgctgtgttactttagagaggctactgctgcagaaccatcagaaaatatctgtttcagccccaattggacatgactctcgggtatgctgttgtttaataatcattacattagacggttcaatctttatccaaagattggacattctcattgcatacatttgagggttaataaaaccaagctcaggcacttcggtattcttttaaactctgaatgatgtcccctgtagaatgtgaatgatttccagcccgtgtttaccatgtcctaaaacacgcatatttatgggtgcctacaagatggcacagaatgaatcaaaaccataatctcttaaaaaatatgtaccacactgcagttgtgcttgttgataatttaaaaagcacgtttacatgtggtgcgcctattctaggtctccttgatttccttcttccctttgtaccaccactccatgggctgtagcatcctttaagtatggaggccctgctccatgtcatagtaaccatcaacacacactcccatccccaattaactgtagtttgcaaaattatatatgtgttgcattgtagaggctcattggcataagtattcctgtagacaaacacttattaaaatctagaggtcttcttttttgtattttggtggcactattttattttctgagatctcaaacttttacactttcatggcccttgtgagctcagacccccttattctgtgcctgcagagcccaacgcaggaaccgtccctgttgttggccttgcattgatggtgggcctgtcctgtgtggggtgcagtaaatgtgagtcttcctgcagccttcttgctttcatccagaagcaaatttggtcaaaacaagaagccatctgctcctctcttggggatgcctgtagcagcaagtcaggatttttatctccttggattgcaacattcagggccttctgcgtggtagatccctactgagctttggggccagcaaaagtctagatgggtatgattaactggtgaggaatttcagaagccagatgcagttactgttccaaccatgaggcttttccagtcacattgagtcaatgctaaggcattttcctggcatacactttttttaacccttcctaaaatcacaaaaccaaagggttttaaccctttgagtttgagatctcagaaaataaaatagtgccaccaaaatacaaaaaagacctctagattttaatgtgaccacaacctttttaccttttttaaacttcaatttagactctattttttttctataaatgattctgtctgtagtaacgtttatctatctttgcatcttccgactttattaatttttcttcaacccacaacttgatctaagtctttttcttgaccatttgtaaggcttctcattctaatgtgttttaaggatttccttttatttttacctattgctgacacccttcttgctagtgctgttttgtccacctatactgaggtacaccattgattttaatttttttttcttcttgtttttaagactggagtcttggtctgtcctccaggctggagtgcagtggtgtgatctcagctcactgtaacctccgcctcttggattcaagcaattctcgtgcctcagcctctaagtagctggggttacagacatgtgccaccacgcccagctaatttttgtatgttttttttagtagagatgagatttcaccatgttggccagtctggtctcaaactcctgacctcaagtgatccacctgccttggcctcccaaagtgctgggatgataggcgtgagccaccgcgcccggccctgattttaatttggggcatattccacttgactttaaaaggtcgctttgttctttacaaaactttgcacttcgtaaaaagtgtgttctttgatgaacttgtctattcttttctgaaattttcagcattgactttatggtaactattaacaatcagtctaattttttgttactatttattttttacagcatcaatatataatttatgtggagaagtcaatgaacagacttggcatttttaagtgattctatactccaaatactggtcattgagaaaagtttgtttgagagatgatcataggggaaggggtcgcatgaatattgtatcgtctaatgtattgaagtttatttttatttttatttttattttagttttttgagacggagtctcgctttgtcgcccaggctggagtgcagtggcccgatctcggctcactgtaagctccacctcccgggttcatgccattctcctgcctcagcctcccaagtagctgggactacaggtgcccaccaccatgcccggctaatttgttgtatttttagtagagatggggtttcactgcgttagccaggatggtctcgatctactgacctcgtgatccgcccgcctcggcctcccaaagtgctgggattacaggcgtgagccacagcgcccggccaaaatattttaacctgaatttttggtgtttgaagagtggttttgagcgatctacacgctgttagatttagattttgctgcatagaacagaaaacctaaaactaaaatggcttacgtgagagaaaaggttaactcttcatatgttaaattccagaaatgggttctagggctgatctggtagttgaggggtgtgatcaagtttccagatttcagatatccaaaataccctgattttatcattacacattgtatacaaatatcaaaatatcacatgtacccccaaaatatataatgtgattatatgtcaatacaaaaccattaaatatcccaaataccccgattttatcattacacattgtatacaagtatcaaaatatcacatgtacccccaaaatacataaaatggttatacatcaatacaaaagcattaaacatcccaaataccctgattttattattacacattgtatacaagtatcaacatatcacatgtacccctaaaatatataaaatgattttacatcaatacaaaaccattaaatatatgttaaaaagatttcagactcatctctcagctcttccttccacagtgttggtttctgtggaagggtccaaacagcggctggagctccaactgtctttgtctagcagcaggaggaggtagggaagaacaaaagctttctcagaaggccatgcagtatttcagctcatattttgttttctagcacctagcagtggagttatgaacatattttagtggaacatatttagtggaacataagcattggatgtgagggtagcggtggtggtggtgaaagttggtccagaaagactcccacatttctggctgggcgatggggtggatgtggttctaatctcttgggagggagcagaggagaaagaaggttcgggtaggtgagaaaggtagtgatgagctcacttggggaaatgttgagtttgaaggacatcctgttgggtcagttgtagaccactatgtgcatgagtctgaagctcaagaaataatctagtgggcgctagagtgtagacaaagaatcctcagtatcccagagggctttcaaaacccttcaactggatgggtctcccagcaagtgcaccaagactgaattggtgagcgccaaggatgacagccccttgaacctttggaggaagtcatggacagagggcagaggagaggagcttaggttgggacttggagatgagaaagctaggtctagggagaattaaagggaagctgtgactggggagtaagctcatgataagaaacagctgtgttttctttagcttcatttttgccattcggctattattttttggtattatggaaacatcgcaaacatgaacattctaactttagaggtgggcaggcaggagataatcagaagggcgccacaagaccatgtaatctgctcaagagggagcatttaagtctttattgtcttatttaatttttataaatcccctctcatgtaaaggtattattatccctattttgtacttgtgtaaaaacaggctcagaggttaggttacttgctgtgtgccatgcagcagaaagggatggaactcttgcttgacacccaagcacgttttttggagatgttctctgtggatgttgtgccttggcaccacacctcctggggagtttaacctgtgtgtccagagtttttgacctggtacgtgctgtgtagcaacctgtctctttgctctcaggcagaatacatagaagaaggaattagagttaagaaacaaaaagactatcttgtttcccaatcttaaaaagttcccatgtgacattgtttttcactgataatattgttgcaaaccaaaaagttcctgattaatctttgcaatatcctttagacctgaatcacactctcatctgatcataggaactgagtcactagtgaagtaccttcatctaagactctgggcaggggtcagtcaagtttttcttaaagggccagataataataaatactttagactttcccagccatgcagtttctgttgcagctcctcaactctgctcttgagtgtgaaagcacttgctgataataggtcaatgaatggccatggctgtgttccaataaaacttcaggtagcccagtatactcacctctcctctagaagaaaagacgaattctagaatttctagctcataatctggcaagtaaaatcatctgacaaaatcataaaataatcaattggtttttttttcattattttcaaagttcaaaatatttagttactttaaaaaggacttatctctgtaatgattatttatttgtttatttgtatttacttttttaagacagggtctcactctgtcactcaggctggagtgcagtggcgtgataatggctcactacagcctcaccctactgggctcaagtaattctcccaccttagccttcccagtagctgggactacaggcgtacgccaccatgcccagctaattctttgtattttttgcagagatgggggtctcactatgtttcccaggctggtctggaacccctggactgaagtgatcctcctaccttggcctcccaaagtgctgggattacaggcatgagctaccatgtctggcttataatgatcttttacaagtgtaatgatggttttaggagaacagatgcacagtatgacattagattccccactttcttctctattttgtttctttctgtgtagtcttgctggaattctgattaatcatggtccttcccaatctctcagaatgacccatatctttcagaactattgtcttaatcatgaagaccctcaattaccaaattgagaagtttatgttgctttgaccaccatctgtgtgcatcccgatacctggggttacagactggaatctgtgatcaagggagagcgtggatacttgctttattaacaaatttacgtgtgttttactcaccagtgatgagaagaccaccgacacattcaggaggctttatggaacagagaaatccttcaacattaattactgtatctagtgtttttttttgttttatgcaaaagatgtacgtagctattttggaaaagcattataagtagaacttgcatgttgatagctttaggatgtgttcagattacaaatctccatttgtactatacttgagtttttggtaatttgaactagactagatctttaagggaaaaaatatatatatattttttctcagagaacaaaccaggaaaactttagcatcttgaaggtaaattagtgttgattatgacaagtgagaataattaatttgcaaatagtcataagcagattatctttttagcagttagttctcatttatttgtaattgaaaaaacaaatatctgctaacataattgtaattcatcttttgtgttcatttttaaagtgcctgttcctttccagacaagctgcactcatgcatggcagtgttacaagggtgggcttgtagactgagtttgggaagtgctatgaatctactaggatcacctatcacaatcaagattccataattccaagtaaaggaagatgcttttgcctgacttagatgatggttcggattctagtgtagttttttttggaaatgcagttctgtcactttcacagctatgttaccaatgaaataagtctgtgtgaataagtaaagggaggtaagttgataaaagcattcagtttgcaacggatctgcagtgcatcatggtgctcaatacagtttttgaataatcacatttatttatcagattttaattggagaagatttgcagatcccacctttgaatcattacaaagtatgaatcctgtcatctagaatactgaaatttagtatgtaccatgcttaggactgatccgaagacattttaggtttgaataagaaaaaagaggcaaagaggcccaatataaaaacagagagaataaatcttaaagcaccatctatgttggatatccttattgcaagagatcccacattggacagacgatttcagttgacaccaaaggaagtaagaattttctgagactgacgggtatcttggtaaatgttccctctttgcaggtggtcgtagataatttcaattcaccctctgctttcttcgaactgacgaaggtgtagctttgatattttcccctcttcatgttacatgatcatttattctaactggcaacattttatttatttatttatttggacccattgttccaaacatgtaaaagatttggacctgcaaatctttctatgttagtatttattgtattagtgtttgagatacctggaattttggagatgaagtgactcaatccatttattttctagaagaaatcttagagatgtgagggacaggtgtagattcacaagagctaggacatgctgttctaacaaaaatcctccttccctttggttcaatttttaatctattttaggtccttccatttcacattaatgacatagtaatctacccacttaaaactataataagttcactaggtttttttttcccattaaaacagactcatgagtcatagtttactattactttccaagcttttattctgtattttccttaacatctgagaatcatgtggaatctgtggtacctgtgctaatctgaagccactatgtattttgaaacctcttgccccacgttggatgcctggtgtcgatcaggaagtgttgcaacgtgtaacacatcactcaccactcttgacggtgttacagggtaggaagttgtagagatgcaaggctgactgtggacgtaaataatagaacattcagaatcatcactgaggcagagttttgccagcttgtataaatcaggtaaatttgctcatgttaaatgcatctctttcacttccaccttctgtttcaagttgacaatgtttgcatttgactcctattaagacactatgcctttcctcaaacatatctgttgcaatttcatgatttcttaagactaatgctgtcaccagtatatttcatttagcaacgtcctgcacaccagagtaggtttctagatagggaactcaagatactcacatgaaggaggcatgggctgagtgtgtagggtagaaacttggtctttgaatcagaaggaatttgcacccagctgagggaatcttgaggtgtcctcttgttgacatctgaggtttcacatgctcttcatttgtctggtatctgcatgacttgcctgcaccccacttttttgcagcttccaccttggcccatatctcagagcacaaacaatttgaaggacattccaaggtgaatccagaaaactacattaggaaagatttaggagcctgcctagttcttttctactctaactcttgtggaatagattgatttttctgttgtagtttatttgttaaaattgtttaaggccacactgtcagaatttagctcacaggctagcatcggctctgctacctttgacccttttaagagtcatagttttcctatgatgtagaatggaaagatctatgggcagtcagggtcgacctttgggtgccctgcttctagtgcagtgcctggatgtttgtgtcatttctcaaataaataagtgaatttgagtgctcaatagaatctagttgtgtgtatacattattactgcacgtttgtgcaaaaactaaaaattatagacataaaggatacaattctagtcatttatattacaaatgaaataaccaaccctgggaggttaacagtggcttgtttaatgtcagcagctagtttagtggctgtaacgagactgaatcccaagttgaacagaaatcgtttaaaaattcaagttttaaaaatcaagtacaatgaatgatattggctgcttttctaaggagagtgattctctaagcctataggaaaaaaatactggtggctgacttgtggccttaattgcatgctaaggttgaggaaggatctccagtaattctttgctttactttgtgtttgaatataggattttatagctgtgggtggaaaaaaaaatatttctagggaaaaggtttcctgataatttaactgaaccccaaacaagtgaactgggtgaaacagaatatggcacatggtcctttatcacaacctttctacctaacctaaagccagtaagatggaaatagtttggaaaagacactatctgctgaggttgctttaaatagagtctctcctgggacttagaacacattttgaggctcattctcccttttgatagtttcttggagacatttttctgtatatactggatgcaggcatttgtattcctggatggcattcatcttttaccagtgtgaggaggctgtttttatcattgtgagatttattaacatttacttgctcttagaatctacagattatataacagatattgcaggctataacctagaatttaaatattaaaaagagtgattaaaaaacaaaacaaaaagagtgattctttttgttataattgaatcaattcctattgtttacctgtgttgaatacagtaattggtcccaccgatcttttacttaatcttgaaattcagagttattctctattctacatgaatggagctgggaaatggttttaggtaaatgaaggtaaaatgtactgaatacctactgtgtgtagtgagctgaactatatcctttactgcaaatattccatttaatgctaacaaccacctccccttctattttcttctgtaggcaaaggttgagttcactgaggctgactccctgccccaggtcacatagctgaacgtggtctctcatccccttgcctatgtgctttttttcacagagtatattggattgagatccaaaacttattggcaagccatcaaagttccgcttgaactaaaagaggaaaacctgttttctgaaatgtgaactttaattacataaaatggaggcttctgaaaacagtattgtctcacagggcaggctataaaccttgaacaatggaagcttcatgttgtgtaagaaaggagcttacagagccaaacagcaccgttcctgtgtttcccctgccagtgacaggagcatctggggtagcaaggggactgcccagggcctggtgttatcatcaggggttcttgaaattgctcacactgctcttgcttttccatacaggctgcttatatcttgcctcctctggctaaactgtggtactaacaagacggcagttggctgctggggaaattctgatagggagagtaggtctgggtcattctaatttatttgtggctctggtagcaaagagaactcaaaggcagtatatctctatagctgatgagatttcactggggccgacaccacctgttctgtgcacactcacttctcggttagacctggctaggttttctgctgtgcttgtttttagctttgtaaccagcagtaatcacattggtctttgggattttcatgtttcaggttttacagatgatgcaaaagcattagttttcaaagtcactgcaaatgctatgtatatgtatttctcaacatgcagcccctgcactacttgttttggtgttacctggtcaacttggtagaaaatagattcttgggactcatgctgagcccactaattaattatcttgggaaagagataacactccccccacctatatttttaacaagcttctcagatgattcctgtttacataaatttcactcccaatattagacacgttatacagaaacattggcaaataagaggttgaagtgactttgttctgagtcacatcatgagtaaggggcagttggtattgaattctagtttgtgtttcatccccaagtctggggatgaaatgcatcgtctctggaattccatggctggggacaaataccctctgttatgtttttaaatgtcacagccctctgggattcacaccttgggtgttgccttgtggctcagtgcctggttttttaggtagctaaagggctcaaatttgaagaaggcaaaagtgtggagcagaaagtgagcatggacaatttggggtgaagcatttgcttgagaaatctggggagatacatttgaatttataaggagggaccagatgacaaatgtcattgaaagtcacactatgctgtttgaactcaacatggcagtagaatctggaaatggcacactgcatatgtattagtccattttcatgctgctgataaagataaacccgagactgggcaatttacaaaagaaagaggtttattggacttatagttccacatggctagggaggcctcacaatcatgggggaaggtgaaaggcaaggatgagcaagtcatgtcttacatggatggtggcaggcaaaaagagaacttgtgcagggaaactcctgtttttaaaaccatcaactctcgtgagactcattcaccaccacaaaaacagtgcaggaaagacctgcccccataatttaatcacctcccacggggttcctcccagtacacacgggaattgtgggagttacaattcaagatgagatttgggtggggacacagccaaaccatatcagcatagtattcatctttggattgaatacattcttttattaatttggcaaacatttaccaaacccctgttagtgctgaacatgacaaaggaaaaagcaaattctttgtttagtatgtcaaggacatacataaaggattttagatggttttcactgaaaatcattaaaatatgaaatagcattaggactgaaaatactacatttgctgttggtgccaaaaaaaattgatacatgaaaatgtcaatatgtaagaattttaggtggtcgtgaaaagtgtgaaccagccctttatttttggagacaggaactactggaaataaattgcagttatttctggagtaggcaggcttcccctcttctccctaatcaccagcctgtctttgtcaggtggccacattgatcgagcctgtcatggctccaagcccatcatggtctgtctagtccattaagaagcagcagaaatatctcagctgctctaagtgttttccttgtgccttattttgagctctgtttggagaattctaaattagttgtggtgatatgatctattagtttcctggtctatggaagagagaaatggtacagaaatgatctctgcagtttgcatttcctgagcgggttgtggttcacttactccctttattgaccaaaagagcaggtgattcatgtaaaatcatatgttaggctgcaataactggggcttttgaaaggaccacccgttaagtgcaaataaataacaatgtgaacacacagcagactctaaaaagcatattagactattgacgagaatgaataatgcatgtttcctgatagtgtttgctacatgtggcaggatgtattaaaaaagaagaaaaaaccttctgcattagtgtctatacagtcaacctttacattcaaggagaagcttaactattatctgccacctttatagaagcattatcaagtcagggttttatggttaaatcttgtagaatcaattgggagattcacctttaaaaattggtctctacaatatgcacatagtaacaaaagaaaaaaaaatcttgacaatatcattgtagtgatttctgcattctggtctttaattatgtgctgagtcccggtggcagagagtttgtgatcatggatggcgcctggaacttgaccgcatttcatttaatgcaacggaaatttcagttacaatttagtttattgatttttttctactgctggtgaaaggtaagttacataacccaacttctagtcagggactcttccatcttacagttcataatttgcaaattagatgttcccgtctgtagtgagtctgtgatgtttatctagtacccattgtgcacgaacaaccacaaagcaaaatctttgttctgaagcctcagattgatgttgaataaatattgaagaaacgccctaggtcaagattcaagtgaagtcaggcaagcctattttactgctaaaactttgaacgcaatccaacaatataaaagggttaagcaggcctggcctggtggtggctaacacctgtaatcccagcactttgggagaccagggtgggaggattgattgcttgagctcaggagttaaagactagcctgggaaatatagtgagactcccatctctaaaaattttttaaaaatctattttataaataaaatgtaaaaggcttaggcaatgatgatttagataaatgaccaaagatggatggtttatctaaagaccaagaccagattgcacagtaaggacacaataagaagttcttaagaattaaggttcgtttttttacatttatgtagggaatacacatgtggatgctgactgtgcatttccttatctcagaagctcctaaaaccatttataatgtagcaagcctgtttgctttacttttgcataccggttgtttcacacaataagacctctccaggtttagtggtcactgtggcagttcttcagcattttagttgttgaaatgtctgtccctttcccagtacttaagaaatcctaagtgttgtaatacgagagtactatgaaggtaaatgactgcagaatgaaatactgtaaggtaaaggactatttcaaagagacatgatctaacgtttactatgtggagatcttgtggagccattactctggaataattcagatttaaccatattcatgtaaaagagagcatgttgagttggaataaccatcagatttggaatttatctcattggcttctctcccccccaccgtctgcataacaaagtgttccacttcagcaaacatctggacttaactttctgaatctcccagaactttgcgctgtcaagattcctccctcaactttccactttagttttgatttttatcctttgtctcatcctatcttcccatagatattttcagttcttgcaaatccttgtaaacaaataaaagtcttgccaaaattgcacgtttctgccgttaccaacactctctggtattgaccattgtggctttctcttctggcgcctcatgtgctctgtgagccctttcaaagaggcaggtgtcttcactcctttatcgaagtgactctagggtccgttgttgacagcccatcaccggatcaatttgcgccatctcttcttatcttttgtaacctttttgtagcagcatatgcctcctccaagctgctttgtaaaattttctcttttgattttttgagacatcatcttttcctttctaactcctacaagagctgtttttctgctttctgtcatctaagtaattttccctattaggcaacctttatagatttagagtccatcttttctcactccaagtatctgagagtcctctccttcctcttaaatatcaatagggttttctaataatctcaactggacatttcctctttgttccctttgacataaacactttgcatttacccatcctcatctgtagaaagcttatttgttcttcagggcaacttatcatgatggctaagaaaccttaacgttaacctggttgagtgagccagagtgcctgcattcacctcacacctccacaaataactggttggttgatcttgggcaagttatttatctcctatgtgtctttgtttcctcatgctaaagtggggatagtaataattcctccctcgtagaactgatgagatagctaatgagttgatatgtgtaaagataagtatcaaaacagtcttatgctgagtgtatatgaataagtattttttatatttctactgtctcagactctaccttctctataaatctttctgatcctgatctcaactaatcgtcatctcttctggtgaaactattgcttttccttctcttgataacgtttgacttactctcatttatattttaattgtcttcatatgtcttctccctcttgctagactaggctcttcactcacttatgttccctgtcacgtcttacacctaaagatatttcctcactaagatcaaatcttcttttttttttttttgagaaggagtcttgctctggcgcccaggctggagtgcagtggcacgatctcggctcactgcaagctccgcctccctggttcatgccattctcctgcctcagcctcccgagtaactgagactacaggcacccgccaccacgcccggctaattttttatattttttagtagagatggggtttcaccgtattagccaggatggtctcgatctcctgacctcatgatccgcccgccttggcctctcaaagtgctgggattacaggcgtgagccactgcacccagccctaatcaaatctatttttctagactaattctgaacatcagaagttcttgagatcactctggatgttgacttttacaaattatattgtgacagagtctcattgtgtatctttatggggtacaaagtagtattacattttttttttaacatttcttttctttccttttctaatagagacagggtctcactattttgcccaggctggtctcaaactcctgggctcaagtgatcctcttgcttcggcctcccaaagtcctgggattacaggcatgagccactgcaccccagctggtattacaatttttgaataaaatgtggtaagataaaattaaggtaatgaacattgctcacctcagatatttatgcattttggatttttgatgagagatccccaaagaccctgtgccagtgcatctgtaggactagactgggcttcatgaagaataatggaggcgtttcttgtgattaatcttcttttgtattagggactttctgactttgtagatccaactcatctggatcacctctttgggaaaatttggttgtagaaatgtgagtgtgtgctaatctgtgtatgtgtactttgttccctcaagtttgttgcatatctttatcatacttttttacagtgaggaacatttttattgtgacaaggtaataaattgtctcgaatgttttctctaaatttttactcatgatatttcaaaggtttacctagaaacttagacatgggttggcacctcttaggacagtttcctctccccagaggacatctgcatcctctaaatgatggggatgttattagaattgctaaatatgagccttggctgaatcagacaccgacaacaataatttcaacaatggaaattgccaccaggcactgaagacttgacaagtcactgcgacagagcacatactggttggaatactaaaaatacacatgggctgcattacctttatttcattttcctcttttagaataaaaattttgacctttaaaagaaaagggccagtcacttgttgaaaatagaaattgctggacataaatattttagatttctttcttctgacagtattgaaaatactttgtatgtgttgtgcttttttattgtttgtaaattcttttctttatataaaaaagaattttttccctcttattccaacatttgcttcaggtaggtttgaccaatatcttggattctttccacttgtgtaattataaacccaacttgttggtgatggatttgcttgtagatgcaactcagttgtaactttgtgttacttaaaaaaaattgttagtggattggctggtgtataattcttgttaacctttgcagctctgaattagtagatgatagttgcttgtccaagggtacacagttaatgtgctgcagccacacattgaactttgtcttgaaactttgattcattccctgcatctttgtaagcagatatatttttttctgattataagatggaaacagaatgaaaaggagatgacttcttgtcttgattgcaatgatgaagctgcaatcaaaccagaaataacactccagtctctgacacacattgttgcatatttatgttgggttttgctatagacttggagccatcttctccattgtcatctgtctgatctcatccttaataactttattctgctggttctagatgaaatggttaataaatccaaatttgcattttcaaagtagaaactttatagtggatagggcctttgtatgattcaaactgccctatataggaagtgcttagatattataataatgcagggaaagaagatataaataggaatggcatgtaaagtagtgaacatagtaactttcaaatcatatggagaaatttgatatcattttcttattttatatggcagttaatgtatgccagtgattcttattacattacagaaccatggtattgttaattgacagagcaacgtaaatgtaaaaatgtgttaatgaaaaatctaattggagatatcatggagcacatgtggtaattatgggaaggacccagtgcatgtaacccccttggcttgtgggtggataactcctggaaattgtgcattgtgcaaggttgcattttctgaagaagggcaatggtatattggggggcttgccatttttcatccaaaaaccatatcatgcgaagacttgaataacattaaataatggatgtaaccaacaaagtgctactgaaatgaaggttcaacagtccacaaatggtgagcctctgcatatcaaaccttctaaaattaagtgtataggctgagataaacattagcatttcattacccataattttgcttgccgccaaaaatgcagaagggctcttgagagctatttggatgaccctggtctctggagggcatcttttagaaacctgatggcatgtcatcacttagacctgccacaaagcactgaaatcctcttaatggtggtaatgataacactattgtttttcctacttgatactccttttgagccaacatttctccctttcttgctctcctatatccaaacaacctgtttagtaagtgttttatttaaaggactaaagaaaggaagtaataaatgatagttctcagaaacctcacatttcattgcaaagctattagaacaacttccagctcaaatgttaattagataccgttctgatatatagggttgtgtctttaaatactgcaggagtcttgacccaaacccaaagaatgcataggcagcagggctgacccatgcgttttcacttggtaccagcaaagggatttgagaacccttagtcactgcaaggggcagaagtgtcttctattaaggacataataagtcctgctgaagaacaggtcagctcagtaactccagtcgccacagcagttgttggtcaaattgggttcctctggtagcaccacagagcaagggacactaacccgttgggcttcccgattcattgctgtcactttggggcagaatggaggtatgatttaaatcctacagtgcgggatattttcaccctggtgtgtctctgacagtgtcctgctatagaagtttaaggaacagtatatgtggcttcaatttccataagttaaaaatgcaggaaaaagattatgtctggaggagtagattttcagaaaaagcggttttaaggatgatgaaaagagaatttattcaaaatgagaggcattttttctgttccttcgccacctctccctcccttgcttgctctgttctgtctccctccctccttatttcattctcactcttgctcttgttcatctaagagtaatgaatctttgccttattgtgtttgaattcaccatggcctccttgttgatattcctaaagttggcattaaaagctatatctggggtggggcatggtggctcacgcctgtaatcccagcactttaggtgtctgaggcaagcgaatcacctgaggtcaggagtttgagactagcttggccaacatggtgaaaccccgtctctactaaaatactaaaaatacaaaaattagccaggtgtggtggtgcacaggtaatcccagctactcaggaggctgaggcacgagaatcacttgaaccgagaggcgcaagttgcagtgagccgagatcgcgccactgcgctccagcctgagcgacagagcagcaagatcctgtctcaaaaaaaaaaaaaaaaaaaaaaaaatctgacctggaactctttctcaatgtaggaactccaaacataatctttcaaaatgttgcctgtggatagctctgtaaaaatggtgatacaccctggtttcgttttgttaagtggttgtattgcgctggaaatttggggctgagaacttttcccactttatttttctcaacgtttctacataacactaaactgaatatcacattagttttcgagaattgtgattagcccttgtgagattgatttaaactatatttgaaaggataatttaaagtatctgtaggctgatactacttagcatcgcataaaaggaaaagatggtttgattgtttttaggaagcacgcttgctcaaagagattgggtcttgcaaggcaggaccaccagtgattggtagatgacccatcaaaagtaggtaaaggggaaacaaccgagtgtcttccacagccagggcactgtgagctagttctgcgtgagtgaagctgatgaaacctgacttttctgacagttatgacgctatttgagatgtcttcagtgagccattccatcctgactcattccttacagagtttacacattattctgagaagattttctaaatataattgaaaatgttaaaaaacattctgtgctttaataaatatttggatgtcattttacaatttttgtttgtatttttttttgctttttttttttgagacgaagtcttgctctgtcaccaggccccaggctggagtgcggtggcacgatctcatcccgctgcaacctccgcctcccgggttcaagcgattttcctgcttcagcctcccaaatagctgggactatagacgcacgccactgcacctggctaatttttgtatttttagtagagacgaggtttcaccatgttggccacgcttgtcttgaactctcaaccttgtgatcctcctgcctcagcttcccaaagtgctgggattacaggcgtgagccaccgctcccagcccttttttttttttttttttttttttgctttttaaaattttttttatttctaattttcagcaagcttcacgttgaaatgacgtatgaagacatccaatcatagtaatagcagctggttgtcaatttaatcttcagggtgtaagatgatatatttttatgacagtgattagtagatgtattgaactctgttgataataccacttgtttctttatcttgcattgcattttatttagtgccagaagcattcataaaattatttttccccctagaaagagaatacactttaagtatcgttgttttactgattaggaaatttgagatccagagtgggaggtaaccactgagtctcatagcactttgttgacagtggagaccaacccctagcacacctatcgtttttagcaaacttcatactgagtgctaggtggcttgtttttgacacaagtcaaacaaataaaatatttcataatttatttttttcttatacggcattttgctatctactcttccacctttattttctcaaactgaaagactgaattttaatacagtaggctgtgctaatggagtggtgtaaggtaagaaaaaatgtaagtgcgtattaggaaagttctggatgagttggtcatgtcagcagccaaatcatggttgtttagtgaaattatttgctattaagtgtgtacagaggggatcctattatagtttgtaagaattgtggttttttagtgaaattcttagctattaaaatagcgtgtacagaggggatctggacccagtgtgtcctgtggattctgtgtacaaaataaacacagcagatgatctgaagggataaggaatatcttctgtgttccctgacaggcttcctggagaaaaaggaacaagcaattggaaaactcggtagaggagattgtctctattacagagtgtcttcattataacatcagatttcaaattatttctcacctttactctcccaaagaaattttcatattaaatttaagctctcaatagtaagaaggagtttactggtttttctttttggggggtttgctattatagcaatgcataggaccacatatttttcctataaatagaaatttctgtaaaataagaaatacttgttagctctaagactcaacagaattgttttaatttgctttggaggaagaattaaaacctattttttaggcctatgttcattctttatttttatttattgattttttaaccagatcctctgaagacactaaagtgtagttgtctctgaggacaaaaacaaacgaacaaacaacaacaaaaaaacttcaccttaccttgctgacctagctatgaagttgctcttttatggccttcctaggcatgaagaagttagagaaacttgtctttaaaagtttcatgaacgttcataaatgtacacgtttatttattagggaacatgatttgctgccaataaacgaaagaaagatttgtgtcaggactgagtgtgtagtctaagctaacatcagttgtaggaagtgagactcagaaggtgatgggtatgtattccacggatgcaaacactttttgagtttcctggcaggtgtaggcaaaaaaggaccaggctggtgtgatgaataatcacttccaaccctccctctgaccttttcaggcctccctctgatttgcctgctttccagcccaggttgctctgatcaactcacagtgctcgaatggcacactctcttctctcttcaccgaaaagcagaactttatcacatttcttccagaatagctgtttctagccaggtgggattaattggatggaggcttggttttataaggtggaagggaataaaactagtcacaagatagttgtgggggattttaaagatgtaagttagtatctctattaagctatttgacatatgataaacaaacagtattcttctgcaggaagtagcaaaacaaaatgacatttgacaatgttagcttctctcattaacatggtgtgtttagcttattagccaagataagctgtagaccaggtttttaccctttacacgtgaaatttgaggcattaaatgagtaaaagaattgggattccttttccatcttggtgatcaaaacacttttctcttgtttccttggaccaagtgatgaagaggtggtgaggggacagcaaaaaatgcaattacatttcttctctttatttttattttttgagacaagagttttgctcttgttgcccagggtggagtgcaattgtgcgatctcggctcaccgcaacctccacttcccaggttcaagcgattctcctgcctcagcctcctgagtagctgggattacaggcatgcaccaccacatctggctaattttgtatttttagtagagacggggtttctccatgttggtcaggctggtcttcaactcttgacctcagatgatctgcccgcctcggcctcccaaagtgctaggattacaggtgtgagccaccacacacagccccctcttctctttctctgatcactccaatactcgttctaattgggaggaatggatttaactaatctttggtttttatctctgtgtagaaactgatttcttggcagaatggactgttaatatatttaaaaagactgaaaacctcttagaatcttaagtagtaaaatattcagagcgaagaagtagagattttacctttacaatatggtgacaacagataactgtaaaaacttctttttcaaatagaaccagcaggagcatgcatggaacacatataccaaacatctttctgataacattaaacatttttaaaagatgttaaatgttcttttcattgtggtgcttcagattcctgattctagaacttgtgtgtgtggaacctgtgtgctaactattctgttggaatttaccagcaaagaattatctaagaattttcaaactaaatgatgggggaaggaactaacatttttgcagtccctggaaatgtaaaatgttgtactcacatggcctcacattaaattcccattaaattgacatttaaaaaaatttttttattttaaaggaaacggaaggacgatgagattaaataacttgcccaaggattctgggtgggtgtgctgtggaggtgggatttttatactctgccatgacccaatgtgcaaagtctccttgaaattcaattcttgtccaatatttaccagattaaactcagtgaaattcctctctctactactgtttatggttattttctatgccaatattaatcggtaagcctgtcattttcgcccttgtggcatcgagttttctttgaaggacagcttttttgtttgctttttgttttttgttttgctttgttttgttttgaggcagggtctcactctgcccaggccatagtgcagtggctgatctcggctcactgcaacctctgcctcccaggctcaagcctccccagtagctgggatgataggtgtgcaccaccacgcctggctaatttttgtaatttttgtagagacggggttttgccatgttggccaggttgatcttgaactcctgacctcaagtgatccacccacctcggcctcccaaagtgctgggattacaggtgtgagccaccacacccggtctttaaaggacagctccagaattagcttttttctgagatgtcataacagaggtcagttttagtgtttggttaatacataaacacatagaactgaggtcagtgccttcagcattggtctgaattttgttgctgtgctacaaaattctagtgaatttcctagttgtggagaaatgcaaagtcagtctcggccttttcagtagagctttggtttagtgacttttttagttgaagtgacttttcagcatggattgtctattgctgtatttttttaactgctttattttaatcttagaagttccaacttctaactttctataccaaatgatgtctttcaaaattagaagtaacagaaatgaatgaaatcggctgggcatggtggctcacgcctgtaatcccagcatttcgggaggccgaagtgggcagatcacctgaggtcaggagttcaagaccagcctggccaacatggtgaaacctcatctgtgctaaaaatactaaaattagctgggcatggtggcaggctcctgtaatctcagctactcgggaggctgggcaggagaattgcttgaacctgggaggcggaggttgcagtgagccgagattgtgctgctgctgcactccagcctaggtgacagactgagactgcatttcaagaaaaaaaaaaaaaaaaagaaagaaaagaaaaagaatgaaatcattaaactgtatgctcattgctttctcttggtaaaaagcttctctggcaaataggaagatgaaatgttgagaggcagaggatactcaaaatactgaatttcttccttattccctcaacacctgagttctgaactaccccaggtggcaccatccttgtaacccaggcgcataacattcctgggctgagagctttgccccattgccggcttctcagtggagttccttctatcccctctgtggtgccttcctgccttactgaattttgctggacttggcctttgctcatagtacagctccacgaactcagctagtaggatttccttatgggtaaaaggagaatgtgaatggcatactaccccatggtgctgttgagattacatattttatgtaatatatttatgagtaggattgtgcttgatacctaggaaacagttaatagatgttgtaggcaaggtcttcacgcatatggtcttgggaaataatgagacaatcctgcttttcgagggttcaggtgggtcctatgagcgtccctctgatacctcaatataaccagtagaggtaaattagcaaaatgccaggggcgtttgttaactttgtttgaaaaacaaactggattgagacatattttcttacttataacttgattacgtgtgttcattttcgacccagaaacaaatcgcagctgggcataaaacaaagaatagaatgtggccttaactatgtgtcagaaaagccagttttagtaactcagattttaggtttgtaacccaacatgactttaaacctctggggattgctgtatagaatttcatgctgtgggtgtcagaaagcagcaccccatgttatacttactggaccccctatgtttgcactttatcttagtgtctgtaatttctaagttggtgtatgggtttctattactggattgagagaagatcctaaaggcagacctgttaggtttcctgtttcttatttagctcaacctaagggacccaatgtattttgaaatatcagagctgaaattatatttggtataggccattcgtgcatactgactagttaagaattcaatggaagagttataaactataatttaaaatattgatccggtttatttctttatattttcatgtactgctctgtgattgcttataggatgaaagtatatttaattgtattttttgccatgtatgtttgtttctacttttgagggagaaataatcaaaaattaaatccaatagagcctgtctgaagcaggttatgaccaattgccacccccttttctaaaaaaaaaggacatgtaagagaatcttaggtaacatcataaagacctttcttgagaatatgaatagtattttagtcaaatttcagtttattgcttttctcttcccccaactactgaagtcattccagattagaaattagcatatagaatggtgagaaaaattttgactgaagaattcattttcttccaccaaaaactcgacttttaataagataaatataggttatttcactgtcatgaatcttgattccaaggaagtaataaacagcaaatccttcttttctaaataagggatgtatatatttgcaaagctggcaaaggagtgtgtacagttctgccagtggacctgacctgggcaaaaccaggtggttcccccctgctctttctaacctcccttggggtccagggcaaggctgatgctctctcctgggcttttctcatttatgggactaacgaggatagtataatgtgatttctgccacgcttcatccaggaaaagcaaccattttctttgcaaaagcaacatttcagtggattttcactgatgtgtggtgacagaaaaatggacttgaaggaaaaaaaatgcaccaagagtctgtttagctctggccatggttctggatgaaatttattatttataaatttataatttattatttatctggtttaatgcttggttttcagaggagagaatttcaagattcaaatacaaatggagtttcttgtgccagaatgaaagcggcagccttaagacagttgttaatgaccattgaacgtgcatgtatagtttgaattctaaccaggaaattgataatgaacaacaattttatgttactatagaactactttttttaagctgaaaataccttgaaatgaatttatgtcagacacagccagcaagagacactggctttttcataactgcccctgcttggtaagaagatagactgaatcctgtttaccttacgtgtttgaagatgtgtttgctgatataacacacacacatatgtagcagagagagtttgtagtttgagattattttctagaaatctgtgatttgtgctttattacagctgtgcagtttctccagaaagcattttaaaattgcaacgcctttctctagctctaacctgggtctaagatggctgcccctgccatgctgcacatgcctgttccacgcttggtgtttgcatcagacatgggattgcagatgaaaaacagatagcgctcctcctctccagggttgacaaactttgcctctaatcactgattctttgatctcagcttattattccctcaactggctatgattagaagctccacaactgagctccttaatttcttttcgtgacacattgttttaaaaactcatttaaaattattgcagaactcacttaattgagctccttaatgtttttgattctttttaaaatttttaacttttaattgtgtaataacagtgtaatataataatttaactgattacttgggcgtggtgggtacacctgtgggcccagctgtgtgggaggctgaggcgggaggatcccctgagcccaggaggtggaggctgcagtgagaagagatctcaccacggcattccagcctgggtaacatagcgagaccctgtctcaaaaataaatacatgaataaattgaatttaactgtgcctaactatagtttaccatgccacccctttggggtgtgcagtgcagcaggcccagaaccccttgctttgcaaaatgcagctttttgtggtccccacacttgcctagtaaccgccgttttgttttgttttgtgtttgcttccagaactccaagatgggaggcaagctcagcaagaagaagaagggctacaatgtgaacgacgagaaagccaaggagaaagacaagaaggccgagggcgcggcgacggaagaggaggggaccccgaaggagagtgagccccaggcggccgcagagcccgccgaggccaaggagggcaaggagaagcccgaccaggacgccgagggcaaggccgaggagaaggagggcgagaaggacgcggcggctgccaaggaggaggccccgaaggcggagcccgagaagacggagggcgcggcagaggccaaggctgagcccccgaaggcgcccgagcaggagcaggcggcccccggccccgctgcgggcggcgaggcccccaaagctgctgaggccgccgcggccccggccgagagcgcggcccctgccgccggggaggagcccagcaaggaggaaggggaacccaaaaagactgaggcgcccgcagctcctgccgcccaggagaccaaaagtgacggggccccagcttcagactcaaaacccggcagctcggaggctgccccctcttccaaggagacccccgcagccacggaagcgcctagttccacacccaaggcccagggccccgcagcctctgcagaagagcccaagccggtggaggccccggcagctaattccgaccaaaccgtaaccgtgaaagagtgacaaggacagcctataggaaaaacaataccacttaaaacaatctcctctctctctctctctctctctctctatctctctctctatctcctctctctctctcctctcctatctctcctctctctctctcctatactaacttgtttcaaattggaagtaatgatatgtattgcccaaggaaaaatacaggatgttgtcccatcaagggagggagggggtgggagaatccaaatagtatttttgtggggaaatatctaatataccttcagtcaactttaccaagaagtcctggatttccaagatccgcgtctgaaagtgcagtacatcgtttgtacctgaaactgccgccacatgcactcctccaccgctgagagttgaatagcttttcttctgcaatgggagttgggagtgatgcgtttgattctgcccacagggcctgtgccaaggcaatcagatctttatgagagcagtattttctgtgttttctttttaatttacagcctttcttattttgatatttttttaatgttgtggatgaatgccagctttcagacagagcccacttagcttgtccacatggatctcaatgccaatcctccattcttcctctccagatatttttgggagtgacaaacattctctcatcctacttagcctacctagatttctcatgacgagttaatgcatgtccgtggttgggtgcacctgtagttctgtttattggtcagtggaaatgaaaaaaaaaaaaaaaaaaagtctgcgttcattgcagttccagtttctcttccattctgtgtcacagacaccaacacaccactcattggaaaatggaaaaaaaaaacaaaaaaaaaacaaaaaaatgtacaatggatgcattgaaattatatgtaattgtataaatggtgcaacagtaataaagttaaacaattaaaaagaa

SEQ ID NO: 23 - protein, 8 aa, artificial sequence
  epitope tag
DYKDDDDK

SEQ ID NO: 24 - protein, 4 aa, artificial sequence
  epitope tag
DYKD

SEQ ID NO: 25 - protein, 9 aa, artificial sequence
  epitope tag
MDFKDDDDK

SEQ ID NO: 26 - protein, 9 aa, artificial sequence
  epitope tag
MDYKAFDNL

SEQ ID NO: 27 - protein, 9 aa, Artificial sequence
  epitope tag
YPYDVPDYA

SEQ ID NO: 28 - protein, 6 aa, artificial sequence
  affinity tag
HHHHHH

SEQ ID NO: 29 - protein, 10 aa, artificial sequence
  epitope tag
EQKLISEEDL

SEQ ID NO: 30 - DNA, 7 bp, artificial sequence
  promoter feature
tataaaa

SEQ ID NO: 31 - DNA, 9 bp, artificial sequence
  promoter feature
ggccaatct

SEQ ID NO: 32 - protein, 19 aa, Homo sapiens
IGDLQAFQGHGAGNLAGLK

SEQ ID NO: 33 - protein, 10 aa, Homo sapiens
VILDLTPNYR

SEQ ID NO: 34 - protein, 12 aa, Homo sapiens
LLTSFLPAQLLR

SEQ ID NO: 35 - protein, 14 aa, Homo sapiens
GQSEDPGSLLSLFR

SEQ ID NO: 36 - protein, 21 aa, Homo sapiens
ADLLLSTQPGREEGSPLELER

SEQ ID NO: 37 - protein, 15 aa, Homo sapiens
CKTEAPAAPAAQETK

SEQ ID NO: 38 - protein, 211 aa, Pan troglodytes
MGVPRCSLLSLQLCSAGRSQTCGDSSQEQNRKLQPRAAGFKDKAAKQNSKMGGKLSKKKKGYNVNDEKAKEKDKKAEGAATEEEGTPKESRGEGGREGRGGCQGGGPEGGAREDGGRGPAAGEEPSKEEGEPKKTEAPAAPAAQETKSDGAPASDSKPGSSEAAPSSKETPAATEAPSSTPKAQGPAASAEEPKPVEAPAANSDQTVAVKE

SEQ ID NO: 39 - protein, 232 aa, Canis familiaris
MGGKLSKKKKGYNVNDEKAKDKDKKAEGAGTEEEGTPKESEPQAAAESAEVKEGKEEKADKDGPDAAAKPEDKEGDKDTAAAKEDAPKAEPEPTEAEGAADGKAEPPQKAEAEPAAGDAPQAAEPEPATQAAAAAGEQGQEPGEPAKTEAPAAPGAQETKSDGAPASDSKPSSGGSSSAEAAPSSKESPAATAAQAQSSAAAQAQSSAAAPGAPADDAPPAGHAEQAVALAD

SEQ ID NO: 40 - protein, 220 aa, Rattus norvegicus
MGSKLSKKKKGYNVNDEKAKDKDKKAEGAGTEEEGTQKESEPQAAADATEVKESAEEKPKDAADGEAKAEEKEADKAAAKEEAPKAEPEKSEGAAEEQPEPAPAPEQEAAAPGPAAGGEAPKAGEASAESTGAADGAPQEEGEAKKTEAPAAGPEAKSDAAPAASDSKPSTEPAPSSKETPAASEAPSSAAKAPAPAAPAAEPQAEAPVASSEQSVAVKE

SEQ ID NO: 41 - protein, 226 aa, Mus musculus
MGGKLSKKKKGYNVNDEKAKDKDKKAEGAGTEEEGTPKESEPQAAADATEVKESTEEKPKDAADGEAKAEEKEADKAAAAKEEAPKAEPEKSEGAAEEQPEPAPAPEQEAAAPGPAAGGEAPKAGEASAESTGAADGAAPEEGEAKKTEAPAAAGPEAKSDAAPAASDSKPSSAEPAPSSKETPAASEAPSSAAKAPAPAAPAAAEPQAEAPAAAASSEQSVAVKE

SEQ ID NO: 42 - protein, 244 aa, Gallus gallus
MGGKLSKKKKGYSVNDEKAKDKDKKAEGAATEEEETPKEAEDAQQTTETTEVKENNKEEKVEKDAQVSANKTEEKEGEKEKTVTQEEAQKAEPEKSEAVVDAKVEPQKNNEQAPKQEEPAAASAPAASSEAPKTSEPSSDAKASQPSEATAPSKADDKSKEEGEAKKTEAPATPAAQETKSEVAPASDSKPSSSEAAPSSKETVAATAAPSSTAKASDPSAPPEEAKPSEAPATNSDQTIAVQD

SEQ ID NO: 43 - DNA, 36 bp, Artificial Sequence
  primer
acgacgacgtctcccatgggaggcaagctcagcaag

SEQ ID NO: 44 - DNA, 40 bp, Artificial Sequence
  primer
cgagagacgtctcctcgagctctttcacggttacggtttg

SEQ ID NO: 45 - DNA, 16 bp, Artificial Sequence
  primer
gtaaaacgacggccag

SEQ ID NO: 46 - DNA, 17 bp, Artificial Sequence
  primer
caggaaacagctatgac

SEQ ID NO: 47 - DNA, 681 bp, Homo Sapiens
atgggaggcaagctcagcaagaagaagaagggctacaatgtgaacgacgagaaagccaaggagaaagacaagaaggccgagggcgcggcgacggaagaggaggggaccccgaaggagagtgagccccaggcggccgcagagcccgccgaggccaaggagggcaaggagaagcccgaccaggacgccgagggcaaggccgaggagaaggagggcgagaaggacgcggcggctgccaaggaggaggccccgaaggcggagcccgagaagacggagggcgcggcagaggccaaggctgagcccccgaaggcgcccgagcaggagcaggcggcccccggccccgctgcgggcggcgaggcccccaaagctgctgaggccgccgcggccccggccgagagcgcggcccctgccgccggggaggagcccagcaaggaggaaggggaacccaaaaagactgaggcgcccgcagctcctgccgcccaggagaccaaaagtgacggggccccagcttcagactcaaaacccggcagctcggaggctgccccctcttccaaggagacccccgcagccacggaagcgcctagttccacacccaaggcccagggccccgcagcctctgcagaagagcccaagccggtggaggccccggcagctaattccgaccaaaccgtaaccgtgaaagag

SEQ ID NO: 48 - DNA, 20 bp, Artificial Sequence
  primer
taatacgactcactataggg

SEQ ID NO: 49 - DNA, 17 bp, Artificial Sequence
  primer
ctagttattgctcagcg

SEQ ID NO: 50 - DNA, 42 bp, Homo sapiens
aagactgaggcgcccgcagctcctgccgcccaggagaccaaa

SEQ ID NO: 51 - DNA, 84 bp, Homo sapiens
gctgctgaggccgccgcggccccggccgagagcgcggcccctgccgccggggaggagcccagcaaggaggaaggggaacccaaa

SEQ ID NO: 52 - DNA, 39 bp, Homo sapiens
gcggagcccgagaagacggagggcgcggcagaggccaag

SEQ ID NO: 53 - DNA, 72 bp, Homo sapiens
gctgagcccccgaaggcgcccgagcaggagcaggcggcccccggccccgctgcgggcggcgaggcccccaaa

SEQ ID NO: 54 - DNA, 57 bp, Homo sapiens
gcgcccgagcaggagcaggcggcccccggccccgctgcgggcggcgaggcccccaaa

SEQ ID NO: 55 - DNA, 84 bp, Homo sapiens
gcccagggccccgcagcctctgcagaagagcccaagccggtggaggccccggcagctaattccgaccaaaccgtaaccgtgaaa

SEQ ID NO: 56 - DNA, 42 bp, Homo sapiens
gagaagcccgaccaggacgccgagggcaaggccgaggagaag

SEQ ID NO: 57 - DNA, 42 bp, Homo sapiens
gagacccccgcagccacggaagcgcctagttccacacccaag

SEQ ID NO: 58 - DNA, 63 bp, Homo sapiens
agtgacggggccccagcttcagactcaaaacccggcagctcggaggctgccccctcttccaag